SEQUENCE LISTING (5 sequences)

SEQ ID NO: 1 - DNA, 34 bp, Artificial Sequence
  Description of Artificial Sequence Note = Synthetic Construct
gacgacgacaagatggctcagtggaaccagctgc

SEQ ID NO: 2 - DNA, 40 bp, Artificial Sequence
  Description of Artificial Sequence Note = Synthetic Construct
gaggagaagcccggttatcacatgggggaggtagcacact

SEQ ID NO: 3 - DNA, 20 bp, Artificial Sequence
  Description of Artificial Sequence Note = Synthetic Construct
atgggtttcatcagcaagga

SEQ ID NO: 4 - DNA, 24 bp, Artificial Sequence
  Description of Artificial Sequence Note = Synthetic Construct
tcacctacagtactttccaaatgc

SEQ ID NO: 5 - DNA, 24 bp, Artificial Sequence
  Description of Artificial Sequence Note = Synthetic Construct
agcttcatttcccgtaaatcccta

(19) United States
(12) Patent Application Publication
(10) Pub. No.: US 2013/0172340 A1
(43) Pub. Date: Jul. 4, 2013

(54) SUBSTITUTED 2-(9H-PURIN-9-YL) ACETIC ACID ANALOGUES AS INHIBITORS OF STAT3

(71) Applicants: James Turkson, Orlando, FL (US); Patrick Gunning, Mississauga (CA)

(72) Inventors: James Turkson, Orlando, FL (US); Patrick Gunning, Mississauga (CA)

(73) Assignee: University of Central Flordia Research Foundation,, Orlando, FL (US)

(21) Appl. No.: 13/806,646

(22) PCT Filed: Jun. 22, 2011
(86) PCT No.: PCT/US11/41538

§ 371 Date: Mar. 15, 2013

Related U.S. Application Data
(60) Provisional application No. 61/357,141, filed on Jun. 22, 2010.

(51) Int. Cl. C07D 473/40; C07D 473/18; C07D 473/16

(52) U.S. Cl. CPC C07D 473/40; C07D 473/16; C07D 473/18
USPC 514/234.2; 544/277; 514/263.4; 514/263.23; 544/118; 544/276; 514/263.38; 435/375

(57) ABSTRACT

In one aspect, the invention relates to substituted purine analogs, derivatives thereof, and related compounds, which are useful as inhibitors of STAT protein activity; synthetic methods for making the compounds; pharmaceutical compositions comprising the compounds; and methods of treating disorders of uncontrolled cellular proliferation associated with a STAT protein activity dysfunction using the compounds and compositions. This abstract is intended as a scanning tool for purposes of searching in the particular art and is not intended to be limiting of the present invention.

### CROSS-REFERENCE TO RELATED APPLICATIONS

This application claims the benefit of U.S. Application No. 61/357,141, filed Jun. 22, 2010, which is hereby incorporated by reference in its entirety.

### STATEMENT REGARDING FEDERALLY SPONSORED RESEARCH

This invention was made with government support under grant numbers CA106439 and CA128865 awarded by the National Cancer Institute of the National Institutes of Health. The United States government has certain rights in the invention.

### BACKGROUND

STAT proteins were originally discovered as latent cytoplasmic transcription factors that mediate cytokine and growth factor responses (30, 31). Seven members of the family, STAT1, STAT2, STAT3, STAT4, STAT5a and STAT5b, and STATE, mediate several physiological effects including growth and differentiation, survival, development and inflammation. STATs are SH2 domain-containing proteins. Upon ligand binding to cytokine or growth factor receptors. STATs become phosphorylated on critical Tyr residue (Tyr705 for STAT3) by growth factor receptors, cytoplasmic Janus kinases (Jaks) or Src family kinases. Two phosphorylated and activated STAT monomers dimerize through reciprocal pTyr-SH2 domain interactions, translocate to the nucleus, and bind to specific DNA-response elements of target genes, thereby inducing gene transcription (30, 31). In contrast to normal STAT signaling, many human solid and hematological tumors harbor aberrant STAT3 activity (3 and 32-36 for reviews).

Constitutive STAT3 activity mediates dysregulated growth and survival, angiogenesis, as well as suppresses the host's immune surveillance of the tumor, making constitutively-active STAT3 a critical molecular mediator of carcinogenesis and tumor progression.

Genetic and other molecular evidence reveals persistent Tyr phosphorylation of STAT3 is mediated by aberrant upstream Tyr kinases and shows cancer cell requirement for constitutively-active and dimerized STAT3 for tumor maintenance and progression. Thus, in numerous proof-of-concept studies (5, 6, 7, 19, and 25), inhibition of STAT3 activation or disruption of dimerization induces cancer cell death and tumor regression. How aberrant STAT3 is regulated for meeting the tumor-specific requirements in malignant cells remains undefined. There have been no studies into defining the molecular details of how malignant cells regulate aberrant STAT3 and how this regulation changes upon STAT3 inhibition prior to the onset of phenotypic changes, although knowing these events will facilitate efforts in modulating aberrant STAT3 for managing human cancers. Small-molecule STAT3 inhibitors thus provide tools for probing the molecular dynamics of the cellular processing of STAT3 to understand STAT3's role as a signaling intermediate and a molecular mediator of the events leading to carcinogenesis and malignant progression.

Despite advances in drug discovery directed to identifying inhibitors of STAT3 activity, there is still a scarcity of compounds that are both potent, efficacious, and selective activators of the STAT3 and also effective in the treatment of cancer and other diseases associated with STAT3 dysfunction and diseases in which the STAT3 is involved. These needs and other needs are satisfied by the present invention.

### SUMMARY

In accordance with the purpose(s) of the invention, as embodied and broadly described herein, the invention, in one aspect, relates to compounds useful useful as inhibitors of STAT. In a further aspect, the disclosed compounds and products of disclosed methods of making are modulators of STAT activity, methods of making same, pharmaceutical compositions comprising same, and methods of treating disorders associated with a STAT activity dysfunciton using same. In one aspect, the invention relates to compounds. In a further aspect, the present invention relates to compounds that bind to a STAT protein and negatively modulate STAT activity. The compounds can, in one aspect, exhibit subtype selectivity. In a further aspect, the compounds exhibit selectivity for the STAT3 member of the STAT protein family.

Disclosed are compounds having a structure represented by a formula:

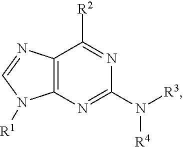

wherein R¹ is selected from H and (CH₂)ₘC═OR⁵, wherein m is an integer from 0-3; wherein R⁵ is selected from OR⁶ and NR⁷R⁸; wherein R⁶ is selected from hydrogen and C1-C8 alkyl; wherein each of R⁷ and R⁸ is independently selected from hydrogen and C1-C8 alkyl; wherein R² is selected from halogen, OR⁹, and NR¹⁰R¹¹; wherein R⁹ is an optionally substituted group selected from C1-C8 alkyl, C3-C6 cycloalkyl, C3-C6 heterocycloalkyl, (CH₂)ₙR¹², and Ar¹; wherein n is an integer from 0-3; wherein R¹² is an optionally substituted group selected from Ar¹, C3-C6 cycloalkyl and C3-C6 heterocycloalkyl; wherein Ar¹ is selected from an optionally substituted monocyclic aryl and monocyclic heteroaryl; wherein R¹⁰ is selected from hydrogen and an optionally substituted group selected from C1-C8 alkyl, C3-C6 cycloalkyl, C3-C6 heterocycloalkyl, (CH₂)ₚR¹³, and Ar²; wherein p is an integer from 0-3; wherein R¹³ is an optionally substituted group selected from Ar², C3-C6 cycloalkyl and C3-C6 heterocycloalkyl; wherein Ar² is selected from an optionally substituted monocyclic aryl and monocyclic heteroaryl; wherein R¹¹ is selected from hydrogen and C1-C8 alkyl; wherein R³ is selected from hydrogen and an optionally substituted group selected from C1-C8 alkyl, (CH₂)_{q}R¹⁴, and C═O(CH₂)ₚA¹⁴; wherein q is an integer from 0-3; wherein R¹⁴ is selected from hydrogen and an optionally substituted group selected from C1-C8 alkyl, C3-C6 cycloalkyl, C3-C6 heterocycloalkyl, and Ar³; wherein Ar³ is selected from an optionally substituted monocyclic aryl and monocyclic heteroaryl; wherein R⁴ is selected from hydrogen and C═OOR¹⁵; wherein R¹⁵ is selected from hydrogen and optionally substituted C1-C8 alkyl; wherein R³ and R⁴ are optionally covalently bonded and, together with the intermediate carbon, comprise an optionally substituted 3- to 7-membered heterocycloalkyl; and wherein R¹⁰ and R¹¹ are optionally covalently bonded and, together with the intermediate carbon, comprise an optionally substituted 3- to 7-membered heterocycloalkyl; or a pharmaceutically acceptable salt, hydrate, solvate, or polymorph thereof.

Also disclosed are pharmaceutical compositions comprising a therapeutically effective amount of a disclosed compound and a pharmaceutically acceptable carrier.

Disclosed are methods for the treatment of a disorder associated with STAT activity dysfunction in a mammal comprising the step of administering to the mammal a therapeutically effective amount of a compound having a structure represented by a formula:

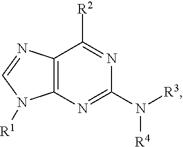

wherein R¹ is selected from H and (CH₂)ₘC═OR⁵, wherein m is an integer from 0-3; wherein R⁵ is selected from OR⁶ and NR⁷R⁸; wherein R⁶ is selected from hydrogen and C1-C8 alkyl; wherein each of R⁷ and R⁸ is independently selected from hydrogen and C1-C8 alkyl; wherein R² is selected from halogen, OR⁹, and NR¹⁰R¹¹; wherein R⁹ is an optionally substituted group selected from C1-C8 alkyl, C3-C6 cycloalkyl, C3-C6 heterocycloalkyl, (CH₂)ₙR¹², and Ar¹; wherein n is an integer from 0-3; wherein R¹² is an optionally substituted group selected from Ar¹, C3-C6 cycloalkyl and C3-C6 heterocycloalkyl; wherein Ar¹ is selected from an optionally substituted monocyclic aryl and monocyclic heteroaryl; wherein R¹⁰ is selected from hydrogen and an optionally substituted group selected from C1-C8 alkyl, C3-C6 cycloalkyl, C3-C6 heterocycloalkyl, (CH₂)ₚR¹³, and Ar²; wherein p is an integer from 0-3; wherein R¹³ is an optionally substituted group selected from Ar², C3-C6 cycloalkyl and C3-C6 heterocycloalkyl; wherein Ar² is selected from an optionally substituted monocyclic aryl and monocyclic heteroaryl; wherein R¹¹ is selected from hydrogen and C1-C8 alkyl; wherein R³ is selected from hydrogen and an optionally substituted group selected from C1-C8 alkyl, (CH₂)_{q}R¹⁴, and C═O(CH₂)_{q}R¹⁴; wherein q is an integer from 0-3; wherein R¹⁴ is selected from hydrogen and an optionally substituted group selected from C1-C8 alkyl, C3-C6 cycloalkyl, C3-C6 heterocycloalkyl, and Ar³; wherein Ar³ is selected from an optionally substituted monocyclic aryl and monocyclic heteroaryl; wherein R⁴ is selected from hydrogen and C═OOR¹⁵; wherein R¹⁵ is selected from hydrogen and optionally substituted C1-C8 alkyl; wherein R³ and R⁴ are optionally covalently bonded and, together with the intermediate carbon, comprise an optionally substituted 3- to 7-membered heterocycloalkyl; and wherein R¹⁰ and R¹¹ are optionally covalently bonded and, together with the intermediate carbon, comprise an optionally substituted 3- to 7-membered heterocycloalkyl; or a pharmaceutically acceptable salt, hydrate, solvate, or polymorph thereof.

Also disclosed are method for inhibition of STAT activity in a mammal comprising the step of administering to the mammal a therapeutically effective amount of least one compound having a structure represented by a formula:

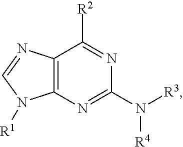

wherein R¹ is selected from H and (CH₂)ₘC═OR⁵, wherein m is an integer from 0-3; wherein R⁵ is selected from OR⁶ and NR⁷R⁸; wherein R⁶ is selected from hydrogen and C1-C8 alkyl; wherein each of R⁷ and R⁸ is independently selected from hydrogen and C1-C8 alkyl; wherein R² is selected from halogen, OR⁹, and NR¹⁰R¹¹; wherein R⁹ is an optionally substituted group selected from C1-C8 alkyl, C3-C6 cycloalkyl, C3-C6 heterocycloalkyl, (CH₂)ₙR¹², and Ar¹; wherein n is an integer from 0-3; wherein R¹² is an optionally substituted group selected from Ar¹, C3-C6 cycloalkyl and C3-C6 heterocycloalkyl; wherein Ar¹ is selected from an optionally substituted monocyclic aryl and monocyclic heteroaryl; wherein R¹⁰ is selected from hydrogen and an optionally substituted group selected from C1-C8 alkyl, C3-C6 cycloalkyl, C3-C6 heterocycloalkyl, (CH₂)ₚR¹³, and Ar²; wherein p is an integer from 0-3; wherein R¹³ is an optionally substituted group selected from Ar², C3-C6 cycloalkyl and C3-C6 heterocycloalkyl; wherein Ar² is selected from an optionally substituted monocyclic aryl and monocyclic heteroaryl; wherein R¹¹ is selected from hydrogen and C1-C8 alkyl; wherein R³ is selected from hydrogen and an optionally substituted group selected from C1-C8 alkyl, (CH₂)_{q}R¹⁴, and C═O(CH₂)_{q}R¹⁴; wherein q is an integer from 0-3; wherein R¹⁴ is selected from hydrogen and an optionally substituted group selected from C1-C8 alkyl, C3-C6 cycloalkyl, C3-C6 heterocycloalkyl, and Ar³; wherein Ar³ is selected from an optionally substituted monocyclic aryl and monocyclic heteroaryl; wherein R⁴ is selected from hydrogen and C═OOR¹⁵; wherein R¹⁵ is selected from hydrogen and optionally substituted C1-C8 alkyl; wherein R³ and R⁴ are optionally covalently bonded and, together with the intermediate carbon, comprise an optionally substituted 3- to 7-membered heterocycloalkyl; and wherein R¹⁰ and R¹¹ are optionally covalently bonded and, together with the intermediate carbon, comprise an optionally substituted 3- to 7-membered heterocycloalkyl; or a pharmaceutically acceptable salt, hydrate, solvate, or polymorph thereof.

Also disclosed are methods for inhibiting STAT activity in at least one cell, comprising the step of contacting the at least one cell with an effective amount of least one compound having a structure represented by a formula:

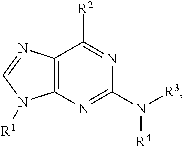

wherein R¹ is selected from H and (CH₂)ₘC═OR⁵, wherein m is an integer from 0-3; wherein R⁵ is selected from OR⁶ and NR⁷R⁸; wherein R⁶ is selected from hydrogen and C1-C8 alkyl; wherein each of R⁷ and R⁸ is independently selected from hydrogen and C1-C8 alkyl; wherein R² is selected from halogen, OR⁹, and NR¹⁰R¹¹; wherein R⁹ is an optionally substituted group selected from C1-C8 alkyl, C3-C6 cycloalkyl, C3-C6 heterocycloalkyl, (CH₂)ₙR¹², and Ar¹; wherein n is an integer from 0-3; wherein R¹² is an optionally substituted group selected from Ar¹, C3-C6 cycloalkyl and C3-C6 heterocycloalkyl; wherein Ar¹ is selected from an optionally substituted monocyclic aryl and monocyclic heteroaryl; wherein R¹⁰ is selected from hydrogen and an optionally substituted group selected from C1-C8 alkyl, C3-C6 cycloalkyl, C3-C6 heterocycloalkyl, (CH₂)ₚR¹³, and Ar²; wherein p is an integer from 0-3; wherein R¹³ is an optionally substituted group selected from Ar², C3-C6 cycloalkyl and C3-C6 heterocycloalkyl; wherein Ar² is selected from an optionally substituted monocyclic aryl and monocyclic heteroaryl; wherein R¹¹ is selected from hydrogen and C1-C8 alkyl; wherein R³ is selected from hydrogen and an optionally substituted group selected from C1-C8 alkyl, (CH₂)_{q}R¹⁴, and C═O(CH₂)_{q}R¹⁴; wherein q is an integer from 0-3; wherein R¹⁴ is selected from hydrogen and an optionally substituted group selected from C1-C8 alkyl, C3-C6 cycloalkyl, C3-C6 heterocycloalkyl, and Ar³; wherein Ar³ is selected from an optionally substituted monocyclic aryl and monocyclic heteroaryl; wherein R⁴ is selected from hydrogen and C═OOR¹⁵; wherein R¹⁵ is selected from hydrogen and optionally substituted C1-C8 alkyl; wherein R³ and R⁴ are optionally covalently bonded and, together with the intermediate carbon, comprise an optionally substituted 3- to 7-membered heterocycloalkyl; and wherein R¹⁰ and R¹¹ are optionally covalently bonded and, together with the intermediate carbon, comprise an optionally substituted 3- to 7-membered heterocycloalkyl; or a pharmaceutically acceptable salt, hydrate, solvate, or polymorph thereof.

Also disclosed are uses of a compound having a structure represented by a formula:

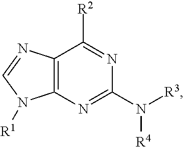

wherein R¹ is selected from H and (CH₂)ₘC═OR⁵, wherein m is an integer from 0-3; wherein R⁵ is selected from OR⁶ and NR⁷R⁸; wherein R⁶ is selected from hydrogen and C1-C8 alkyl; wherein each of R⁷ and R⁸ is independently selected from hydrogen and C1-C8 alkyl; wherein R² is selected from halogen, OR⁹, and NR¹⁰R¹¹; wherein R⁹ is an optionally substituted group selected from C1-C8 alkyl, C3-C6 cycloalkyl, C3-C6 heterocycloalkyl, (CH₂)ₙR¹², and Ar¹; wherein n is an integer from 0-3; wherein R¹² is an optionally substituted group selected from Ar¹, C3-C6 cycloalkyl and C3-C6 heterocycloalkyl; wherein Ar¹ is selected from an optionally substituted monocyclic aryl and monocyclic heteroaryl; wherein R¹⁰ is selected from hydrogen and an optionally substituted group selected from C1-C8 alkyl, C3-C6 cycloalkyl, C3-C6 heterocycloalkyl, (CH₂)ₚR¹³, and Ar²; wherein p is an integer from 0-3; wherein R¹³ is an optionally substituted group selected from Ar², C3-C6 cycloalkyl and C3-C6 heterocycloalkyl; wherein Ar² is selected from an optionally substituted monocyclic aryl and monocyclic heteroaryl; wherein R¹¹ is selected from hydrogen and C1-C8 alkyl; wherein R³ is selected from hydrogen and an optionally substituted group selected from C1-C8 alkyl, (CH₂)_{q}R¹⁴, and C═O(CH₂)_{q}R¹⁴; wherein q is an integer from 0-3; wherein R¹⁴ is selected from hydrogen and an optionally substituted group selected from C1-C8 alkyl, C3-C6 cycloalkyl, C3-C6 heterocycloalkyl, and Ar³; wherein Ar³ is selected from an optionally substituted monocyclic aryl and monocyclic heteroaryl; wherein R⁴ is selected from hydrogen and C═OOR¹⁵; wherein R¹⁵ is selected from hydrogen and optionally substituted C1-C8 alkyl; wherein R³ and R⁴ are optionally covalently bonded and, together with the intermediate carbon, comprise an optionally substituted 3- to 7-membered heterocycloalkyl; and wherein R¹⁰ and R¹¹ are optionally covalently bonded and, together with the intermediate carbon, comprise an optionally substituted 3- to 7-membered heterocycloalkyl; or a pharmaceutically acceptable salt, hydrate, solvate, or polymorph thereof.

Also disclosed are pharmaceutical compositions comprising a pharmaceutically acceptable carrier and an effective amount of a compound represented by a formula:

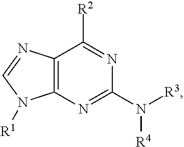

wherein R¹ is selected from H and (CH₂)ₘC═OR⁵, wherein m is an integer from 0-3; wherein R⁵ is selected from OR⁶ and NR⁷R⁸; wherein R⁶ is selected from hydrogen and C1-C8 alkyl; wherein each of R⁷ and R⁸ is independently selected from hydrogen and C1-C8 alkyl; wherein R² is selected from halogen, OR⁹, and NR¹⁰R¹¹; wherein R⁹ is an optionally substituted group selected from C1-C8 alkyl, C3-C6 cycloalkyl, C3-C6 heterocycloalkyl, (CH₂)ₙR¹², and Ar¹; wherein n is an integer from 0-3; wherein R¹² is an optionally substituted group selected from Ar¹, C3-C6 cycloalkyl and C3-C6 heterocycloalkyl; wherein Ar¹ is selected from an optionally substituted monocyclic aryl and monocyclic heteroaryl; wherein R¹⁰ is selected from hydrogen and an optionally substituted group selected from C1-C8 alkyl, C3-C6 cycloalkyl, C3-C6 heterocycloalkyl, (CH₂)ₚR¹³, and Ar²; wherein p is an integer from 0-3; wherein R¹³ is an optionally substituted group selected from Ar², C3-C6 cycloalkyl and C3-C6 heterocycloalkyl; wherein Ar² is selected from an optionally substituted monocyclic aryl and monocyclic heteroaryl; wherein R¹¹ is selected from hydrogen and C1-C8 alkyl; wherein R³ is selected from hydrogen and an optionally substituted group selected from C1-C8 alkyl, (CH₂)_{q}R¹⁴, and C═O(CH₂)_{q}R¹⁴; wherein q is an integer from 0-3; wherein R¹⁴ is selected from hydrogen and an optionally substituted group selected from C1-C8 alkyl, C3-C6 cycloalkyl, C3-C6 heterocycloalkyl, and Ar³; wherein Ar³ is selected from an optionally substituted monocyclic aryl and monocyclic heteroaryl; wherein R⁴ is selected from hydrogen and C═OOR¹⁵; wherein R¹⁵ is selected from hydrogen and optionally substituted C1-C8 alkyl; wherein R³ and R⁴ are optionally covalently bonded and, together with the intermediate carbon, comprise an optionally substituted 3- to 7-membered heterocycloalkyl; and wherein R¹⁰ and R¹¹ are optionally covalently bonded and, together with the intermediate carbon, comprise an optionally substituted 3- to 7-membered heterocycloalkyl; or a pharmaceutically acceptable salt, hydrate, solvate, or polymorph thereof.

Also disclosed are kits comprising at least one compound having a structure represented by a formula:

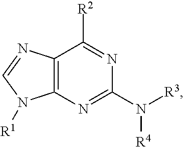

wherein R¹ is selected from H and (CH₂)ₘC═OR⁵, wherein m is an integer from 0-3; wherein R⁵ is selected from OR⁶ and NR⁷R⁸; wherein R⁶ is selected from hydrogen and C1-C8 alkyl; wherein each of R⁷ and R⁸ is independently selected from hydrogen and C1-C8 alkyl; wherein R² is selected from halogen, OR⁹, and NR¹⁰R¹¹; wherein R⁹ is an optionally substituted group selected from C1-C8 alkyl, C3-C6 cycloalkyl, C3-C6 heterocycloalkyl, (CH₂)ₙR¹², and Ar¹; wherein n is an integer from 0-3; wherein R¹² is an optionally substituted group selected from Ar¹, C3-C6 cycloalkyl and C3-C6 heterocycloalkyl; wherein Ar¹ is selected from an optionally substituted monocyclic aryl and monocyclic heteroaryl; wherein R¹⁰ is selected from hydrogen and an optionally substituted group selected from C1-C8 alkyl, C3-C6 cycloalkyl, C3-C6 heterocycloalkyl, (CH₂)ₚR¹³, and Ar²; wherein p is an integer from 0-3; wherein R¹³ is an optionally substituted group selected from Ar², C3-C6 cycloalkyl and C3-C6 heterocycloalkyl; wherein Ar² is selected from an optionally substituted monocyclic aryl and monocyclic heteroaryl; wherein R¹¹ is selected from hydrogen and C1-C8 alkyl; wherein R³ is selected from hydrogen and an optionally substituted group selected from C1-C8 alkyl, (CH₂)_{q}R¹⁴, and C═O(CH₂)_{q}R¹⁴; wherein q is an integer from 0-3; wherein R¹⁴ is selected from hydrogen and an optionally substituted group selected from C1-C8 alkyl, C3-C6 cycloalkyl, C3-C6 heterocycloalkyl, and Ar³; wherein Ar³ is selected from an optionally substituted monocyclic aryl and monocyclic heteroaryl; wherein R⁴ is selected from hydrogen and C═OOR¹⁵; wherein R¹⁵ is selected from hydrogen and optionally substituted C1-C8 alkyl; wherein R³ and R⁴ are optionally covalently bonded and, together with the intermediate carbon, comprise an optionally substituted 3- to 7-membered heterocycloalkyl; and wherein R¹⁰ and R¹¹ are optionally covalently bonded and, together with the intermediate carbon, comprise an optionally substituted 3- to 7-membered heterocycloalkyl; or a pharmaceutically acceptable salt, hydrate, solvate, or polymorph thereof, and one or more of: (a) at least one agent known to decrease STAT activity; (b) at least one agent known to increase STAT activity; (c) at least one agent know to treat a disease of uncontrolled cellular proliferation; (d) at least one agent known to treat psoriasis; (e) at least one agent known to treat pulmonary arterial hypertension; or (f) instructions for treating a disorder associated with STAT dysfunction.

Also disclosed are methods for manufacturing a medicament comprising combining at least one disclosed compound or at least one disclosed product with a pharmaceutically acceptable carrier or diluent.

Also disclosed are uses of a disclosed compound or a disclosed product in the manufacture of a medicament for the treatment of a STAT activity dysfunction. In a further aspect, disclosed are uses of a disclosed compound or a disclosed product in the manufacture of a medicament for the treatment of a disorder of uncontrolled cellular proliferation.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the invention.

FIG. 1 shows representative pharmacophore modeling data of the binding of STAT3 to small-molecule dimerization inhibitors. The various panels show representative data as follows: (A) STAT3 SH2 domain binding sites and key amino acid residues, (B) STAT3 inhibitors GOLD-docked and overlaid with the STAT3 SH2 domain, (C) Pharmacophore plot identifying the optimal distances for the projection of functionality from the centroid unit, (D) A GOLD docked purine derivative superimposed over the pharmacophore plot and previously docked STAT3 inhibitors, and (E) Low energy GOLD-docked Baa in the STAT3 SH2 domain (pdb: 1BG1). Further discussion on the figure is provided in the Experimental section below.

FIG. 2 shows representative data from immunoprecipitation and immunoblotting analysis for the effects of representative disclosed compounds on intracellular STAT3, Erk^{MAPK}, Src, and STAT1 activation. Briefly, the figure shows immunoblotting analysis of whole-cell lysates of equal total protein prepared from NIH3T3/v-Src and MDA-MB-231 cells As indicated, the cells were treated or untreated with the indicated agents at 40 or 50 μM for the indicated times and probing for pY705STAT3, STAT3, pErk1/2, Erk1/2, pSrc, Src, pSTAT1, STAT1, or pTyr (clone 4G10). Positions of proteins in gel are labeled; control lanes (0) are whole-cell lysates from 0.05% DMSO-treated cells. Data are representative of 3-4 independent determinations. Further discussion on the figure is provided in the Experimental section below.

FIG. 3 shows representative data from immunoprecipitation and immunoblotting analysis for the effects of representative disclosed compounds on intracellular STAT3, Erk^{MAPK}, Src, and STAT1 activation. Briefly, the figure shows immunoblotting analysis of whole-cell lysates of equal total protein prepared from EGF-stimulated NIH3T3/hEGFR fibroblasts (panel i)), or of STAT1 immune complexes prepared from EGF-stimulated NIH3T3/hEGFR fibroblasts (panel ii). As indicated, the cells were treated or untreated with the indicated agents at 40 or 50 μM for the indicated times and probing for pY705STAT3, STAT3, pErk1/2, Erk1/2, pSrc, Src, pSTAT1, STAT1, or pTyr (clone 4G10). Positions of proteins in gel are labeled; control lanes (0) are whole-cell lysates from 0.05% DMSO-treated cells. Data are representative of 3-4 independent determinations. Further discussion on the figure is provided in the Experimental section below.

FIG. 4 shows representative data on the effect of representative disclosed compounds in selectively suppressing viability of malignant cells that harbor persistently active STAT3. Briefly, human breast (MDA-MB-231), pancreatic (Panc-1), and prostate (DU145) cancer cells, the v-Src transformed (NIH3T3/v-Src) fibroblasts or their normal counterparts (NIH3T3), and the murine thymic stromal epithelial cells (TE-71) were treated or not treated with 30-500 μM of the representative disclosed compounds, as indicated, for 48 h and assessed for viability using CyQuant cell proliferation kit. Values, mean (SD of 3 independent experiments each in triplicate.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### A. DEFINITIONS

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or can not occur, and that the description includes instances where said event or circumstance occurs and instances where it does not.

As used herein, the term “STAT” and “signal transducer and activator of transcription” can be used interchangeably, and refer to a protein family comprising at least the following members: STAT1, 2, 3, 4, 5a, 5b, and 6. The STAT family of proteins are latent cytoplasmic transcription factors that mediate cellular responses to cytokines, growth factors, and other polypeptide ligands.

As used herein, the terms “STAT3,” “signal transducer and activator of transcription 3 (acute-phase response),” and “signal transducer and activator of transcription 3” can be used interchangeably and refer to a a transcription factor encoded by a gene designated in human as the STAT3 gene, which has a human gene map locus of 17q21 and described by Entrez Gene cytogenetic band: 17q21.31; Ensembl cytogenetic band: 17q21.2; and, HGNC cytogenetic band: 17q21. The term STAT3 refers to a human protein that has 770 amino acids and has a molecular weight of about 88,068. The term is inclusive of splice isoforms or variants, and also inclusive of that protein referred to by such alternative designations as: APRF, MGC16063, Acute-phase response factor, DNA-binding protein APRF, HIES as used by those skilled in the art to that protein encoded by human gene STAT3. The term is also inclusive of the non-human ortholog or homolog thereof.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed methods, the subject has been diagnosed with a need for treatment of one or more oncological disorders or cancers prior to the administering step. In some aspects of the disclosed method, the subject has been diagnosed with a need for inhibition or negative modulation of STAT3 prior to the administering step. In some aspects of the disclosed method, the subject has been diagnosed with a need for treatment of one or more oncological disorders or cancers associated with STAT3 dysfunction prior to the administering step.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with a disorder treatable by STAT3 inhibition” means having been subjected to a physical examination by a person of skill, for example, a physician, and found to have a condition that can be diagnosed or treated by a compound or composition that can inhibit or negatively modulate STAT3. As a further example, “diagnosed with a need for inhibition of STAT3” refers to having been subjected to a physical examination by a person of skill, for example, a physician, and found to have a condition characterized by a dysfunction in STAT3 activity. Such a diagnosis can be in reference to a disorder, such as an oncological disorder or disease, cancer and/or disorder of uncontrolled cellular proliferation and the like, as discussed herein. For example, the term “diagnosed with a need for inhibition of STAT3 activity” refers to having been subjected to a physical examination by a person of skill, for example, a physician, and found to have a condition that can be diagnosed or treated by inhibition of STAT3 activity. For example, “diagnosed with a need for modulation of STAT3 activity” means having been subjected to a physical examination by a person of skill, for example, a physician, and found to have a condition that can be diagnosed or treated by modulation of STAT3 activity, e.g. negative modulation. For example, “diagnosed with a need for treatment of one or more disorder of uncontrolled cellular proliferation associated with STAT3 dysfunction” means having been subjected to a physical examination by a person of skill, for example, a physician, and found to have one or disorders of uncontrolled cellular proliferation, e.g. a cancer, associated with STAT3 dysfunction.

As used herein, the phrase “identified to be in need of treatment for a disorder,” or the like, refers to selection of a subject based upon need for treatment of the disorder. For example, a subject can be identified as having a need for treatment of a disorder (e.g., a disorder related to STAT3 activity) based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “contacting” as used herein refers to bringing a disclosed compound and a cell, target STAT3 protein, or other biological entity together in such a manner that the compound can affect the activity of the target (e.g., spliceosome, cell, etc.), either directly; i.e., by interacting with the target itself, or indirectly; i.e., by interacting with another molecule, co-factor, factor, or protein on which the activity of the target is dependent.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side affects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “EC₅₀,” is intended to refer to the concentration of a substance (e.g., a compound or a drug) that is required for 50% agonism or activation of a biological process, or component of a process, including a protein, subunit, organelle, ribonucleoprotein, etc. In one aspect, an EC₅₀ can refer to the concentration of a substance that is required for 50% agonism or activation in vivo, as further defined elsewhere herein. In a further aspect, EC₅₀ refers to the concentration of agonist or activator that provokes a response halfway between the baseline and maximum response.

As used herein, “IC₅₀,” is intended to refer to the concentration of a substance (e.g., a compound or a drug) that is required for 50% inhibition of a biological process, or component of a process, including a protein, subunit, organelle, ribonucleoprotein, etc. In some contexts, an IC₅₀ can refer to the plasma concentration of a substance that is required for 50% inhibition in vivo, as further defined elsewhere herein. More commonly, IC₅₀ refers to the half maximal (50%) inhibitory concentration (IC) of a substance required to inhibit a process or activity in vitro.

As used herein, “STAT3 IC₅₀” refers to the concentration of a substance (e.g., a compound or a drug) that is required for 50% inhibition of a STAT3 activity. In some contexts, an IC₅₀ can refer to the plasma concentration of a substance that is required for 50% inhibition of an in vivo activity or process as further defined elsewhere herein, e.g. tumor growth in an animal or human. In other contexts, STAT3 IC₅₀ refers the half maximal (50%) inhibitory concentration (IC) of a substance or compound required to inhibit a process or activity an in vitro context, e.g. a cell-free or cell-based assay. For example, the STAT3 IC₅₀ can be in the context of the half-maximal concentration required to inhibit cell growth. As discussed below, the response is measured in a cell-line with aberrant STAT3 activity. In a yet further aspect, the response is measured in a cell-line with persistently active STAT3. In an even further aspect, the response is measured in a cell-line selected from a human breast cancer, human pancreatic cancer, and human prostate cancer. In a still further aspect, the response is measured in a cell-line selected from. MDA-MB-231, Panc-1, and DU-145. In a yet further aspect, the response is measured in a cell-line transfected with v-Src. In an even further aspect, the cell-line transfected with v-Src is a permanent cell-line. Alternatively, the STAT3 IC₅₀ is the half-maximal concentration required to inhibit STAT3 activity in a cell-free assay, e.g. an electrophoretic mobility shift assay (“EMSA”).

As used herein, the term “STAT3 K_{D}” refers to the binding affinity of a compound or substance for the STAT3 determined in an in vitro assay. The KD of a substance for a protein can be determined by a variety of methods known to one skilled in the art, e.g. equilibrium dialysis, analytical ultracentrifugation and surface plasmon resonance (“SPR”) analysis. As typically used herein, STAT3 K_{D} is defined as the ratio of association and dissociation rate constants determined using SPR analysis using purified STAT3 protein.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dode cyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by the formula (CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or -OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aryl” as used herein is a group that contains any carbon-based aromatic group including, but not limited to, benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein. The term “biaryl” is a specific type of aryl group and is included in the definition of “aryl.” Biaryl refers to two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula NA¹A², where A¹ and A² can be, independently, hydrogen or alkyl, cycloalkyl, alkenyl, cycloalkenyl, alkynyl, cycloalkynyl, aryl, or heteroaryl group as described herein.

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula —N(-alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula C(O)OH.

The term “ester” as used herein is represented by the formula —OC(O)A¹ or C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The term “halide” as used herein refers to the halogens fluorine, chlorine, bromine, and iodine.

The term “heterocycle,” as used herein refers to single and multi-cyclic aromatic or non-aromatic ring systems in which at least one of the ring members is other than carbon. Heterocycle includes azetidine, dioxane, furan, imidazole, isothiazole, isoxazole, morpholine, oxazole, oxazole, including, 1,2,3-oxadiazole, 1,2,5-oxadiazole and 1,3,4-oxadiazole, piperazine, piperidine, pyrazine, pyrazole, pyridazine, pyridine, pyrimidine, pyrrole, pyrrolidine, tetrahydrofuran, tetrahydropyran, tetrazine, including 1,2,4,5-tetrazine, tetrazole, including 1,2,3,4-tetrazole and 1,2,4,5-tetrazole, thiadiazole, including, 1,2,3-thiadiazole, 1,2,5-thiadiazole, and 1,3,4-thiadiazole, thiazole, thiophene, triazine, including 1,3,5-triazine and 1,2,4-triazine, triazole, including, 1,2,3-triazole, 1,3,4-triazole, and the like.

The term “hydroxyl” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” as used herein is represented by the formula —N₃.

The term “nitro” as used herein is represented by the formula —NO₂.

The term “nitrile” as used herein is represented by the formula —CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{o}; —(CH₂)₀₋₄OR^{o}; —O(CH₂)₀₋₄R^{o}, —O—(CH₂)₀₋₄C(O)OR^{o}; —(CH₂)₀₋₄CH(OR)₂; —(CH₂)₀₋₄SR^{o}; —(CH₂)₀₋₄Ph, which may be substituted with RO; —(CH₂)₀₋₄—O—(CH₂)₀₋₁Ph which may be substituted with R^{o}; —CH═CHPh, which may be substituted with R^{o}; —(CH₂)₀₋₄—O—(CH₂)₀₋₁-pyridyl which may be substituted with R^{o}; —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR^{o}₂; —N(R^{o})C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄C(O)R^{o}; —OC(O)(CH₂)₀₋₄SR—, SC(S)SRO; —(CH₂)₀₋₄SC(O)R^{o}; —(CH₂)₀₋₄C(O)NR₂; —C(S)NR^{o}₂; —C(S)SR^{o}; —SC(S)SR^{o}, —(CH₂)₀₋₄C(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR^{o}₂; —(CH₂)₀₋₄S(O)R^{o}; —N(R^{o})S(O)₂NR^{o}₂; —N(R^{o})S(O)₂R^{o}; —N(OR^{o})R^{o}; —C(NH)NR^{o}₂; —P(O)₂R^{o}; —P(O)R^{o}₂; —OP(O)R^{o}₂; —OP(O)(OR^{o})₂; SiR^{o}₃; —(C₁₋₄ straight or branched alkylene)O—N(R^{o})₂; or —(C₁₋₄ straight or branched alkylene)C(O)O—N(R^{o})₂, wherein each R^{o} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms), are independently halogen, —(CH₂)₀₋₂R., -(haloR.), —(CH₂)₀₋₂OH, —(CH₂)₀₋₂OR., —(CH₂)₀₋₂CH(OR.)₂; —O(haloR.), —CN, —N₃, —(CH₂)₀₋₂C(O)R., —(CH₂)₀₋₂C(O)OH, —(CH₂)₀₋₂C(O)OR., —(CH₂)₂SR., —(CH₂)₀₋₂SH, —(CH₂)₀₋₂NH₂, —(CH₂)₀₋₂NHR., —(CH₂)₀₋₂NR.₂, —NO₂, —SiR.₃, —OSiR₁₃, —C(O)SR., —(C₁₋₄ straight or branched alkylene)C(O)OR., or —SSR. wherein each R. is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{o} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R₁₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R. include halogen, —R., -(haloR.), —OH, —OR., —O(haloR.), —CN, —C(O)OH, —C(O)OR., —NH₂, —NHR., —NR.₂, or —NO₂, wherein each R. is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R., -(haloR.), —OH, —OR., —O(haloR.), —CN, —C(O)OH, —C(O)OR., —NH₂, —NHR., —NR.², or —NO₂, wherein each R. is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, brosylate, and halides.

The terms “hydrolysable group” and “hydrolysable moiety” refer to a functional group capable of undergoing hydrolysis, e.g., under basic or acidic conditions. Examples of hydrolysable residues include, without limitatation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure

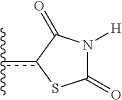

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5, 6,7,8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

“Inorganic radicals,” as the term is defined and used herein, contain no carbon atoms and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

Compounds described herein comprise atoms in both their natural isotopic abundance and in non-natural abundance. The disclosed compounds can be isotopically-labelled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of the present invention and prodrugs thereof can generally be prepared by carrying out the procedures below, by substituting a readily available isotopically labelled reagent for a non-isotopically labelled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvate or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an α-hydrogen can exist in an equilibrium of the keto form and the enol form.

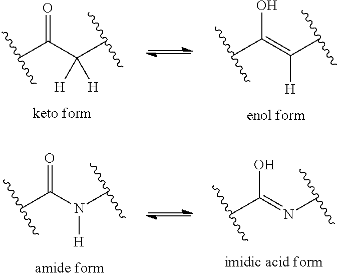 [SMILES: *CC(*)=O ; */C=C(/*)O ; *NC(*)=O ; */N=C(/*)O]

Likewise, amides with an N-hydrogen can exist in an equilibrium of the amide form and the imidic acid form. Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

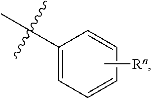

which is understood to be equivalent to a formula:

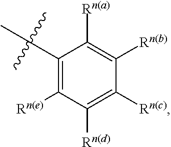

wherein n is typically an integer. That is, Rⁿ is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance R^{n(a)} is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds can not be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C—F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. COMPOUNDS

In one aspect, the invention relates to compounds useful as inhibitors of STAT. In a further aspect, the disclosed compounds and products of disclosed methods of making are modulators of STAT activity. More specifically, in one aspect, the present invention relates to compounds that bind to a STAT protein and negatively modulate STAT activity. The compounds can, in one aspect, exhibit subtype selectivity. In a further aspect, the compounds exhibit selectivity for the STAT3 member of the STAT protein family.

In one aspect, the disclosed compounds and products of disclosed methods of making exhibit inhibition of STAT3 activity. In a further aspect, the disclosed compounds and products of disclosed methods of making exhibit inhibition of STAT3 activity in an electrophoretic mobility shift assay (“EMSA”). In a still further aspect, the disclosed compounds and products of disclosed methods of making bind to the STAT3 protein. In a yet further aspect, the disclosed compounds and products of disclosed methods of making inhibit cell-growth. In an even further aspect, the disclosed compounds and products of disclosed methods of making inhibit in vitro cell growth. In a still further aspect, the disclosed compounds and products of disclosed methods of making inhibit growth in a cell-line selected from a breast cancer, prostate cancer, and prostate cancer. In a yet further aspect, the cell-line is derived from a human source. In an even further aspect, the cell-line is selected from DU-145, Panc-1, and MDA-MB-231. In a yet further aspect, the cell-line is the NIH3T3 cell-line transfected with v-Src.

In one aspect, the compounds of the invention are useful in the treatment oif a disorder of uncontrolled cellular proliferation associated with STAT dysfunction and other diseases in which a STAT protein is involved, as further described herein. In a further aspect, the STAT protein is STAT3.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

1. Structure

In one aspect, the invention relates to a compound having a structure represented by a formula:

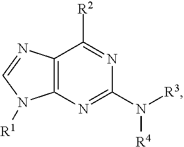

wherein R¹ is selected from H and (CH₂)ₘC═OR⁵, wherein m is an integer from 0-3; wherein R⁵ is selected from OR⁶ and NR⁷R⁸; wherein R⁶ is selected from hydrogen and C1-C8 alkyl; wherein each of R⁷ and R⁸ is independently selected from hydrogen and C1-C8 alkyl; wherein R² is selected from halogen, OR⁹, and NR¹⁰R¹¹; wherein R⁹ is an optionally substituted group selected from C1-C8 alkyl, C3-C6 cycloalkyl, C3-C6 heterocycloalkyl, (CH₂)ₙR¹², and Ar′; wherein n is an integer from 0-3; wherein R¹² is an optionally substituted group selected from Ar¹, C3-C6 cycloalkyl and C3-C6 heterocycloalkyl; wherein Ar′ is selected from an optionally substituted monocyclic aryl and monocyclic heteroaryl; wherein R¹⁰ is selected from hydrogen and an optionally substituted group selected from C1-C8 alkyl, C3-C6 cycloalkyl, C3-C6 heterocycloalkyl, (CH₂)ₚR¹³, and Ar²; wherein p is an integer from 0-3; wherein R¹³ is an optionally substituted group selected from Ar², C3-C6 cycloalkyl and C3-C6 heterocycloalkyl; wherein Ar2 is selected from an optionally substituted monocyclic aryl and monocyclic heteroaryl; wherein R¹¹ is selected from hydrogen and C1-C8 alkyl; wherein R³ is selected from hydrogen and an optionally substituted group selected from C1-C8 alkyl, (CH₂)_{q}R¹⁴, and C═O(CH₂)_{q}R¹⁴; wherein q is an integer from 0-3; wherein R¹⁴ is selected from hydrogen and an optionally substituted group selected from C1-C8 alkyl, C3-C6 cycloalkyl, C3-C6 heterocycloalkyl, and Ar³; wherein Ar³ is selected from an optionally substituted monocyclic aryl and monocyclic heteroaryl; wherein R⁴ is selected from hydrogen and C—OOR¹⁵; wherein R¹⁵ is selected from hydrogen and optionally substituted C1-C8 alkyl; wherein R³ and R⁴ are optionally covalently bonded and, together with the intermediate carbon, comprise an optionally substituted 3- to 7-membered heterocycloalkyl; and wherein R¹⁰ and R¹¹ are optionally covalently bonded and, together with the intermediate carbon, comprise an optionally substituted 3- to 7-membered heterocycloalkyl; or a pharmaceutically acceptable salt, hydrate, solvate, or polymorph thereof.

In a further aspect, the compound has a structure represented by a formula:

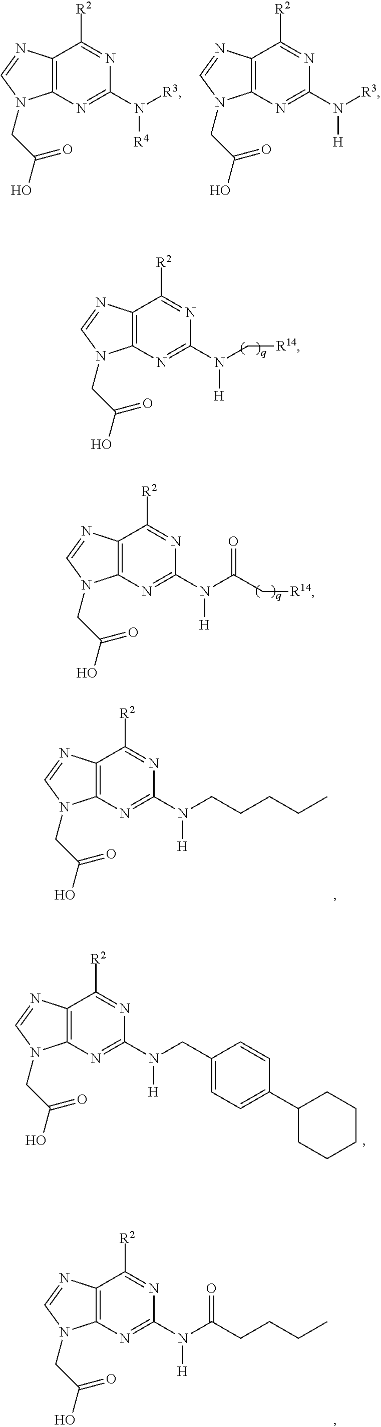

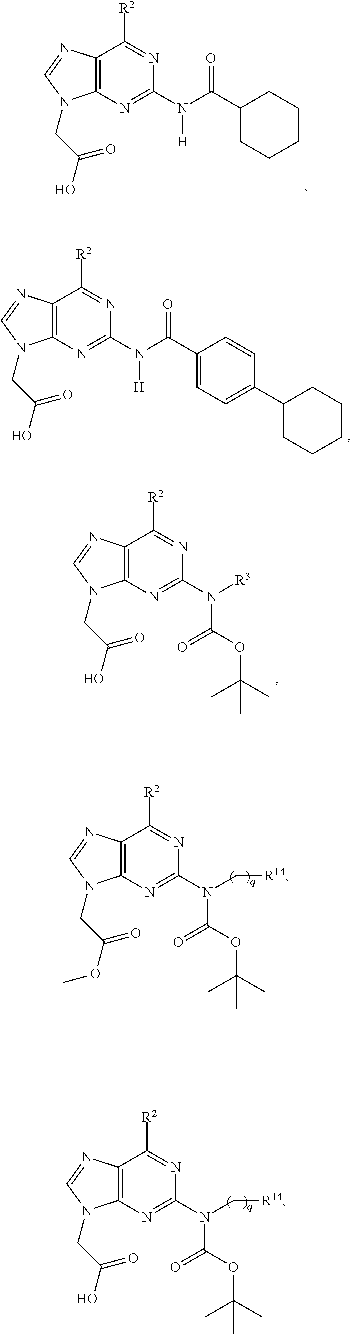

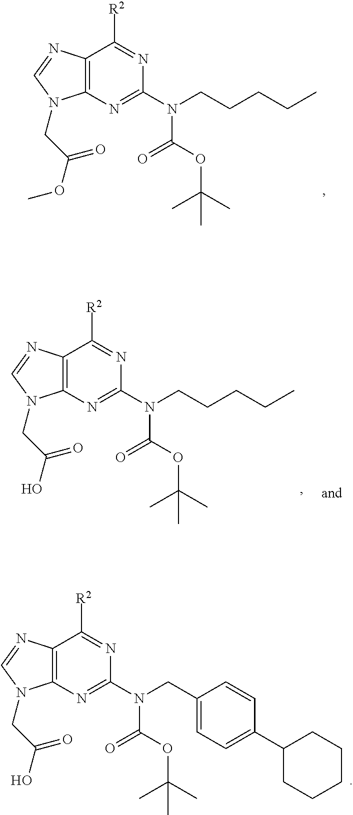

In a further aspect, the compound has a structure represented by a formula:

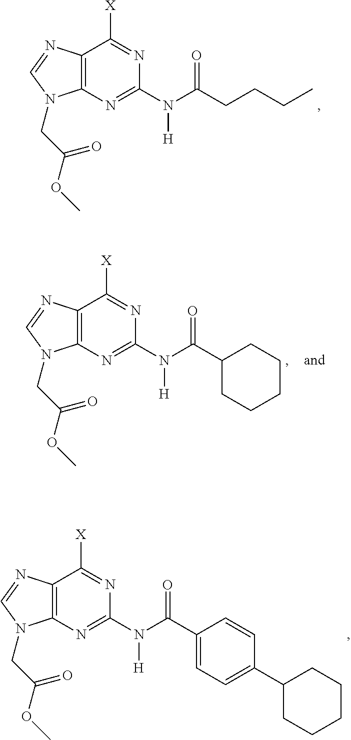

wherein X is halogen. In a yet further aspect, X is chloro.

In a further aspect, the compound has a structure represented by a formula:

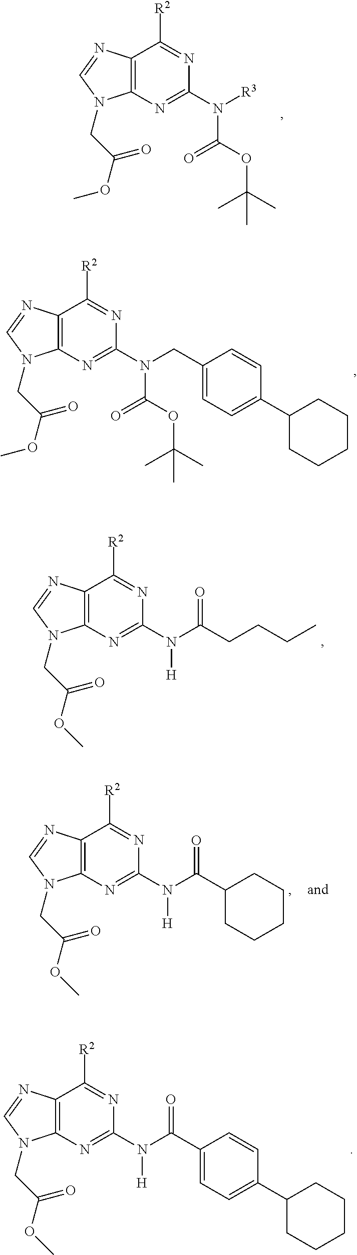

In a further aspect, the compound has a structure represented by a formula:

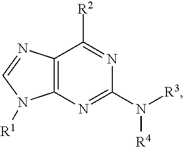

wherein R¹ is selected from hydrogen and a structure represented by:

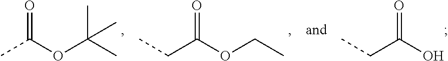

wherein R² is selected from halogen and structure represented by: a formula:

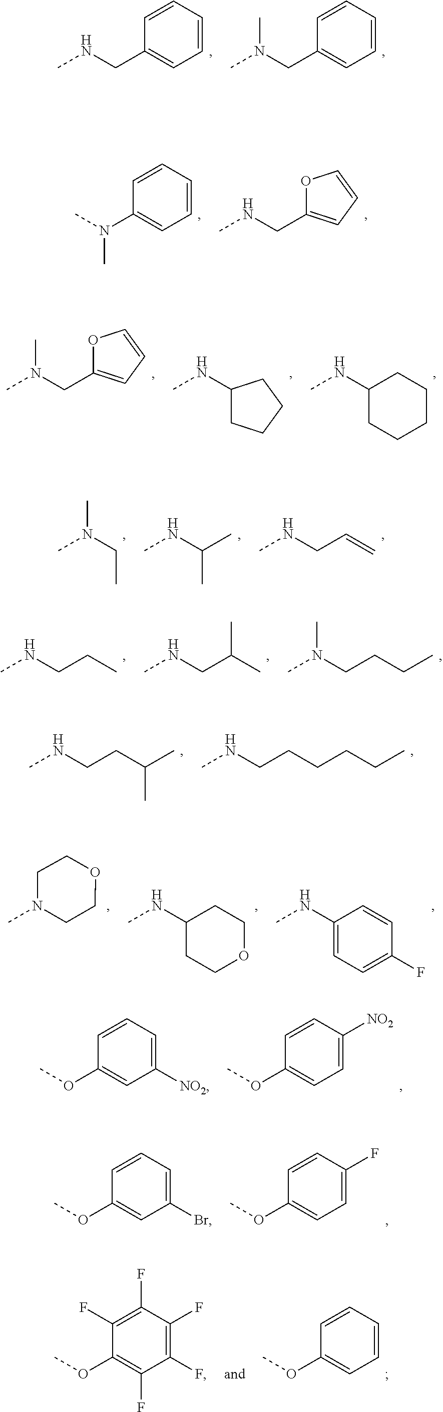

wherein R³ is selected from hydrogen and a structure represented by:

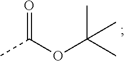

and, wherein R⁴ is selected from hydrogen and a structure represnted by:

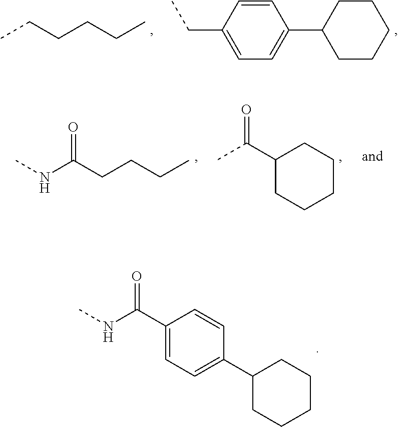

In a further aspect, the compound has a structure represented by a formula:

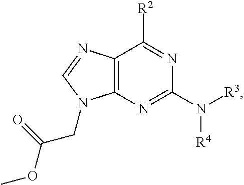

wherein R² is selected from halogen and structure represented by:

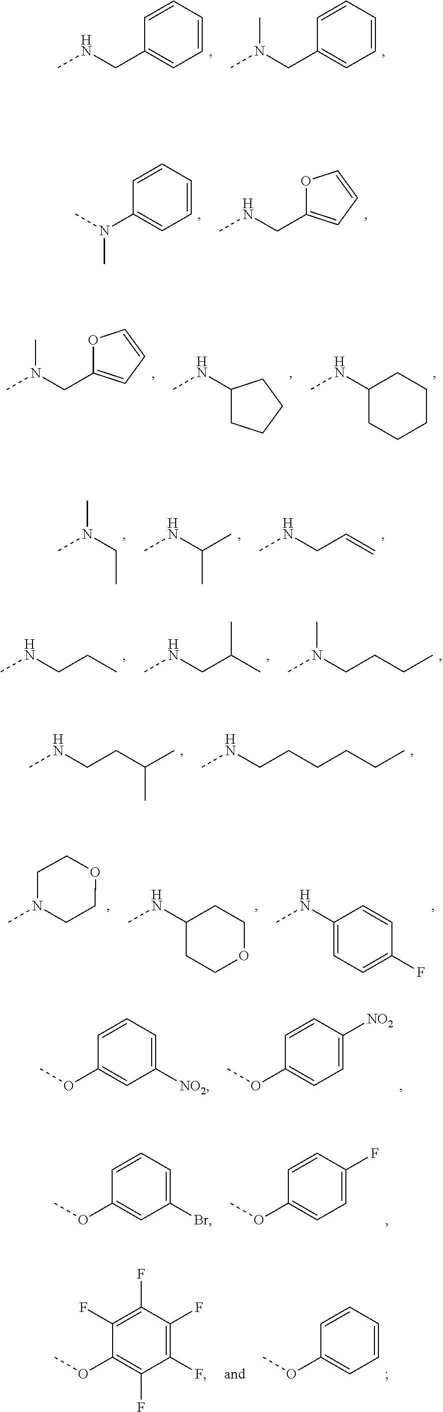

wherein R³ is selected from hydrogen and a structure represented by:

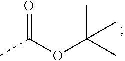

and, wherein R⁴ is selected from hydrogen and a structure represented by:

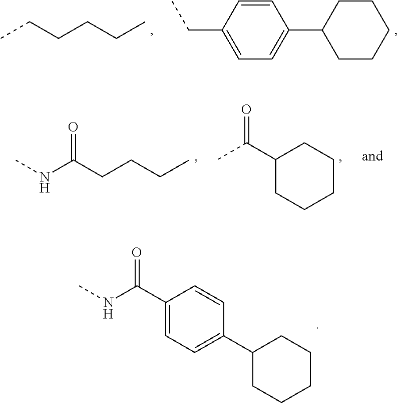

In a further aspect, the compound has a structure represented by a formula:

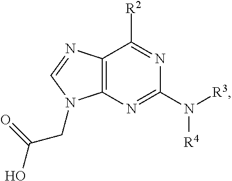

wherein R² is selected from halogen and a structure represented by:

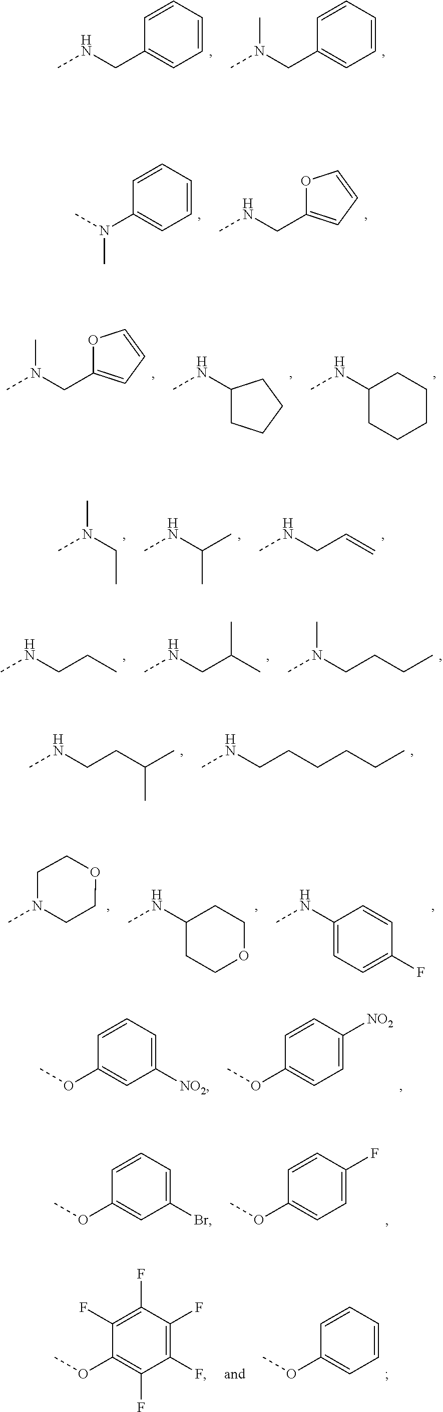

wherein R³ is selected from hydrogen and a structure represented by:

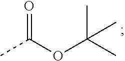

and, wherein R⁴ is selected from hydrogen and a structure represented by:

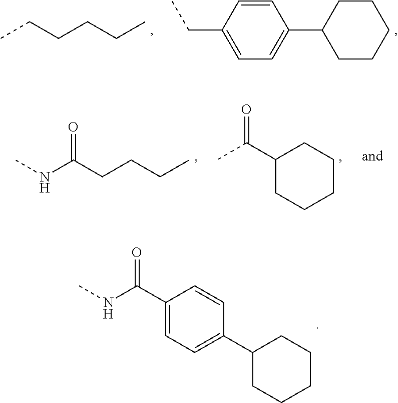

In a further aspect, the compound has a structure represented by a formula:

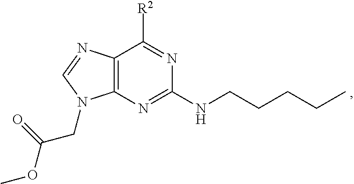

wherein R² is selected from halogen and a structure represented by:

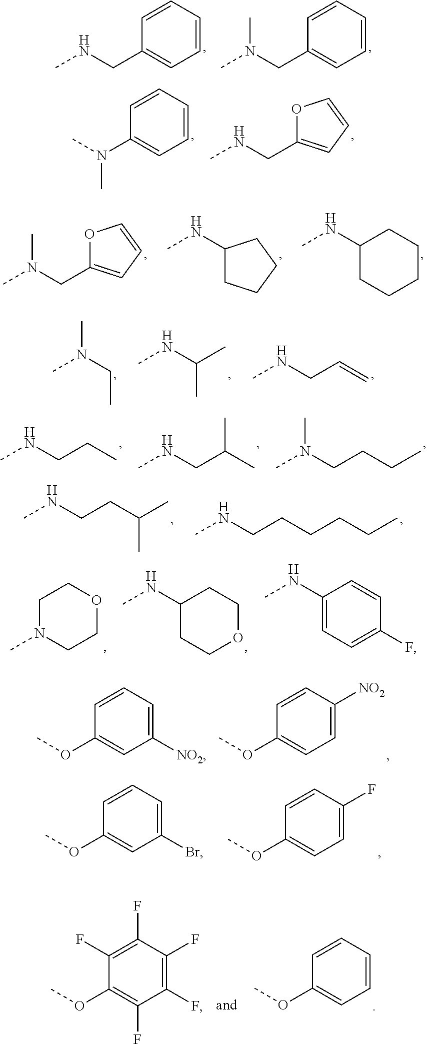

In a further aspect, the compound has a structure represented by a formula:

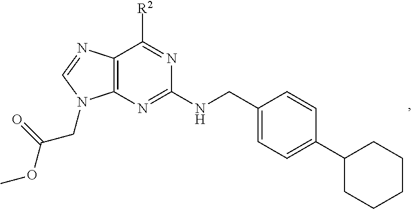

wherein R² is selected from halogen and a structure represented by:

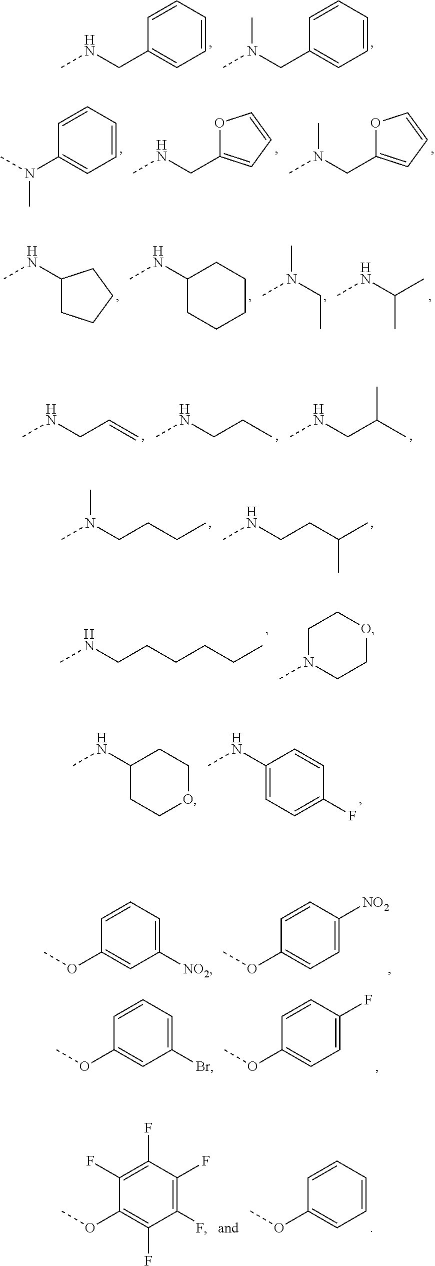

In a further aspect, the compound has a structure represented by a formula:

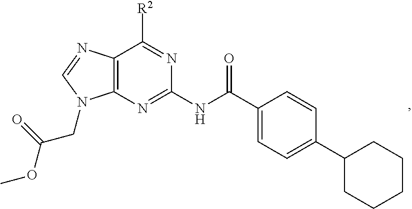

wherein R² is selected from halogen and a structure represented by:

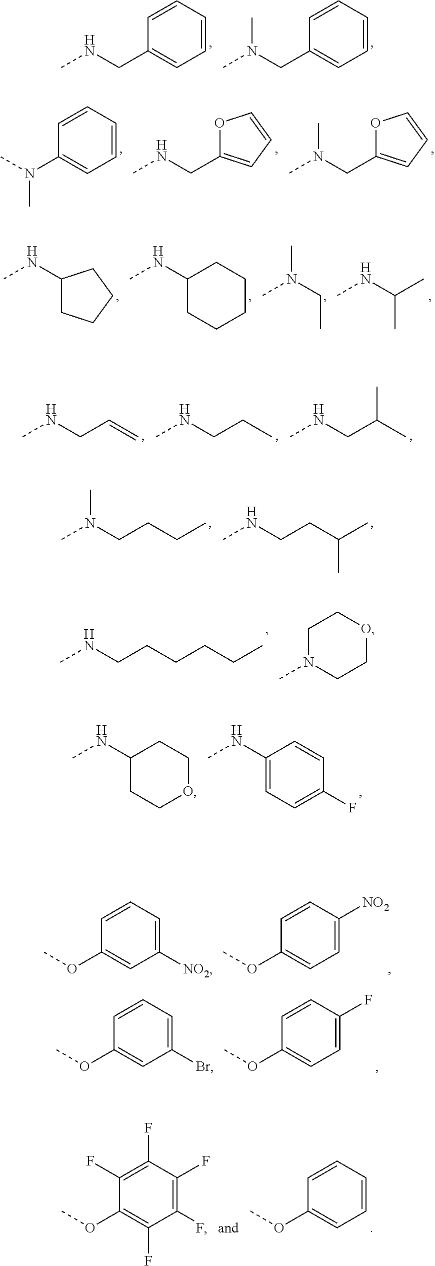

In a further aspect, the compound has a structure represented by a formula:

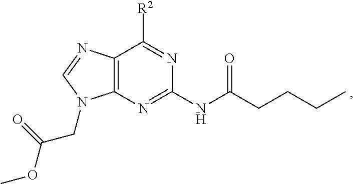

wherein R² is selected from halogen and a structure represented by:

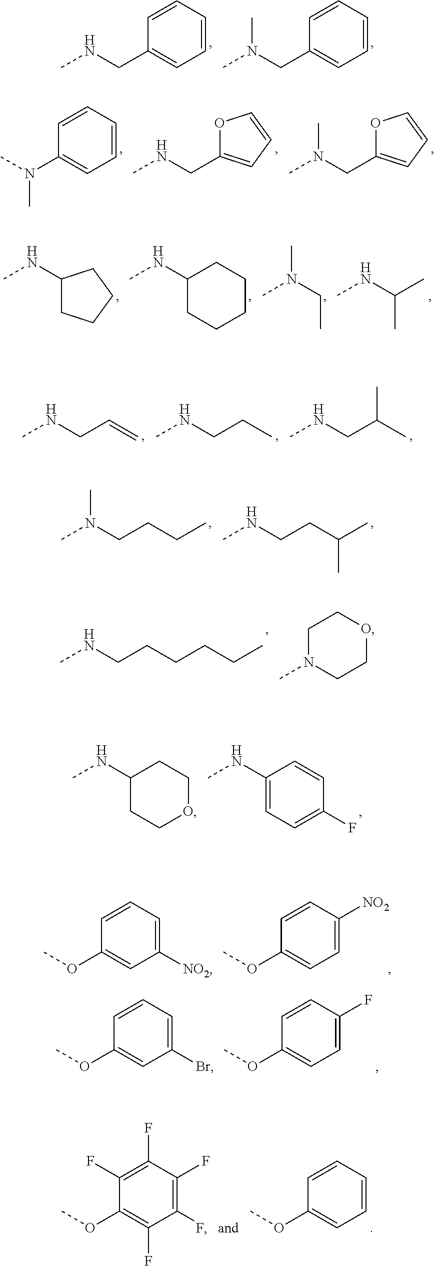

In a further aspect, the compound has a structure represented by a formula:

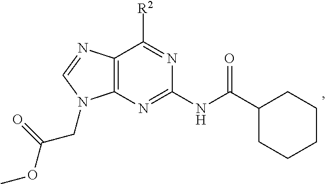

wherein R² is selected from halogen and a structure represented by:

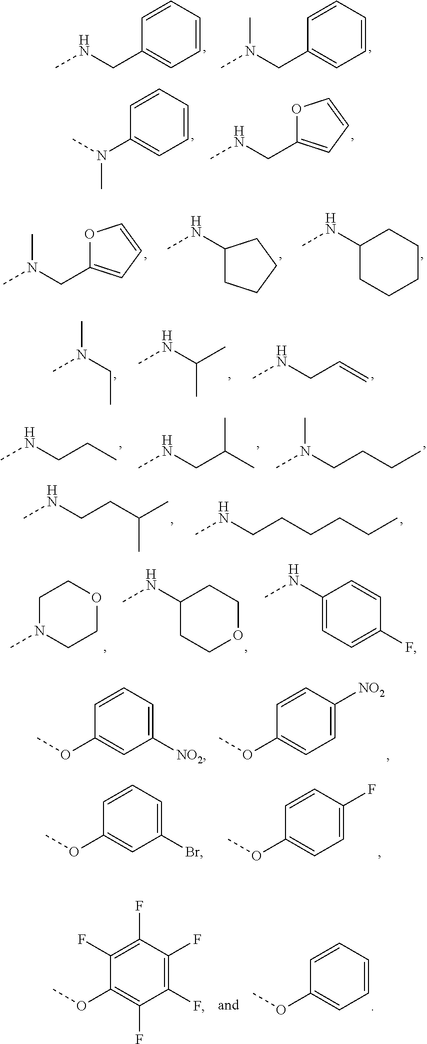

In a further aspect, the compound has a structure represented by a formula:

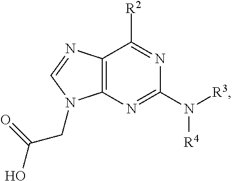

wherein R² is selected from halogen and a structure represented by:

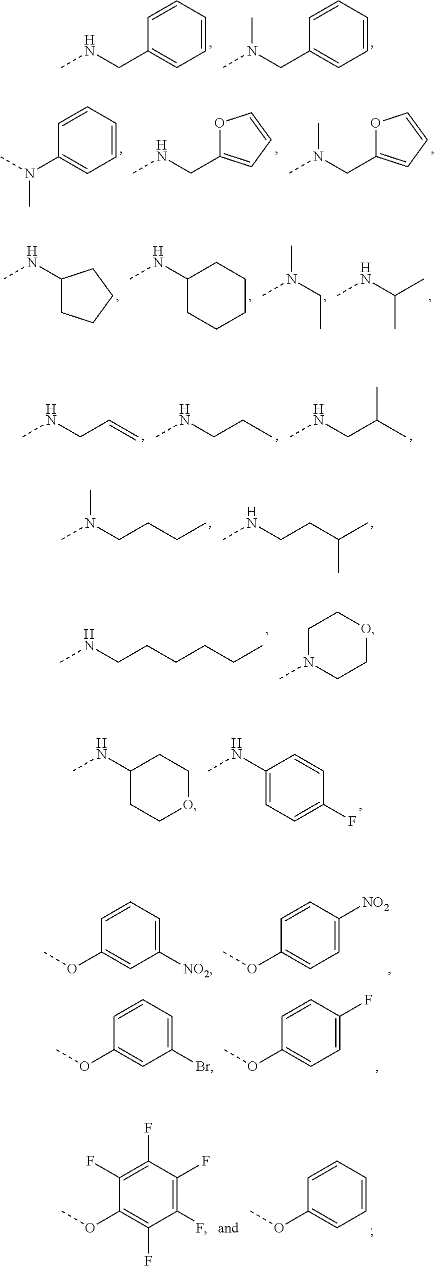

wherein R³ is selected from hydrogen and a structure represented by:

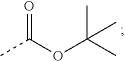

wherein R⁴ is selected from hydrogen and a structure represented by:

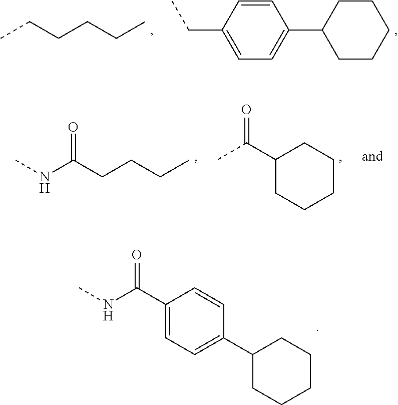

In a further aspect, the compound has a structure represented by a formula:

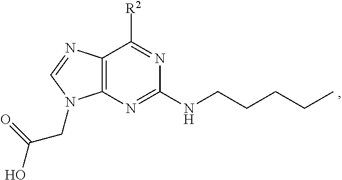

wherein R² is selected from halogen and a structure represented by:

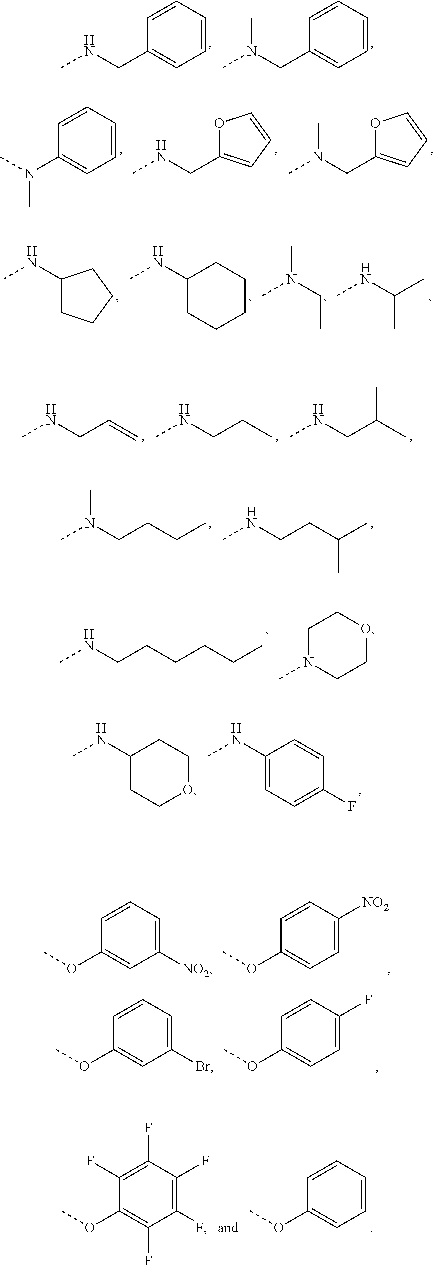

In a further aspect, the compound has a structure represented by a formula:

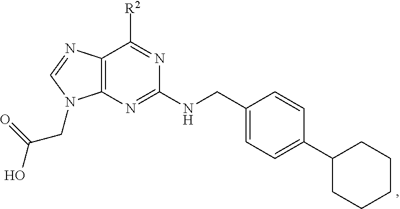

wherein R² is selected from halogen and a structure represented by:

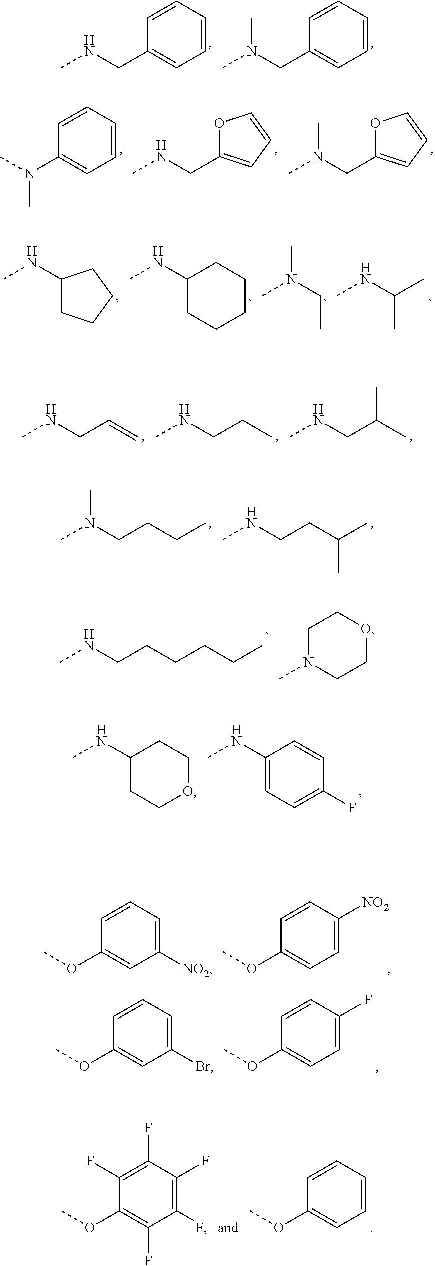

In a further aspect, the compound has a structure represented by a formula:

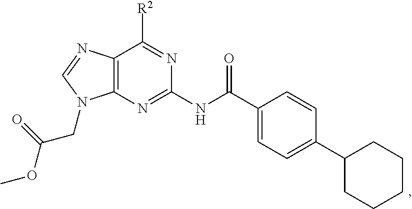

wherein R² is selected from halogen and a structure represented by:

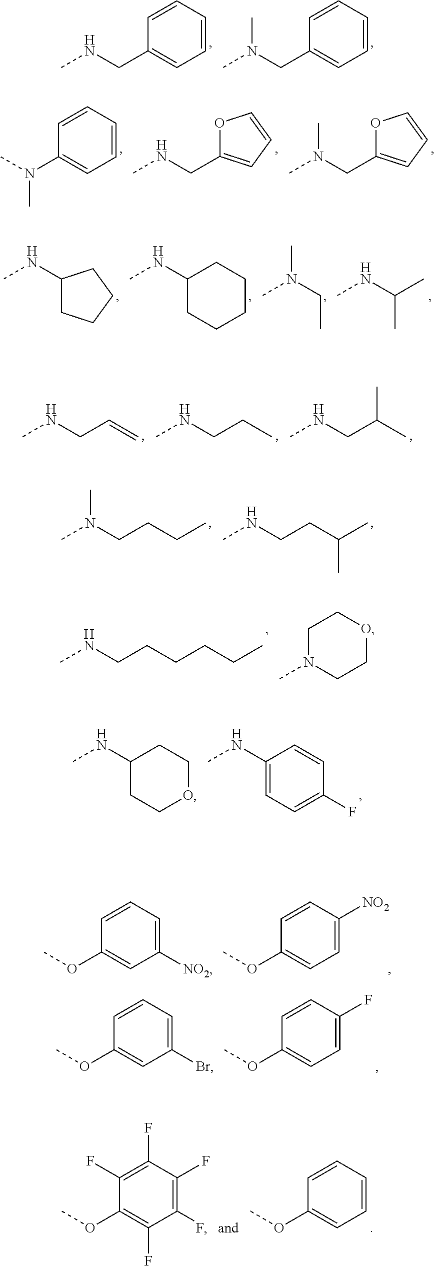

In a further aspect, the compound has a structure represented by a formula:

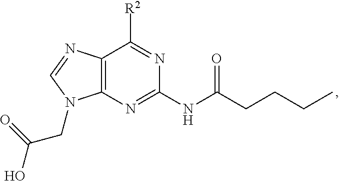

wherein R² is selected from halogen and a structure represented by:

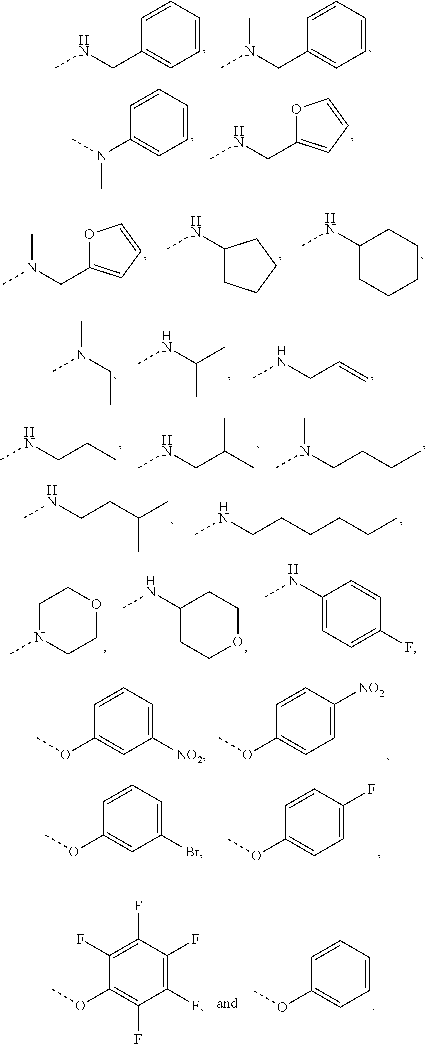

In a further aspect, the compound has a structure represented by a formula:

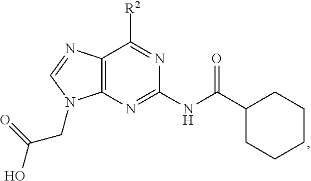

wherein R² is selected from halogen and a structure represented by:

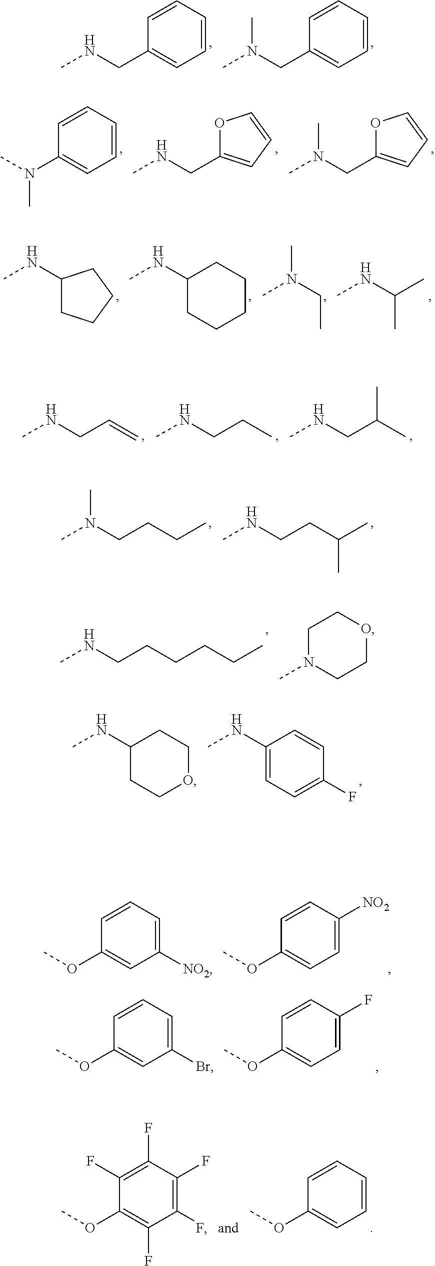

a. Ar¹ Groups

In various aspects, Ar¹ is selected from an optionally substituted monocyclic aryl and monocyclic heteroaryl. In a further aspect, Ar¹ is an optionally substituted monocyclic aryl. In a yet further aspect, Ar¹ is an optionally substituted monocyclic heteroaryl. In an even further aspect, Ar¹ is monosubstituted. In a still further aspect, Ar¹ is unsubstituted.

In a further aspect, Ar¹ is substituted with 0-4 substituents. In a still further aspect, Ar¹ is substituted with 0-3 substituents. In an even further aspect, Ar¹ is substituted with 1-5 substituents In a yet further aspect, Ar¹ is substituted with 1-4 substituents. In a further aspect, Ar¹ is substituted with 1-3 substituents. In a yet further aspect, Ar¹ is substituted with 1-2 substituents. In a yet further aspect, Ar¹ is substituted with 2 substituents. In a still further aspect, Ar¹ is substituted with 3 substituents. In a yet further aspect, Ar¹ is substituted with 4 substituents. In a still further aspect, Ar¹ is substituted with 5 substituents.

In a further aspect, Ar¹ is an optionally substituted monocyclic aryl. In a yet further aspect, Ar¹ is an optionally substituted monocyclic heteroaryl. In a yet further aspect, wherein Ar¹ is either phenyl substituted with 0-5 substituents independently selected from halo, cyano, hydroxyl, nitro, amino, C1-C6 haloalkyoxy, C1-C3 alkylamine, and C1-C3 dialkylamino, C1-C3 haloalkyl, and C1-C3 polyhaloalkyl, or is monocyclic heteroaryl substituted with 0-5 substituents independently selected from halo, cyano, hydroxyl, amino, C1-C6 haloalkyoxy, C1-C3 alkylamine, and C1-C3 dialkylamino, C1-C3 haloalkyl, and C1-C3 polyhaloalkyl.

In a further aspect, Ar¹ is phenyl substituted with 0-5 substituents independently selected from halo, cyano, hydroxyl, nitro, amino, C1-C6 haloalkyoxy, C1-C3 alkylamine, and C1-C3 dialkylamino, C1-C3 haloalkyl, and C1-C3 polyhaloalkyl. In a still further aspect, Ar¹ is phenyl substituted with 0-5 substituents independently selected from halo, cyano, hydroxyl, nitro, and amino. In a still further aspect, Ar¹ is phenyl substituted with 0-5 substituents independently selected from halo, cyano, hydroxyl, nitro, amino, methyl, CF₃, CF₂H, CFH₂, CCl₃, CCl₂H, and CClH₂.

In a further aspect, Ar¹ is phenyl monosubstituted with a group selected from halo, cyano, hydroxyl, nitro, and amino. In a still further aspect, Ar¹ is phenyl monosubstituted with a group selected from halo and nitro. In a yet further aspect, Ar¹ is phenyl monosubstituted with nitro. In a yet further aspect, Ar¹ is phenyl monosubstituted with halo. In an even further aspect, substituted with 0-5 halo substituents. In a still further aspect, Ar¹ is phenyl monosubstituted with a group selected from halo, cyano, hydroxyl, nitro, amino, methyl, CF₃, CF₂H, CFH₂, CCl₃, CCl₂H, and CClH₂.

In a further aspect, Ar¹ is monocyclic heteroaryl substituted with 0-5 substituents independently selected from halo, cyano, hydroxyl, amino, C1-C6 haloalkyoxy, C1-C3 alkylamine, and C1-C3 dialkylamino, C1-C3 haloalkyl, and C1-C3 polyhaloalkyl. In a still further aspect, Ar¹ is monocyclic heteroaryl substituted with 0-5 substituents independently selected from halo, cyano, hydroxyl, nitro, and amino. In a yet further aspect, Ar¹ is monocyclic heteroaryl monosubstituted with a group selected from halo and nitro. In an even further aspect, Ar¹ is monocyclic heteroaryl monosubstituted with nitro. In a still further aspect, Ar¹ is monocyclic heteroaryl monosubstituted with halo. In a yet further aspect, Ar¹ is moncyclic heteroaryl substituted with 0-5 halo substituents. In an even further aspect, Ar¹ is monocyclic heteroaryl substituted with 0-5 substituents independently selected from halo, cyano, hydroxyl, nitro, amino, methyl, CF₃, CF₂H, CFH₂, CCl₃, CCl₂H, and CClH₂. In a still further aspect, Ar¹ is monocyclic heteroaryl monosubstituted with a group selected from halo, cyano, hydroxyl, nitro, and amino. In a yet further aspect, Ar¹ is monocyclic heteroaryl monosubstituted with a group selected from halo, cyano, hydroxyl, nitro, amino, methyl, CF₃, CF₂H, CFH₂, CCl₃, CCl₂H, and CClH₂.

B. Ar² Groups

In various aspects, Ar² is selected from an optionally substituted monocyclic aryl and monocyclic heteroaryl. In a further aspect, Ar² is an optionally substituted monocyclic aryl. In a yet further aspect, Ar² is an optionally substituted monocyclic heteroaryl. In an even further aspect, Ar² is monosubstituted. In a still further aspect, Ar² is unsubstituted.

In a further aspect, Ar² is substituted with 0-4 substituents. In a still further aspect, Ar² is substituted with 0-3 substituents. In an even further aspect, Ar² is substituted with 1-5 substituents In a yet further aspect, Ar² is substituted with 1-4 substituents. In a further aspect, Ar² is substituted with 1-3 substituents. In a yet further aspect, Ar² is substituted with 1-2 substituents. In a yet further aspect, Ar² is substituted with 2 substituents. In a still further aspect, Ar² is substituted with 3 substituents. In a yet further aspect, Ar² is substituted with 4 substituents. In a still further aspect, Ar² is substituted with 5 substituents.

In a further aspect, Ar² is an optionally substituted monocyclic aryl. In a yet further aspect, Ar² is an optionally substituted monocyclic heteroaryl. In a yet further aspect, wherein Ar² is either phenyl substituted with 0-5 substituents independently selected from halo, cyano, hydroxyl, nitro, amino, C1-C6 haloalkyoxy, C1-C3 alkylamine, and C1-C3 dialkylamino, C1-C3 haloalkyl, and C1-C3 polyhaloalkyl, or is monocyclic heteroaryl substituted with 0-5 substituents independently selected from halo, cyano, hydroxyl, amino, C1-C6 haloalkyoxy, C1-C3 alkylamine, and C1-C3 dialkylamino, C1-C3 haloalkyl, and C1-C3 polyhaloalkyl.

In a further aspect, Ar² is phenyl substituted with 0-5 substituents independently selected from halo, cyano, hydroxyl, nitro, amino, C1-C6 haloalkyoxy, C1-C3 alkylamine, and C1-C3 dialkylamino, C1-C3 haloalkyl, and C1-C3 polyhaloalkyl. In a still further aspect, Ar² is phenyl substituted with 0-5 substituents independently selected from halo, cyano, hydroxyl, nitro, and amino. In a still further aspect, Ar² is phenyl substituted with 0-5 substituents independently selected from halo, cyano, hydroxyl, nitro, amino, methyl, CF₃, CF₂H, CFH₂, CCl₃, CCl₂H, and CClH₂.

In a further aspect, Ar² is phenyl monosubstituted with a group selected from cyclopropyl, cyclobutyl, cyclopentyl, and cyclohexyl. In a still further aspect, Ar² is phenyl monosubstituted with a group selected from aziridine, azetidine, pyrrolidine, and piperidine. In a yet further aspect, Ar² is phenyl monosubstituted with a group selected from pyrollidinyl, tetrahydrofuranyl, tetrahydrothiophene, piperidinyl, tetrahydro-2H-pyran, and tetrahydro-2H-thiopyran.

In a further aspect, Ar² is phenyl substituted with 0-3 substituents independently selected from halo, cyano, hydroxyl, nitro, amino, C1-C6 haloalkyoxy, C1-C3 alkylamine, and C1-C3 dialkylamino, C1-C3 haloalkyl, and C1-C3 polyhaloalkyl. In a yet further aspect, Ar² is phenyl substituted with 0-3 substituents independently selected from halo, cyano, hydroxyl, nitro, and amino. In a yet further aspect, Ar² is phenyl substituted with 0-3 substituents independently selected from halo, cyano, hydroxyl, nitro, amino, methyl, CF₃, CF₂H, CFH₂, CCl₃, CCl₂H, and CClH₂.

In a further aspect, Ar² is phenyl monosubstituted with a group selected from halo, cyano, hydroxyl, nitro, and amino. In a still further aspect, Ar² is phenyl monosubstituted with a group selected from halo and nitro. In a yet further aspect, Ar² is phenyl monosubstituted with nitro. In a yet further aspect, Ar² is phenyl monosubstituted with halo. In an even further aspect, substituted with 0-5 halo substituents. In a still further aspect, Ar² is phenyl monosubstituted with a group selected from halo, cyano, hydroxyl, nitro, amino, methyl, CF₃, CF₂H, CFH₂, CCl₃, CCl₂H, and CClH₂. In a further aspect, Ar² is phenyl monosubstituted with a group selected from halo, cyano, hydroxyl, nitro, and amino. In a yet further aspect, Ar² is phenyl monosubstituted with a group selected from halo and nitro. In an even further aspect, Ar² is phenyl monosubstituted with nitro. In a yet further aspect, Ar² is phenyl monosubstituted with halo.

In a further aspect, Ar² is monocyclic heteroaryl substituted with 0-5 substituents independently selected from halo, cyano, hydroxyl, amino, C1-C6 haloalkyoxy, C1-C3 alkylamine, and C1-C3 dialkylamino, C1-C3 haloalkyl, and C1-C3 polyhaloalkyl. In a still further aspect, Ar² is monocyclic heteroaryl substituted with 0-5 substituents independently selected from halo, cyano, hydroxyl, nitro, and amino. In a yet further aspect, Ar² is moncyclic heteroaryl substituted with 0-5 halo substituents. In an even further aspect, Ar² is monocyclic heteroaryl substituted with 0-5 substituents independently selected from halo, cyano, hydroxyl, nitro, amino, methyl, CF₃, CF₂H, CFH₂, CCl₃, CCl₂H, and CClH₂.

In a further aspect, Ar² is monocyclic heteroaryl monosubstituted with a group selected from halo and nitro. In an even further aspect, Ar² is monocyclic heteroaryl monosubstituted with nitro. In a still further aspect, Ar² is monocyclic heteroaryl monosubstituted with halo. In a still further aspect, Ar² is monocyclic heteroaryl monosubstituted with a group selected from halo, cyano, hydroxyl, nitro, and amino. In a yet further aspect, Ar² is monocyclic heteroaryl monosubstituted with a group selected from halo, cyano, hydroxyl, nitro, amino, methyl, CF₃, CF₂H, CFH₂, CCl₃, CCl₂H, and CClH₂.

In a further aspect, Ar² is monocyclic heteroaryl substituted with 0-3 substituents independently selected from halo, cyano, hydroxyl, nitro, amino, methyl, CF₃, CF₂H, CFH₂, CCl₃, CCl₂H, and CClH₂.

c. Ar³ Groups

In various aspects, Ar³ is selected from an optionally substituted monocyclic aryl and monocyclic heteroaryl. In a further aspect, Ar³ is an optionally substituted monocyclic aryl. In a yet further aspect, Ar³ is an optionally substituted monocyclic heteroaryl. In an even further aspect, Ar³ is monosubstituted. In a still further aspect, Ar³ is unsubstituted.

In a further aspect, Ar³ is substituted with 0-4 substituents. In a still further aspect, Ar³ is substituted with 0-3 substituents. In an even further aspect, Ar³ is substituted with 1-5 substituents In a yet further aspect, Ar³ is substituted with 1-4 substituents. In a further aspect, Ar³ is substituted with 1-3 substituents. In a yet further aspect, Ar³ is substituted with 1-2 substituents. In a yet further aspect, Ar³ is substituted with 2 substituents. In a still further aspect, Ar³ is substituted with 3 substituents. In a yet further aspect, Ar³ is substituted with 4 substituents. In a still further aspect, Ar³ is substituted with 5 substituents.

In a further aspect, Ar³ is an optionally substituted monocyclic aryl. In a yet further aspect, Ar³ is an optionally substituted monocyclic heteroaryl. In a yet further aspect, wherein Ar³ is either phenyl substituted with 0-5 substituents independently selected from halo, cyano, hydroxyl, nitro, amino, C1-C6 haloalkyoxy, C1-C3 alkylamine, and C1-C3 dialkylamino, C1-C3 haloalkyl, and C1-C3 polyhaloalkyl, or is monocyclic heteroaryl substituted with 0-5 substituents independently selected from halo, cyano, hydroxyl, amino, C1-C6 haloalkyoxy, C1-C3 alkylamine, and C1-C3 dialkylamino, C1-C3 haloalkyl, and C1-C3 polyhaloalkyl.

In a further aspect, Ar³ is either phenyl substituted with 0-3 substituents independently selected from halo, cyano, hydroxyl, nitro, amino, C1-C6 haloalkyoxy, C1-C3 alkylamine, and C1-C3 dialkylamino, C1-C3 haloalkyl, C1-C3 polyhaloalkyl, C3-C6 cycloalkyl, and C3-C6 heterocycloalkyl, or is monocyclic heteroaryl substituted with 0-3 substituents independently selected from halo, cyano, hydroxyl, amino, C1-C6 haloalkyoxy, C1-C3 alkylamine, and C1-C3 dialkylamino, C1-C3 haloalkyl, and C1-C3 polyhaloalkyl, C3-C6 cycloalkyl, and C3-C6 heterocycloalkyl.

In a further aspect, Ar³ is phenyl substituted with 0-5 substituents independently selected from halo, cyano, hydroxyl, nitro, amino, C1-C6 haloalkyoxy, C1-C3 alkylamine, and C1-C3 dialkylamino, C1-C3 haloalkyl, and C1-C3 polyhaloalkyl. In a still further aspect, Ar³ is phenyl substituted with 0-5 substituents independently selected from halo, cyano, hydroxyl, nitro, and amino. In a still further aspect, Ar³ is phenyl substituted with 0-5 substituents independently selected from halo, cyano, hydroxyl, nitro, amino, methyl, CF₃, CF₂H, CFH₂, CCl₃, CCl₂H, and CClH₂. In a still further aspect, Ar³ is phenyl monosubstituted with a group selected from halo and nitro. In a yet further aspect, Ar³ is phenyl monosubstituted with nitro. In a yet further aspect, Ar³ is phenyl monosubstituted with halo. In an even further aspect, Ar³ is phenyl substituted with 0-5 halo substituents

In a further aspect, Ar³ is phenyl substituted with 1-3 substituents independently selected from halo, cyano, hydroxyl, nitro, amino, C1-C6 haloalkyoxy, C1-C3 alkylamine, and C1-C3 dialkylamino, C1-C3 haloalkyl, C1-C3 polyhaloalkyl, C3-C6 cycloalkyl, and C3-C6 heterocycloalkyl. In a yet further aspect, Ar³ is phenyl substituted with 1-3 substituents independently selected C3-C6 cycloalkyl, and C3-C6 heterocycloalkyl. In a still further aspect, Ar³ is phenyl substituted with 1-3 substituents independently selected from cyclopropyl, cyclobutyl, cyclopentyl, and cyclohexyl. In a yet further aspect, Ar³ is phenyl substituted with 1-3 substituents independently selected from aziridine, azetidine, pyrrolidine, and piperidine. In a still further aspect, Ar³ is phenyl substituted with 1-3 substituents independently selected from pyrollidinyl, tetrahydrofuranyl, tetrahydrothiophene, piperidinyl, tetrahydro-2H-pyran, and tetrahydro-2H-thiopyran. In a yet further aspect, Ar³ is phenyl monosubstituted with a group selected C3-C6 cycloalkyl, and C3-C6 heterocycloalkyl. In an even further aspect, Ar³ is phenyl substituted with 1-3 substituents independently selected from halo, cyano, hydroxyl, nitro, and amino. In a yet further aspect, Ar³ is phenyl substituted with 1-3 substituents independently selected from halo, cyano, hydroxyl, nitro, amino, methyl, CF₃, CF₂H, CFH₂, CCl₃, CCl₂H, and CClH₂.

In a further aspect, Ar³ is phenyl monosubstituted with a group selected from cyclopropyl, cyclobutyl, cyclopentyl, and cyclohexyl. In a still further aspect, Ar³ is phenyl monosubstituted with a group selected from aziridine, azetidine, pyrrolidine, and piperidine. In a yet further aspect, Ar³ is phenyl monosubstituted with a group selected from pyrollidinyl, tetrahydrofuranyl, tetrahydrothiophene, piperidinyl, tetrahydro-2H-pyran, and tetrahydro-2H-thiopyran.

In a further aspect, Ar³ is monocyclic heteroaryl substituted with 0-5 substituents independently selected from halo, cyano, hydroxyl, amino, C1-C6 haloalkyoxy, C1-C3 alkylamine, and C1-C3 dialkylamino, C1-C3 haloalkyl, and C1-C3 polyhaloalkyl. In a yet further aspect, Ar³ is monocyclic heteroaryl substituted with 1-3 substituents independently selected from halo, cyano, hydroxyl, amino, C1-C6 haloalkyoxy, C1-C3 alkylamine, and C1-C3 dialkylamino, C1-C3 haloalkyl, C1-C3 polyhaloalkyl, C3-C6 cycloalkyl, and C3-C6 heterocycloalkyl. In a still further aspect, Ar³ is monocyclic heteroaryl substituted with 0-5 substituents independently selected from halo, cyano, hydroxyl, nitro, and amino. In a yet further aspect, Ar³ is monocyclic heteroaryl monosubstituted with a group selected from halo and nitro. In an even further aspect, Ar³ is monocyclic heteroaryl monosubstituted with nitro. In a still further aspect, Ar³ is monocyclic heteroaryl monosubstituted with halo. In a yet further aspect, Ar³ is moncyclic heteroaryl substituted with 0-5 halo substituents. In an even further aspect, Ar³ is monocyclic heteroaryl substituted with 0-5 substituents independently selected from halo, cyano, hydroxyl, nitro, amino, methyl, CF₃, CF₂H, CFH₂, CCl₃, CCl₂H, and CClH₂. In a still further aspect, Ar³ is monocyclic heteroaryl monosubstituted with a group selected from halo, cyano, hydroxyl, nitro, and amino. In a yet further aspect, Ar³ is monocyclic heteroaryl monosubstituted with a group selected from halo, cyano, hydroxyl, nitro, amino, methyl, CF₃, CF₂H, CFH₂, CCl₃, CCl₂H, and CClH₂.

In a further aspect, Ar³ is monocyclic heteroaryl substituted with 1-3 substituents independently selected C3-C6 cycloalkyl, and C3-C6 heterocycloalkyl. In a still further aspect, Ar³ is monocyclic heteroaryl substituted with 1-3 substituents independently selected from cyclopropyl, cyclobutyl, cyclopentyl, and cyclohexyl. In an even further aspect, Ar³ is monocyclic heteroaryl substituted with 1-3 substituents independently selected from aziridine, azetidine, pyrrolidine, and piperidine. In a yet further aspect, Ar³ is monocyclic heteroaryl substituted with 1-3 substituents independently selected from pyrollidinyl, tetrahydrofuranyl, tetrahydrothiophene, piperidinyl, tetrahydro-2H-pyran, and tetrahydro-2H-thiopyran.

d. R¹ Groups

In one aspect, R¹ is selected from H and (CH₂)ₘC═OR⁵. In a further aspect, R¹ is hydrogen. In a still further aspect, R¹ is (CH₂)ₘC═OR⁵, wherein m is an integer from 0-3. In an even further aspect, R¹ is C═OR⁵. In a still further aspect, R¹ is (CH₂)₁C═OR⁵. In a yet further aspect, R¹ is (CH₂)₂C═OR⁵. In a yet further aspect, R¹ is (CH₂)₃C═OR⁵.

e. R² Groups

In one aspect, R² is selected from halogen, OR⁹, and NR¹⁰R¹¹. In a further aspect,

R² is selected from OR⁹ and NR¹⁰R¹¹. In a still further aspect, R² is selected from halogen and OR⁹. In a yet further aspect, R² is selected from halogen and NR¹⁰R¹¹. In an even further aspect, R² is halogen. In a still further aspect, R² is OR⁹. In a yet further aspect, R² is NR¹⁰R¹¹.

f. R³ Groups

In one aspect, R³ is selected from hydrogen and an optionally substituted group selected from C1-C8 alkyl, (CH₂)_{q}R¹⁴, and C═O(CH₂)_{q}R¹⁴.

In a further aspect, R³ and R⁴ are optionally covalently bonded and, together with the intermediate carbon, comprise an optionally substituted 3- to 7-membered heterocycloalkyl.

In a further aspect, R³ is hydrogen. In a yet further aspect, R³ is selected from an optionally substituted group selected from C1-C8 alkyl and (CH₂)_{q}R¹⁴, wherein q is an integer from 0-3. In a still further aspect, R³ is selected from an optionally substituted group selected from C1-C8 alkyl and C═OR¹⁴. In a yet further aspect, R³ is selected from an optionally substituted group selected from C1-C8 alkyl and C═O(CH₂)R¹⁴. In an even further aspect, R³ is selected from an optionally substituted group selected from C1-C8 alkyl and C═O(CH₂)₂R¹⁴. In a still further aspect, R³ is selected from an optionally substituted group selected from C1-C8 alkyl and C═O(CH₂)₃R¹⁴.

In a further aspect, R³ is selected from an optionally substituted group selected from (CH₂)_{q}R¹⁴ and C═O(CH₂)_{q}R¹⁴, wherein q is an integer from 0-3. In a still further aspect, R³ is optionally substituted C1-C8 alkyl.

In a further aspect, R³ is optionally substituted (CH₂)_{q}R¹⁴, wherein q is an integer from 0-3. In a further aspect, R³ is optionally substituted (CH₂)R¹⁴. In a further aspect, R³ is optionally substituted (CH₂)₂R¹⁴. In a further aspect, R³ is optionally substituted (CH₂)₃R¹⁴.

In a further aspect, R³ is optionally substituted C═O(CH₂)_{q}R¹⁴, wherein q is an integer from 0-3. In a still further aspect, R³ is optionally substituted C═O(CH₂)R¹⁴. In a still further aspect, R³ is optionally substituted C═O(CH₂)₂R¹⁴. In a still further aspect, R³ is optionally substituted C═O(CH₂)₃R¹⁴.

In a further aspect, R³ and R⁴ are optionally covalently bonded and, together with the intermediate carbon, comprise an optionally substituted 3- to 6-membered heterocycloalkyl. In a yet further aspect, R³ and R⁴ are optionally covalently bonded and, together with the intermediate carbon, comprise an optionally substituted 3-, 5- or 6-membered heterocycloalkyl. In a still further aspect, R³ and R⁴ are optionally covalently bonded and, together with the intermediate carbon, comprise an optionally substituted heterocycloalkyl selected from aziridine, azetidine, pyrollidine, oxazolidine, imidazolidine, pyrazolidine, piperidine, 4-amino piperidine, piperazine, morpholine, and hexahydropyrimidine. In an even further aspect, R³ and R⁴ are optionally covalently bonded and, together with the intermediate carbon, comprise an optionally substituted heterocycloalkyl selected from aziridine, azetidine, pyrollidine, piperidine, 4-amino piperidine, piperazine, and morpholine. In a still further aspect, R³ and R⁴ are optionally covalently bonded and, together with the intermediate carbon, comprise an optionally substituted heterocycloalkyl selected from aziridine, pyrollidine, piperidine, piperazine, and morpholine. In a yet further aspect, R³ and R⁴ are optionally covalently bonded and, together with the intermediate carbon, comprise an optionally substituted aziridine. In a still further aspect, R³ and R⁴ are optionally covalently bonded and, together with the intermediate carbon, comprise an optionally substituted pyrollidine. In an even further aspect, R³ and R⁴ are optionally covalently bonded and, together with the intermediate carbon, comprise an optionally substituted piperidine. In a still further aspect, R³ and R⁴ are optionally covalently bonded and, together with the intermediate carbon, comprise an optionally substituted morpholine.

g. R⁴ Groups

In one aspect, R⁴ is selected from hydrogen and C═OOR¹⁵. In a further aspect, R⁴ is hydrogen. In a still further aspect, R⁴ is C═OOR¹⁵.

h. R⁵ Groups

In one aspect, R⁵ is selected from OR⁶ and NR⁷R⁸. In a further aspect, R⁵ is OR⁶. In a further aspect, R⁵ is NR⁷R⁸.

i. R⁶ Groups

In one aspect, R⁶ is selected from hydrogen and C1-C8 (e.g., C1-C6 or C1-C4) alkyl. In a further aspect, R⁶ is hydrogen. In a further aspect, R⁶ is C1-C8 alkyl, for example, methyl, ethyl, propyl, butyl, penyl, hexyl, heptyl, or octyl.

j. R⁷ Groups

In one aspect, R⁷ is selected from hydrogen and C1-C8 (e.g., C1-C6 or C1-C4) alkyl. In a further aspect, R⁷ is hydrogen. In a further aspect, R⁷ is C1-C8 alkyl, for example, methyl, ethyl, propyl, butyl, penyl, hexyl, heptyl, or octyl.

k. R⁸ Groups

In one aspect, R⁸ is selected from hydrogen and C1-C8 (e.g., C1-C6 or C1-C4) alkyl. In a further aspect, R⁸ is hydrogen. In a further aspect, R⁸ is C1-C8 alkyl, for example, methyl, ethyl, propyl, butyl, penyl, hexyl, heptyl, or octyl.

l. R⁹ Groups

In one aspect, R⁹ is an optionally substituted group selected from C1-C8 alkyl, C3-C6 cycloalkyl, C3-C6 heterocycloalkyl, (CH₂)ₙR¹², and Ar¹. In a further aspect, R⁹ is optionally substituted C1-C8 alkyl. In a further aspect, R⁹ is C1-C8 alkyl, for example, methyl, ethyl, propyl, butyl, penyl, hexyl, heptyl, or octyl. In a further aspect, R⁹ is optionally substituted C3-C6 (e.g., C3, C4, or C5) cycloalkyl. In a further aspect, R⁹ is C3-C6 cycloalkyl. In a further aspect, R¹⁰ is cyclopropyl, cyclobutyl, cyclopentyl, or cyclohexyl.

In a further aspect, R⁹ is optionally substituted C3-C6 heterocycloalkyl.

In a further aspect, R⁹ is optionally substituted C3-C6 heterocycloalkyl. In a further aspect, R⁹ is C3-C6 (e.g., C3, C4, or C5) heterocycloalkyl. In a further aspect, R⁹ is substituted with 1, 2, or 3 heteroatoms selected from O, S, and N. It is understood that such replacement will alter the number of substitutent groups (e.g., each O or S substituted for C will decrease the number of substitutent groups by two, whereas each N substituted for C will decrease the number of substitutent groups by one). In a further aspect, heterocycloalkyl can be oxirane, oxetane, tetrahydrofuran, tetrahydro-2H-pyran, oxepane, oxocane, dioxirane, dioxetane, dioxolane, dioxane, dioxepane, dioxocane, thiirane, thietane, tetrahydrothiophene, tetrahydro-2H-thiopyran, thiepane, thiocane, dithiirane, dithietane, dithiolane, dithiane, dithiepane, dithiocane, oxathiirane, oxathietane, oxathiolane, oxathiane, oxathiepane, oxathiocane, aziridine, azetidine, pyrrolidone, piperidine, azepane, azocane, diaziridine, diazetidine, imidazolidine, piperazine, diazepane, diazocane, hexahydropyrimidine, triazinane, oxaziridine, oxazetidine, oxazolidine, morpholine, oxazepane, oxazocane, thiaziridine, thiazetidine, thiazolidine, thiomorpholine, thiazepane, or thiazocane. In a further aspect, heterocycloalkyl can be monocyclic or bicyclic.

In a further aspect, R⁹ is optionally substituted (CH₂)ₙR¹². In a further aspect, R⁹ is optionally substituted Ar¹. In a further aspect, R⁹ is Ar¹.

In a further aspect, R⁹ is unsubstituted. In a further aspect, R⁹ is substituted with 0-4 groups (e.g., 0-3 groups, 0-2 groups, 0-1 groups, 4 groups, 3 groups, 2 groups, or 1 group). In various aspects, the groups can be selected from halogen (e.g., fluoro, chloro, bromo, or iodo), cyano, hydroxyl, amino, alkylamino, and dialkylamino.

m. R¹⁰ Groups

In one aspect, R¹⁰ is selected from hydrogen and an optionally substituted group selected from C1-C8 alkyl, C3-C6 cycloalkyl, C3-C6 heterocycloalkyl, (CH₂)ₚR¹³, and Ar². In a further aspect, R¹⁰ is hydrogen. In a further aspect, R¹⁰ is optionally substituted C1-C8 alkyl. In a further aspect, R¹⁰ is C1-C8 alkyl, for example, methyl, ethyl, propyl, butyl, penyl, hexyl, heptyl, or octyl. In a further aspect, R¹⁰ is optionally substituted C3-C6 (e.g., C3, C4, or C5) cycloalkyl. In a further aspect, R¹⁰ is C3-C6 cycloalkyl. In a further aspect, R¹⁰ is cyclopropyl, cyclobutyl, cyclopentyl, or cyclohexyl.

In a further aspect, R¹⁰ is optionally substituted C3-C6 heterocycloalkyl. In a further aspect, R¹⁰ is C3-C6 (e.g., C3, C4, or C5) heterocycloalkyl. In a further aspect, R¹⁰ is substituted with 1, 2, or 3 heteroatoms selected from O, S, and N. It is understood that such replacement will alter the number of substitutent groups (e.g., each O or S substituted for C will decrease the number of substitutent groups by two, whereas each N substituted for C will decrease the number of substitutent groups by one). In a further aspect, heterocycloalkyl can be oxirane, oxetane, tetrahydrofuran, tetrahydro-2H-pyran, oxepane, oxocane, dioxirane, dioxetane, dioxolane, dioxane, dioxepane, dioxocane, thiirane, thietane, tetrahydrothiophene, tetrahydro-2H-thiopyran, thiepane, thiocane, dithiirane, dithietane, dithiolane, dithiane, dithiepane, dithiocane, oxathiirane, oxathietane, oxathiolane, oxathiane, oxathiepane, oxathiocane, aziridine, azetidine, pyrrolidone, piperidine, azepane, azocane, diaziridine, diazetidine, imidazolidine, piperazine, diazepane, diazocane, hexahydropyrimidine, triazinane, oxaziridine, oxazetidine, oxazolidine, morpholine, oxazepane, oxazocane, thiaziridine, thiazetidine, thiazolidine, thiomorpholine, thiazepane, or thiazocane. In a further aspect, heterocycloalkyl can be monocyclic or bicyclic.

In a further aspect, R¹⁰ is optionally substituted (CH₂)ₚR¹³. In a further aspect, R¹⁰ is optionally substituted Ar². In a further aspect, R¹⁰ is Ar².

In a further aspect, R¹⁰ is unsubstituted. In a further aspect, R¹⁰ is substituted with 0-4 groups (e.g., 0-3 groups, 0-2 groups, 0-1 groups, 4 groups, 3 groups, 2 groups, or 1 group). In various aspects, the groups can be selected from halogen (e.g., fluoro, chloro, bromo, or iodo), cyano, hydroxyl, amino, alkylamino, and dialkylamino.

n. R¹¹ Groups

In one aspect, R¹¹ is selected from hydrogen and C1-C8 alkyl. In a further aspect, R¹¹ is hydrogen. In a further aspect, R¹¹ is C1-C8 alkyl, for example, methyl, ethyl, propyl, butyl, penyl, hexyl, heptyl, or octyl. In a further aspect, R¹⁰ and R¹¹ are optionally covalently bonded and, together with the intermediate carbon, comprise an optionally substituted 3- to 7-membered heterocycloalkyl.

o. R¹² Groups

In one aspect, R¹² is an optionally substituted group selected from Ar¹, C3-C6 cycloalkyl and C3-C6 heterocycloalkyl. In a further aspect, R¹² is optionally substituted Ar¹. In a further aspect, R¹² is Ar¹. In a further aspect, R¹² is optionally substituted C3-C6 (e.g., C3, C4, or C5) cycloalkyl. In a further aspect, R¹² is C3-C6 cycloalkyl. In a further aspect, R¹² is cyclopropyl, cyclobutyl, cyclopentyl, or cyclohexyl.

In a further aspect, R¹² is optionally substituted C3-C6 heterocycloalkyl. In a further aspect, R¹² is C3-C6 (e.g., C3, C4, or C5) heterocycloalkyl. In a further aspect, R¹² is substituted with 1, 2, or 3 heteroatoms selected from O, S, and N. It is understood that such replacement will alter the number of substitutent groups (e.g., each O or S substituted for C will decrease the number of substitutent groups by two, whereas each N substituted for C will decrease the number of substitutent groups by one). In a further aspect, heterocycloalkyl can be oxirane, oxetane, tetrahydrofuran, tetrahydro-2H-pyran, oxepane, oxocane, dioxirane, dioxetane, dioxolane, dioxane, dioxepane, dioxocane, thiirane, thietane, tetrahydrothiophene, tetrahydro-2H-thiopyran, thiepane, thiocane, dithiirane, dithietane, dithiolane, dithiane, dithiepane, dithiocane, oxathiirane, oxathietane, oxathiolane, oxathiane, oxathiepane, oxathiocane, aziridine, azetidine, pyrrolidone, piperidine, azepane, azocane, diaziridine, diazetidine, imidazolidine, piperazine, diazepane, diazocane, hexahydropyrimidine, triazinane, oxaziridine, oxazetidine, oxazolidine, morpholine, oxazepane, oxazocane, thiaziridine, thiazetidine, thiazolidine, thiomorpholine, thiazepane, or thiazocane. In a further aspect, heterocycloalkyl can be monocyclic or bicyclic.

In a further aspect, R¹² is unsubstituted. In a further aspect, R¹² is substituted with 0-4 groups (e.g., 0-3 groups, 0-2 groups, 0-1 groups, 4 groups, 3 groups, 2 groups, or 1 group). In various aspects, the groups can be selected from halogen (e.g., fluoro, chloro, bromo, or iodo), cyano, hydroxyl, amino, alkylamino, and dialkylamino.

p. R¹³ Groups

In one aspect, R¹³ is an optionally substituted group selected from Ar², C3-C6 cycloalkyl and C3-C6 heterocycloalkyl. In a further aspect, R¹³ is optionally substituted Ar². In a further aspect, R¹³ is Ar². In a further aspect, R¹³ is optionally substituted C3-C6 (e.g., C3, C4, or C5) cycloalkyl. In a further aspect, R¹³ is C3-C6 cycloalkyl. In a further aspect, R¹³ is cyclopropyl, cyclobutyl, cyclopentyl, or cyclohexyl.

In a further aspect, R¹³ is optionally substituted C3-C6 heterocycloalkyl. In a further aspect, R¹³ is C3-C6 (e.g., C3, C4, or C5) heterocycloalkyl. In a further aspect, R¹³ is substituted with 1, 2, or 3 heteroatoms selected from O, S, and N. It is understood that such replacement will alter the number of substitutent groups (e.g., each O or S substituted for C will decrease the number of substitutent groups by two, whereas each N substituted for C will decrease the number of substitutent groups by one). In a further aspect, heterocycloalkyl can be oxirane, oxetane, tetrahydrofuran, tetrahydro-2H-pyran, oxepane, oxocane, dioxirane, dioxetane, dioxolane, dioxane, dioxepane, dioxocane, thiirane, thietane, tetrahydrothiophene, tetrahydro-2H-thiopyran, thiepane, thiocane, dithiirane, dithietane, dithiolane, dithiane, dithiepane, dithiocane, oxathiirane, oxathietane, oxathiolane, oxathiane, oxathiepane, oxathiocane, aziridine, azetidine, pyrrolidone, piperidine, azepane, azocane, diaziridine, diazetidine, imidazolidine, piperazine, diazepane, diazocane, hexahydropyrimidine, triazinane, oxaziridine, oxazetidine, oxazolidine, morpholine, oxazepane, oxazocane, thiaziridine, thiazetidine, thiazolidine, thiomorpholine, thiazepane, or thiazocane. In a further aspect, heterocycloalkyl can be monocyclic or bicyclic.

In a further aspect, R¹³ is unsubstituted. In a further aspect, R¹³ is substituted with 0-4 groups (e.g., 0-3 groups, 0-2 groups, 0-1 groups, 4 groups, 3 groups, 2 groups, or 1 group). In various aspects, the groups can be selected from halogen (e.g., fluoro, chloro, bromo, or iodo), cyano, hydroxyl, amino, alkylamino, and dialkylamino.

q. R¹⁴ Groups

In one aspect, R¹⁴ is selected from hydrogen and an optionally substituted group selected from C1-C8 alkyl, C3-C6 cycloalkyl, C3-C6 heterocycloalkyl, and Ar^{a}. In a further aspect, R¹⁴ is hydrogen. In a further aspect, R¹⁴ is optionally substituted C1-C8 (e.g., C1-C6 or C1-C4) alkyl. In a further aspect, R¹⁴ is C1-C8 alkyl. In a further aspect, R¹⁴ is methyl, ethyl, propyl, butyl, penyl, hexyl, heptyl, or octyl. In a further aspect, R¹⁴ is optionally substituted C3-C6 (e.g., C3, C4, or C5) cycloalkyl. In a further aspect, R¹⁴ is C3-C6 cycloalkyl. In a further aspect, R¹⁴ is cyclopropyl, cyclobutyl, cyclopentyl, or cyclohexyl.

In a further aspect, R¹⁴ is optionally substituted C3-C6 heterocycloalkyl. In a further aspect, R¹⁴ is C3-C6 (e.g., C3, C4, or C5) heterocycloalkyl. In a further aspect, R¹⁴ is substituted with 1, 2, or 3 heteroatoms selected from O, S, and N. It is understood that such replacement will alter the number of substitutent groups (e.g., each O or S substituted for C will decrease the number of substitutent groups by two, whereas each N substituted for C will decrease the number of substitutent groups by one). In a further aspect, heterocycloalkyl can be oxirane, oxetane, tetrahydrofuran, tetrahydro-2H-pyran, oxepane, oxocane, dioxirane, dioxetane, dioxolane, dioxane, dioxepane, dioxocane, thiirane, thietane, tetrahydrothiophene, tetrahydro-2H-thiopyran, thiepane, thiocane, dithiirane, dithietane, dithiolane, dithiane, dithiepane, dithiocane, oxathiirane, oxathietane, oxathiolane, oxathiane, oxathiepane, oxathiocane, aziridine, azetidine, pyrrolidone, piperidine, azepane, azocane, diaziridine, diazetidine, imidazolidine, piperazine, diazepane, diazocane, hexahydropyrimidine, triazinane, oxaziridine, oxazetidine, oxazolidine, morpholine, oxazepane, oxazocane, thiaziridine, thiazetidine, thiazolidine, thiomorpholine, thiazepane, or thiazocane. In a further aspect, heterocycloalkyl can be monocyclic or bicyclic.

In a further aspect, R¹⁴ is optionally substituted Ar³. In a further aspect, R¹⁴ is Ar³.

In a further aspect, R¹⁴ is unsubstituted. In a further aspect, R¹⁴ is substituted with 0-4 groups (e.g., 0-3 groups, 0-2 groups, 0-1 groups, 4 groups, 3 groups, 2 groups, or 1 group). In various aspects, the groups can be selected from halogen (e.g., fluoro, chloro, bromo, or iodo), cyano, hydroxyl, amino, alkylamino, and dialkylamino.

r. R¹⁵ Groups

In one aspect, R¹⁵ is selected from hydrogen and optionally substituted C1-C8 (e.g., C1-C6 or C1-C4) alkyl. In a further aspect, R¹⁵ is selected from hydrogen and C1-C8 alkyl. In a further aspect, R¹⁵ is hydrogen. In a further aspect, R¹⁵ is optionally substituted C1-C8 alkyl. In a further aspect, R¹⁵ is C1-C8 alkyl, for example, methyl, ethyl, propyl, butyl, penyl, hexyl, heptyl, or octyl. In a further aspect, R¹⁵ is unsubstituted. In a further aspect, R¹⁵ is substituted with 0-4 groups (e.g., 0-3 groups, 0-2 groups, 0-1 groups, 4 groups, 3 groups, 2 groups, or 1 group). In various aspects, the groups can be selected from halogen (e.g., fluoro, chloro, bromo, or iodo), cyano, hydroxyl, amino, alkylamino, and dialkylamino.

s. Integer Values (m, n, p, and q)

In one aspect, m is an integer from 0-3. In a further aspect, m is an integer from 0-2. In a yet further aspect, m is an integer that is 0 or 1. In a still further aspect, m is 0. In an even further aspect, m is 1. In a yet further aspect, m is 2. In a still further aspect, m is 3. In an even further aspect, m is an integer from 1-3. In a yet further aspect, m is an integer from 2-3.

In one aspect, n is an integer from 0-3. In a further aspect, n is an integer from 0-2. In a yet further aspect, n is an integer that is 0 or 1. In a still further aspect, n is 0. In an even further aspect, n is 1. In a yet further aspect, n is 2. In a still further aspect, n is 3. In an even further aspect, n is an integer from 1-3. In a yet further aspect, n is an integer from 2-3.

In one aspect, p is an integer from 0-3. In a further aspect, p is an integer from 0-2. In a yet further aspect, p is an integer that is 0 or 1. In a still further aspect, p is 0. In an even further aspect, p is 1. In a yet further aspect, p is 2. In a still further aspect, p is 3. In an even further aspect, p is an integer from 1-3. In a yet further aspect, p is an integer from 2-3.

In one aspect, q is an integer from 0-3. In a further aspect, q is an integer from 0-2. In a yet further aspect, q is an integer that is 0 or 1. In a still further aspect, q is 0. In an even further aspect, q is 1. In a yet further aspect, q is 2. In a still further aspect, q is 3. In an even further aspect, q is an integer from 1-3. In a yet further aspect, q is an integer from 2-3.

T. Halogen (X)

In one aspect, halogen is fluoro, chloro, bromo or iodo. In a still further aspect, halogen is fluoro, chloro, or bromo. In a yet further aspect, halogen is fluoro or chloro. In a further aspect, halogen is fluoro. In an even further aspect, halogen is chloro or bromo. In an even further aspect, halogen is chloro. In a yet further aspect, halogen is iodo. In a still further aspect, halogen is bromo.

It is also contemplated that pseudohalogens (e.g. triflate, mesylate, brosylate, etc.) can be used as leaving groups in place of halogens in certain aspects.

2. Example Compounds

In one aspect, a compound can be present as one or more of the following structures:

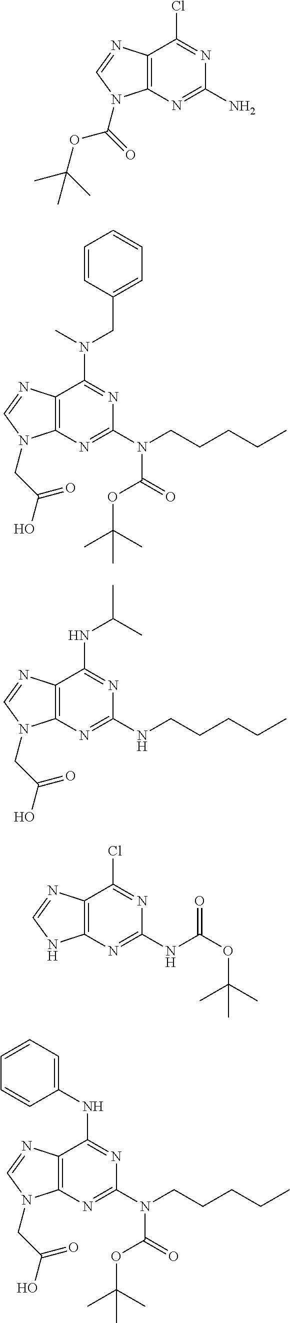

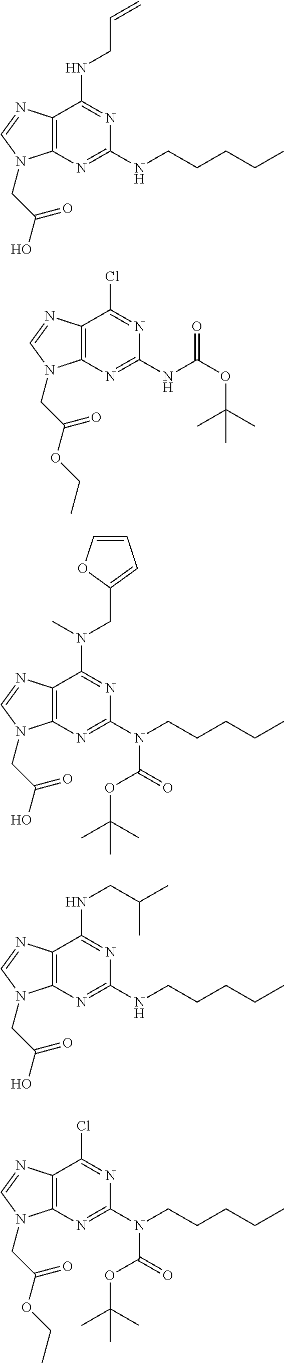

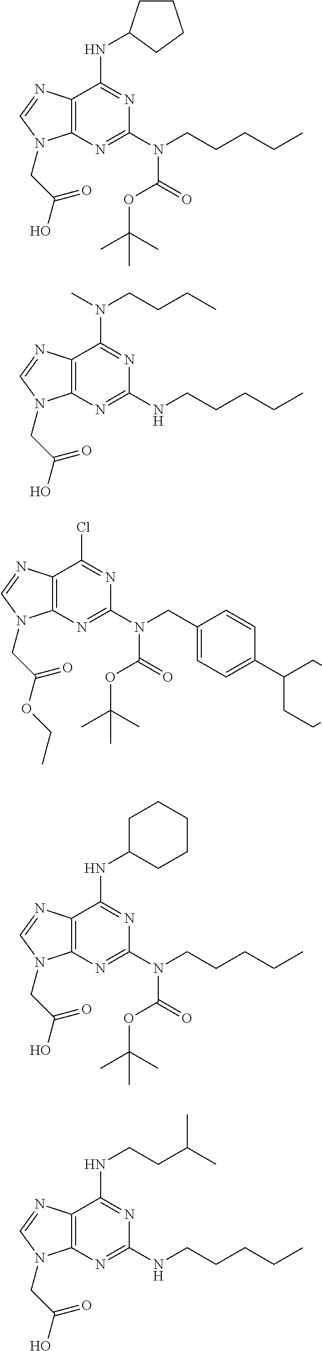

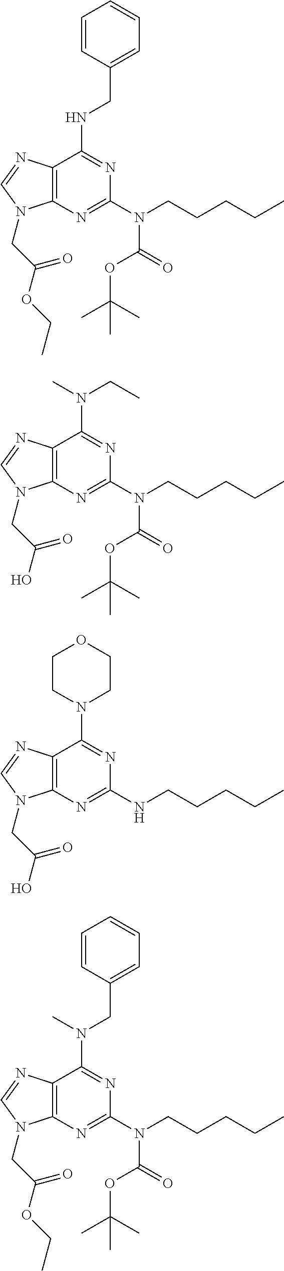

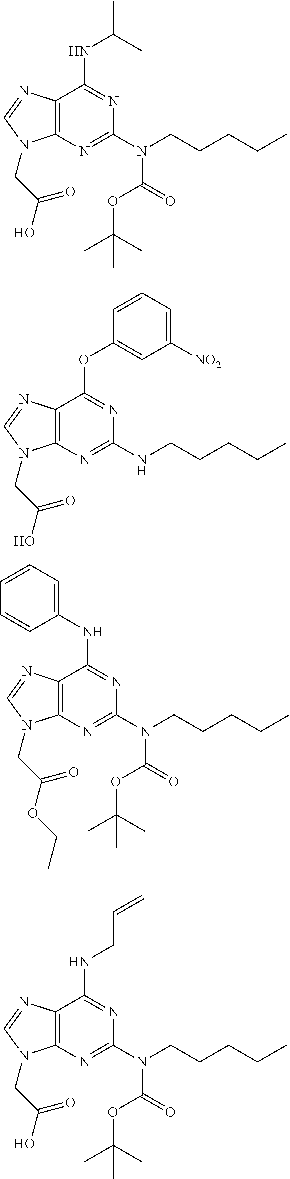

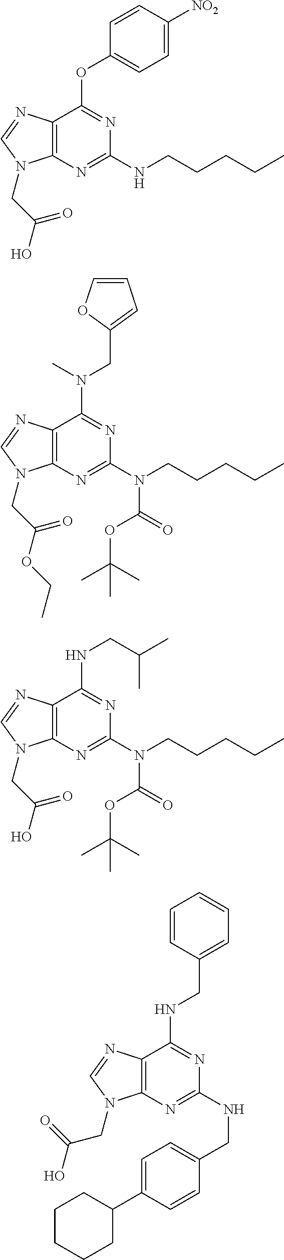

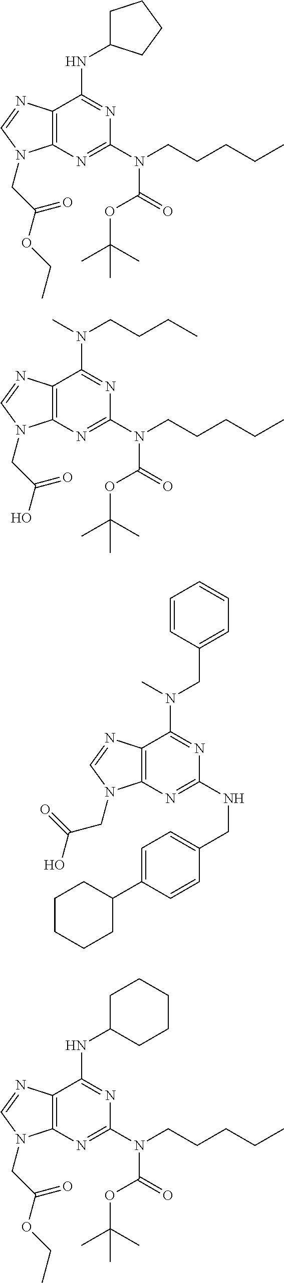

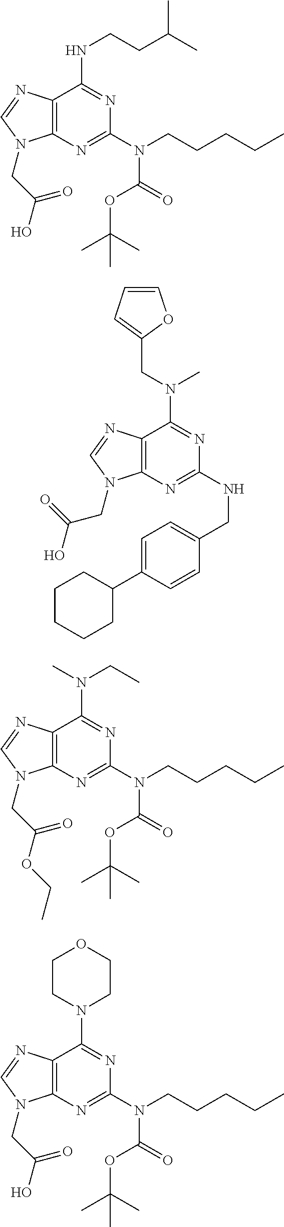

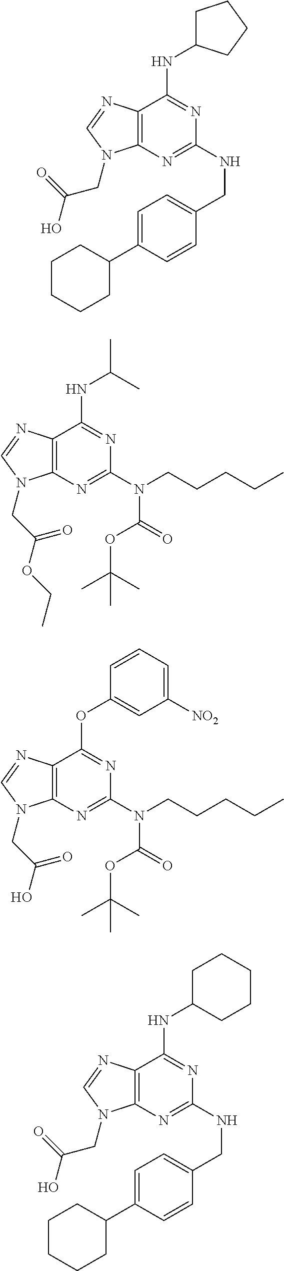

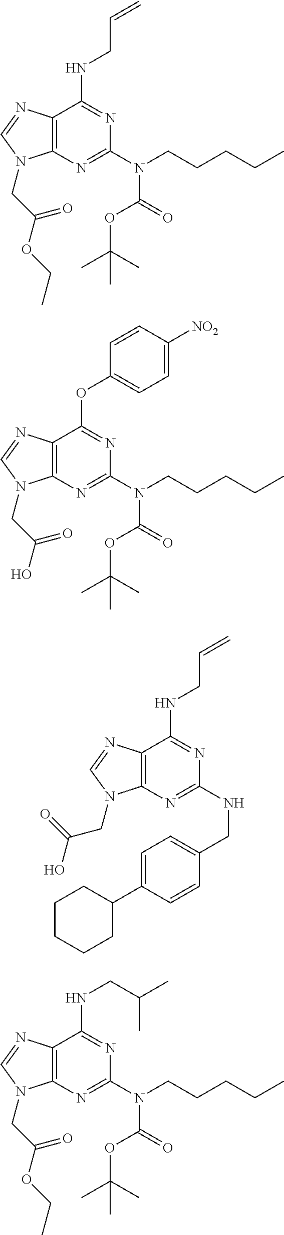

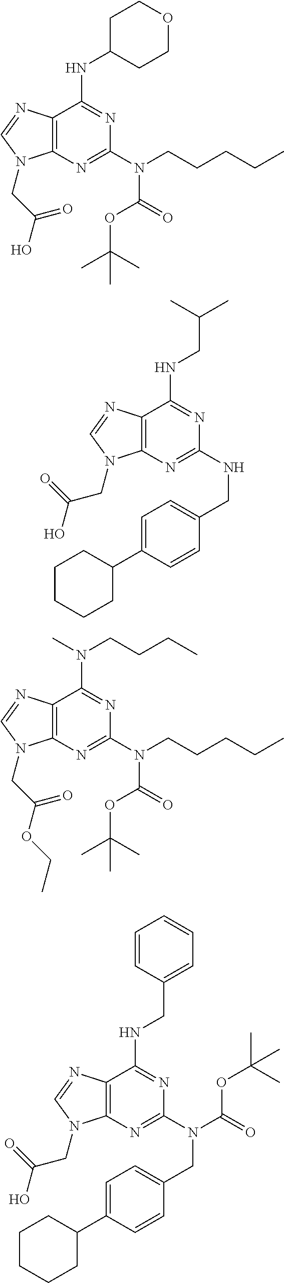

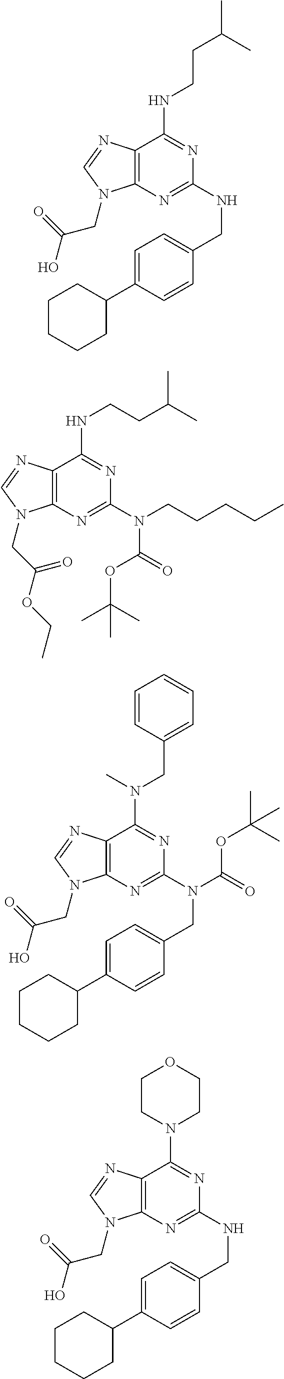 [SMILES: CC(C)CCNc1nc(NCc2ccc(C3CCCCC3)cc2)nc2c1ncn2CC(=O)O ; CCCCCN(C(=O)OC(C)(C)C)c1nc(NCCC(C)C)c2ncn(CC(=O)OCC)c2n1 ; CN(Cc1ccccc1)c1nc(N(Cc2ccc(C3CCCCC3)cc2)C(=O)OC(C)(C)C)nc2c1ncn2CC(=O)O ; O=C(O)Cn1cnc2c(N3CCOCC3)nc(NCc3ccc(C4CCCCC4)cc3)nc21]

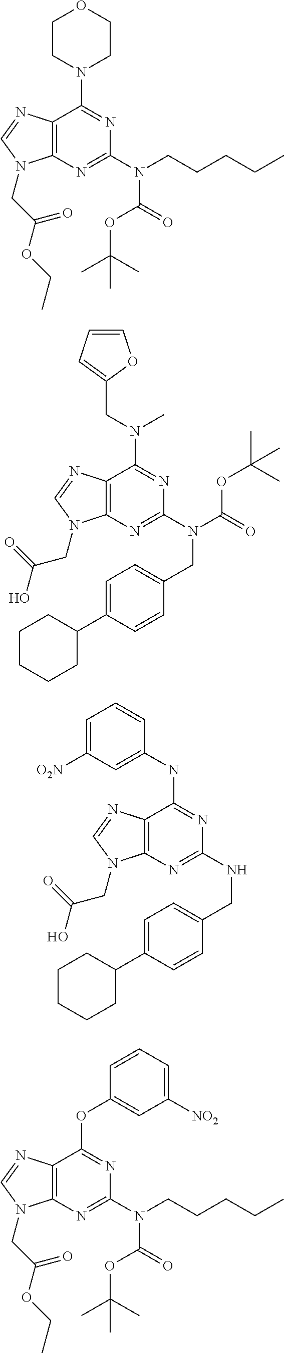

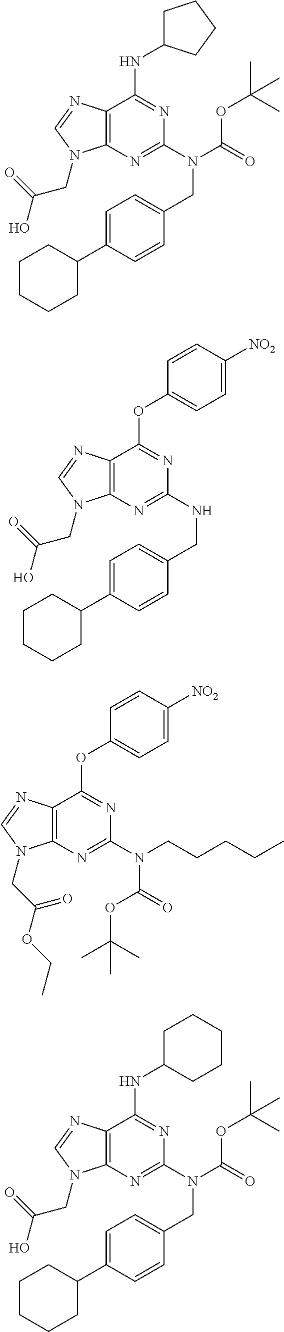 [SMILES: CC(C)(C)OC(=O)N(Cc1ccc(C2CCCCC2)cc1)c1nc(NC2CCCC2)c2ncn(CC(=O)O)c2n1 ; O=C(O)Cn1cnc2c(Oc3ccc([N+](=O)[O-])cc3)nc(NCc3ccc(C4CCCCC4)cc3)nc21 ; CCCCCN(C(=O)OC(C)(C)C)c1nc(Oc2ccc([N+](=O)[O-])cc2)c2ncn(CC(=O)OCC)c2n1 ; CC(C)(C)OC(=O)N(Cc1ccc(C2CCCCC2)cc1)c1nc(NC2CCCCC2)c2ncn(CC(=O)O)c2n1]

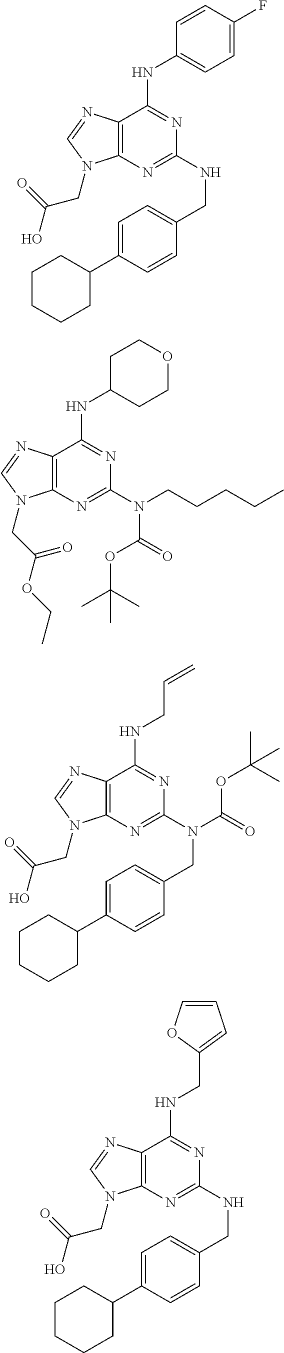 [SMILES: O=C(O)Cn1cnc2c(Nc3ccc(F)cc3)nc(NCc3ccc(C4CCCCC4)cc3)nc21 ; CCCCCN(C(=O)OC(C)(C)C)c1nc(NC2CCOCC2)c2ncn(CC(=O)OCC)c2n1 ; C=CCNc1nc(N(Cc2ccc(C3CCCCC3)cc2)C(=O)OC(C)(C)C)nc2c1ncn2CC(=O)O ; O=C(O)Cn1cnc2c(NCc3ccco3)nc(NCc3ccc(C4CCCCC4)cc3)nc21]

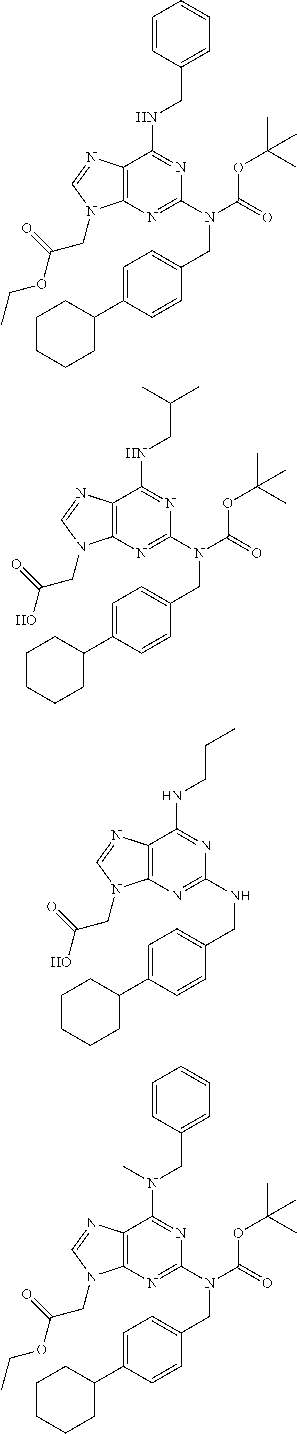

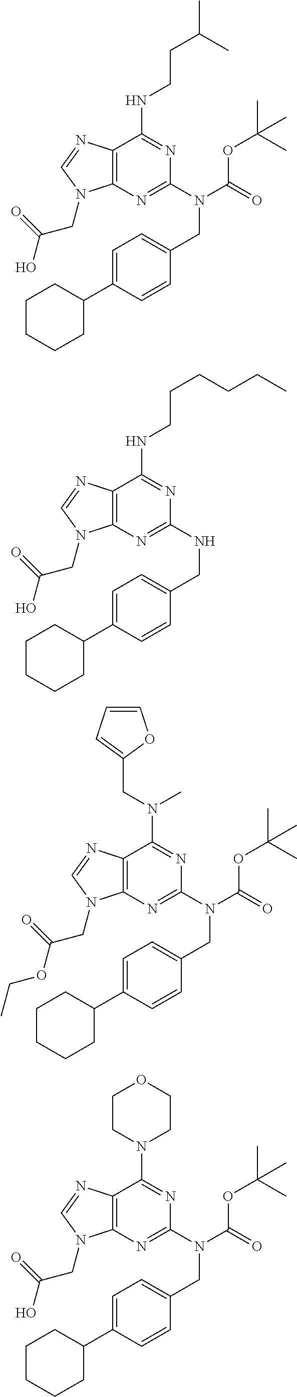

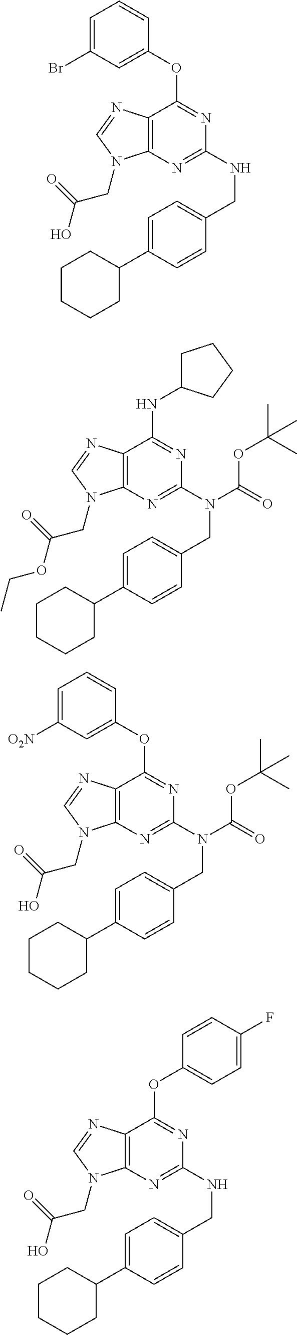

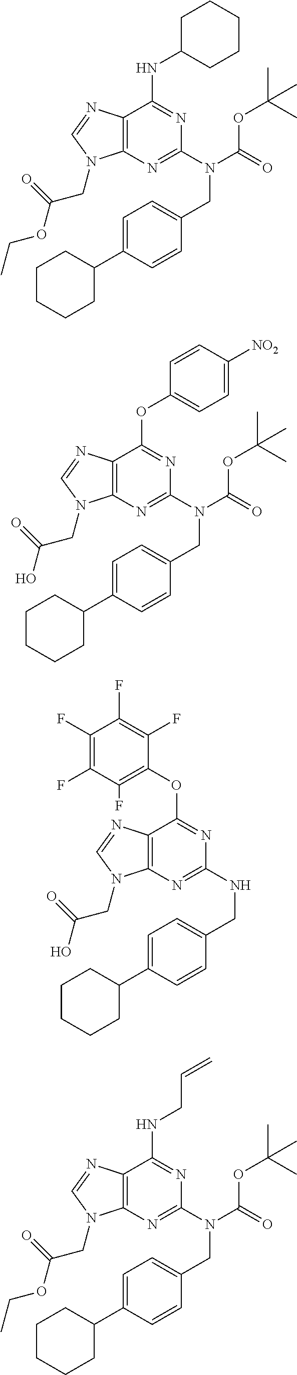

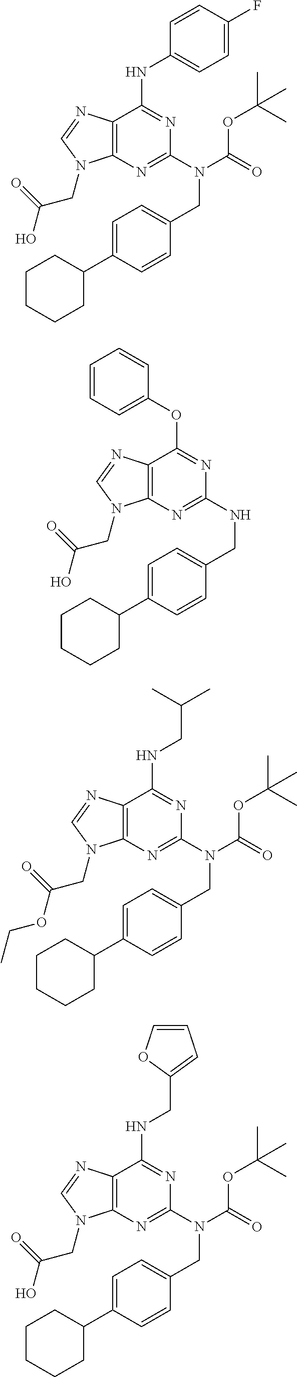

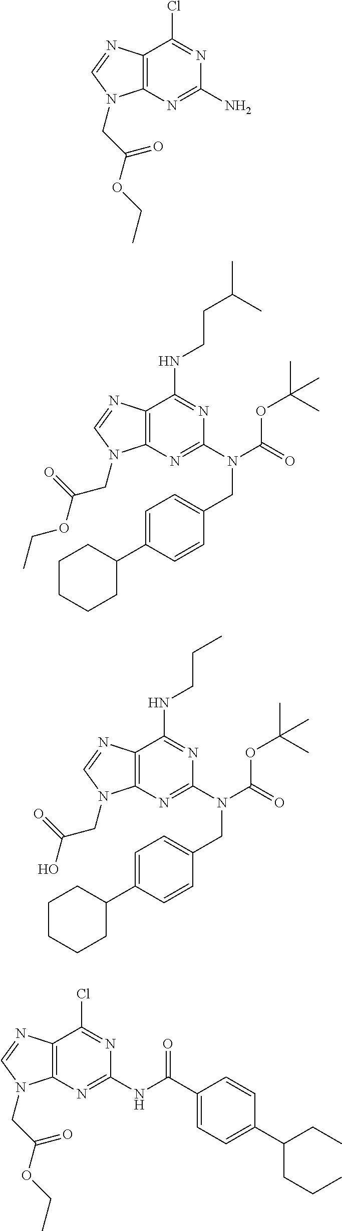

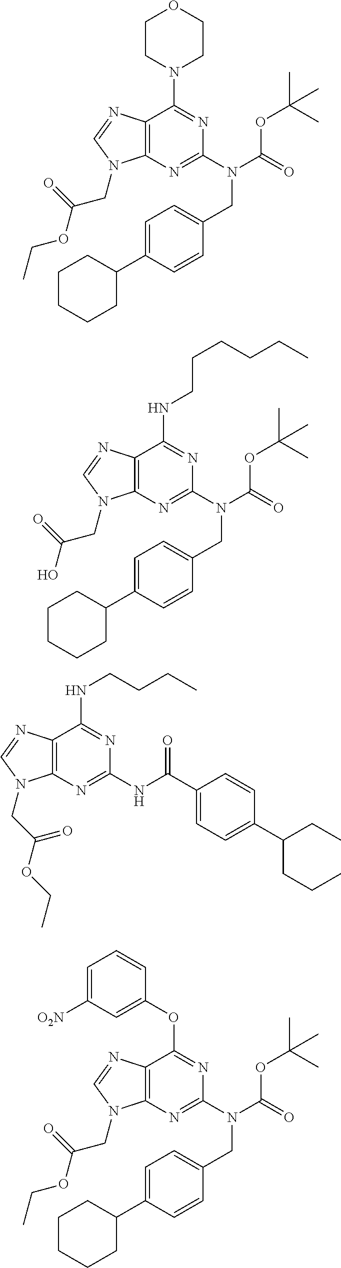

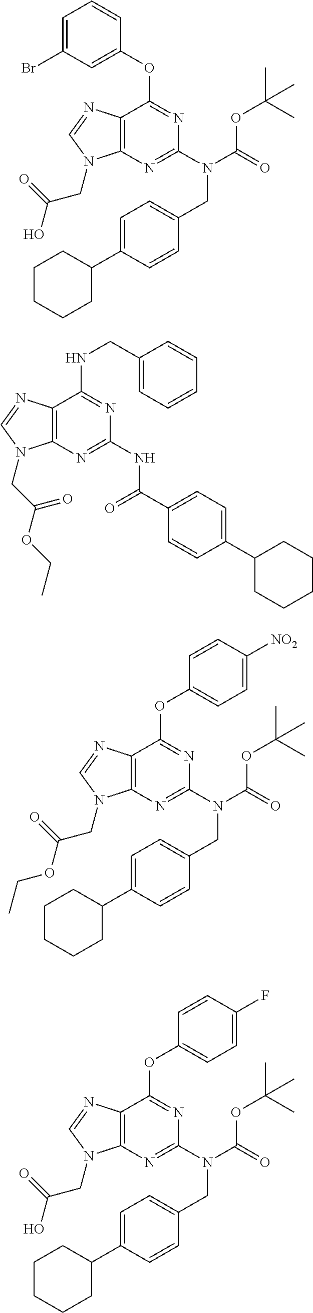

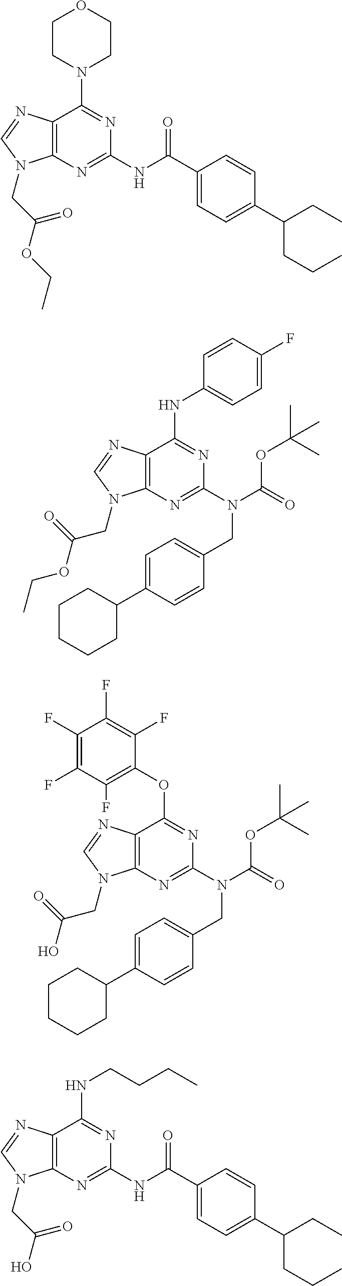

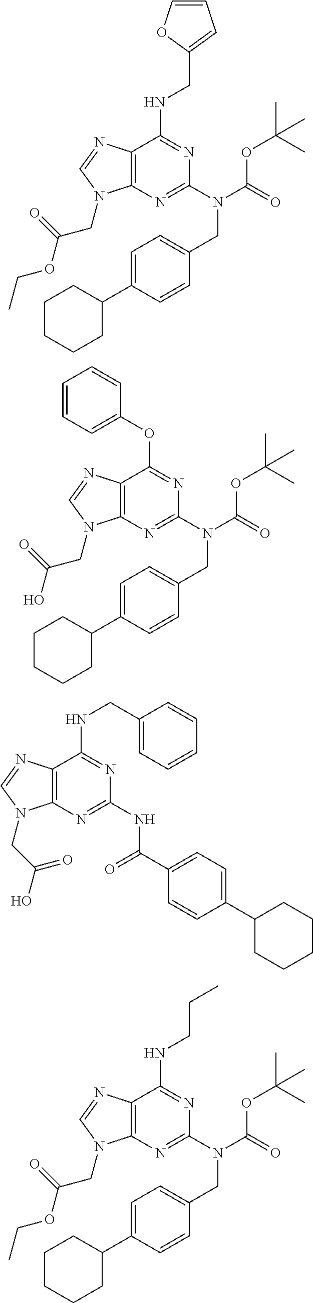

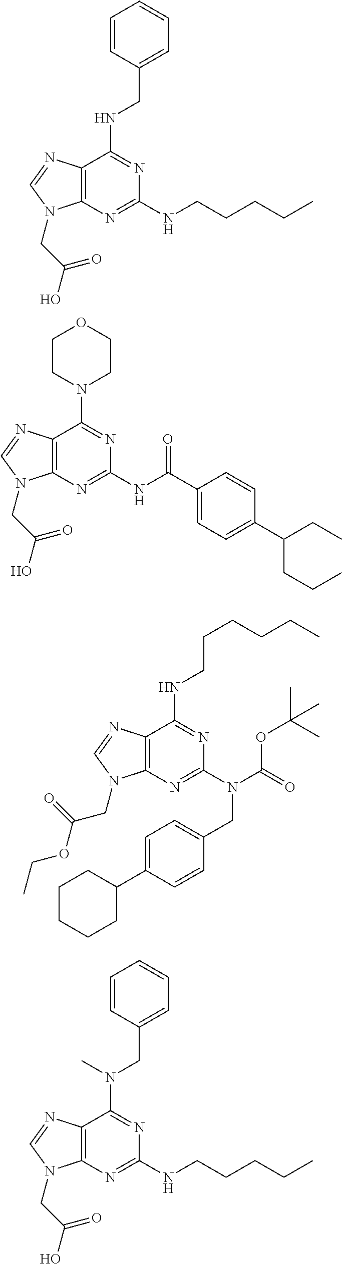

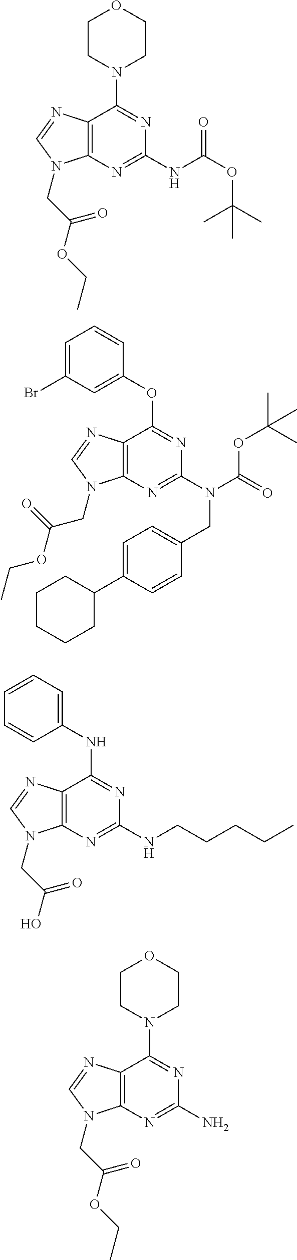

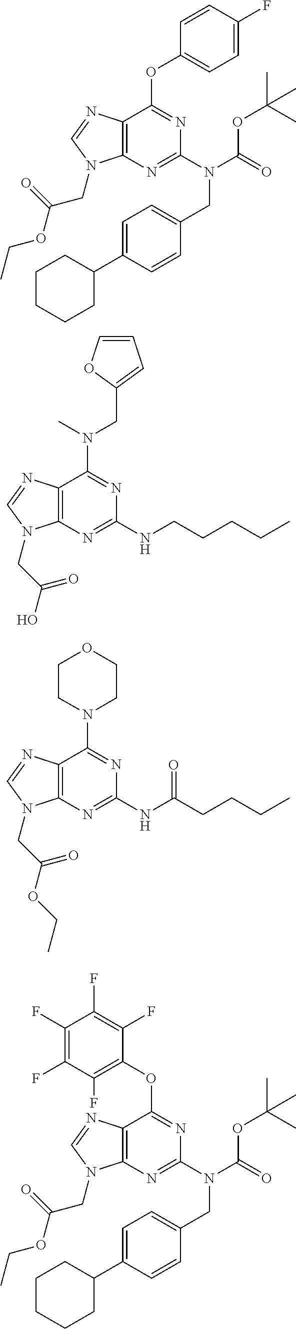

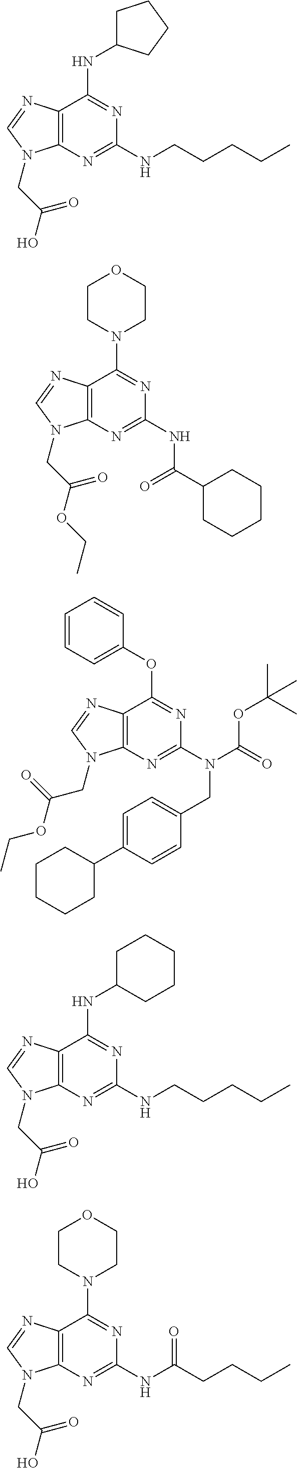

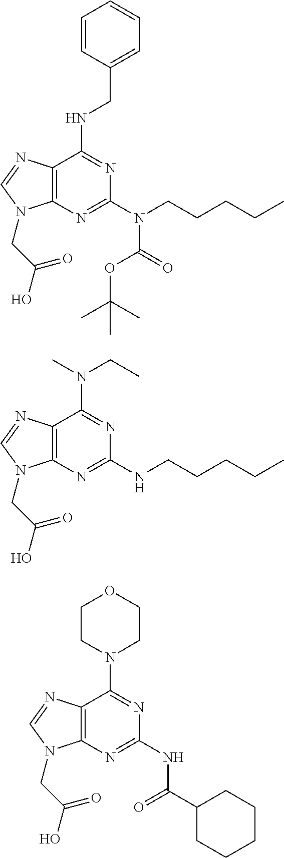

In one aspect, a compound can be present as one or more of the following structures:

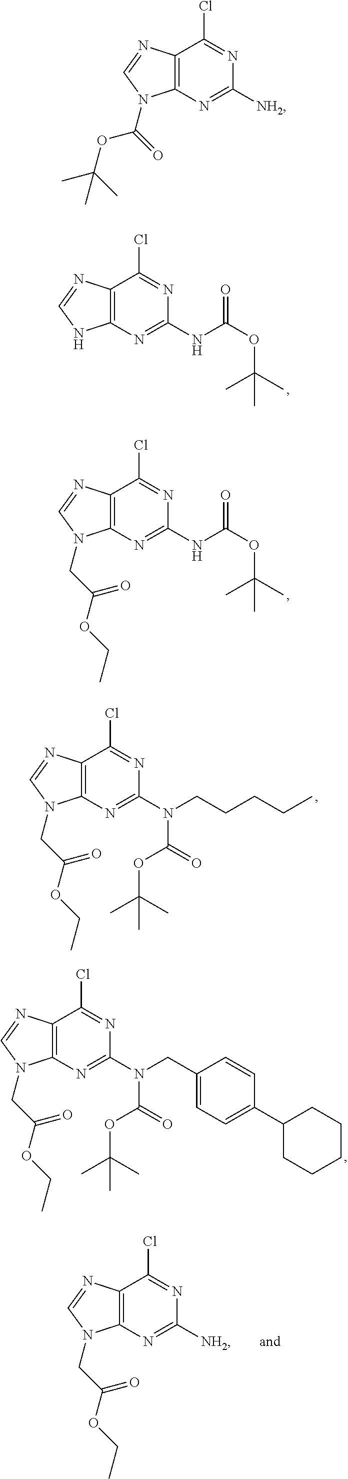

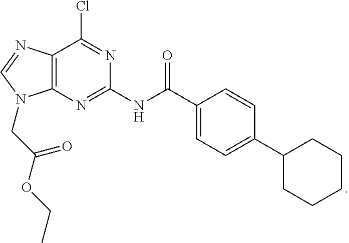 [SMILES: CCOC(=O)Cn1cnc2c(Cl)nc(NC(=O)c3ccc(C4CCCCC4)cc3)nc21]

In one aspect, a compound can be present as one or more of the following structures:

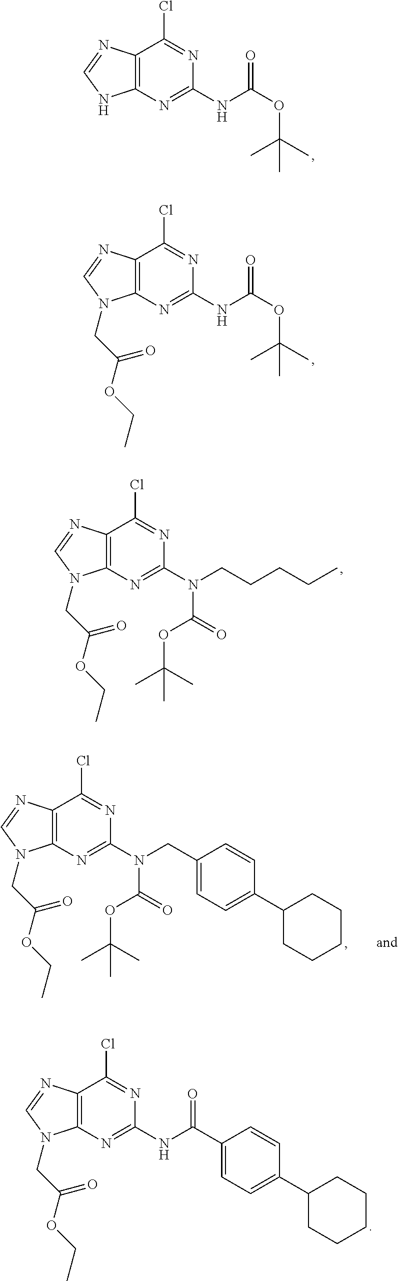

In one aspect, a compound can be present as one or more of the following structures:

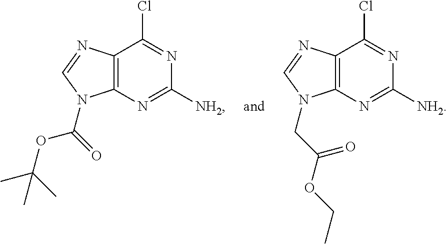 [SMILES: CC(C)(C)OC(=O)n1cnc2c(Cl)nc(N)nc21 ; CCOC(=O)Cn1cnc2c(Cl)nc(N)nc21]

In one aspect, a compound can be present as one or more of the following structures:

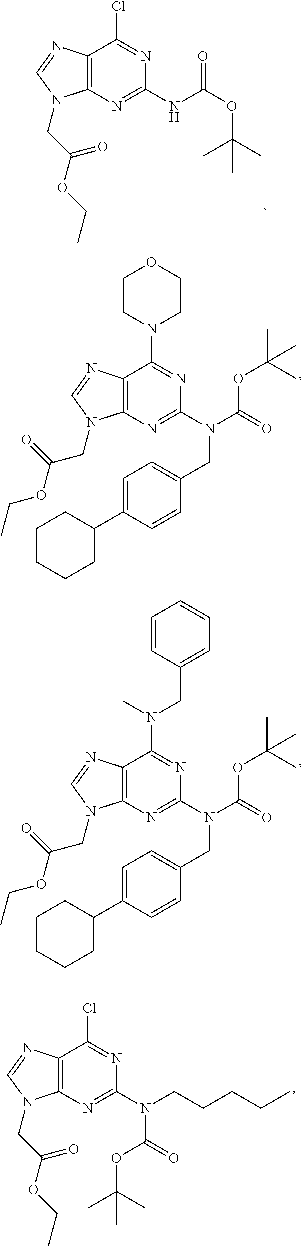

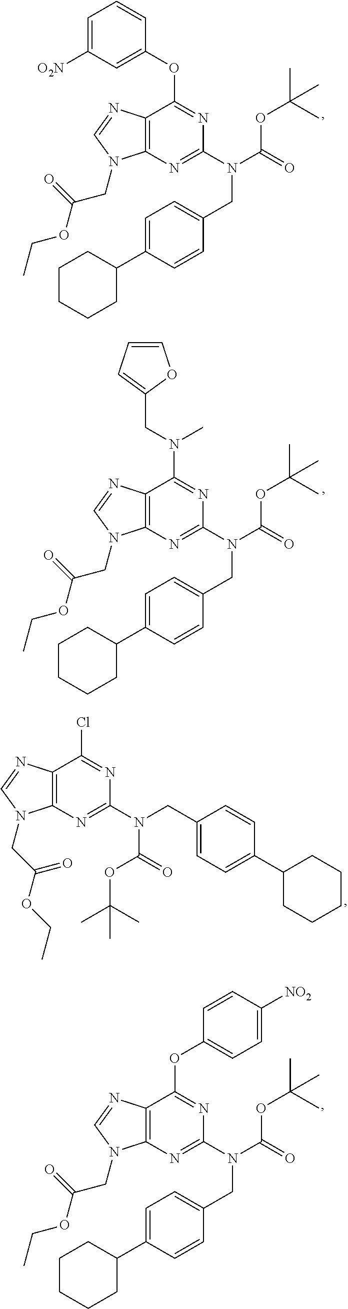

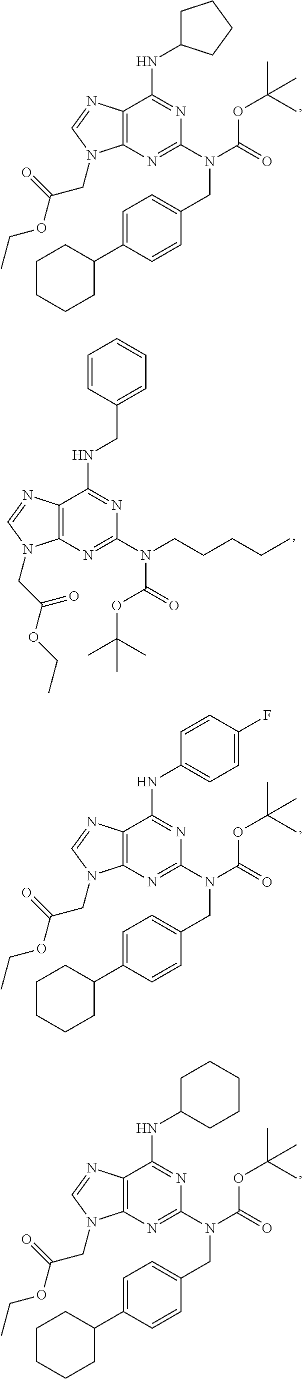

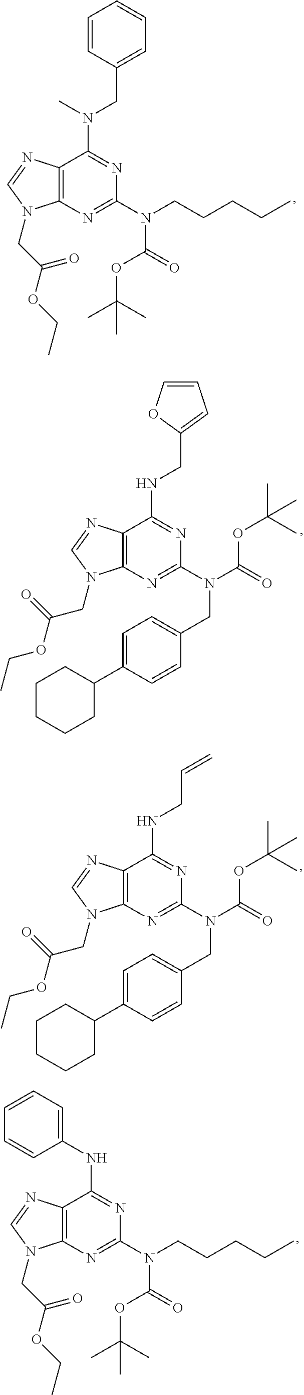

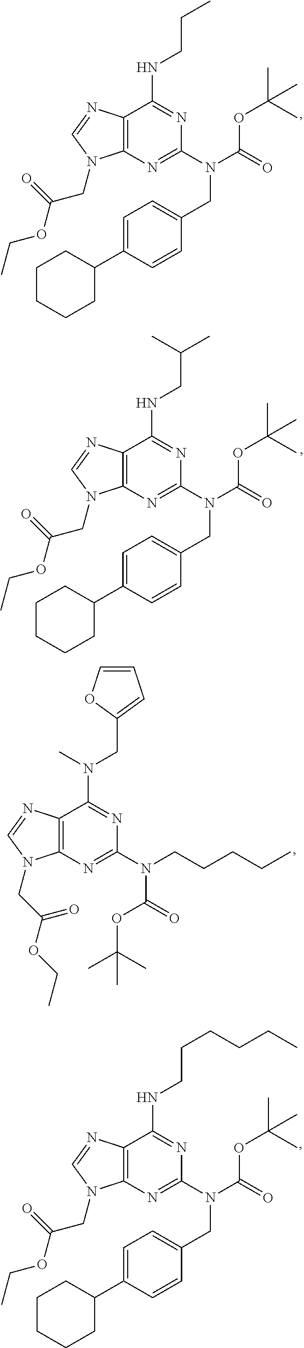

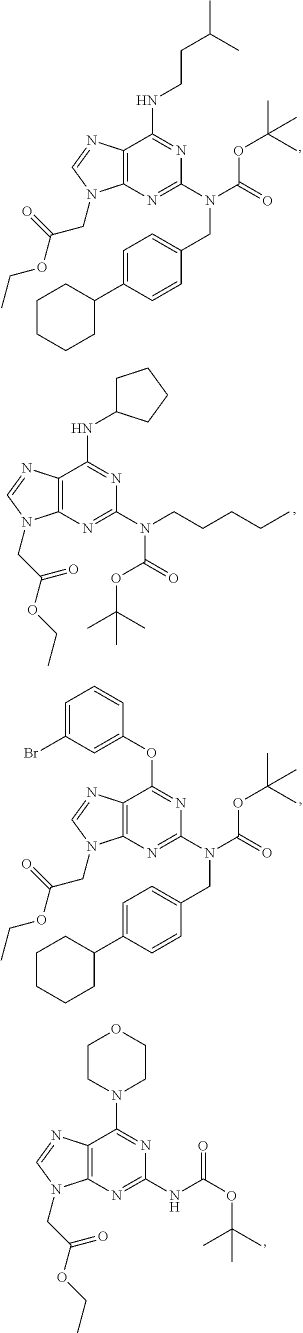

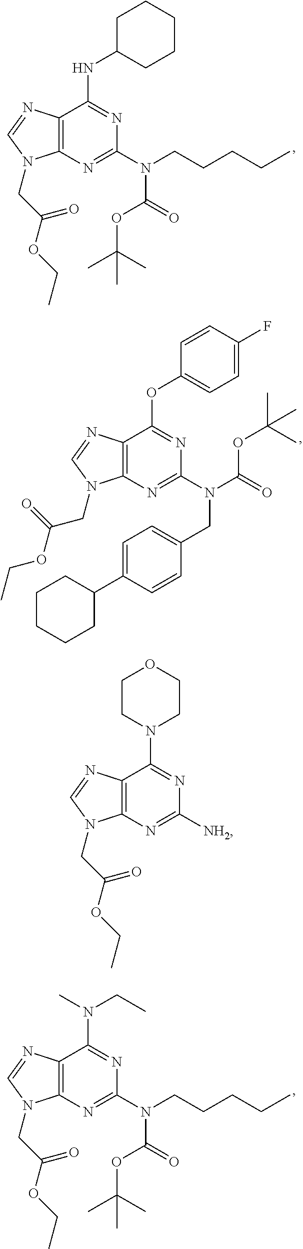

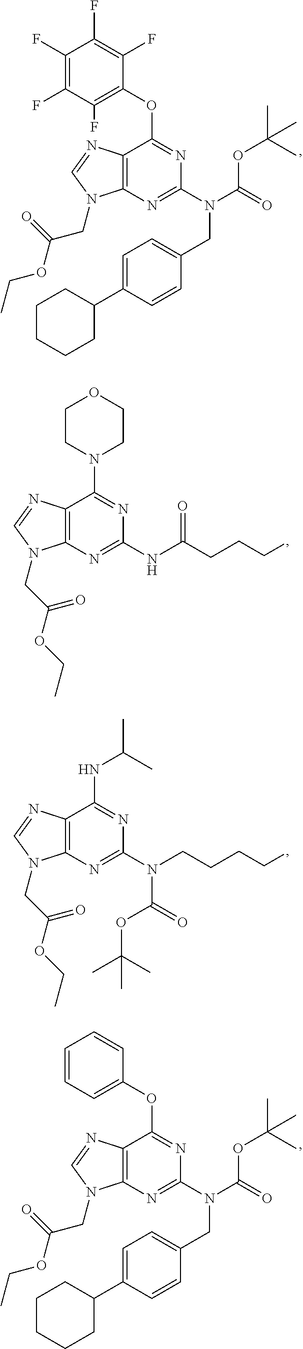

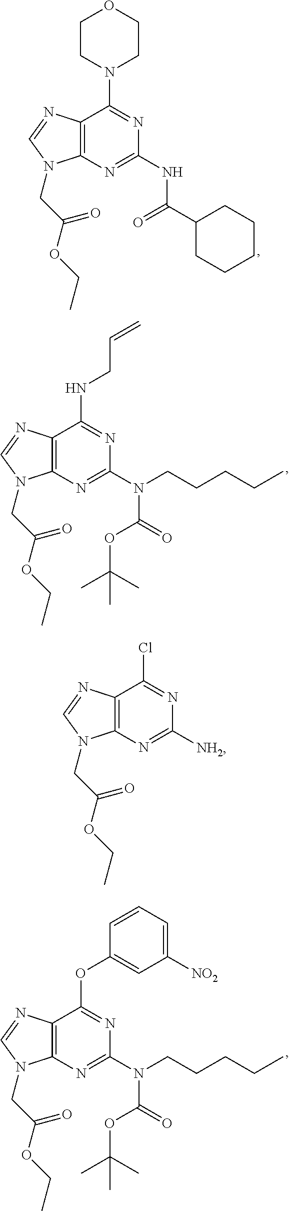

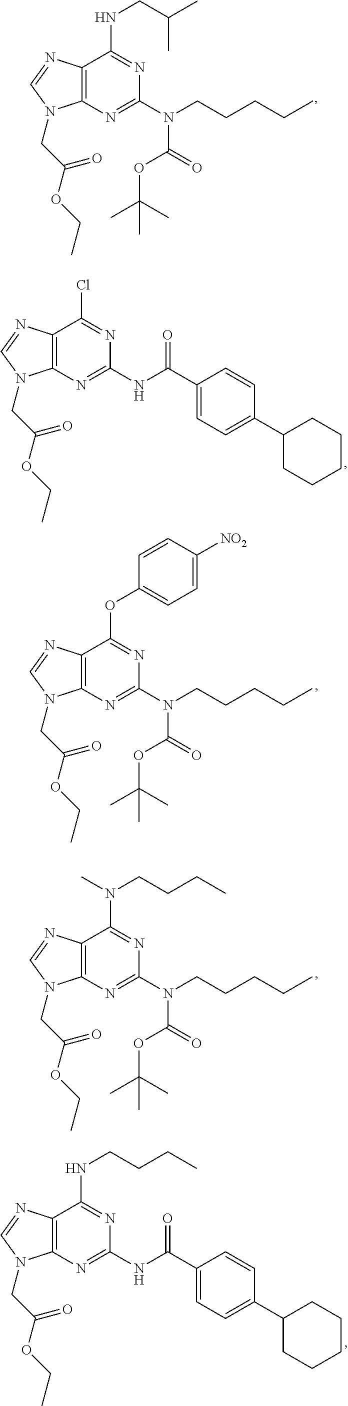

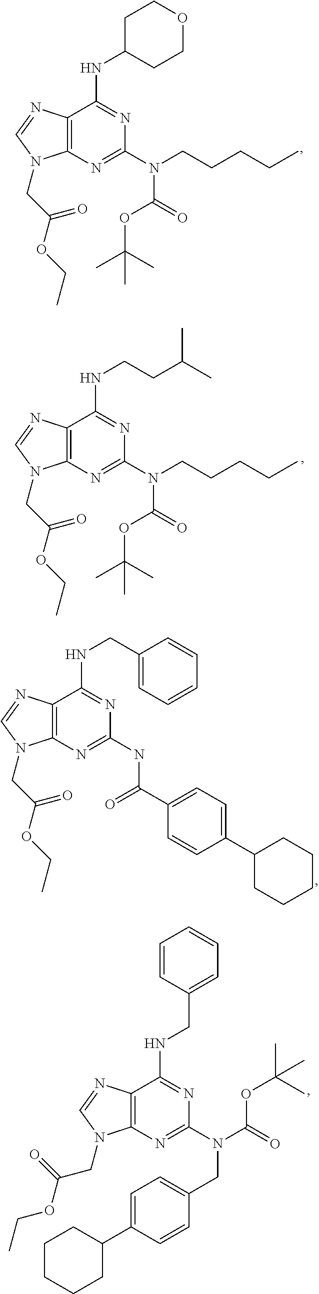

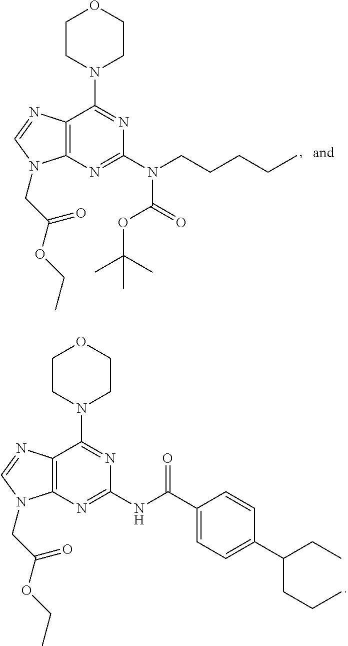

In one aspect, a compound can be present as one or more of the following structures:

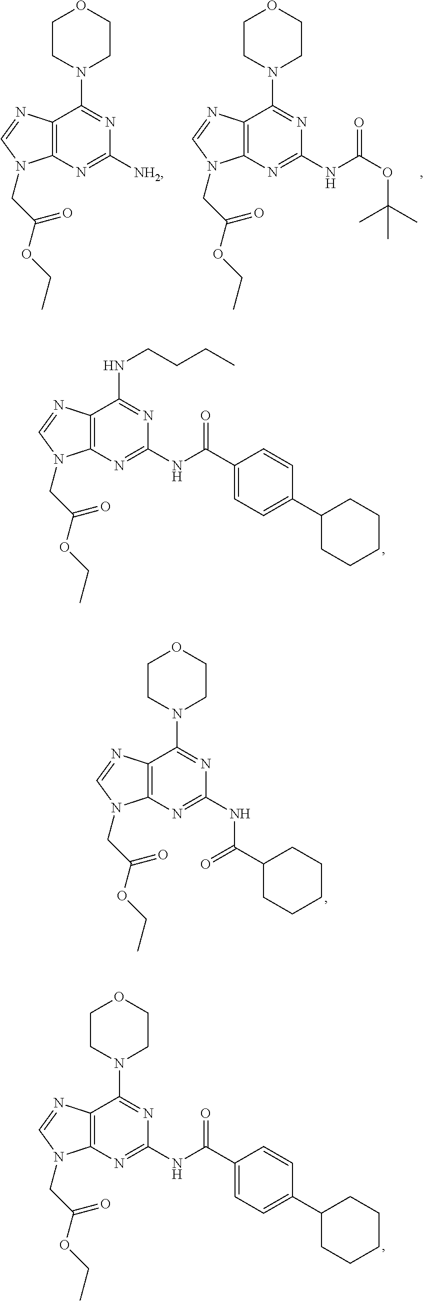 [SMILES: CCOC(=O)Cn1cnc2c(N3CCOCC3)nc(N)nc21 ; CCOC(=O)Cn1cnc2c(N3CCOCC3)nc(NC(=O)OC(C)(C)C)nc21 ; CCCCNc1nc(NC(=O)c2ccc(C3CCCCC3)cc2)nc2c1ncn2CC(=O)OCC ; CCOC(=O)Cn1cnc2c(N3CCOCC3)nc(NC(=O)C3CCCCC3)nc21 ; CCOC(=O)Cn1cnc2c(N3CCOCC3)nc(NC(=O)c3ccc(C4CCCCC4)cc3)nc21]

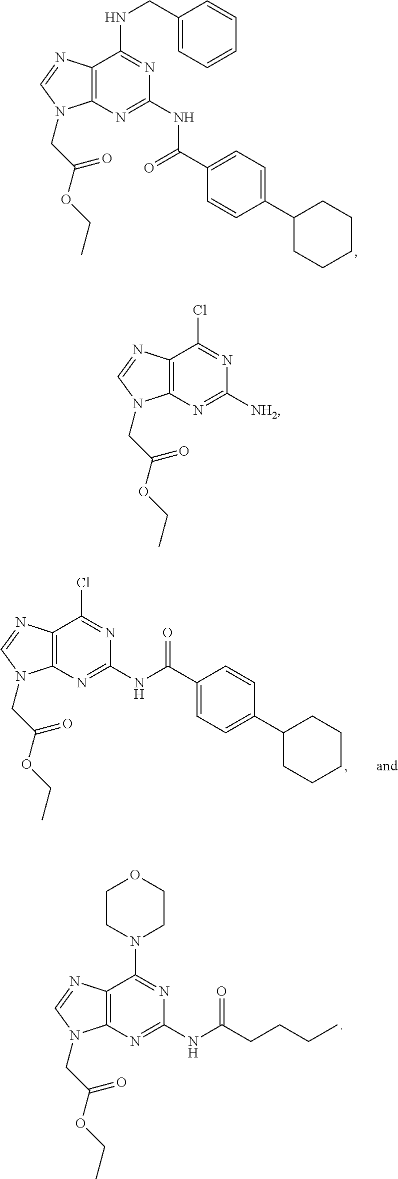 [SMILES: CCOC(=O)Cn1cnc2c(NCc3ccccc3)nc(NC(=O)c3ccc(C4CCCCC4)cc3)nc21 ; CCOC(=O)Cn1cnc2c(Cl)nc(N)nc21 ; CCOC(=O)Cn1cnc2c(Cl)nc(NC(=O)c3ccc(C4CCCCC4)cc3)nc21 ; CCCCC(=O)Nc1nc(N2CCOCC2)c2ncn(CC(=O)OCC)c2n1]

In one aspect, a compound can be present as one or more of the following structures:

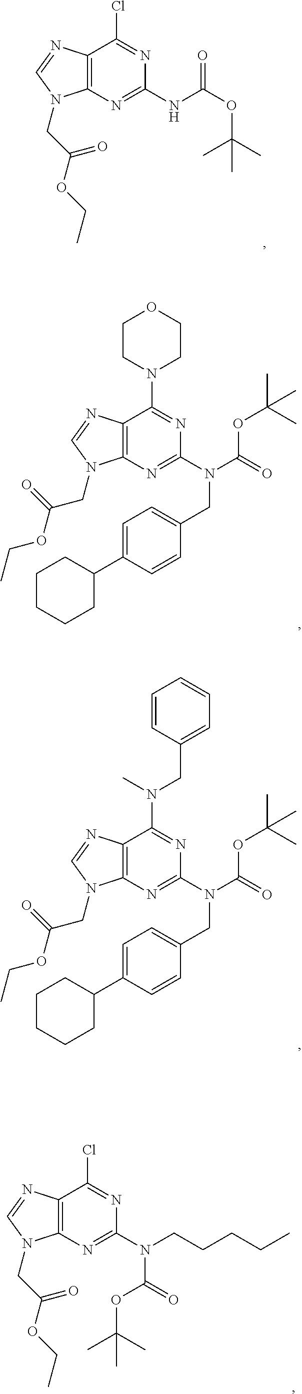

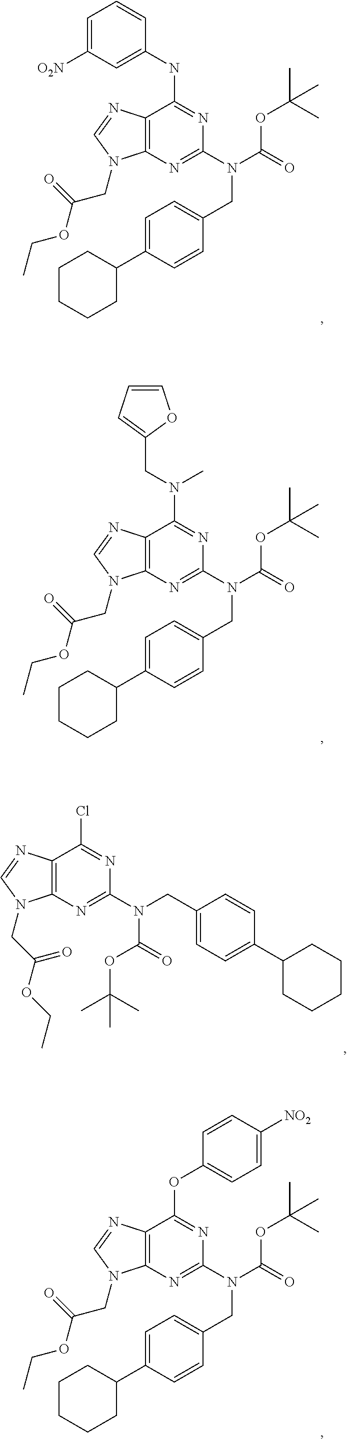

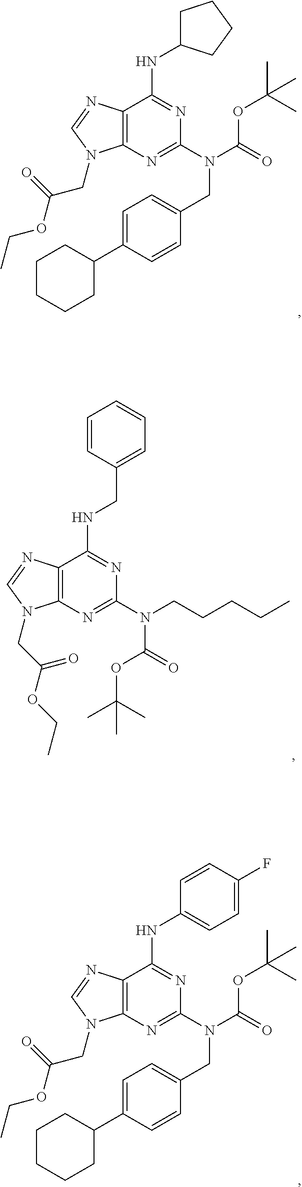

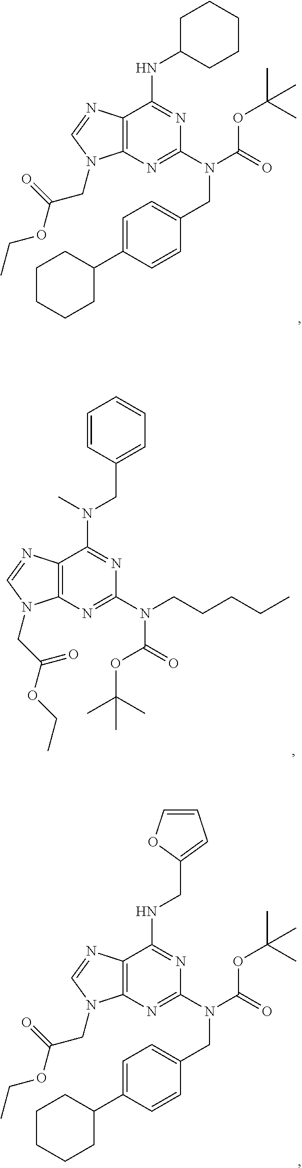

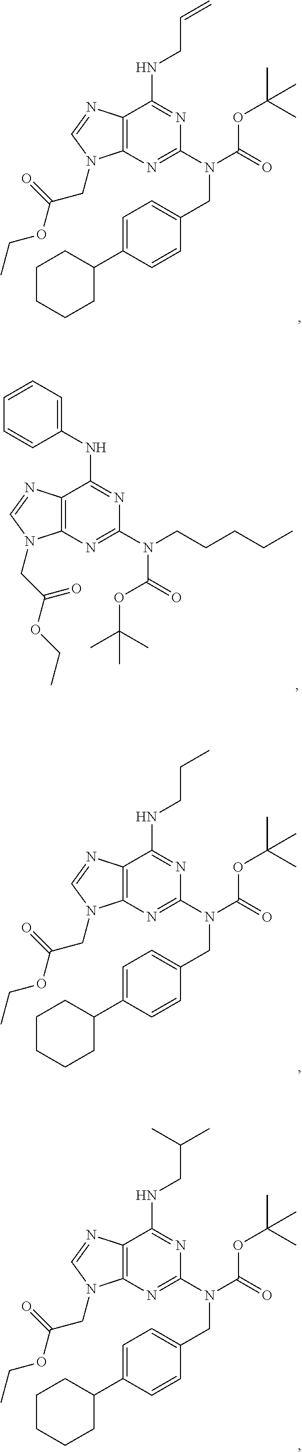

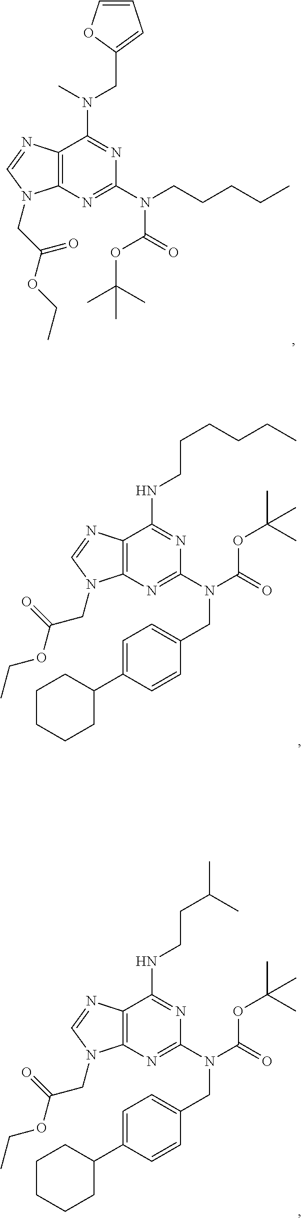

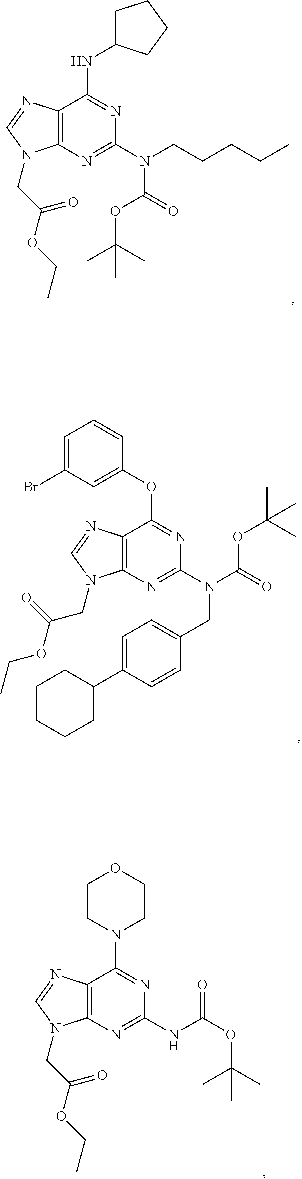

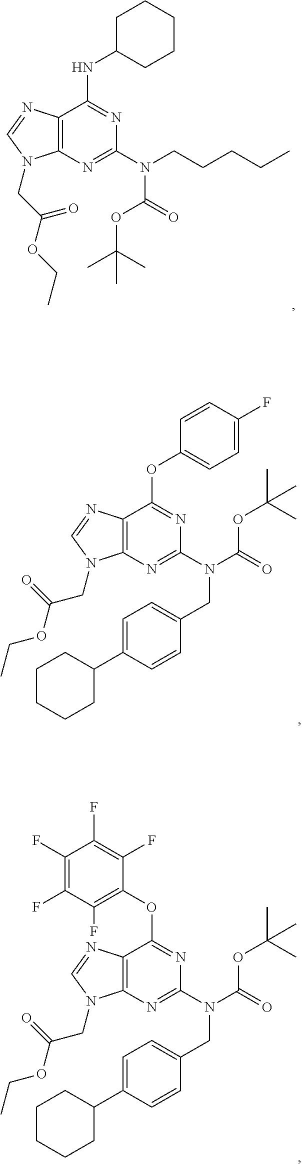

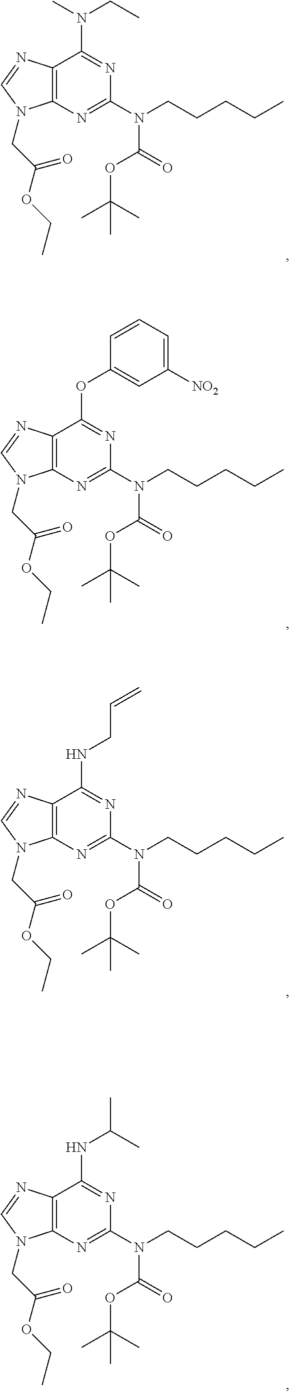 [SMILES: CCCCCN(C(=O)OC(C)(C)C)c1nc(N(C)CC)c2ncn(CC(=O)OCC)c2n1 ; CCCCCN(C(=O)OC(C)(C)C)c1nc(Oc2cccc([N+](=O)[O-])c2)c2ncn(CC(=O)OCC)c2n1 ; C=CCNc1nc(N(CCCCC)C(=O)OC(C)(C)C)nc2c1ncn2CC(=O)OCC ; CCCCCN(C(=O)OC(C)(C)C)c1nc(NC(C)C)c2ncn(CC(=O)OCC)c2n1]

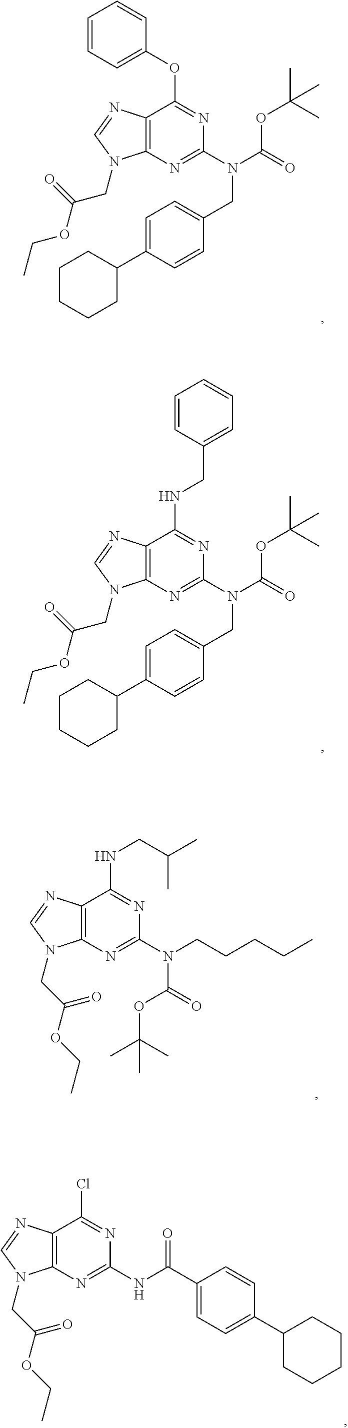

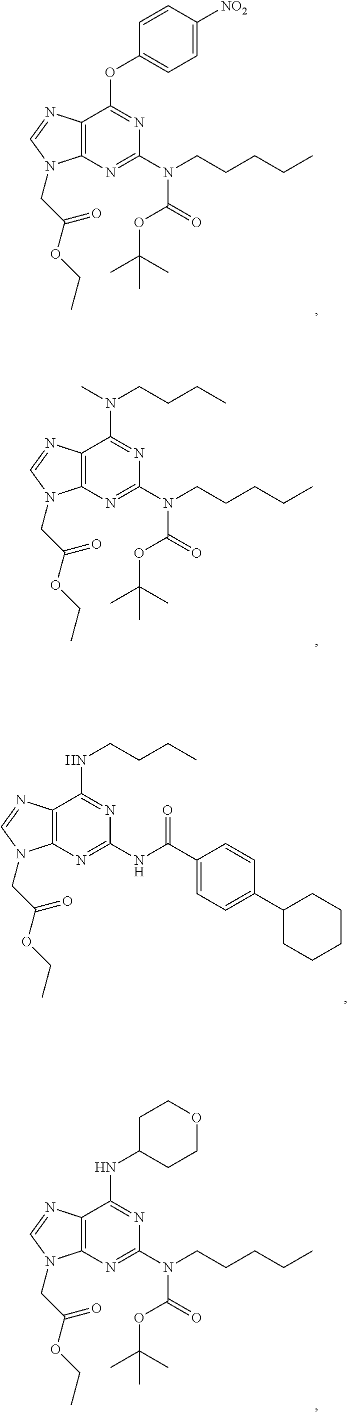 [SMILES: CCCCCN(C(=O)OC(C)(C)C)c1nc(Oc2ccc([N+](=O)[O-])cc2)c2ncn(CC(=O)OCC)c2n1 ; CCCCCN(C(=O)OC(C)(C)C)c1nc(N(C)CCCC)c2ncn(CC(=O)OCC)c2n1 ; CCCCNc1nc(NC(=O)c2ccc(C3CCCCC3)cc2)nc2c1ncn2CC(=O)OCC ; CCCCCN(C(=O)OC(C)(C)C)c1nc(NC2CCOCC2)c2ncn(CC(=O)OCC)c2n1]

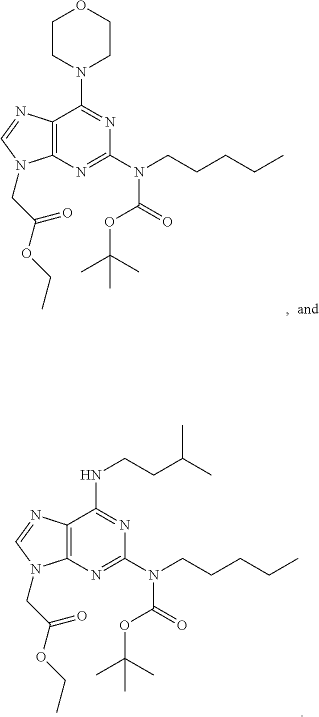 [SMILES: CCCCCN(C(=O)OC(C)(C)C)c1nc(N2CCOCC2)c2ncn(CC(=O)OCC)c2n1 ; CCCCCN(C(=O)OC(C)(C)C)c1nc(NCCC(C)C)c2ncn(CC(=O)OCC)c2n1]

In one aspect, a compound can be present as the following structure:

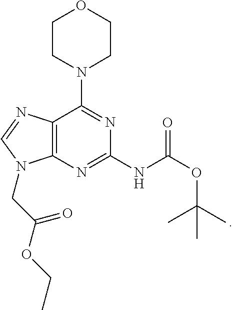 [SMILES: CCOC(=O)Cn1cnc2c(N3CCOCC3)nc(NC(=O)OC(C)(C)C)nc21]

In one aspect, a compound can be present as one or more of the following structures:

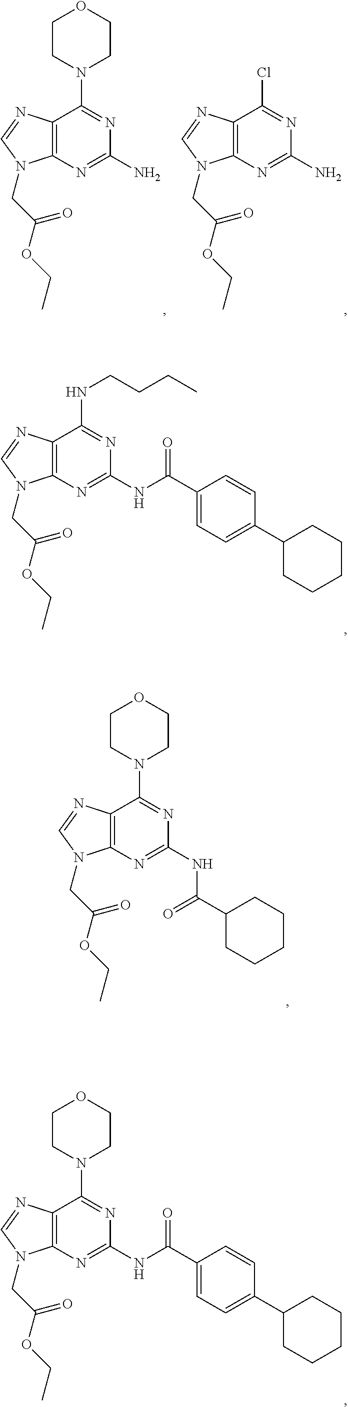 [SMILES: CCOC(=O)Cn1cnc2c(N3CCOCC3)nc(N)nc21 ; CCOC(=O)Cn1cnc2c(Cl)nc(N)nc21 ; CCCCNc1nc(NC(=O)c2ccc(C3CCCCC3)cc2)nc2c1ncn2CC(=O)OCC ; CCOC(=O)Cn1cnc2c(N3CCOCC3)nc(NC(=O)C3CCCCC3)nc21 ; CCOC(=O)Cn1cnc2c(N3CCOCC3)nc(NC(=O)c3ccc(C4CCCCC4)cc3)nc21]

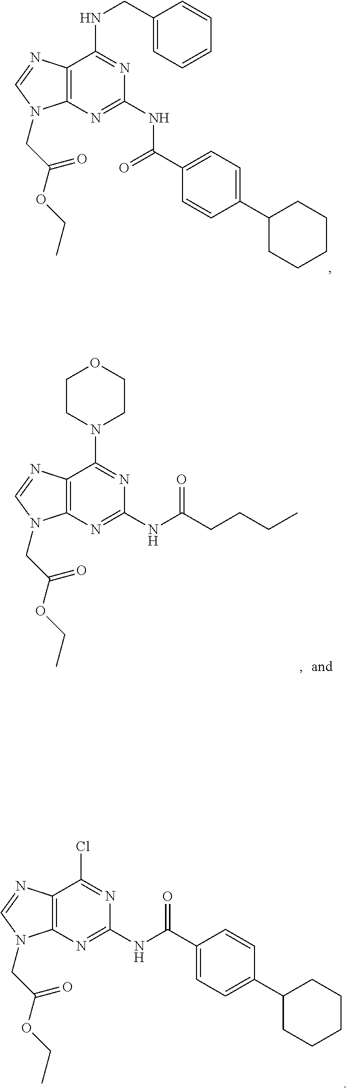 [SMILES: CCOC(=O)Cn1cnc2c(NCc3ccccc3)nc(NC(=O)c3ccc(C4CCCCC4)cc3)nc21 ; CCCCC(=O)Nc1nc(N2CCOCC2)c2ncn(CC(=O)OCC)c2n1 ; CCOC(=O)Cn1cnc2c(Cl)nc(NC(=O)c3ccc(C4CCCCC4)cc3)nc21]

In one aspect, a compound can be present as one or more of the following structures:

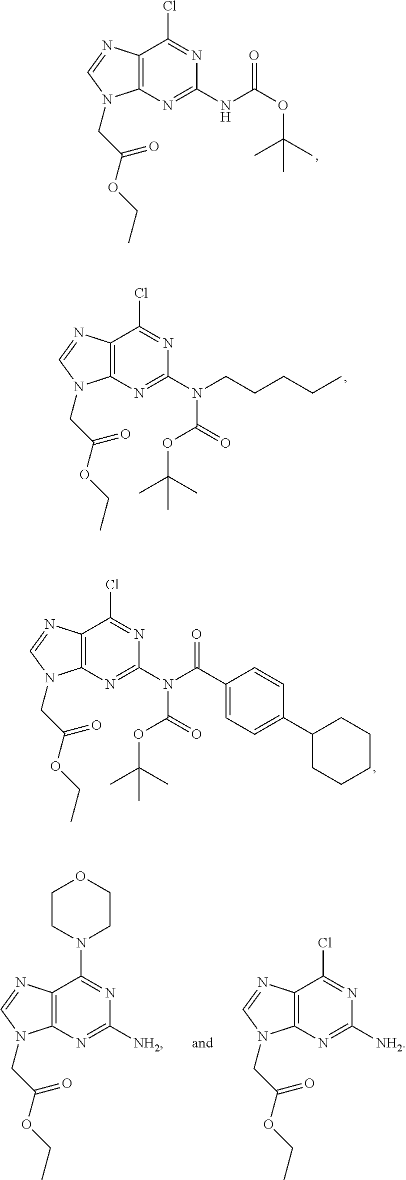 [SMILES: CCOC(=O)Cn1cnc2c(Cl)nc(NC(=O)OC(C)(C)C)nc21 ; CCCCCN(C(=O)OC(C)(C)C)c1nc(Cl)c2ncn(CC(=O)OCC)c2n1 ; CCOC(=O)Cn1cnc2c(Cl)nc(N(C(=O)OC(C)(C)C)C(=O)c3ccc(C4CCCCC4)cc3)nc21 ; CCOC(=O)Cn1cnc2c(N3CCOCC3)nc(N)nc21 ; "and" ; CCOC(=O)Cn1cnc2c(Cl)nc(N)nc21]

In one aspect, a compound can be present as one or more of the following structures:

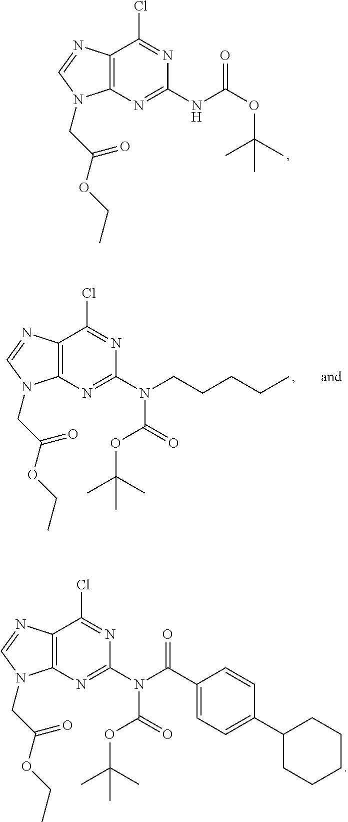 [SMILES: CCOC(=O)Cn1cnc2c(Cl)nc(NC(=O)OC(C)(C)C)nc21 ; CCCCCN(C(=O)OC(C)(C)C)c1nc(Cl)c2ncn(CC(=O)OCC)c2n1 ; CCOC(=O)Cn1cnc2c(Cl)nc(N(C(=O)OC(C)(C)C)C(=O)c3ccc(C4CCCCC4)cc3)nc21]

In one aspect, a compound can be present as the following structure:

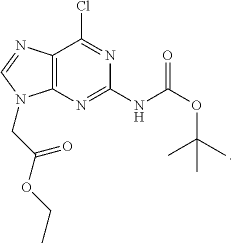 [SMILES: CCOC(=O)Cn1cnc2c(Cl)nc(NC(=O)OC(C)(C)C)nc21]

In one aspect, a compound can be present as the following structure:

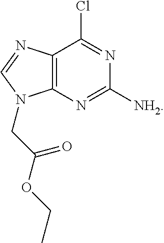 [SMILES: CCOC(=O)Cn1cnc2c(Cl)nc(N)nc21]

In one aspect, a compound can be present as one or more of the following structures:

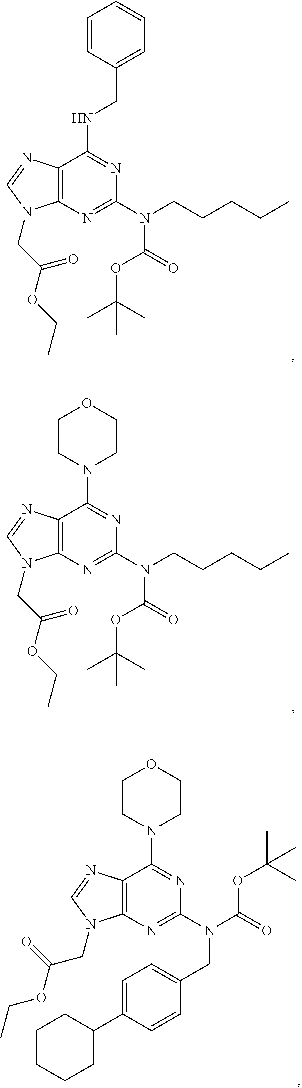

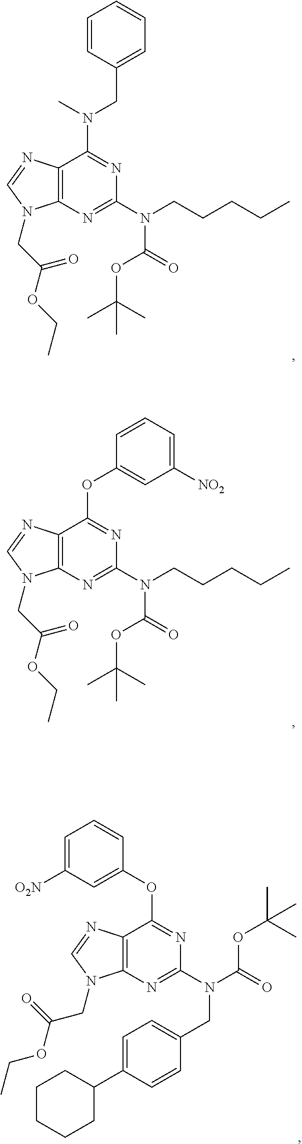

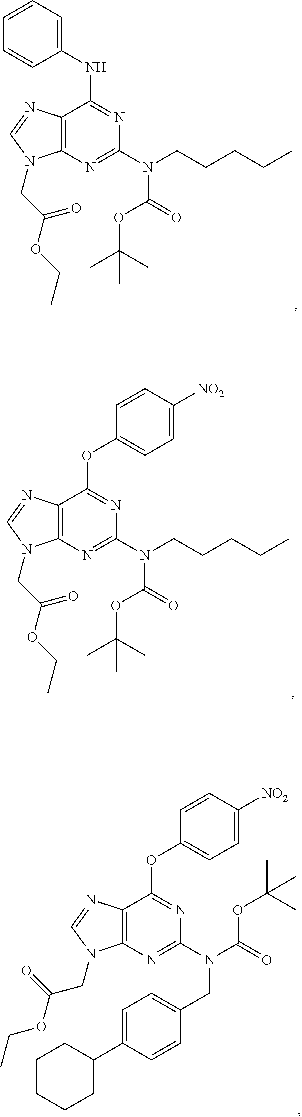

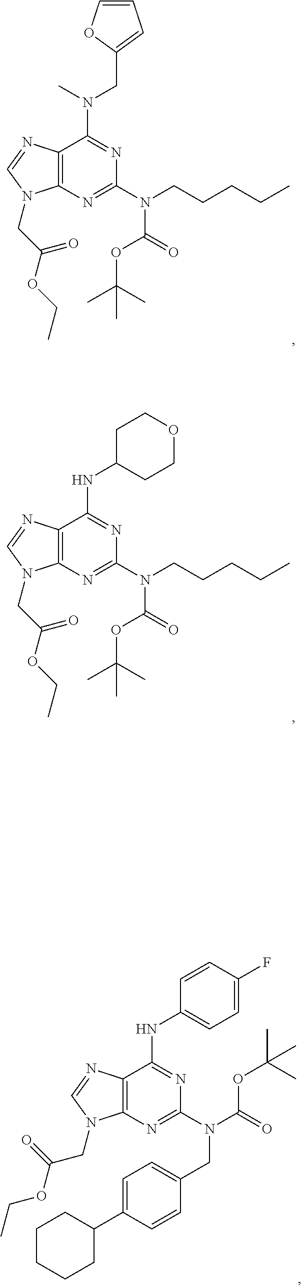

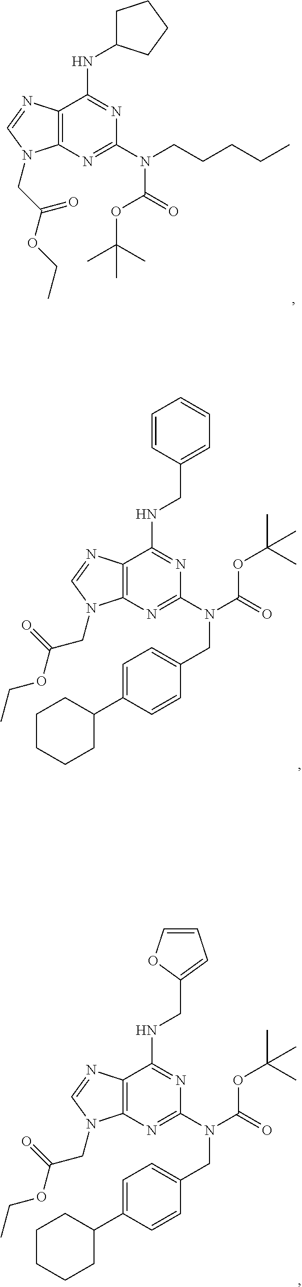

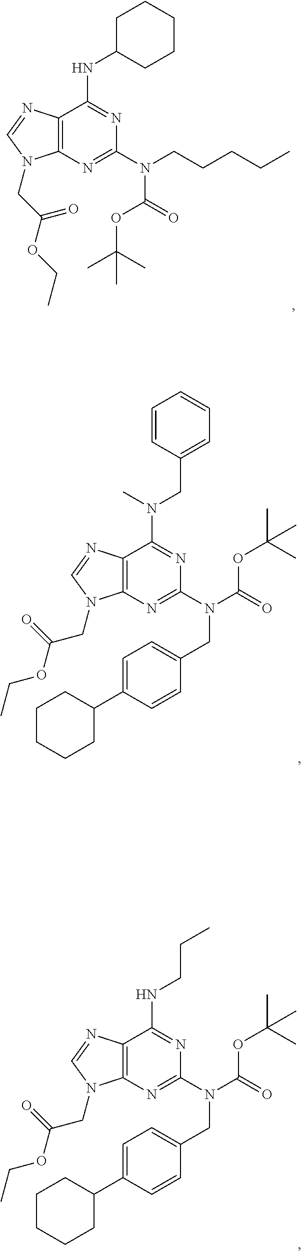

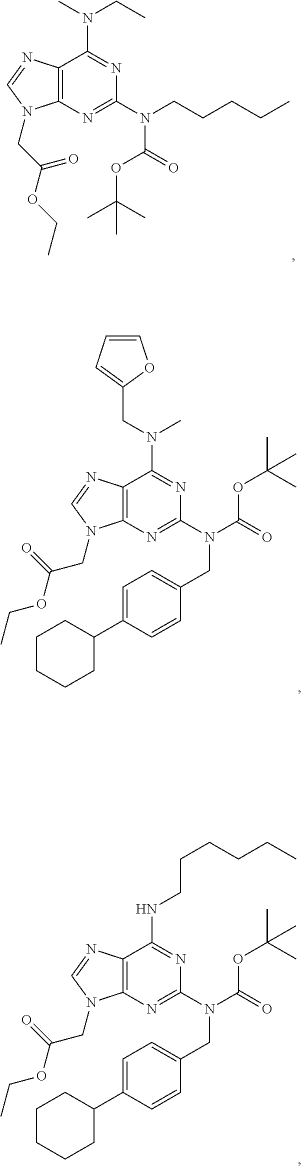

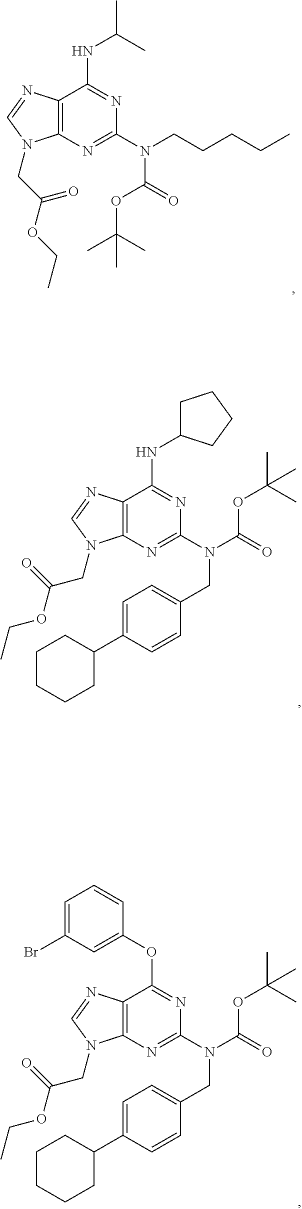

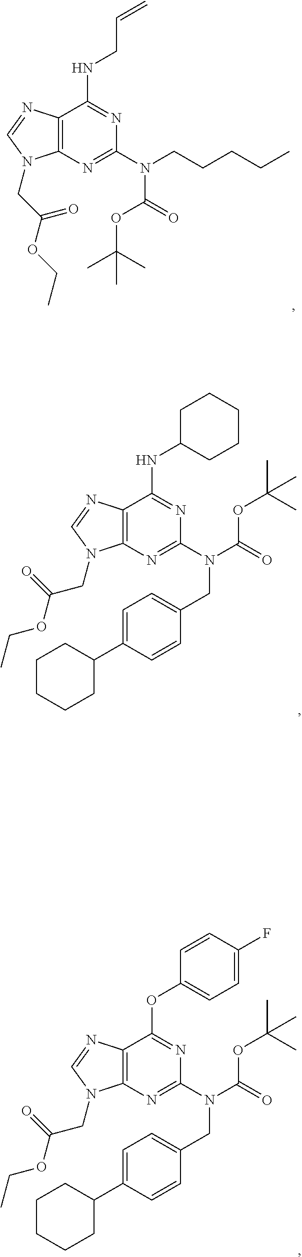

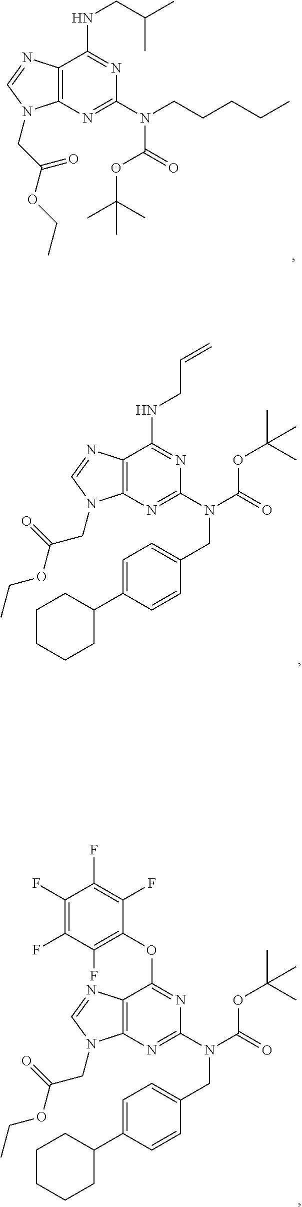

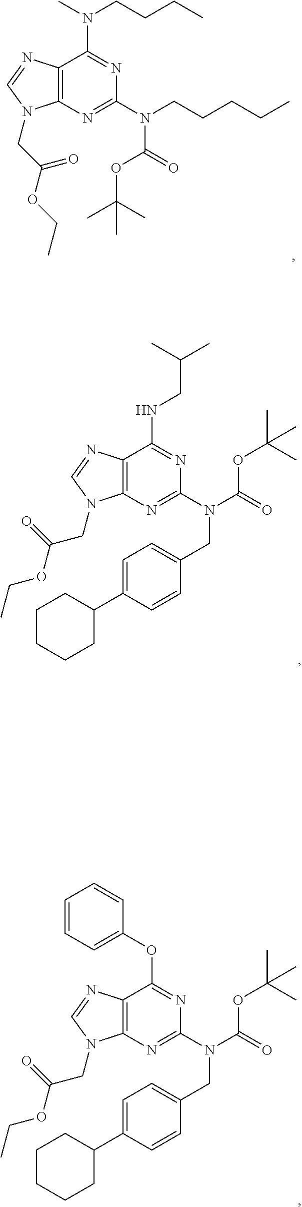

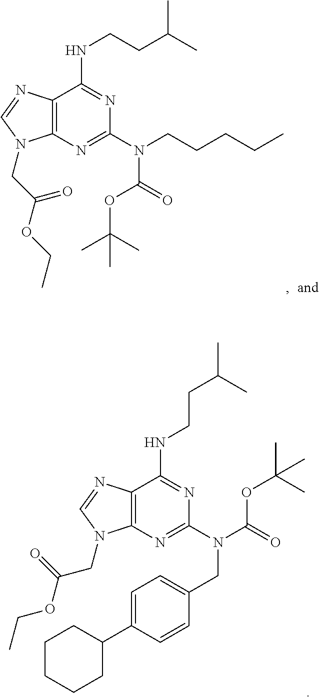

In one aspect, a compound can be present as one or more of the following structures:

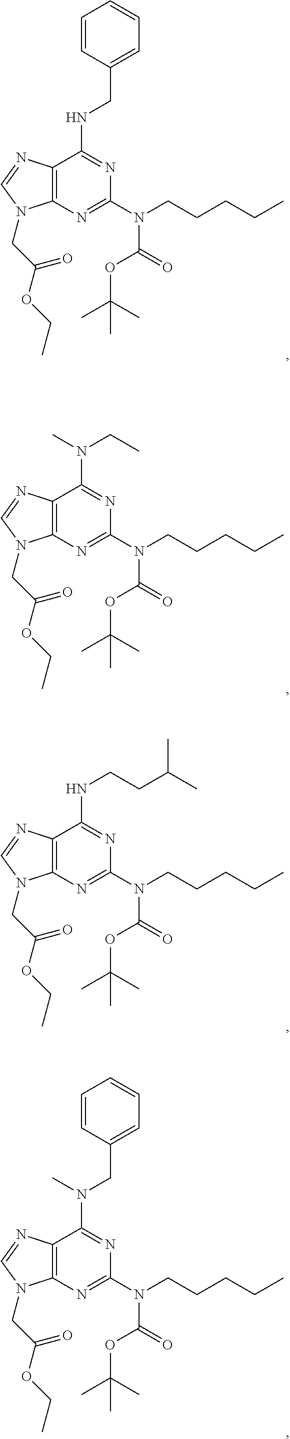 [SMILES: CCCCCN(C(=O)OC(C)(C)C)c1nc(NCc2ccccc2)c2ncn(CC(=O)OCC)c2n1 ; CCCCCN(C(=O)OC(C)(C)C)c1nc(N(C)CC)c2ncn(CC(=O)OCC)c2n1 ; CCCCCN(C(=O)OC(C)(C)C)c1nc(NCCC(C)C)c2ncn(CC(=O)OCC)c2n1 ; CCCCCN(C(=O)OC(C)(C)C)c1nc(N(C)Cc2ccccc2)c2ncn(CC(=O)OCC)c2n1]

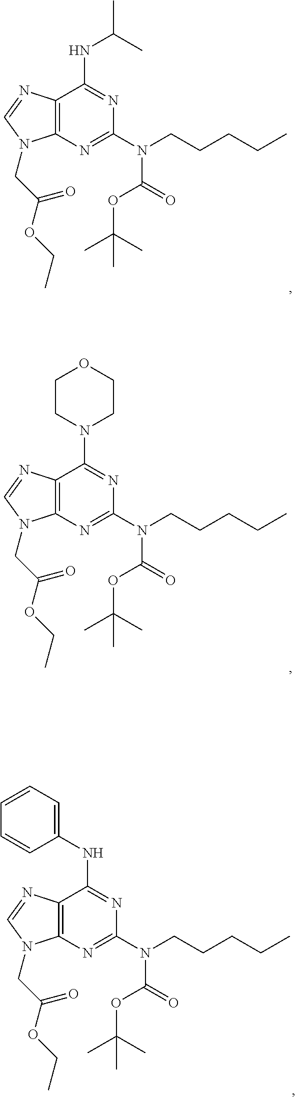 [SMILES: CCCCCN(C(=O)OC(C)(C)C)c1nc(NC(C)C)c2ncn(CC(=O)OCC)c2n1 ; CCCCCN(C(=O)OC(C)(C)C)c1nc(N2CCOCC2)c2ncn(CC(=O)OCC)c2n1 ; CCCCCN(C(=O)OC(C)(C)C)c1nc(Nc2ccccc2)c2ncn(CC(=O)OCC)c2n1]

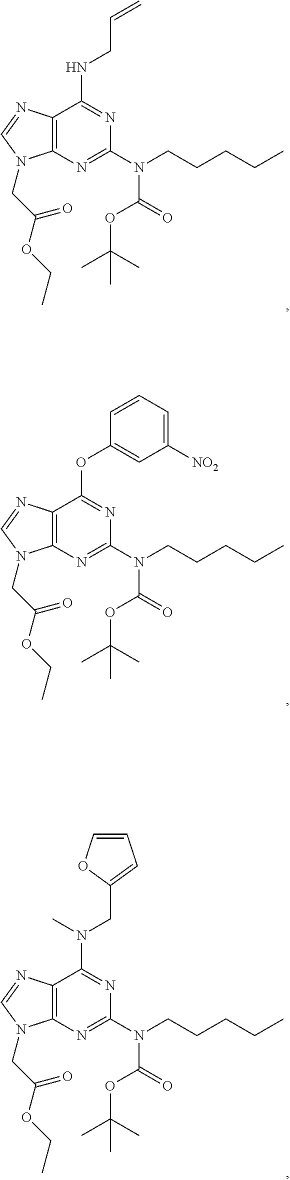 [SMILES: C=CCNc1nc(N(CCCCC)C(=O)OC(C)(C)C)nc2c1ncn2CC(=O)OCC ; CCCCCN(C(=O)OC(C)(C)C)c1nc(Oc2cccc([N+](=O)[O-])c2)c2ncn(CC(=O)OCC)c2n1 ; CCCCCN(C(=O)OC(C)(C)C)c1nc(N(C)Cc2ccco2)c2ncn(CC(=O)OCC)c2n1]

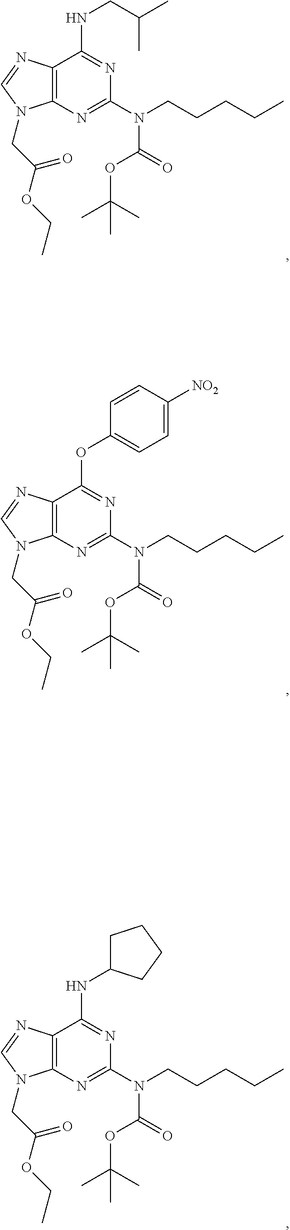 [SMILES: CCCCCN(C(=O)OC(C)(C)C)c1nc(NCC(C)C)c2ncn(CC(=O)OCC)c2n1 ; CCCCCN(C(=O)OC(C)(C)C)c1nc(Oc2ccc([N+](=O)[O-])cc2)c2ncn(CC(=O)OCC)c2n1 ; CCCCCN(C(=O)OC(C)(C)C)c1nc(NC2CCCC2)c2ncn(CC(=O)OCC)c2n1]

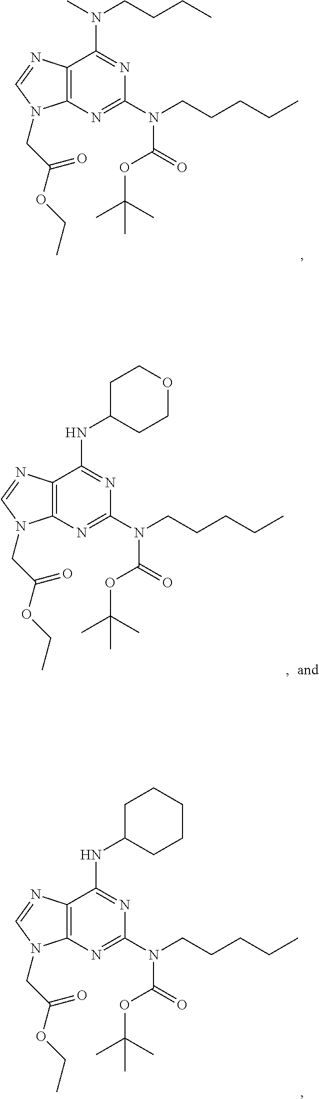 [SMILES: CCCCCN(C(=O)OC(C)(C)C)c1nc(N(C)CCCC)c2ncn(CC(=O)OCC)c2n1 ; CCCCCN(C(=O)OC(C)(C)C)c1nc(NC2CCOCC2)c2ncn(CC(=O)OCC)c2n1 ; CCCCCN(C(=O)OC(C)(C)C)c1nc(NC2CCCCC2)c2ncn(CC(=O)OCC)c2n1]

In one aspect, a compound can be present as one or more of the following structures:

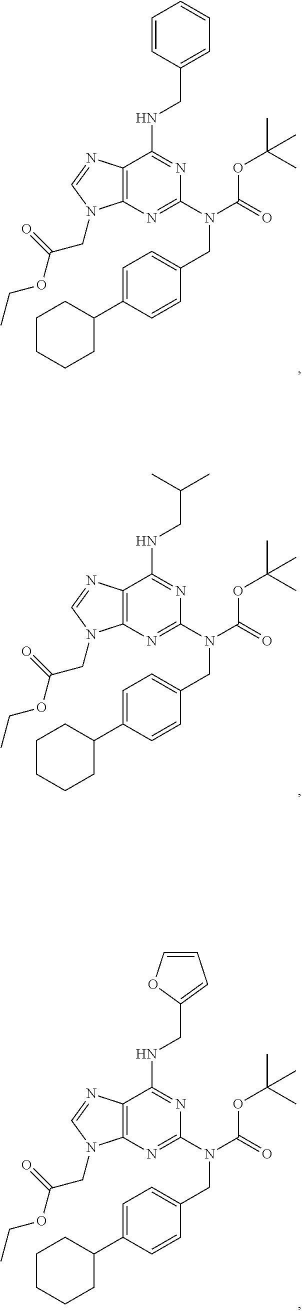

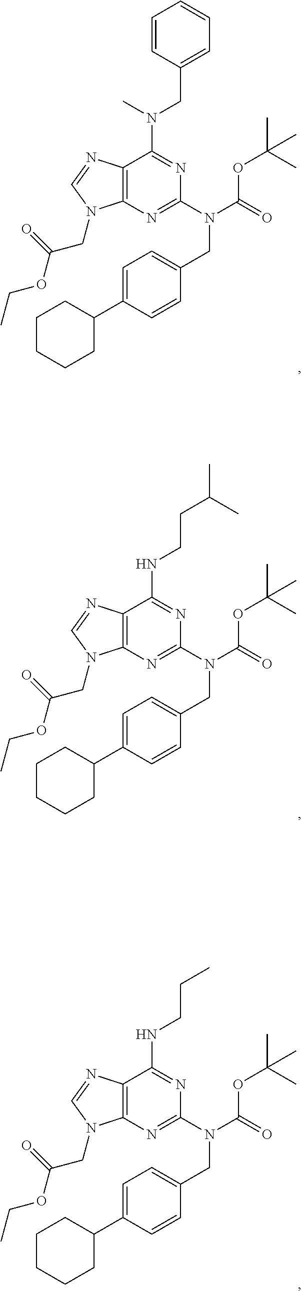

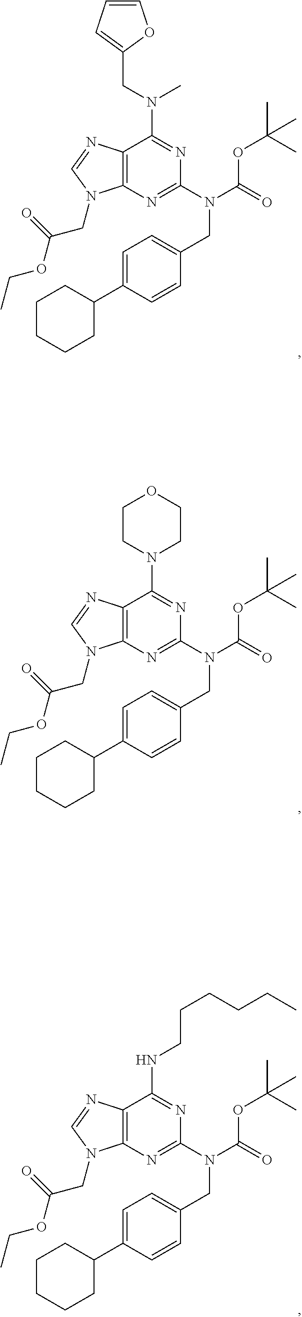

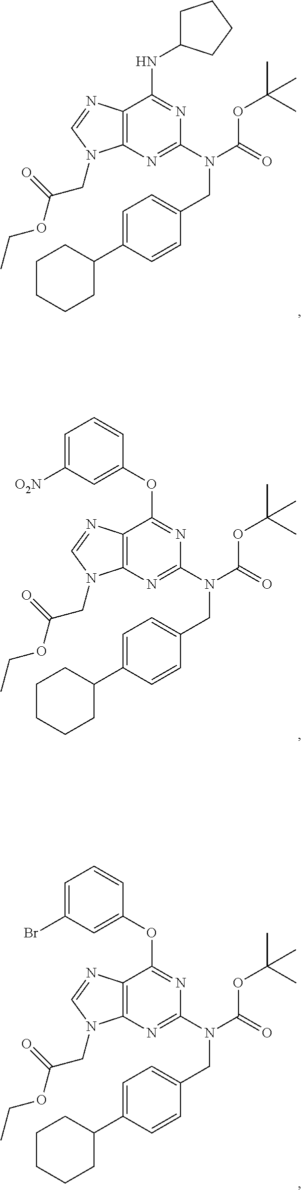

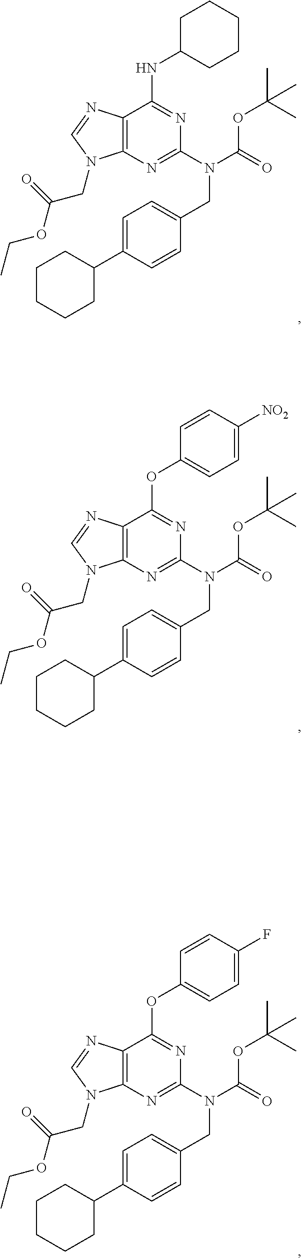

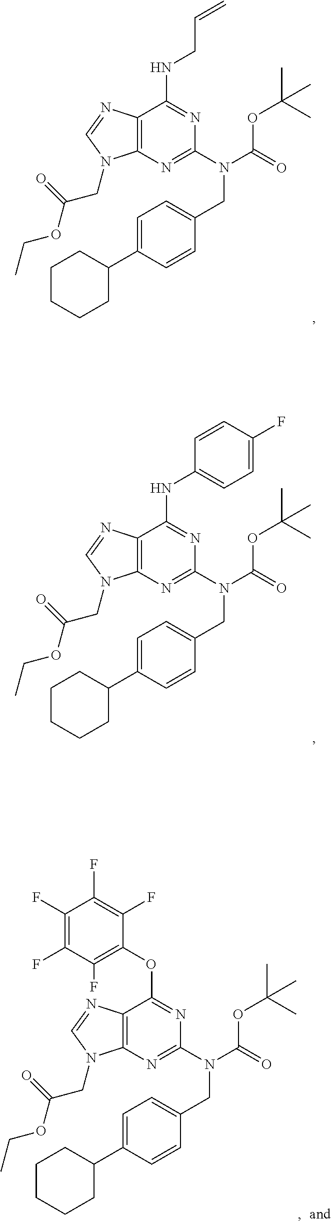

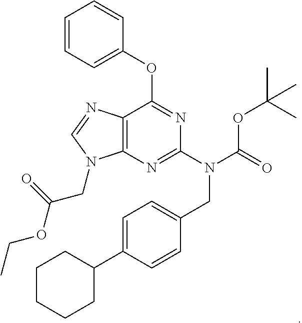 [SMILES: CCOC(=O)Cn1cnc2c(Oc3ccccc3)nc(N(Cc3ccc(C4CCCCC4)cc3)C(=O)OC(C)(C)C)nc21]

In one aspect, a compound can be present as one or more of the following structures:

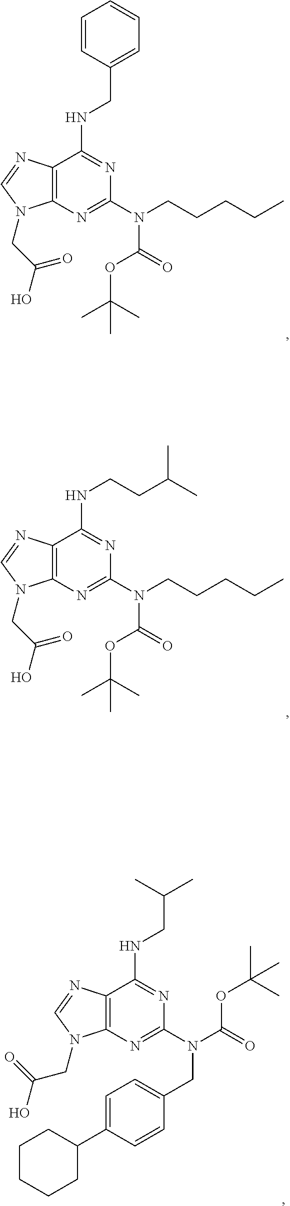 [SMILES: CCCCCN(C(=O)OC(C)(C)C)c1nc(NCc2ccccc2)c2ncn(CC(=O)O)c2n1 ; CCCCCN(C(=O)OC(C)(C)C)c1nc(NCCC(C)C)c2ncn(CC(=O)O)c2n1 ; CC(C)CNc1nc(N(Cc2ccc(C3CCCCC3)cc2)C(=O)OC(C)(C)C)nc2c1ncn2CC(=O)O]

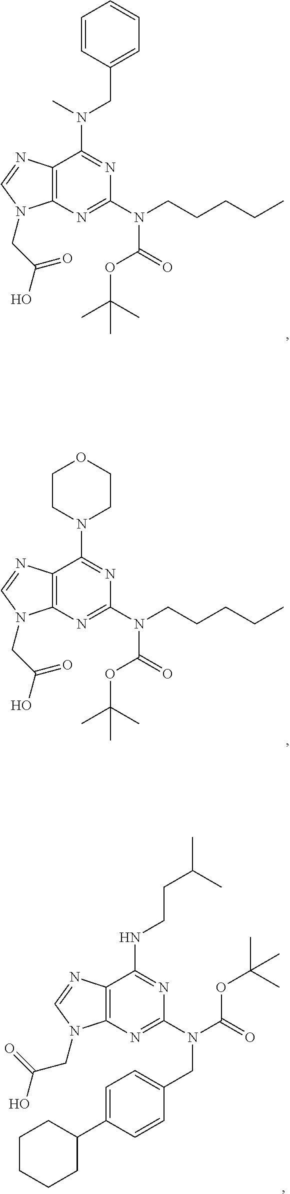 [SMILES: CCCCCN(C(=O)OC(C)(C)C)c1nc(N(C)Cc2ccccc2)c2ncn(CC(=O)O)c2n1 ; CCCCCN(C(=O)OC(C)(C)C)c1nc(N2CCOCC2)c2ncn(CC(=O)O)c2n1 ; CC(C)CCNc1nc(N(Cc2ccc(C3CCCCC3)cc2)C(=O)OC(C)(C)C)nc2c1ncn2CC(=O)O]

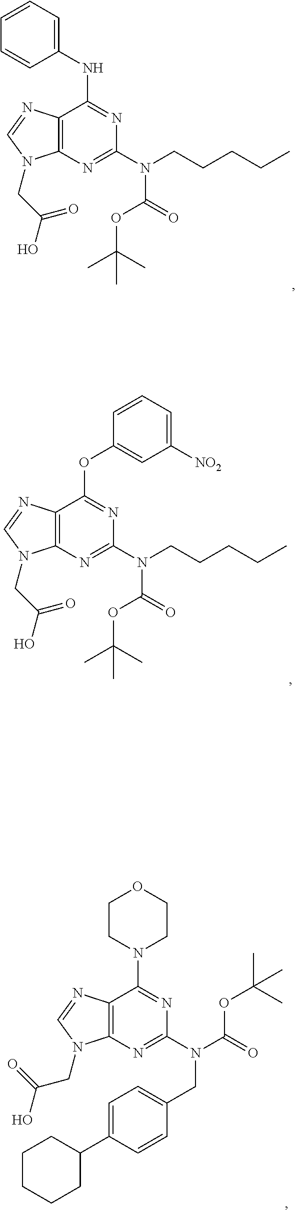 [SMILES: CCCCCN(C(=O)OC(C)(C)C)c1nc(Nc2ccccc2)c2ncn(CC(=O)O)c2n1 ; CCCCCN(C(=O)OC(C)(C)C)c1nc(Oc2cccc([N+](=O)[O-])c2)c2ncn(CC(=O)O)c2n1 ; CC(C)(C)OC(=O)N(Cc1ccc(C2CCCCC2)cc1)c1nc(N2CCOCC2)c2ncn(CC(=O)O)c2n1]

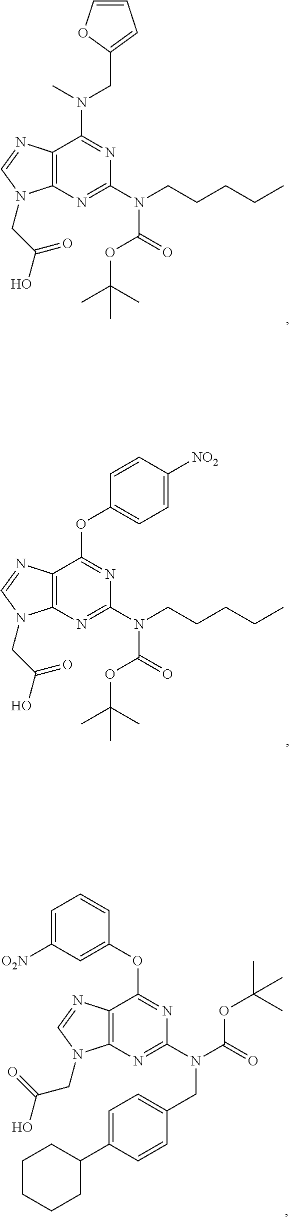 [SMILES: CCCCCN(C(=O)OC(C)(C)C)c1nc(N(C)Cc2ccco2)c2ncn(CC(=O)O)c2n1 ; CCCCCN(C(=O)OC(C)(C)C)c1nc(Oc2ccc([N+](=O)[O-])cc2)c2ncn(CC(=O)O)c2n1 ; CC(C)(C)OC(=O)N(Cc1ccc(C2CCCCC2)cc1)c1nc(Oc2cccc([N+](=O)[O-])c2)c2ncn(CC(=O)O)c2n1]

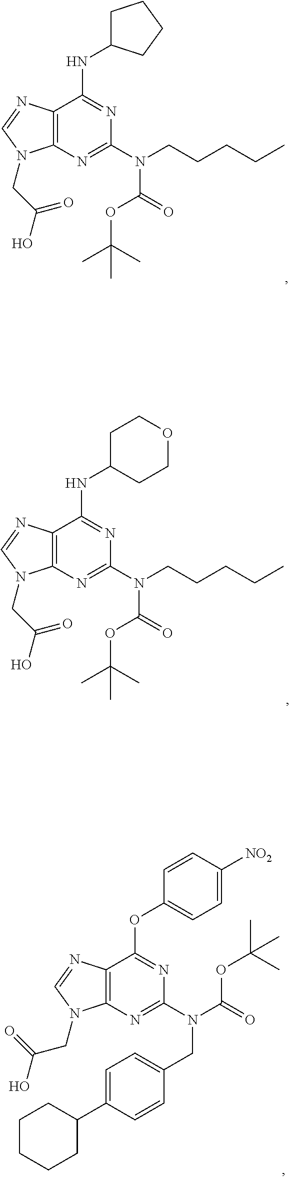 [SMILES: CCCCCN(C(=O)OC(C)(C)C)c1nc(NC2CCCC2)c2ncn(CC(=O)O)c2n1 ; CCCCCN(C(=O)OC(C)(C)C)c1nc(NC2CCOCC2)c2ncn(CC(=O)O)c2n1 ; CC(C)(C)OC(=O)N(Cc1ccc(C2CCCCC2)cc1)c1nc(Oc2ccc([N+](=O)[O-])cc2)c2ncn(CC(=O)O)c2n1]

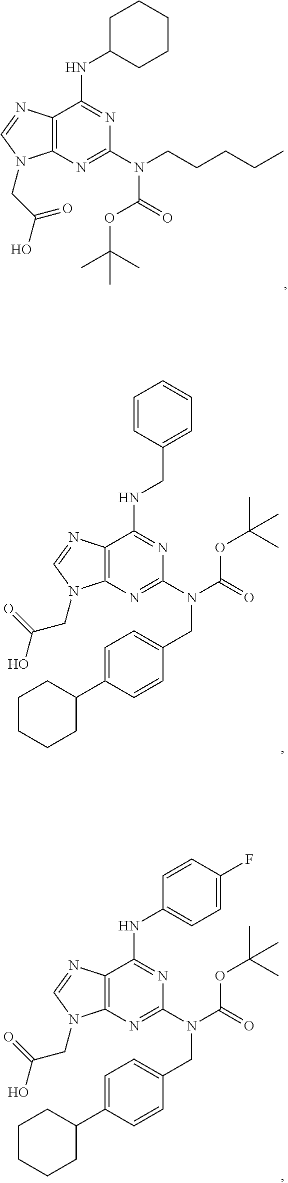 [SMILES: CCCCCN(C(=O)OC(C)(C)C)c1nc(NC2CCCCC2)c2ncn(CC(=O)O)c2n1 ; CC(C)(C)OC(=O)N(Cc1ccc(C2CCCCC2)cc1)c1nc(NCc2ccccc2)c2ncn(CC(=O)O)c2n1 ; CC(C)(C)OC(=O)N(Cc1ccc(C2CCCCC2)cc1)c1nc(Nc2ccc(F)cc2)c2ncn(CC(=O)O)c2n1]

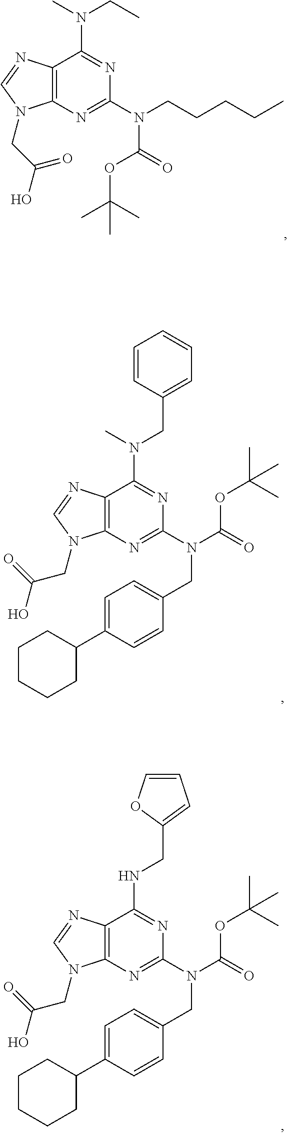 [SMILES: CCCCCN(C(=O)OC(C)(C)C)c1nc(N(C)CC)c2ncn(CC(=O)O)c2n1 ; CN(Cc1ccccc1)c1nc(N(Cc2ccc(C3CCCCC3)cc2)C(=O)OC(C)(C)C)nc2c1ncn2CC(=O)O ; CC(C)(C)OC(=O)N(Cc1ccc(C2CCCCC2)cc1)c1nc(NCc2ccco2)c2ncn(CC(=O)O)c2n1]

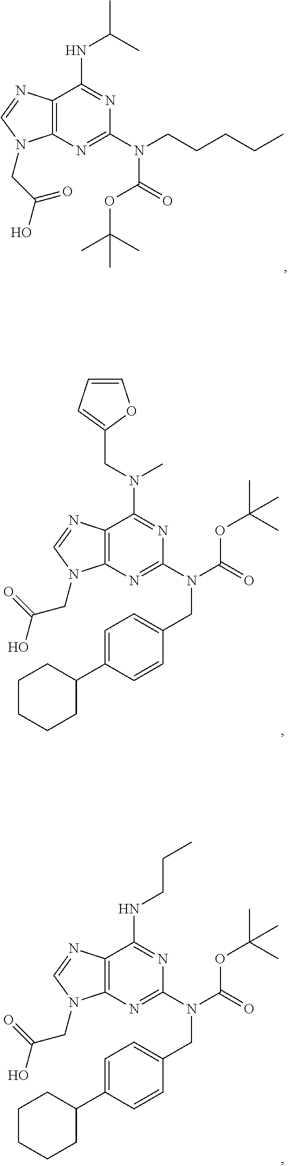 [SMILES: CCCCCN(C(=O)OC(C)(C)C)c1nc(NC(C)C)c2ncn(CC(=O)O)c2n1 ; CN(Cc1ccco1)c1nc(N(Cc2ccc(C3CCCCC3)cc2)C(=O)OC(C)(C)C)nc2c1ncn2CC(=O)O ; CCCNc1nc(N(Cc2ccc(C3CCCCC3)cc2)C(=O)OC(C)(C)C)nc2c1ncn2CC(=O)O]

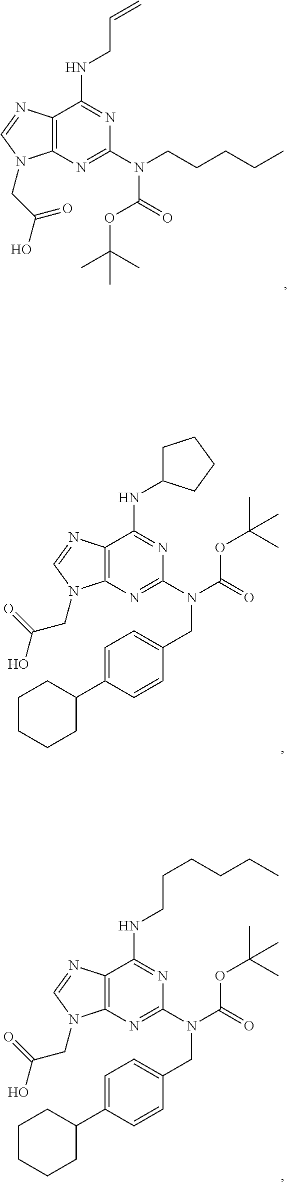 [SMILES: C=CCNc1nc(N(CCCCC)C(=O)OC(C)(C)C)nc2c1ncn2CC(=O)O ; CC(C)(C)OC(=O)N(Cc1ccc(C2CCCCC2)cc1)c1nc(NC2CCCC2)c2ncn(CC(=O)O)c2n1 ; CCCCCCNc1nc(N(Cc2ccc(C3CCCCC3)cc2)C(=O)OC(C)(C)C)nc2c1ncn2CC(=O)O]

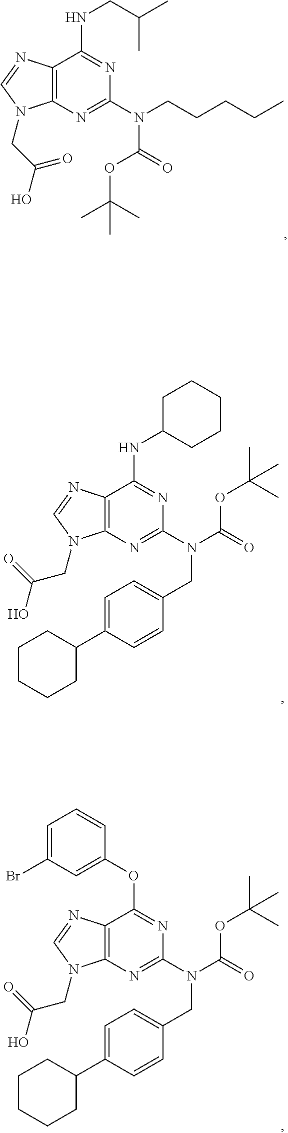 [SMILES: CCCCCN(C(=O)OC(C)(C)C)c1nc(NCC(C)C)c2ncn(CC(=O)O)c2n1 ; CC(C)(C)OC(=O)N(Cc1ccc(C2CCCCC2)cc1)c1nc(NC2CCCCC2)c2ncn(CC(=O)O)c2n1 ; CC(C)(C)OC(=O)N(Cc1ccc(C2CCCCC2)cc1)c1nc(Oc2cccc(Br)c2)c2ncn(CC(=O)O)c2n1]

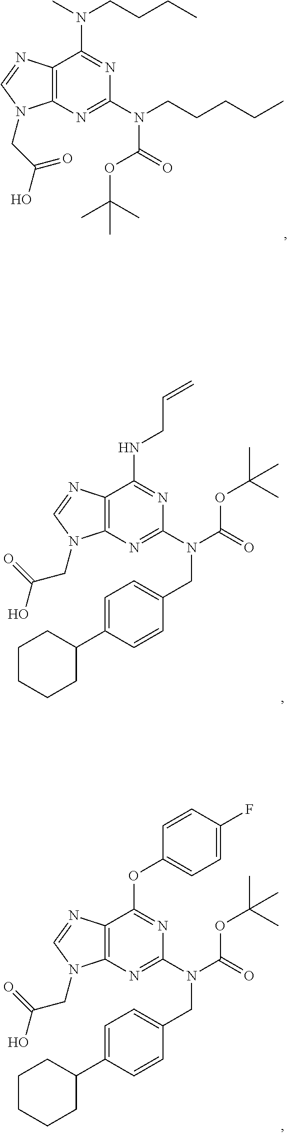 [SMILES: CCCCCN(C(=O)OC(C)(C)C)c1nc(N(C)CCCC)c2ncn(CC(=O)O)c2n1 ; C=CCNc1nc(N(Cc2ccc(C3CCCCC3)cc2)C(=O)OC(C)(C)C)nc2c1ncn2CC(=O)O ; CC(C)(C)OC(=O)N(Cc1ccc(C2CCCCC2)cc1)c1nc(Oc2ccc(F)cc2)c2ncn(CC(=O)O)c2n1]

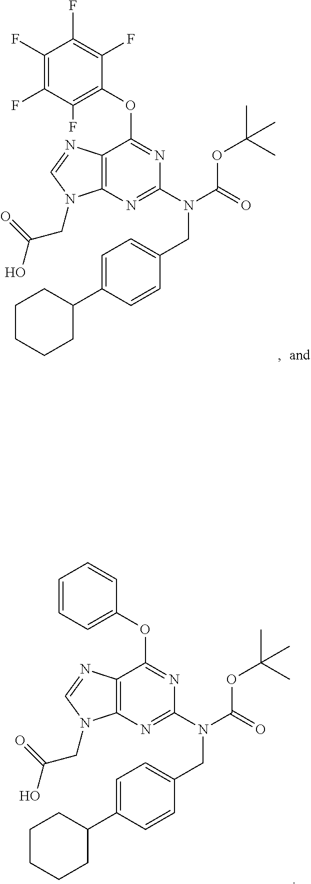 [SMILES: CC(C)(C)OC(=O)N(Cc1ccc(C2CCCCC2)cc1)c1nc(Oc2c(F)c(F)c(F)c(F)c2F)c2ncn(CC(=O)O)c2n1 ; CC(C)(C)OC(=O)N(Cc1ccc(C2CCCCC2)cc1)c1nc(Oc2ccccc2)c2ncn(CC(=O)O)c2n1]

In one aspect, a compound can be present as one or more of the following structures:

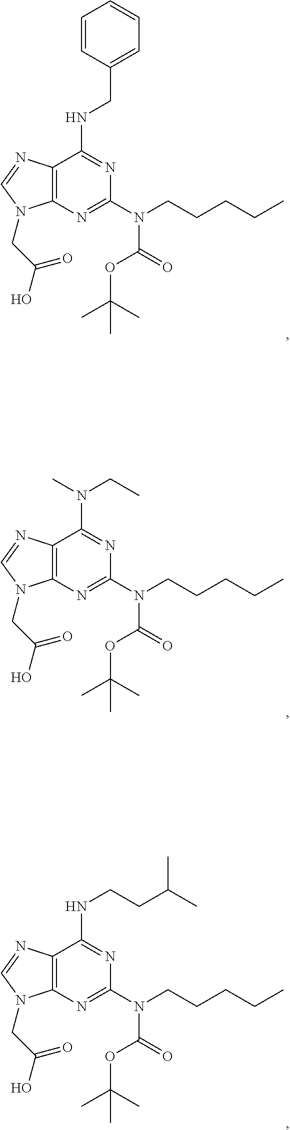 [SMILES: CCCCCN(C(=O)OC(C)(C)C)c1nc(NCc2ccccc2)c2ncn(CC(=O)O)c2n1 ; CCCCCN(C(=O)OC(C)(C)C)c1nc(N(C)CC)c2ncn(CC(=O)O)c2n1 ; CCCCCN(C(=O)OC(C)(C)C)c1nc(NCCC(C)C)c2ncn(CC(=O)O)c2n1]

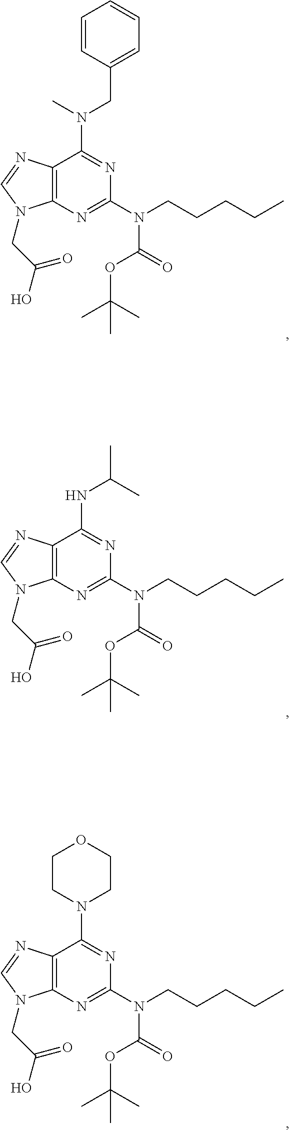 [SMILES: CCCCCN(C(=O)OC(C)(C)C)c1nc(N(C)Cc2ccccc2)c2ncn(CC(=O)O)c2n1 ; CCCCCN(C(=O)OC(C)(C)C)c1nc(NC(C)C)c2ncn(CC(=O)O)c2n1 ; CCCCCN(C(=O)OC(C)(C)C)c1nc(N2CCOCC2)c2ncn(CC(=O)O)c2n1]

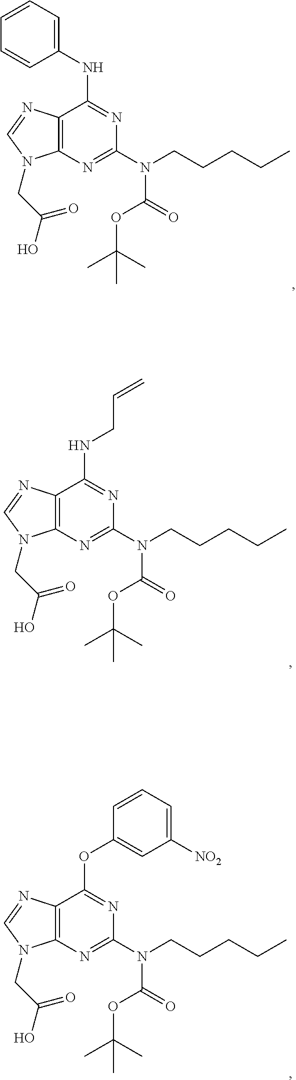 [SMILES: CCCCCN(C(=O)OC(C)(C)C)c1nc(Nc2ccccc2)c2ncn(CC(=O)O)c2n1 ; C=CCNc1nc(N(CCCCC)C(=O)OC(C)(C)C)nc2c1ncn2CC(=O)O ; CCCCCN(C(=O)OC(C)(C)C)c1nc(Oc2cccc([N+](=O)[O-])c2)c2ncn(CC(=O)O)c2n1]

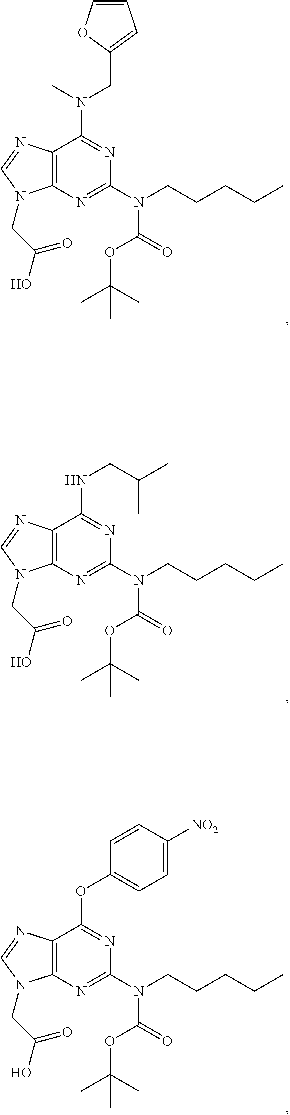 [SMILES: CCCCCN(C(=O)OC(C)(C)C)c1nc(N(C)Cc2ccco2)c2ncn(CC(=O)O)c2n1 ; CCCCCN(C(=O)OC(C)(C)C)c1nc(NCC(C)C)c2ncn(CC(=O)O)c2n1 ; CCCCCN(C(=O)OC(C)(C)C)c1nc(Oc2ccc([N+](=O)[O-])cc2)c2ncn(CC(=O)O)c2n1]

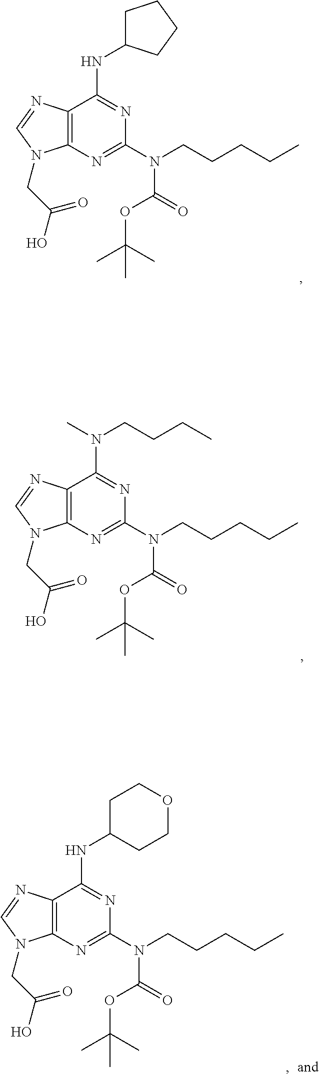 [SMILES: CCCCCN(C(=O)OC(C)(C)C)c1nc(NC2CCCC2)c2ncn(CC(=O)O)c2n1 ; CCCCCN(C(=O)OC(C)(C)C)c1nc(N(C)CCCC)c2ncn(CC(=O)O)c2n1 ; CCCCCN(C(=O)OC(C)(C)C)c1nc(NC2CCOCC2)c2ncn(CC(=O)O)c2n1]

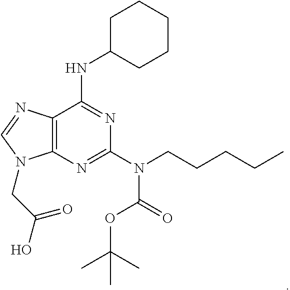 [SMILES: CCCCCN(C(=O)OC(C)(C)C)c1nc(NC2CCCCC2)c2ncn(CC(=O)O)c2n1]

In one aspect, a compound can be present as one or more of the following structures:

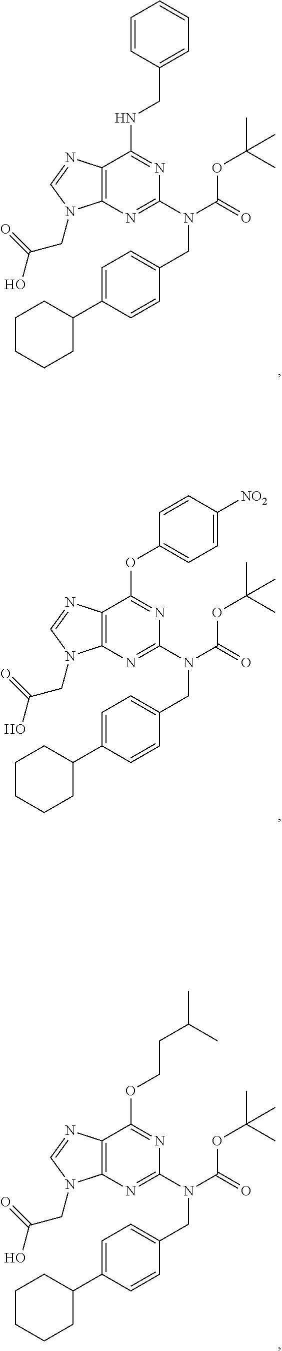 [SMILES: CC(C)(C)OC(=O)N(Cc1ccc(C2CCCCC2)cc1)c1nc(NCc2ccccc2)c2ncn(CC(=O)O)c2n1 ; CC(C)(C)OC(=O)N(Cc1ccc(C2CCCCC2)cc1)c1nc(Oc2ccc([N+](=O)[O-])cc2)c2ncn(CC(=O)O)c2n1 ; CC(C)CCOc1nc(N(Cc2ccc(C3CCCCC3)cc2)C(=O)OC(C)(C)C)nc2c1ncn2CC(=O)O]

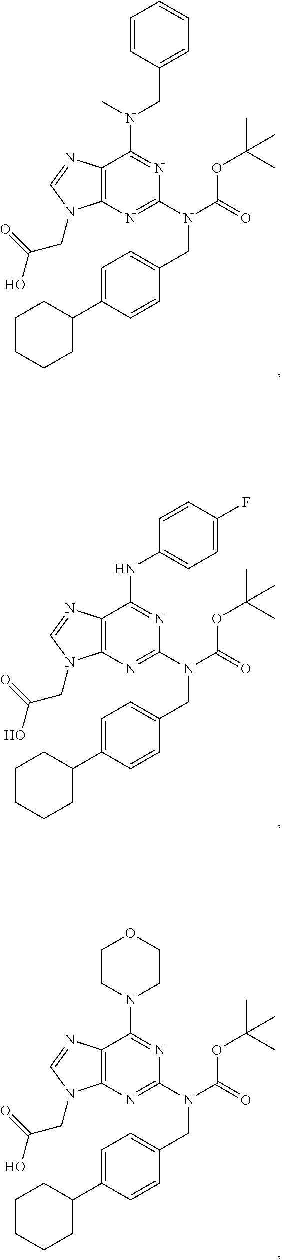 [SMILES: CN(Cc1ccccc1)c1nc(N(Cc2ccc(C3CCCCC3)cc2)C(=O)OC(C)(C)C)nc2c1ncn2CC(=O)O ; CC(C)(C)OC(=O)N(Cc1ccc(C2CCCCC2)cc1)c1nc(Nc2ccc(F)cc2)c2ncn(CC(=O)O)c2n1 ; CC(C)(C)OC(=O)N(Cc1ccc(C2CCCCC2)cc1)c1nc(N2CCOCC2)c2ncn(CC(=O)O)c2n1]

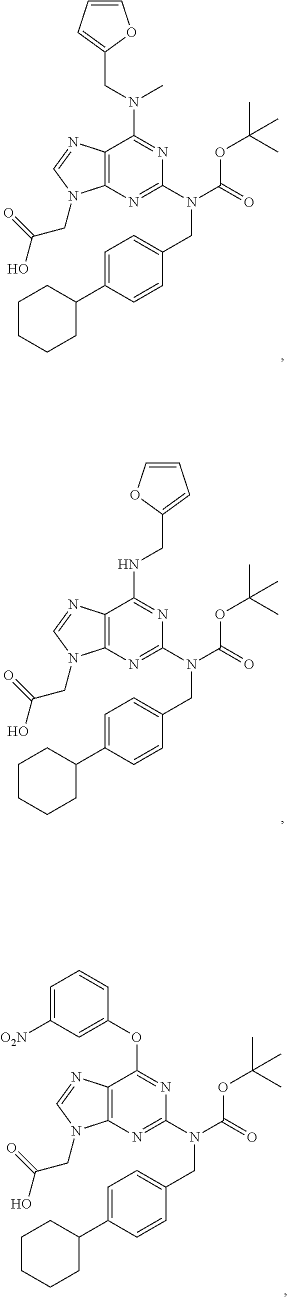 [SMILES: CN(Cc1ccco1)c1nc(N(Cc2ccc(C3CCCCC3)cc2)C(=O)OC(C)(C)C)nc2c1ncn2CC(=O)O ; CC(C)(C)OC(=O)N(Cc1ccc(C2CCCCC2)cc1)c1nc(NCc2ccco2)c2ncn(CC(=O)O)c2n1 ; CC(C)(C)OC(=O)N(Cc1ccc(C2CCCCC2)cc1)c1nc(Oc2cccc([N+](=O)[O-])c2)c2ncn(CC(=O)O)c2n1]

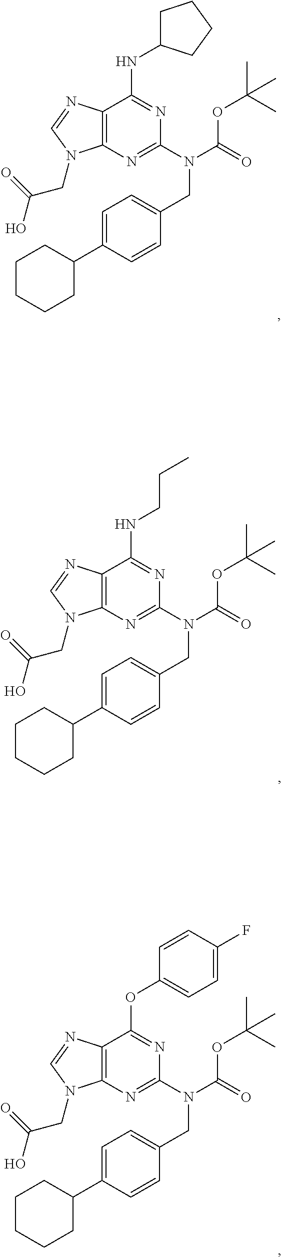 [SMILES: CC(C)(C)OC(=O)N(Cc1ccc(C2CCCCC2)cc1)c1nc(NC2CCCC2)c2ncn(CC(=O)O)c2n1 ; CCCNc1nc(N(Cc2ccc(C3CCCCC3)cc2)C(=O)OC(C)(C)C)nc2c1ncn2CC(=O)O ; CC(C)(C)OC(=O)N(Cc1ccc(C2CCCCC2)cc1)c1nc(Oc2ccc(F)cc2)c2ncn(CC(=O)O)c2n1]

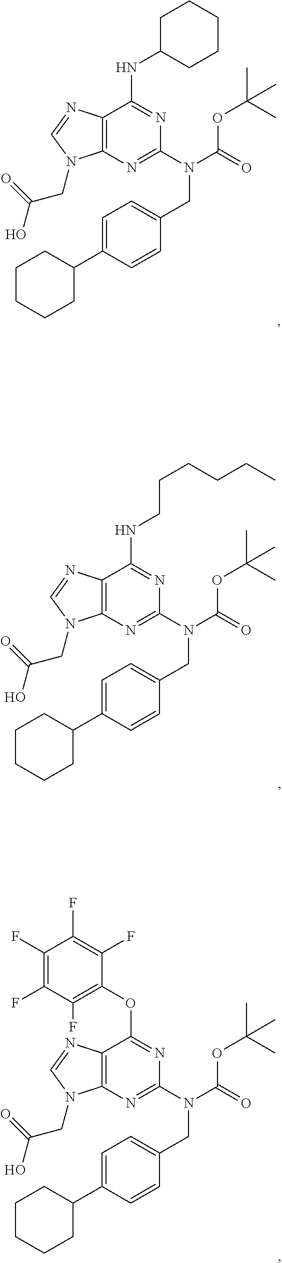 [SMILES: CC(C)(C)OC(=O)N(Cc1ccc(C2CCCCC2)cc1)c1nc(NC2CCCCC2)c2ncn(CC(=O)O)c2n1 ; CCCCCCNc1nc(N(Cc2ccc(C3CCCCC3)cc2)C(=O)OC(C)(C)C)nc2c1ncn2CC(=O)O ; CC(C)(C)OC(=O)N(Cc1ccc(C2CCCCC2)cc1)c1nc(Oc2c(F)c(F)c(F)c(F)c2F)c2ncn(CC(=O)O)c2n1]

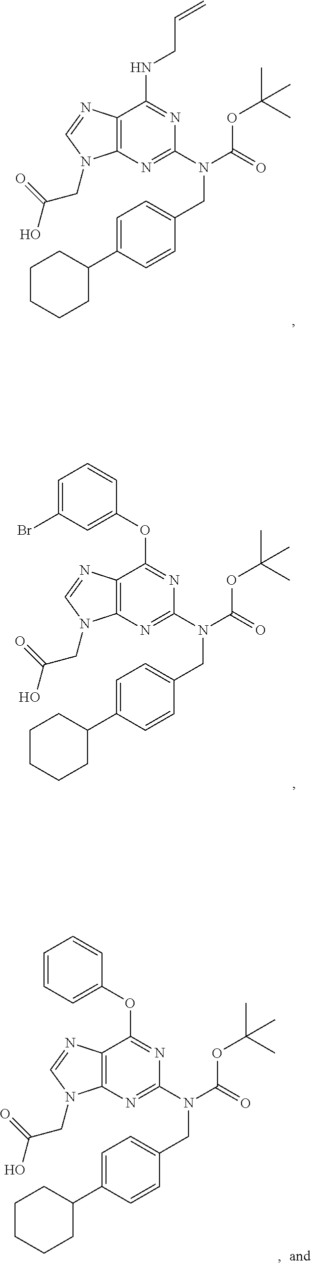 [SMILES: C=CCNc1nc(N(Cc2ccc(C3CCCCC3)cc2)C(=O)OC(C)(C)C)nc2c1ncn2CC(=O)O ; CC(C)(C)OC(=O)N(Cc1ccc(C2CCCCC2)cc1)c1nc(Oc2cccc(Br)c2)c2ncn(CC(=O)O)c2n1 ; CC(C)(C)OC(=O)N(Cc1ccc(C2CCCCC2)cc1)c1nc(Oc2ccccc2)c2ncn(CC(=O)O)c2n1]

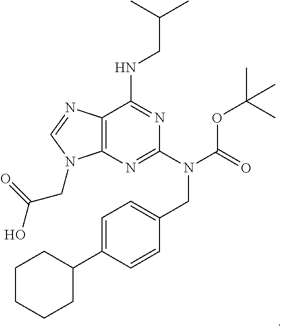 [SMILES: CC(C)CNc1nc(N(Cc2ccc(C3CCCCC3)cc2)C(=O)OC(C)(C)C)nc2c1ncn2CC(=O)O]

In one aspect, a compound can be present as one or more of the following structures:

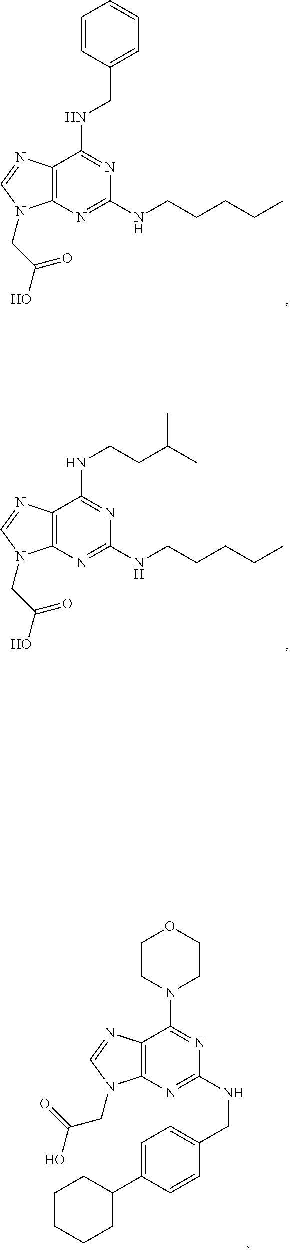 [SMILES: CCCCCNc1nc(NCc2ccccc2)c2ncn(CC(=O)O)c2n1 ; CCCCCNc1nc(NCCC(C)C)c2ncn(CC(=O)O)c2n1 ; O=C(O)Cn1cnc2c(N3CCOCC3)nc(NCc3ccc(C4CCCCC4)cc3)nc21]

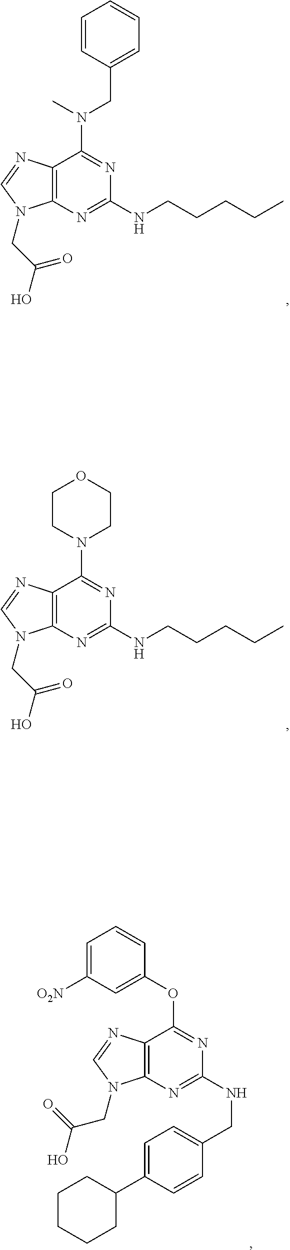 [SMILES: CCCCCNc1nc(N(C)Cc2ccccc2)c2ncn(CC(=O)O)c2n1 ; CCCCCNc1nc(N2CCOCC2)c2ncn(CC(=O)O)c2n1 ; O=C(O)Cn1cnc2c(Oc3cccc([N+](=O)[O-])c3)nc(NCc3ccc(C4CCCCC4)cc3)nc21]

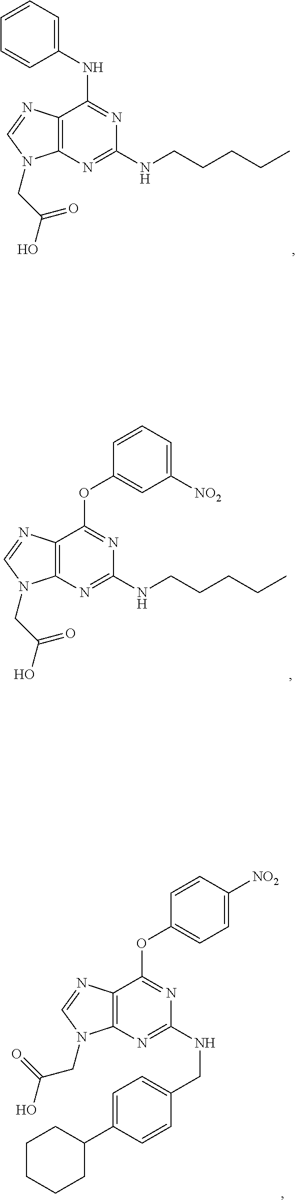 [SMILES: CCCCCNc1nc(Nc2ccccc2)c2ncn(CC(=O)O)c2n1 ; CCCCCNc1nc(Oc2cccc([N+](=O)[O-])c2)c2ncn(CC(=O)O)c2n1 ; O=C(O)Cn1cnc2c(Oc3ccc([N+](=O)[O-])cc3)nc(NCc3ccc(C4CCCCC4)cc3)nc21]

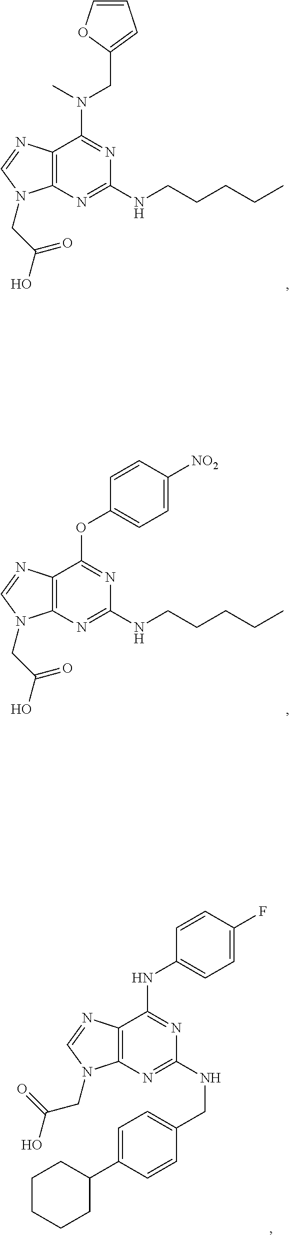 [SMILES: CCCCCNc1nc(N(C)Cc2ccco2)c2ncn(CC(=O)O)c2n1 ; CCCCCNc1nc(Oc2ccc([N+](=O)[O-])cc2)c2ncn(CC(=O)O)c2n1 ; O=C(O)Cn1cnc2c(Nc3ccc(F)cc3)nc(NCc3ccc(C4CCCCC4)cc3)nc21]

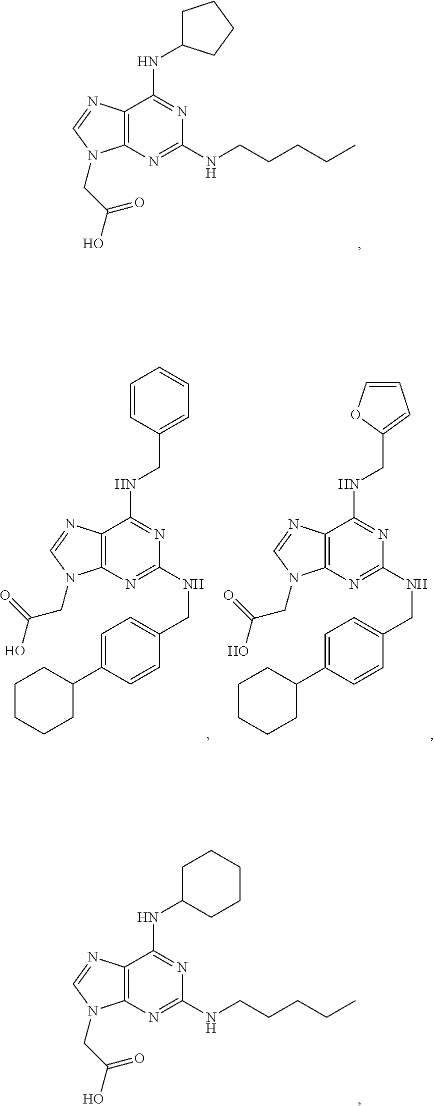

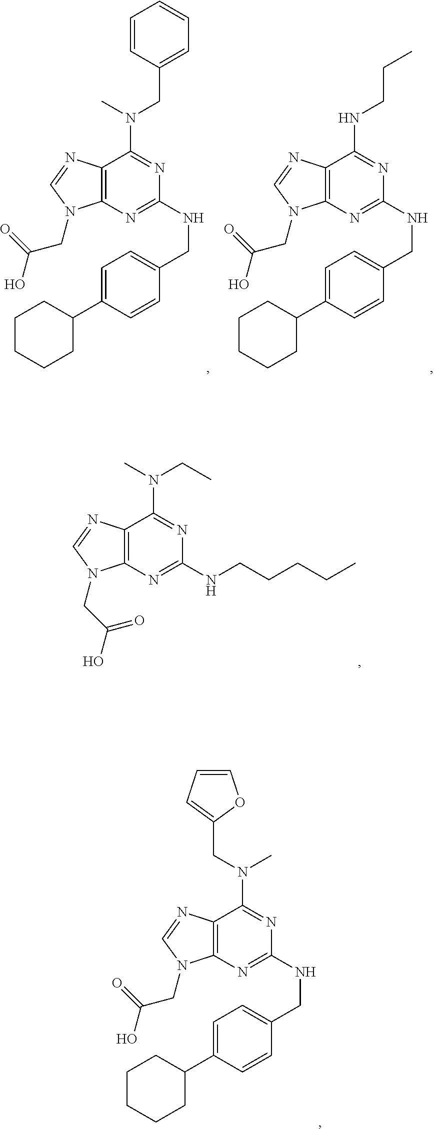

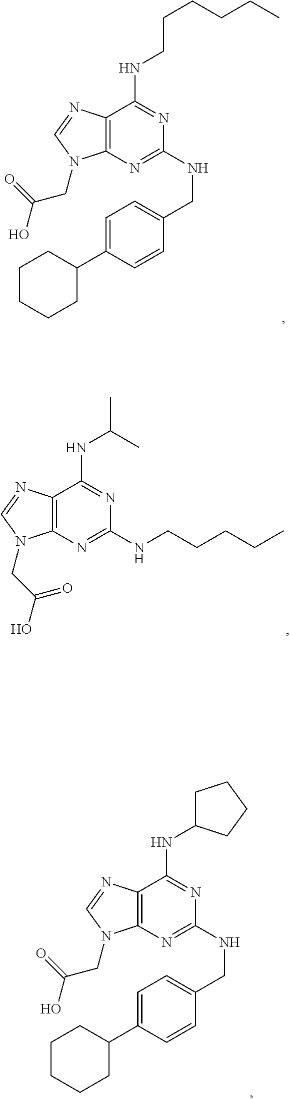 [SMILES: CCCCCCNc1nc(NCc2ccc(C3CCCCC3)cc2)nc2c1ncn2CC(=O)O ; CCCCCNc1nc(NC(C)C)c2ncn(CC(=O)O)c2n1 ; O=C(O)Cn1cnc2c(NC3CCCC3)nc(NCc3ccc(C4CCCCC4)cc3)nc21]

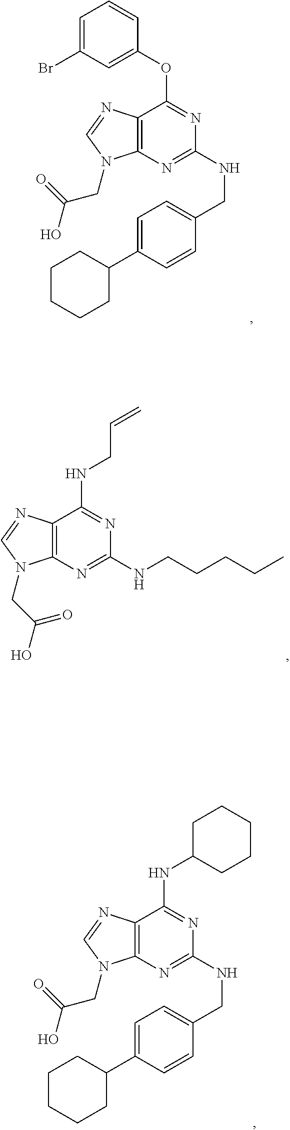 [SMILES: O=C(O)Cn1cnc2c(Oc3cccc(Br)c3)nc(NCc3ccc(C4CCCCC4)cc3)nc21 ; C=CCNc1nc(NCCCCC)nc2c1ncn2CC(=O)O ; O=C(O)Cn1cnc2c(NC3CCCCC3)nc(NCc3ccc(C4CCCCC4)cc3)nc21]

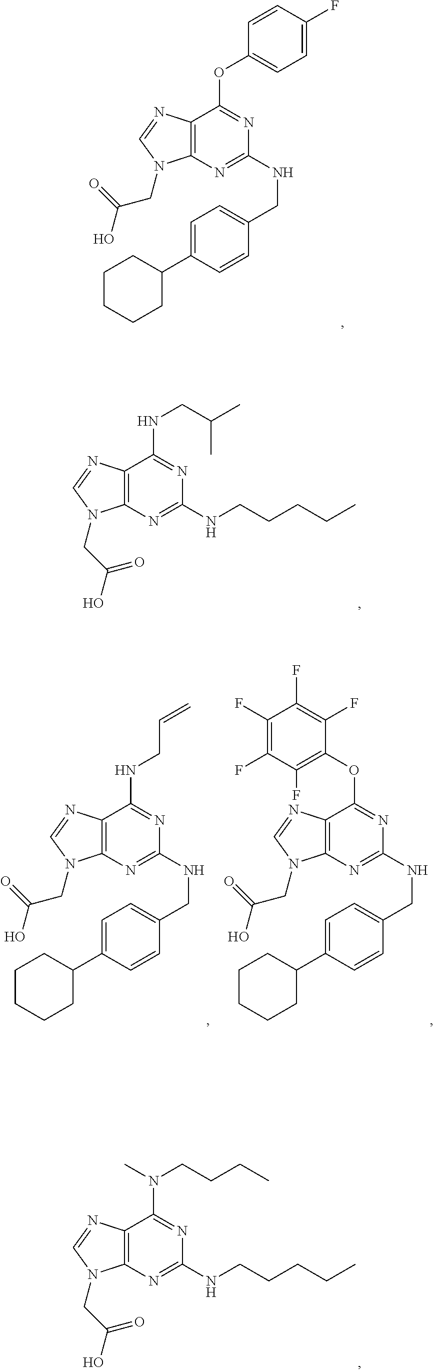

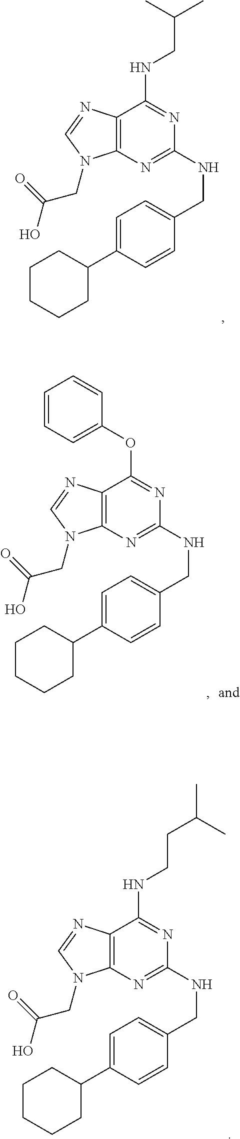 [SMILES: CC(C)CNc1nc(NCc2ccc(C3CCCCC3)cc2)nc2c1ncn2CC(=O)O ; O=C(O)Cn1cnc2c(Oc3ccccc3)nc(NCc3ccc(C4CCCCC4)cc3)nc21 ; CC(C)CCNc1nc(NCc2ccc(C3CCCCC3)cc2)nc2c1ncn2CC(=O)O]

In one aspect, a compound can be present as one or more of the following structures:

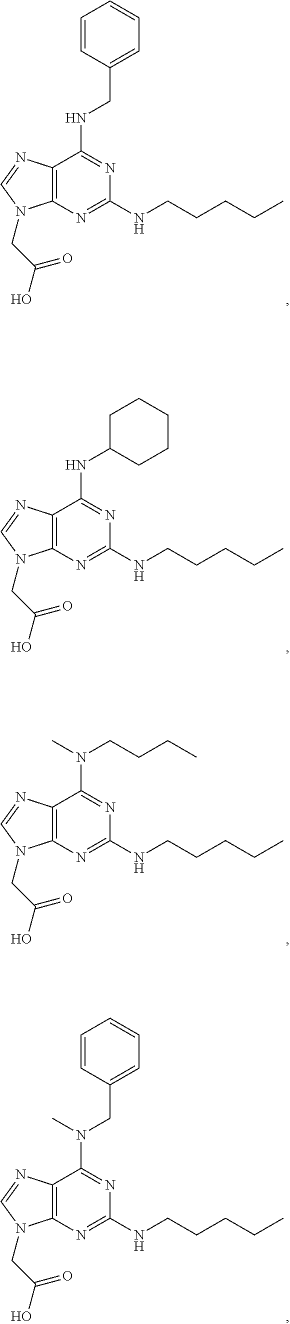 [SMILES: CCCCCNc1nc(NCc2ccccc2)c2ncn(CC(=O)O)c2n1 ; CCCCCNc1nc(NC2CCCCC2)c2ncn(CC(=O)O)c2n1 ; CCCCCNc1nc(N(C)CCCC)c2ncn(CC(=O)O)c2n1 ; CCCCCNc1nc(N(C)Cc2ccccc2)c2ncn(CC(=O)O)c2n1]

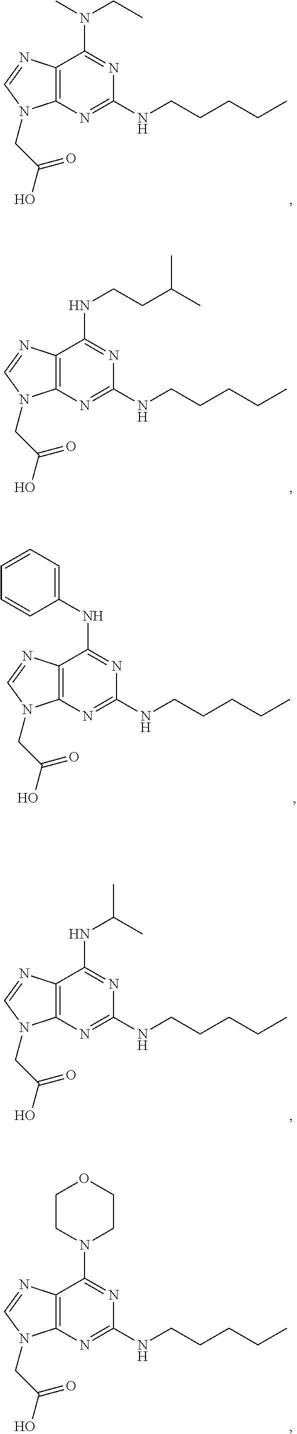 [SMILES: CCCCCNc1nc(N(C)CC)c2ncn(CC(=O)O)c2n1 ; CCCCCNc1nc(NCCC(C)C)c2ncn(CC(=O)O)c2n1 ; CCCCCNc1nc(Nc2ccccc2)c2ncn(CC(=O)O)c2n1 ; CCCCCNc1nc(NC(C)C)c2ncn(CC(=O)O)c2n1 ; CCCCCNc1nc(N2CCOCC2)c2ncn(CC(=O)O)c2n1]

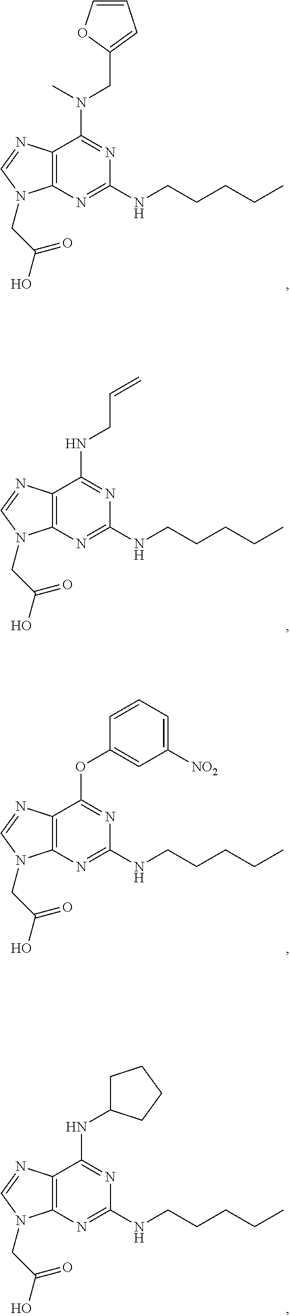 [SMILES: CCCCCNc1nc(N(C)Cc2ccco2)c2ncn(CC(=O)O)c2n1 ; C=CCNc1nc(NCCCCC)nc2c1ncn2CC(=O)O ; CCCCCNc1nc(Oc2cccc([N+](=O)[O-])c2)c2ncn(CC(=O)O)c2n1 ; CCCCCNc1nc(NC2CCCC2)c2ncn(CC(=O)O)c2n1]

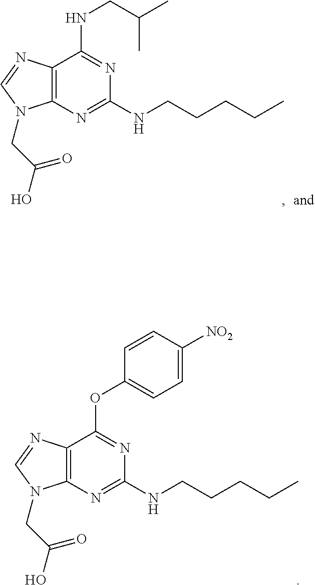 [SMILES: CCCCCNc1nc(NCC(C)C)c2ncn(CC(=O)O)c2n1 ; CCCCCNc1nc(Oc2ccc([N+](=O)[O-])cc2)c2ncn(CC(=O)O)c2n1]

In one aspect, a compound can be present as one or more of the following structures:

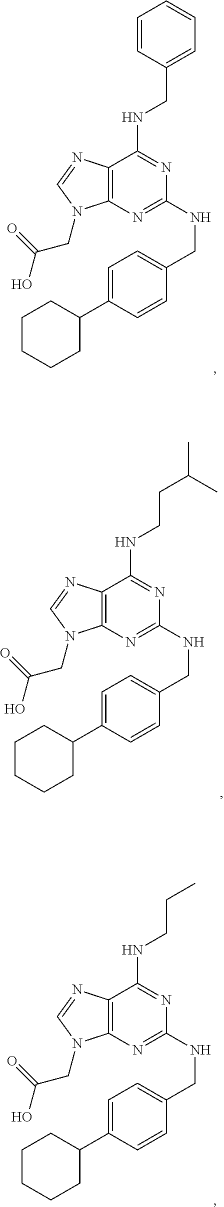 [SMILES: O=C(O)Cn1cnc2c(NCc3ccccc3)nc(NCc3ccc(C4CCCCC4)cc3)nc21 ; CC(C)CCNc1nc(NCc2ccc(C3CCCCC3)cc2)nc2c1ncn2CC(=O)O ; CCCNc1nc(NCc2ccc(C3CCCCC3)cc2)nc2c1ncn2CC(=O)O]

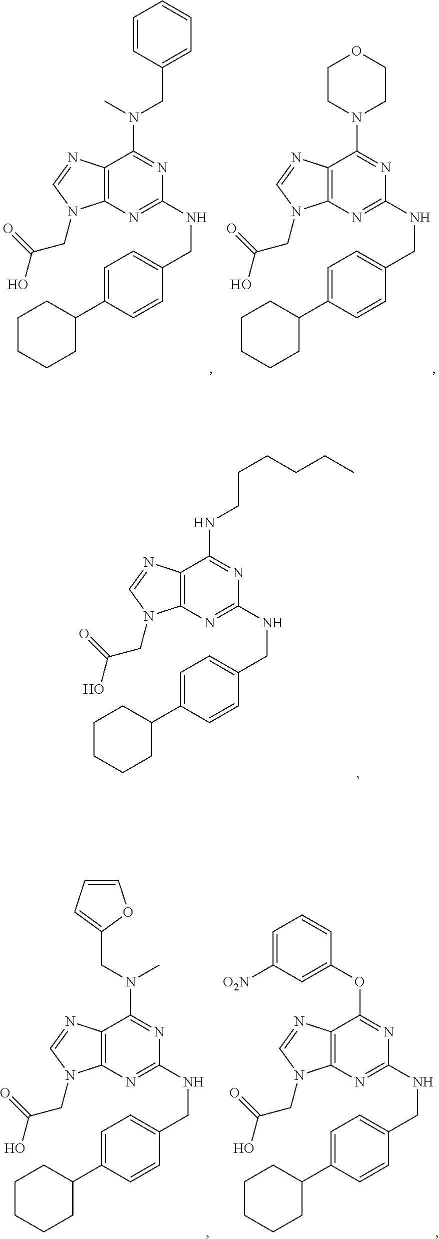

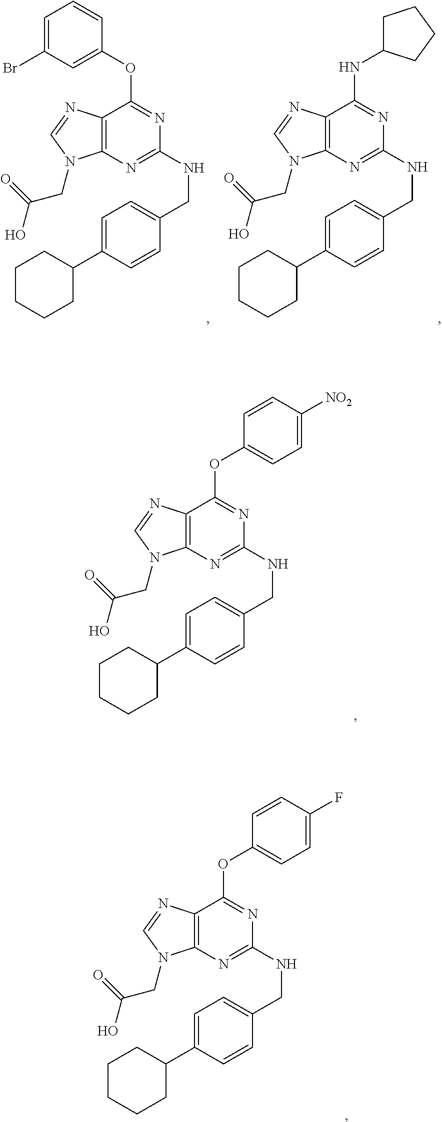

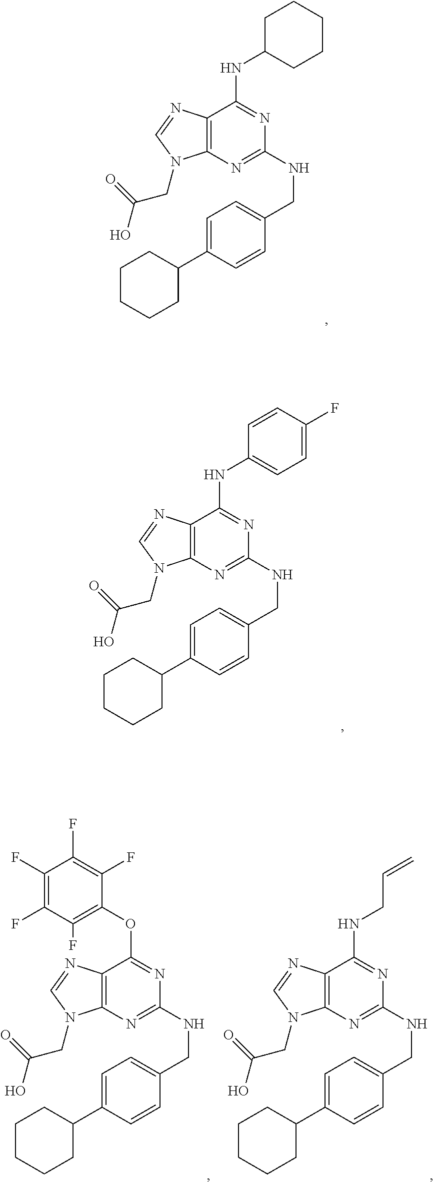

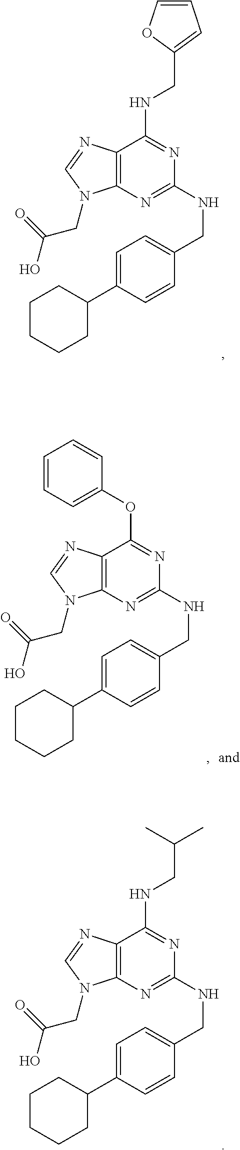 [SMILES: O=C(O)Cn1cnc2c(NCc3ccco3)nc(NCc3ccc(C4CCCCC4)cc3)nc21 ; O=C(O)Cn1cnc2c(Oc3ccccc3)nc(NCc3ccc(C4CCCCC4)cc3)nc21 ; CC(C)CNc1nc(NCc2ccc(C3CCCCC3)cc2)nc2c1ncn2CC(=O)O]

In one aspect, a compound can be present as one or more of the following structures:

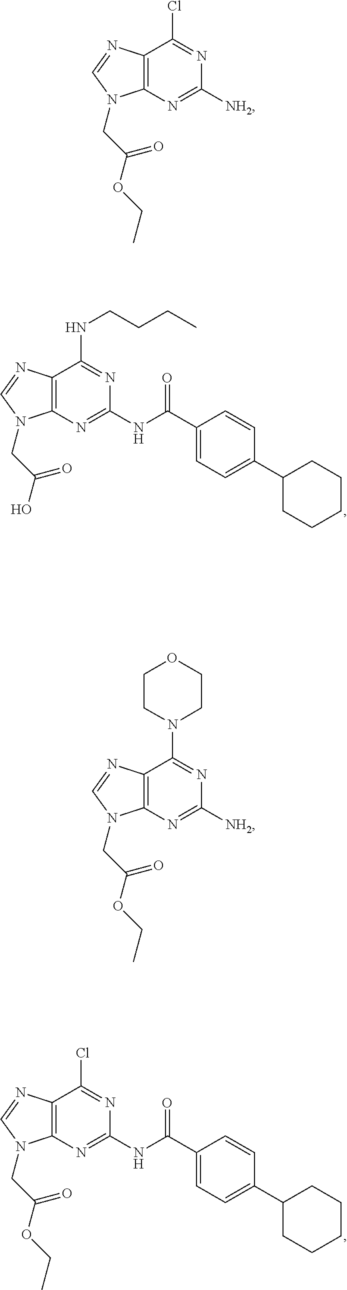 [SMILES: CCOC(=O)Cn1cnc2c(Cl)nc(N)nc21 ; CCCCNc1nc(NC(=O)c2ccc(C3CCCCC3)cc2)nc2c1ncn2CC(=O)O ; CCOC(=O)Cn1cnc2c(N3CCOCC3)nc(N)nc21 ; CCOC(=O)Cn1cnc2c(Cl)nc(NC(=O)c3ccc(C4CCCCC4)cc3)nc21]

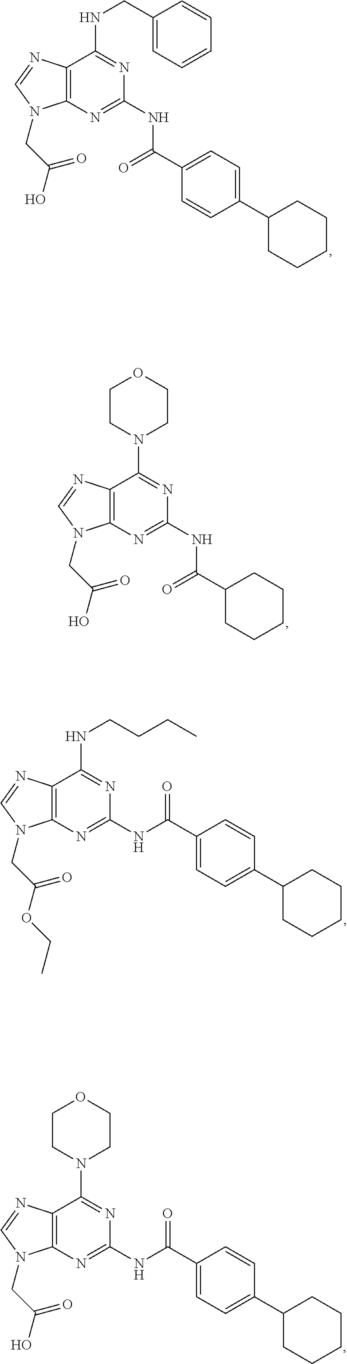 [SMILES: O=C(O)Cn1cnc2c(NCc3ccccc3)nc(NC(=O)c3ccc(C4CCCCC4)cc3)nc21 ; O=C(O)Cn1cnc2c(N3CCOCC3)nc(NC(=O)C3CCCCC3)nc21 ; CCCCNc1nc(NC(=O)c2ccc(C3CCCCC3)cc2)nc2c1ncn2CC(=O)OCC ; O=C(O)Cn1cnc2c(N3CCOCC3)nc(NC(=O)c3ccc(C4CCCCC4)cc3)nc21]

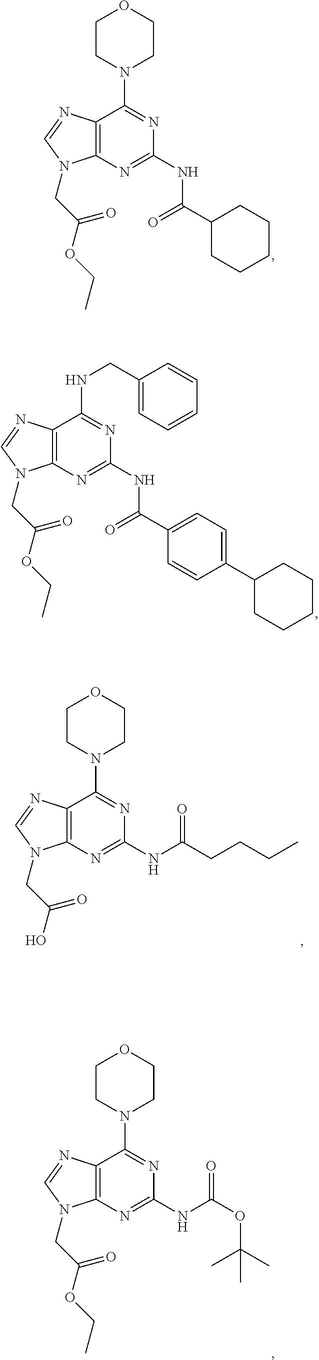 [SMILES: CCOC(=O)Cn1cnc2c(N3CCOCC3)nc(NC(=O)C3CCCCC3)nc21 ; CCOC(=O)Cn1cnc2c(NCc3ccccc3)nc(NC(=O)c3ccc(C4CCCCC4)cc3)nc21 ; CCCCC(=O)Nc1nc(N2CCOCC2)c2ncn(CC(=O)O)c2n1 ; CCOC(=O)Cn1cnc2c(N3CCOCC3)nc(NC(=O)OC(C)(C)C)nc21]

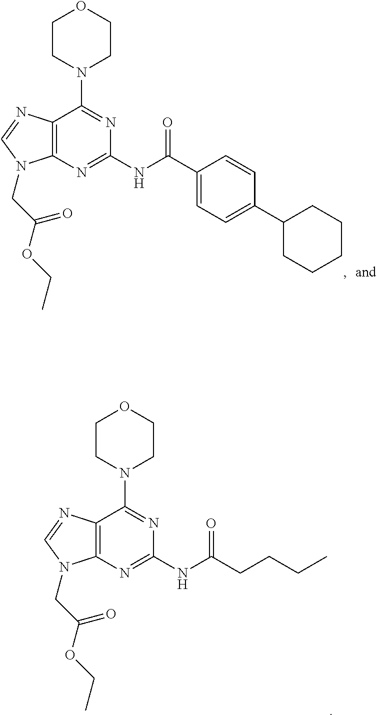 [SMILES: CCOC(=O)Cn1cnc2c(N3CCOCC3)nc(NC(=O)c3ccc(C4CCCCC4)cc3)nc21 ; CCCCC(=O)Nc1nc(N2CCOCC2)c2ncn(CC(=O)OCC)c2n1]

In one aspect, a compound can be present as one or more of the following structures:

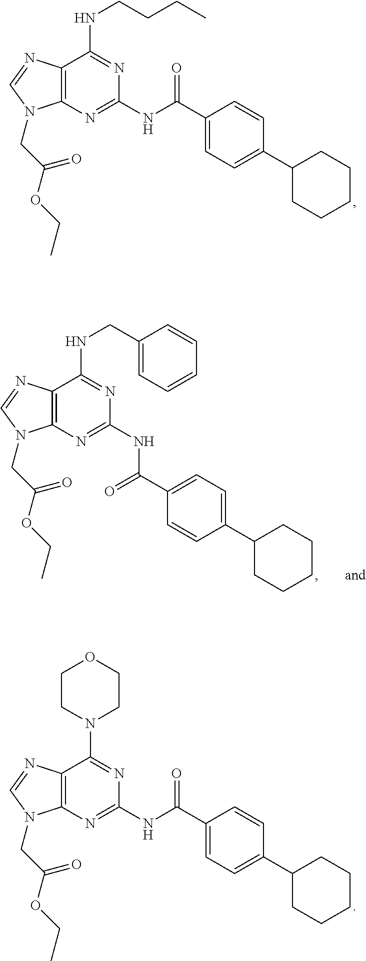 [SMILES: CCCCNc1nc(NC(=O)c2ccc(C3CCCCC3)cc2)nc2c1ncn2CC(=O)OCC ; CCOC(=O)Cn1cnc2c(NCc3ccccc3)nc(NC(=O)c3ccc(C4CCCCC4)cc3)nc21 ; CCOC(=O)Cn1cnc2c(N3CCOCC3)nc(NC(=O)c3ccc(C4CCCCC4)cc3)nc21]

In one aspect, a compound can be present as one or more of the following structures:

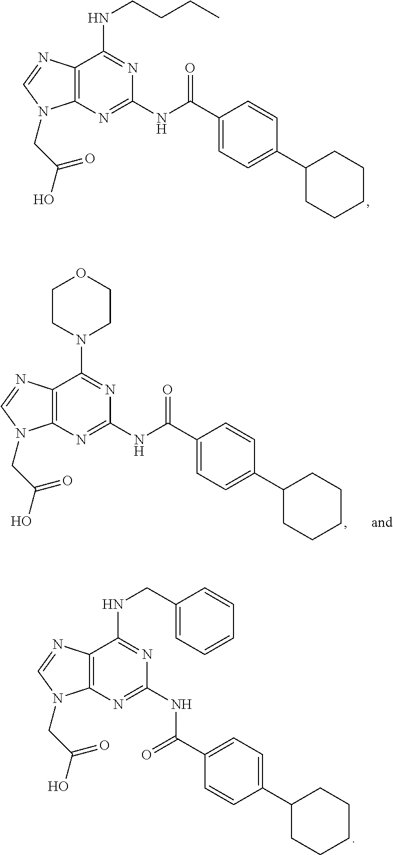 [SMILES: CCCCNc1nc(NC(=O)c2ccc(C3CCCCC3)cc2)nc2c1ncn2CC(=O)O ; O=C(O)Cn1cnc2c(N3CCOCC3)nc(NC(=O)c3ccc(C4CCCCC4)cc3)nc21 ; O=C(O)Cn1cnc2c(NCc3ccccc3)nc(NC(=O)c3ccc(C4CCCCC4)cc3)nc21]

In one aspect, a compound can be present as one or more of the following structures:

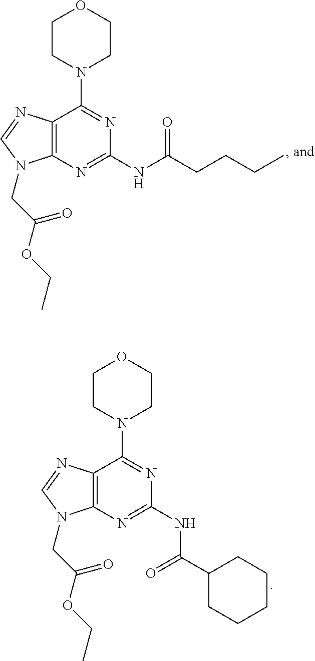 [SMILES: CCCCC(=O)Nc1nc(N2CCOCC2)c2ncn(CC(=O)OCC)c2n1 ; CCOC(=O)Cn1cnc2c(N3CCOCC3)nc(NC(=O)C3CCCCC3)nc21]

In one aspect, a compound can be present as one or more of the following structures:

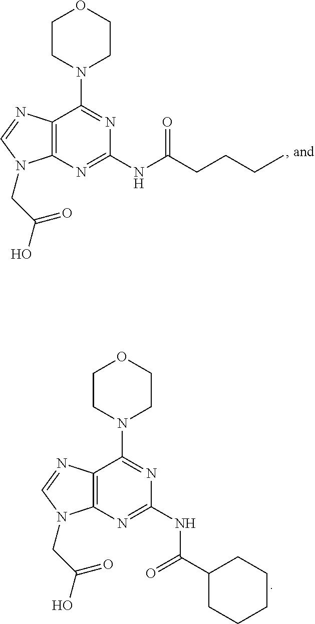 [SMILES: CCCCC(=O)Nc1nc(N2CCOCC2)c2ncn(CC(=O)O)c2n1 ; O=C(O)Cn1cnc2c(N3CCOCC3)nc(NC(=O)C3CCCCC3)nc21]

In one aspect, a compound can be present as one or more of the following structures:

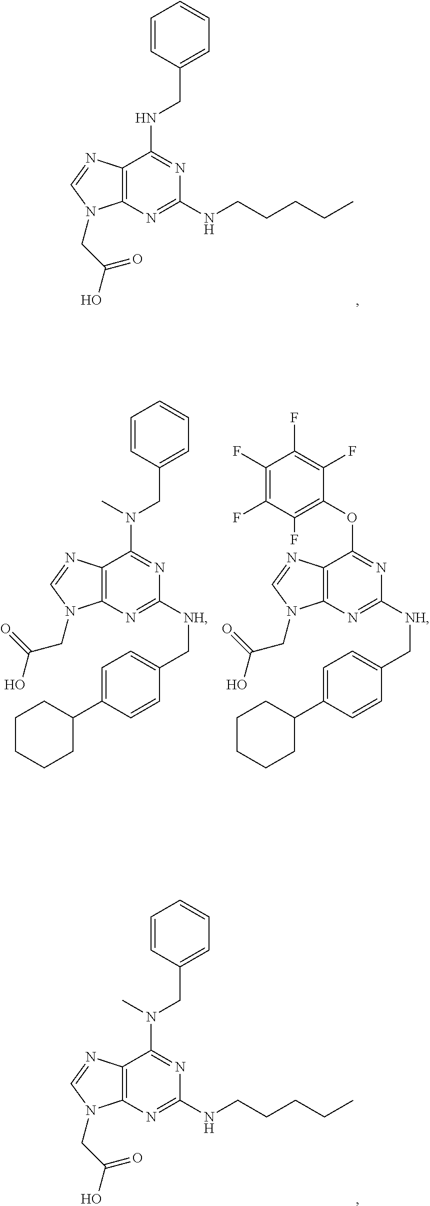

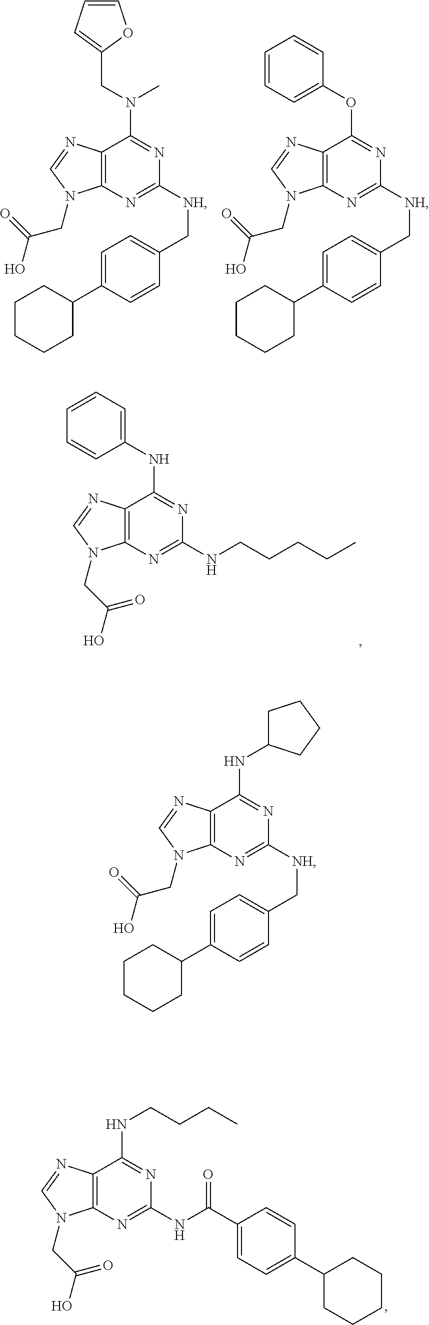

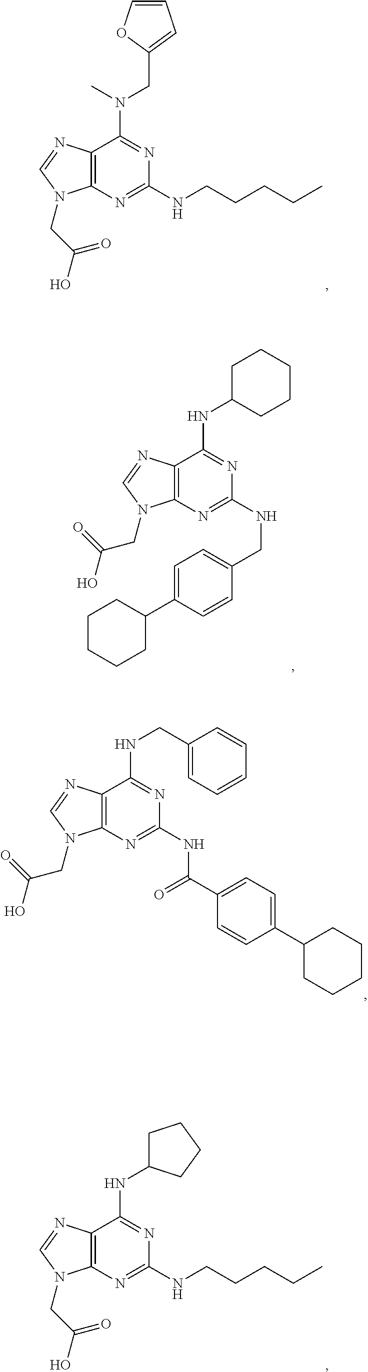 [SMILES: CCCCCNc1nc(N(C)Cc2ccco2)c2ncn(CC(=O)O)c2n1 ; O=C(O)Cn1cnc2c(NC3CCCCC3)nc(NCc3ccc(C4CCCCC4)cc3)nc21 ; O=C(O)Cn1cnc2c(NCc3ccccc3)nc(NC(=O)c3ccc(C4CCCCC4)cc3)nc21 ; CCCCCNc1nc(NC2CCCC2)c2ncn(CC(=O)O)c2n1]

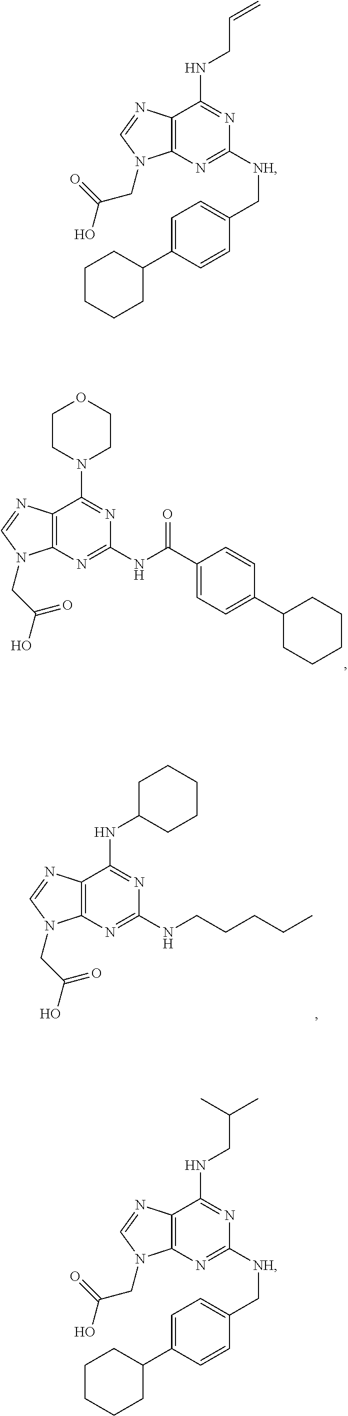 [SMILES: C=CCNc1nc(NCc2ccc(C3CCCCC3)cc2)nc2c1ncn2CC(=O)O ; O=C(O)Cn1cnc2c(N3CCOCC3)nc(NC(=O)c3ccc(C4CCCCC4)cc3)nc21 ; CCCCCNc1nc(NC2CCCCC2)c2ncn(CC(=O)O)c2n1 ; CC(C)CNc1nc(NCc2ccc(C3CCCCC3)cc2)nc2c1ncn2CC(=O)O]

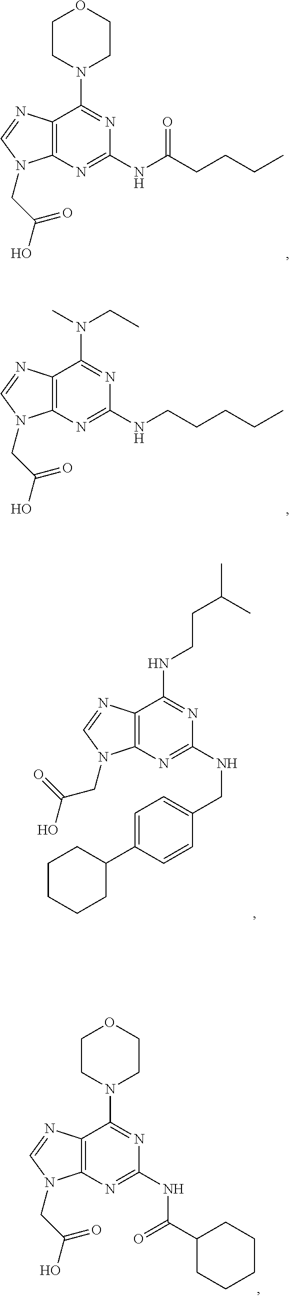 [SMILES: CCCCC(=O)Nc1nc(N2CCOCC2)c2ncn(CC(=O)O)c2n1 ; CCCCCNc1nc(N(C)CC)c2ncn(CC(=O)O)c2n1 ; CC(C)CCNc1nc(NCc2ccc(C3CCCCC3)cc2)nc2c1ncn2CC(=O)O ; O=C(O)Cn1cnc2c(N3CCOCC3)nc(NC(=O)C3CCCCC3)nc21]

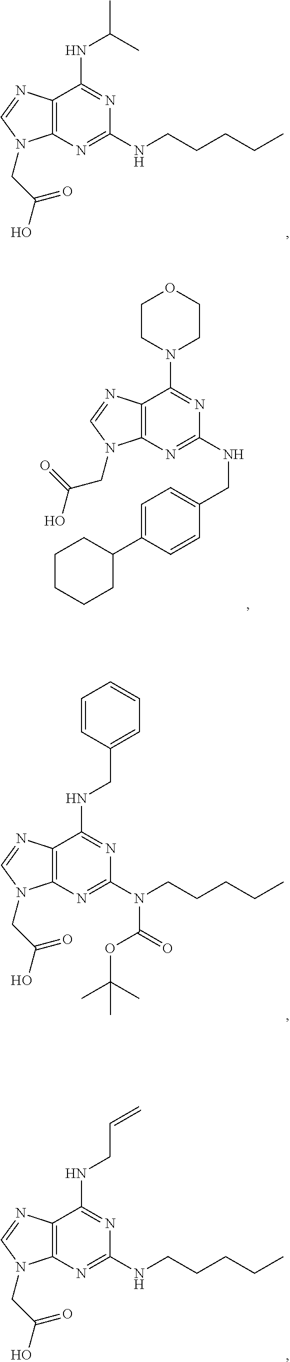 [SMILES: CCCCCNc1nc(NC(C)C)c2ncn(CC(=O)O)c2n1 ; O=C(O)Cn1cnc2c(N3CCOCC3)nc(NCc3ccc(C4CCCCC4)cc3)nc21 ; CCCCCN(C(=O)OC(C)(C)C)c1nc(NCc2ccccc2)c2ncn(CC(=O)O)c2n1 ; C=CCNc1nc(NCCCCC)nc2c1ncn2CC(=O)O]

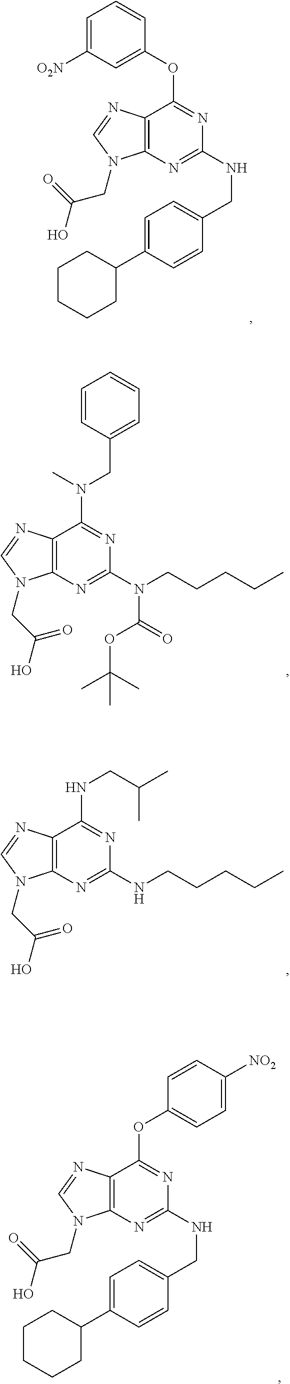 [SMILES: O=C(O)Cn1cnc2c(Oc3cccc([N+](=O)[O-])c3)nc(NCc3ccc(C4CCCCC4)cc3)nc21 ; CCCCCN(C(=O)OC(C)(C)C)c1nc(N(C)Cc2ccccc2)c2ncn(CC(=O)O)c2n1 ; CCCCCNc1nc(NCC(C)C)c2ncn(CC(=O)O)c2n1 ; O=C(O)Cn1cnc2c(Oc3ccc([N+](=O)[O-])cc3)nc(NCc3ccc(C4CCCCC4)cc3)nc21]

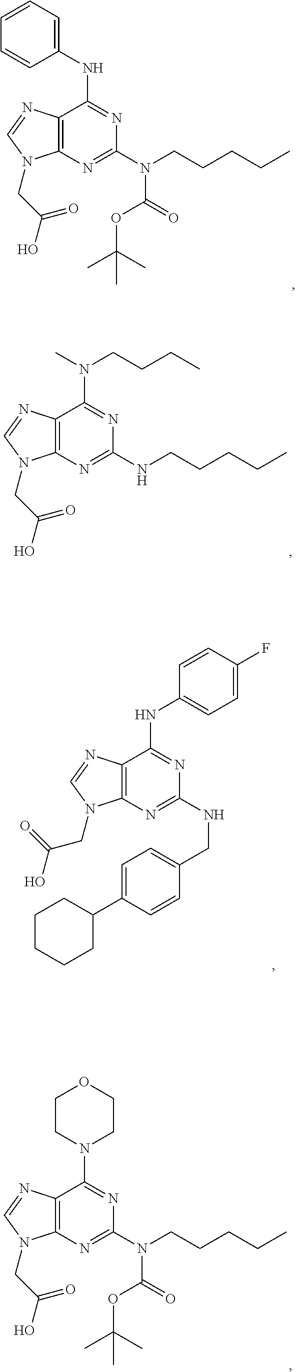 [SMILES: CCCCCN(C(=O)OC(C)(C)C)c1nc(Nc2ccccc2)c2ncn(CC(=O)O)c2n1 ; CCCCCNc1nc(N(C)CCCC)c2ncn(CC(=O)O)c2n1 ; O=C(O)Cn1cnc2c(Nc3ccc(F)cc3)nc(NCc3ccc(C4CCCCC4)cc3)nc21 ; CCCCCN(C(=O)OC(C)(C)C)c1nc(N2CCOCC2)c2ncn(CC(=O)O)c2n1]

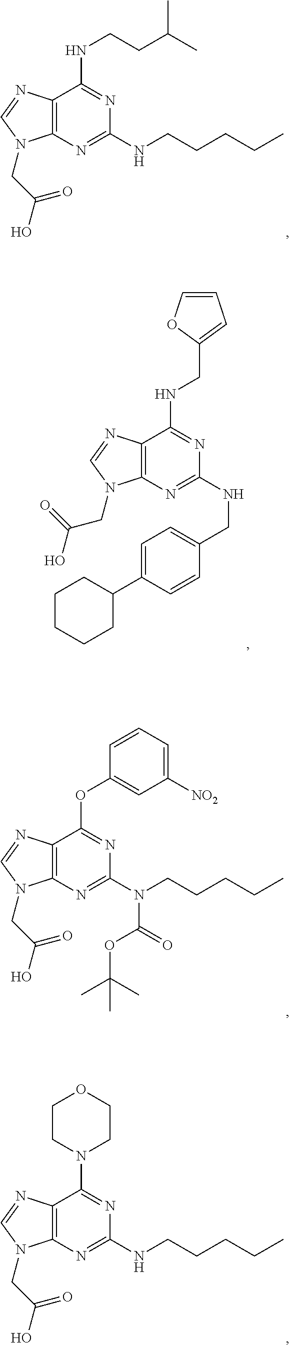 [SMILES: CCCCCNc1nc(NCCC(C)C)c2ncn(CC(=O)O)c2n1 ; O=C(O)Cn1cnc2c(NCc3ccco3)nc(NCc3ccc(C4CCCCC4)cc3)nc21 ; CCCCCN(C(=O)OC(C)(C)C)c1nc(Oc2cccc([N+](=O)[O-])c2)c2ncn(CC(=O)O)c2n1 ; CCCCCNc1nc(N2CCOCC2)c2ncn(CC(=O)O)c2n1]

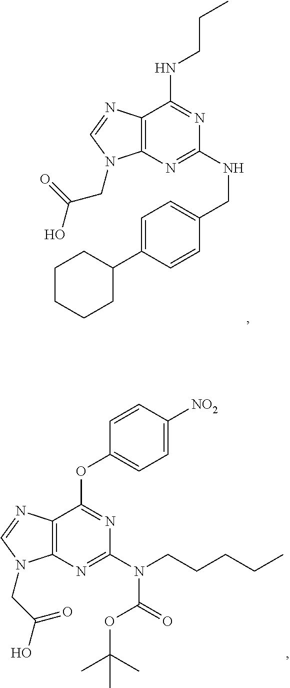 [SMILES: CCCNc1nc(NCc2ccc(C3CCCCC3)cc2)nc2c1ncn2CC(=O)O ; CCCCCN(C(=O)OC(C)(C)C)c1nc(Oc2ccc([N+](=O)[O-])cc2)c2ncn(CC(=O)O)c2n1]

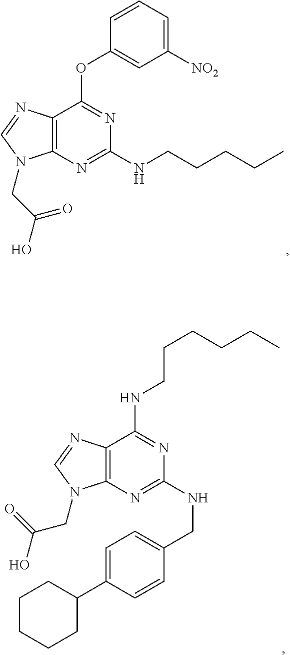 [SMILES: CCCCCNc1nc(Oc2cccc([N+](=O)[O-])c2)c2ncn(CC(=O)O)c2n1 ; CCCCCCNc1nc(NCc2ccc(C3CCCCC3)cc2)nc2c1ncn2CC(=O)O]

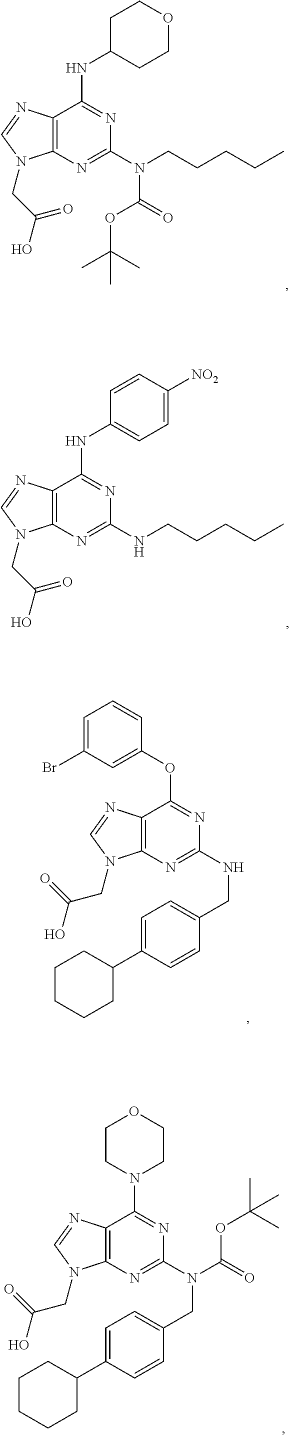 [SMILES: CCCCCN(C(=O)OC(C)(C)C)c1nc(NC2CCOCC2)c2ncn(CC(=O)O)c2n1 ; CCCCCNc1nc(Nc2ccc([N+](=O)[O-])cc2)c2ncn(CC(=O)O)c2n1 ; O=C(O)Cn1cnc2c(Oc3cccc(Br)c3)nc(NCc3ccc(C4CCCCC4)cc3)nc21 ; CC(C)(C)OC(=O)N(Cc1ccc(C2CCCCC2)cc1)c1nc(N2CCOCC2)c2ncn(CC(=O)O)c2n1]

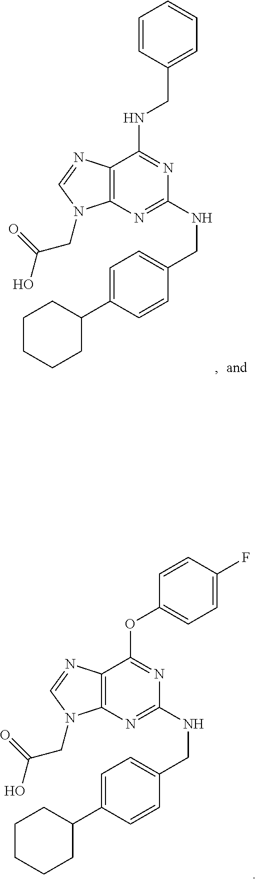 [SMILES: O=C(O)Cn1cnc2c(NCc3ccccc3)nc(NCc3ccc(C4CCCCC4)cc3)nc21 ; O=C(O)Cn1cnc2c(Oc3ccc(F)cc3)nc(NCc3ccc(C4CCCCC4)cc3)nc21]

In one aspect, a compound can be present as one or more of the following structures:

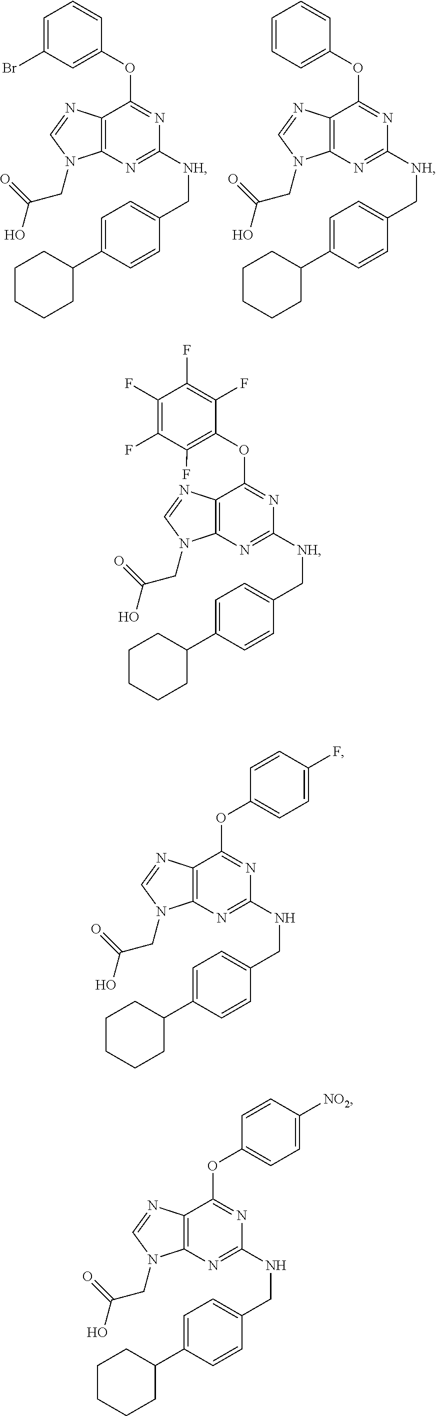 [SMILES: O=C(O)Cn1cnc2c(Oc3cccc(Br)c3)nc(NCc3ccc(C4CCCCC4)cc3)nc21 ; O=C(O)Cn1cnc2c(Oc3ccccc3)nc(NCc3ccc(C4CCCCC4)cc3)nc21 ; O=C(O)Cn1cnc2c(Oc3c(F)c(F)c(F)c(F)c3F)nc(NCc3ccc(C4CCCCC4)cc3)nc21 ; O=C(O)Cn1cnc2c(Oc3ccc(F)cc3)nc(NCc3ccc(C4CCCCC4)cc3)nc21 ; O=C(O)Cn1cnc2c(Oc3ccc([N+](=O)[O-])cc3)nc(NCc3ccc(C4CCCCC4)cc3)nc21]

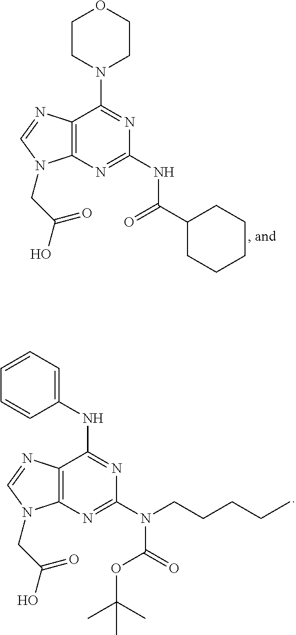 [SMILES: O=C(O)Cn1cnc2c(N3CCOCC3)nc(NC(=O)C3CCCCC3)nc21 ; CCCCCN(C(=O)OC(C)(C)C)c1nc(Nc2ccccc2)c2ncn(CC(=O)O)c2n1]

In one aspect, a compound can be present as one or more of the following structures:

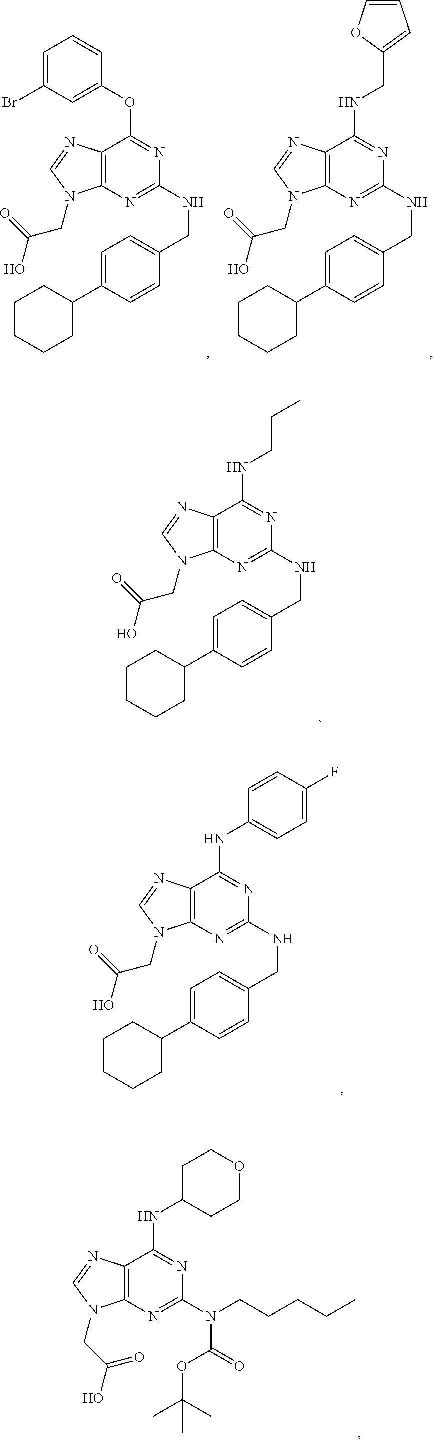

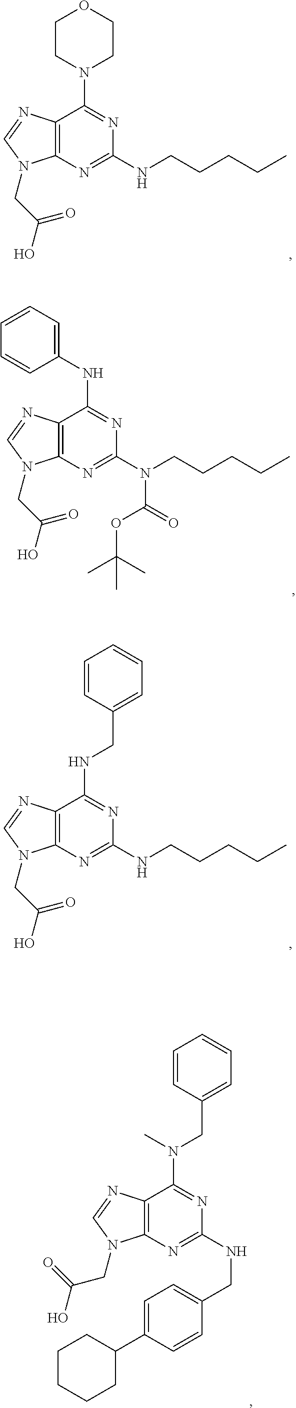 [SMILES: CCCCCNc1nc(N2CCOCC2)c2ncn(CC(=O)O)c2n1 ; CCCCCN(C(=O)OC(C)(C)C)c1nc(Nc2ccccc2)c2ncn(CC(=O)O)c2n1 ; CCCCCNc1nc(NCc2ccccc2)c2ncn(CC(=O)O)c2n1 ; CN(Cc1ccccc1)c1nc(NCc2ccc(C3CCCCC3)cc2)nc2c1ncn2CC(=O)O]

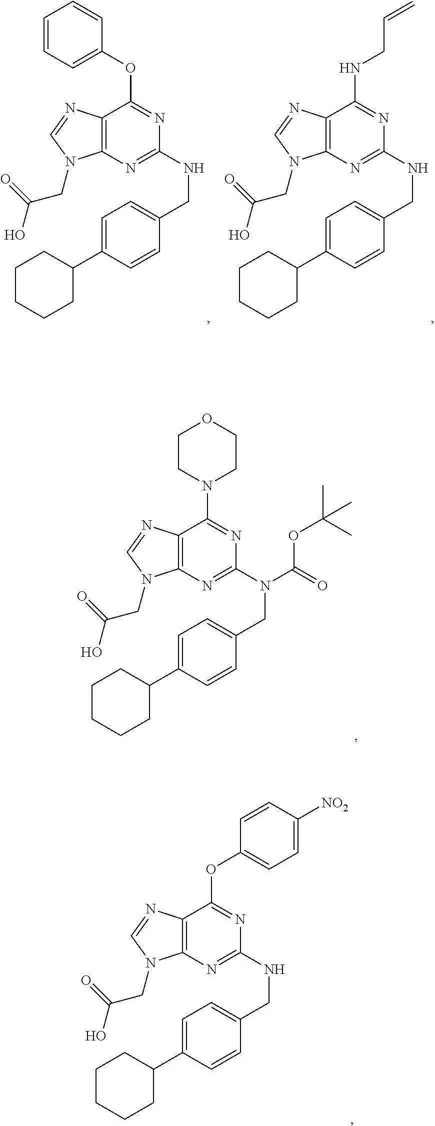

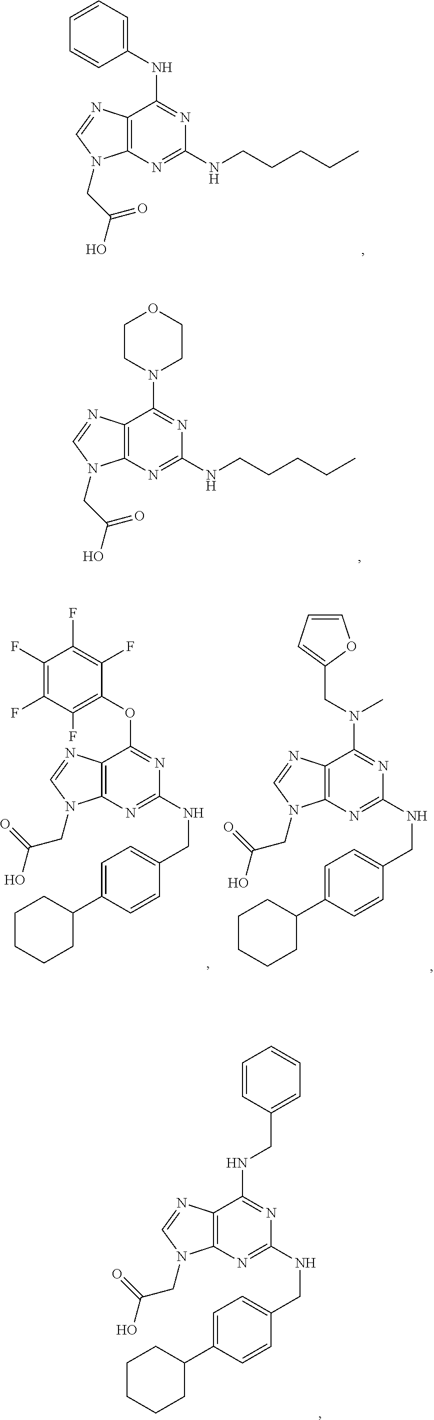

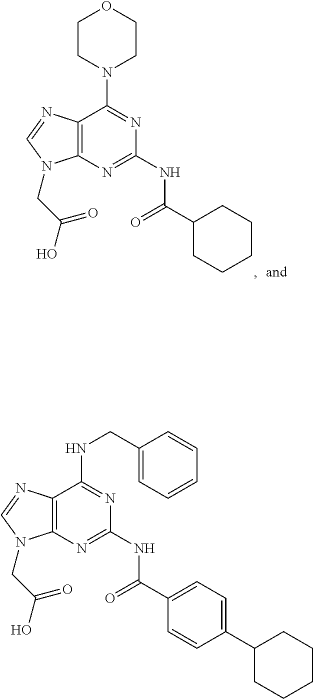 [SMILES: O=C(O)Cn1cnc2c(N3CCOCC3)nc(NC(=O)C3CCCCC3)nc21 ; O=C(O)Cn1cnc2c(NCc3ccccc3)nc(NC(=O)c3ccc(C4CCCCC4)cc3)nc21]

In one aspect, a compound can be present as one or more of the following structures:

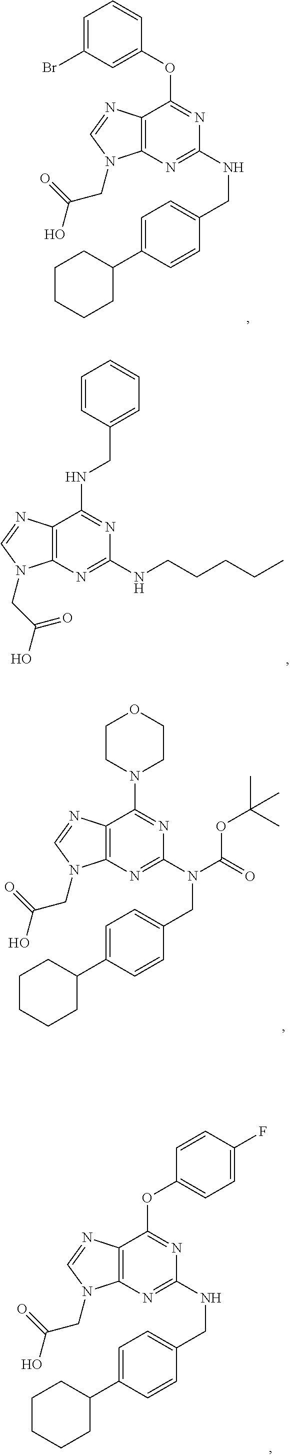 [SMILES: O=C(O)Cn1cnc2c(Oc3cccc(Br)c3)nc(NCc3ccc(C4CCCCC4)cc3)nc21 ; CCCCCNc1nc(NCc2ccccc2)c2ncn(CC(=O)O)c2n1 ; CC(C)(C)OC(=O)N(Cc1ccc(C2CCCCC2)cc1)c1nc(N2CCOCC2)c2ncn(CC(=O)O)c2n1 ; O=C(O)Cn1cnc2c(Oc3ccc(F)cc3)nc(NCc3ccc(C4CCCCC4)cc3)nc21]

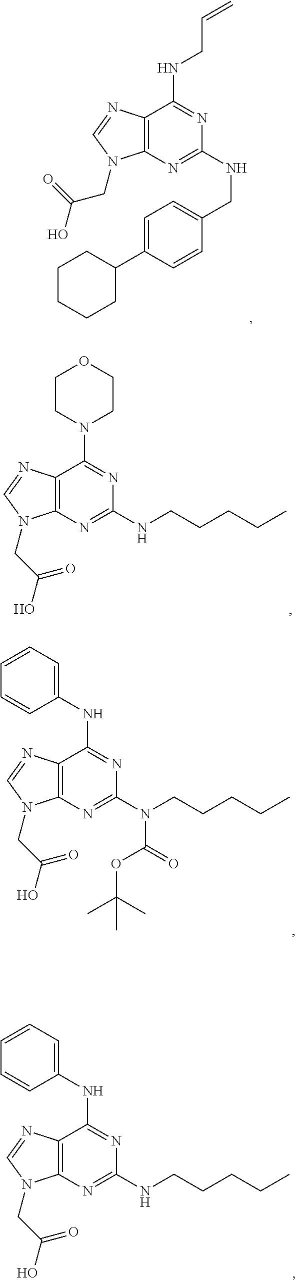

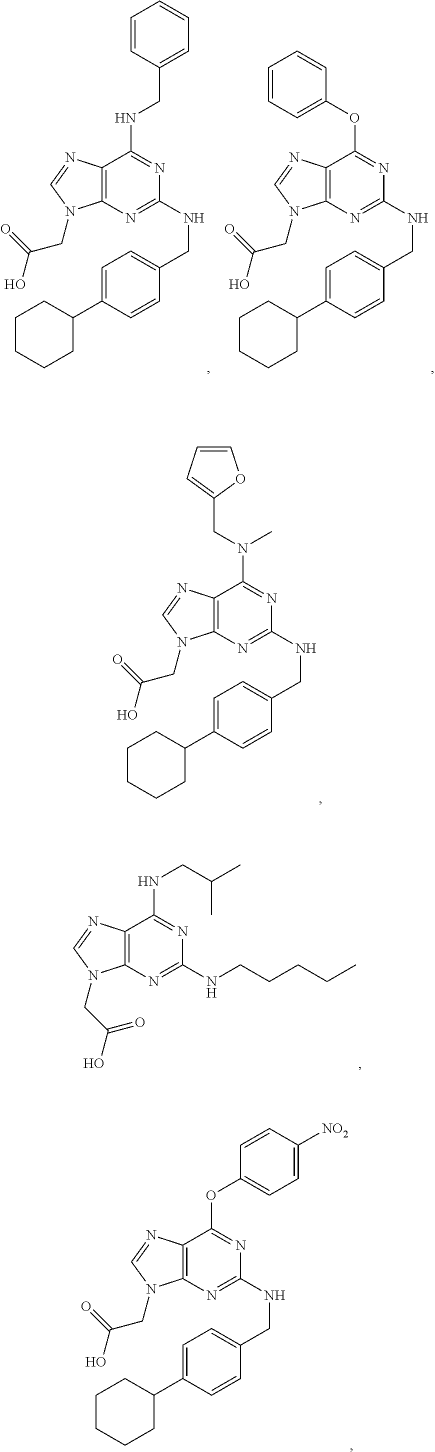

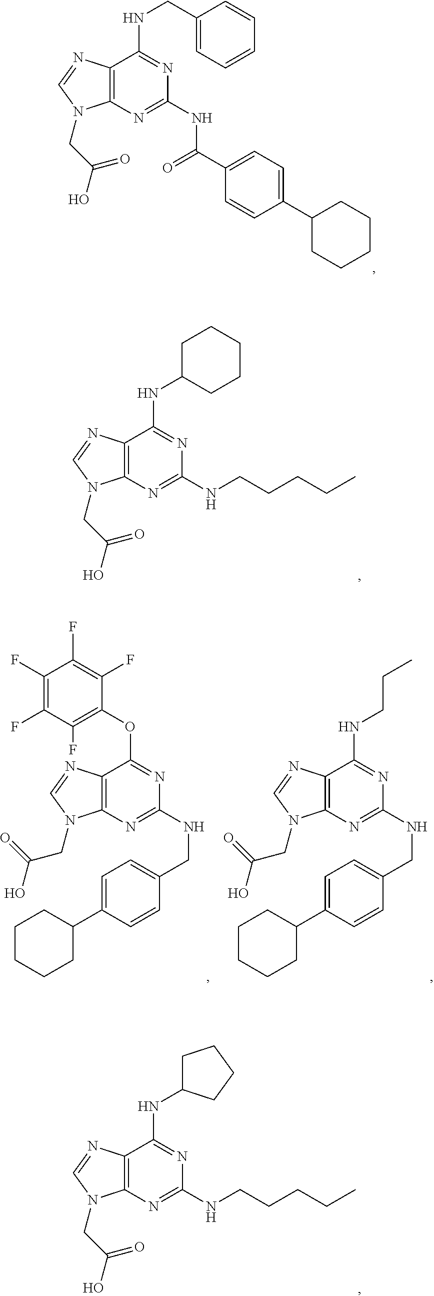

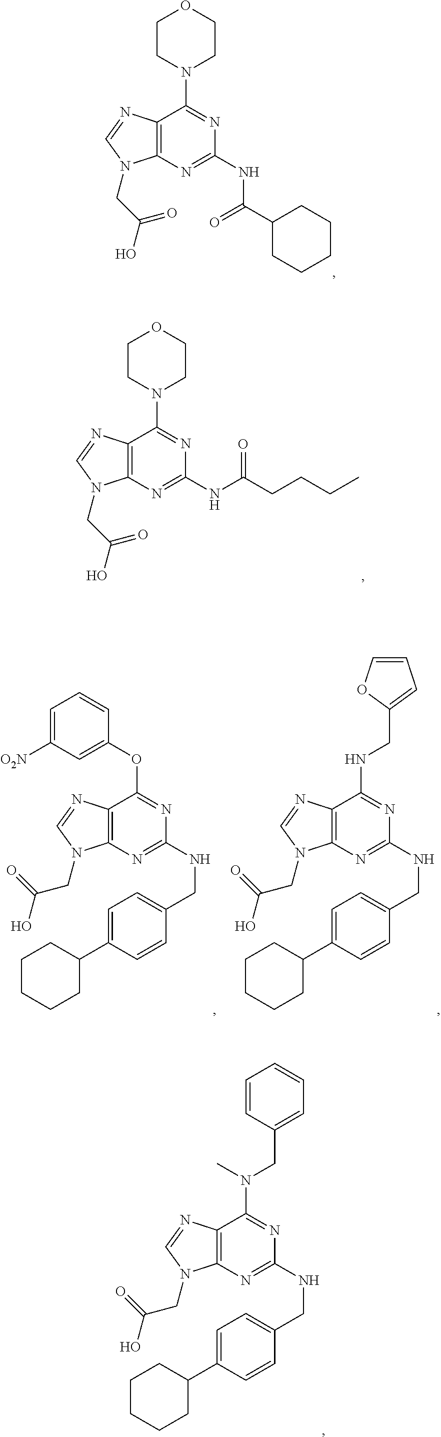

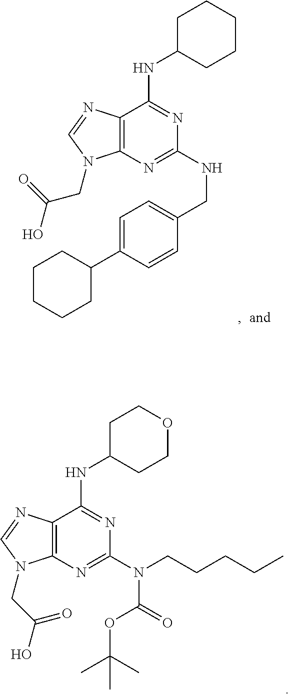 [SMILES: O=C(O)Cn1cnc2c(NC3CCCCC3)nc(NCc3ccc(C4CCCCC4)cc3)nc21 ; CCCCCN(C(=O)OC(C)(C)C)c1nc(NC2CCOCC2)c2ncn(CC(=O)O)c2n1]

In one aspect, a compound can be present as one or more of the following structures:

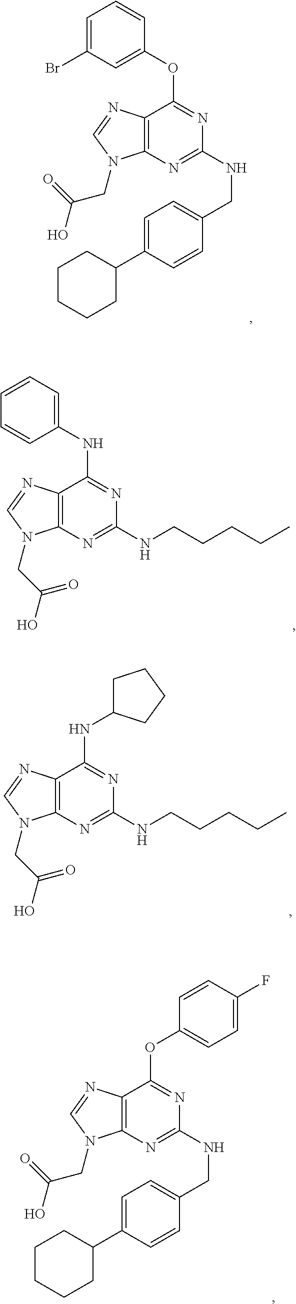 [SMILES: O=C(O)Cn1cnc2c(Oc3cccc(Br)c3)nc(NCc3ccc(C4CCCCC4)cc3)nc21 ; CCCCCNc1nc(Nc2ccccc2)c2ncn(CC(=O)O)c2n1 ; CCCCCNc1nc(NC2CCCC2)c2ncn(CC(=O)O)c2n1 ; O=C(O)Cn1cnc2c(Oc3ccc(F)cc3)nc(NCc3ccc(C4CCCCC4)cc3)nc21]

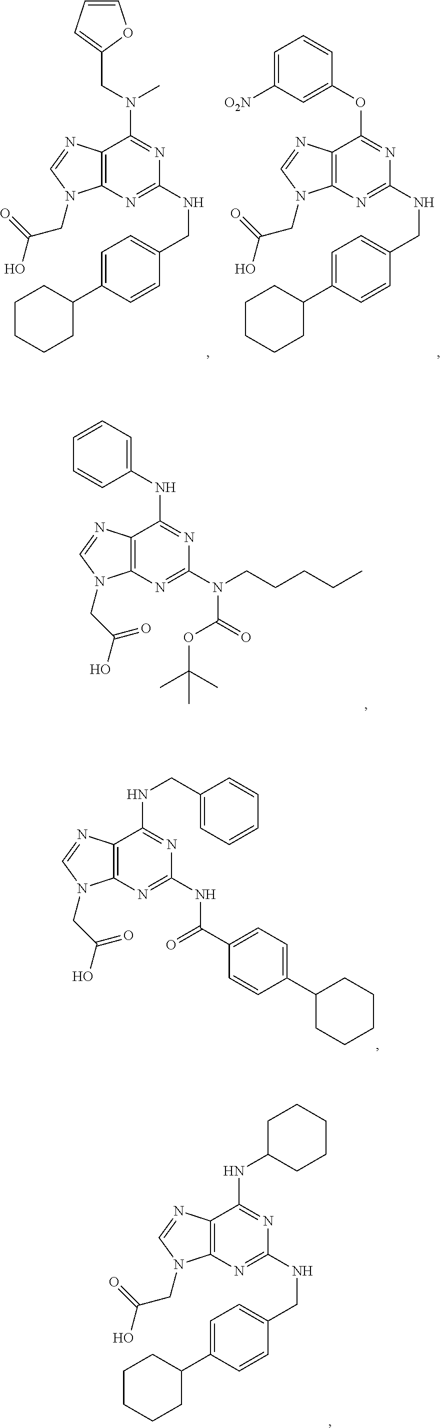 [SMILES: CN(Cc1ccco1)c1nc(NCc2ccc(C3CCCCC3)cc2)nc2c1ncn2CC(=O)O ; O=C(O)Cn1cnc2c(Oc3cccc([N+](=O)[O-])c3)nc(NCc3ccc(C4CCCCC4)cc3)nc21 ; CCCCCN(C(=O)OC(C)(C)C)c1nc(Nc2ccccc2)c2ncn(CC(=O)O)c2n1 ; O=C(O)Cn1cnc2c(NCc3ccccc3)nc(NC(=O)c3ccc(C4CCCCC4)cc3)nc21 ; O=C(O)Cn1cnc2c(NC3CCCCC3)nc(NCc3ccc(C4CCCCC4)cc3)nc21]

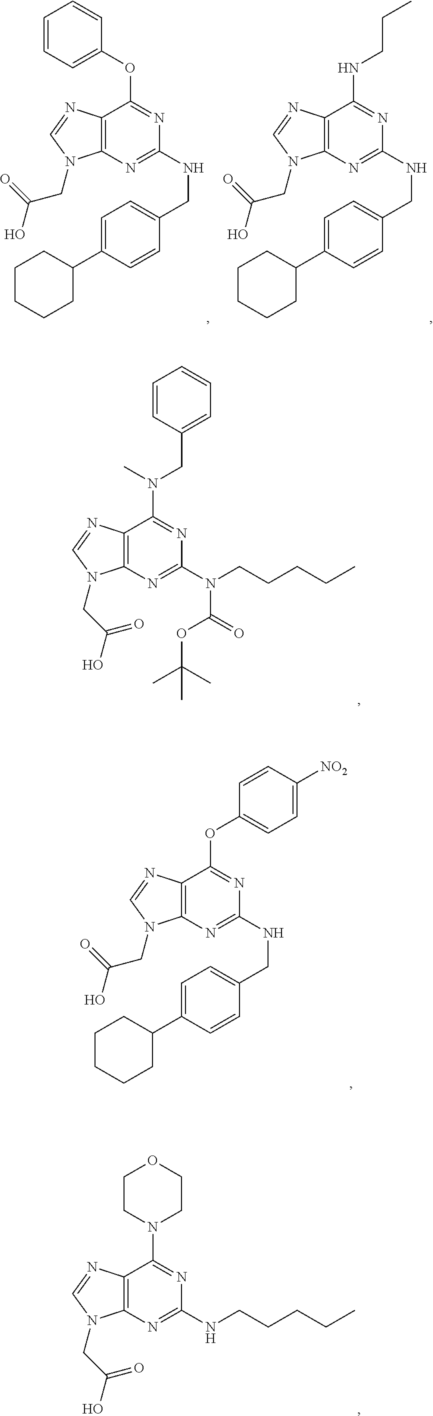

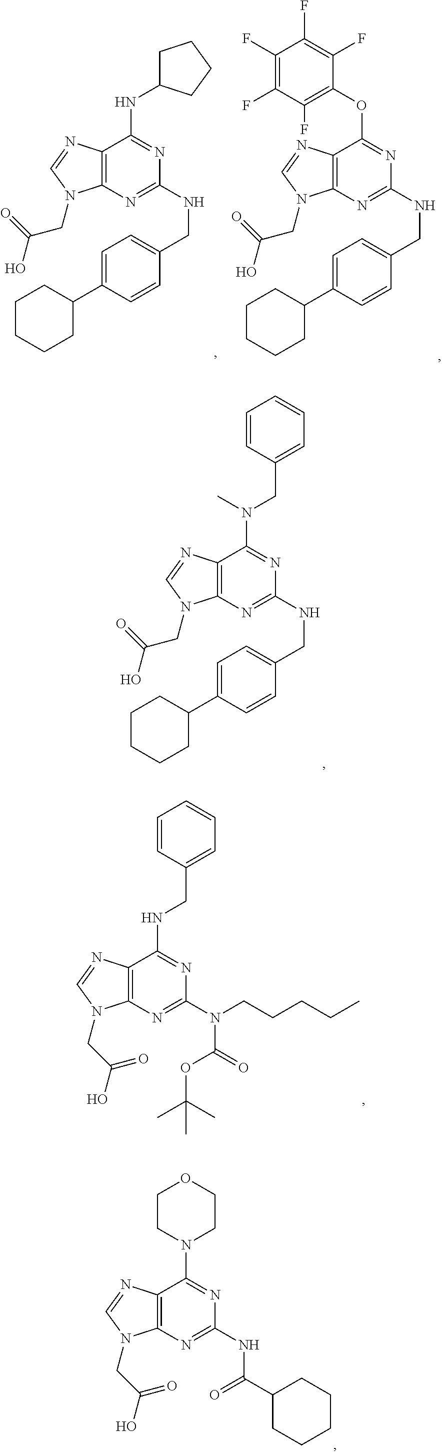

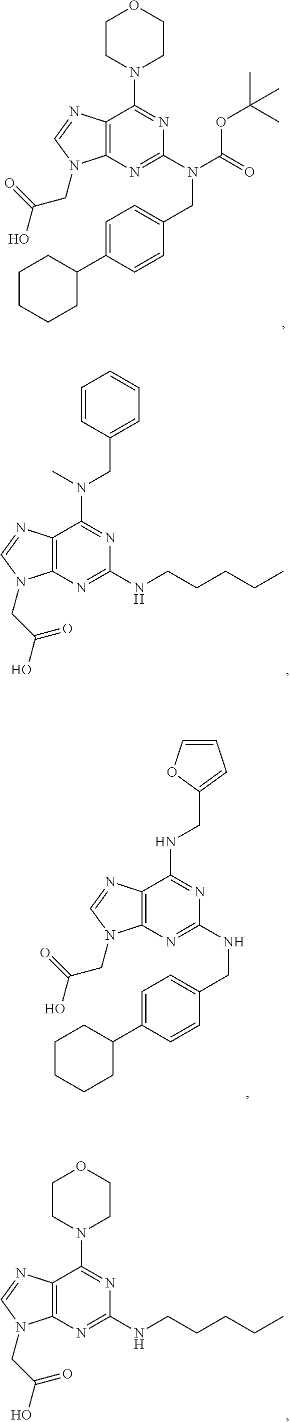 [SMILES: CC(C)(C)OC(=O)N(Cc1ccc(C2CCCCC2)cc1)c1nc(N2CCOCC2)c2ncn(CC(=O)O)c2n1 ; CCCCCNc1nc(N(C)Cc2ccccc2)c2ncn(CC(=O)O)c2n1 ; O=C(O)Cn1cnc2c(NCc3ccco3)nc(NCc3ccc(C4CCCCC4)cc3)nc21 ; CCCCCNc1nc(N2CCOCC2)c2ncn(CC(=O)O)c2n1]

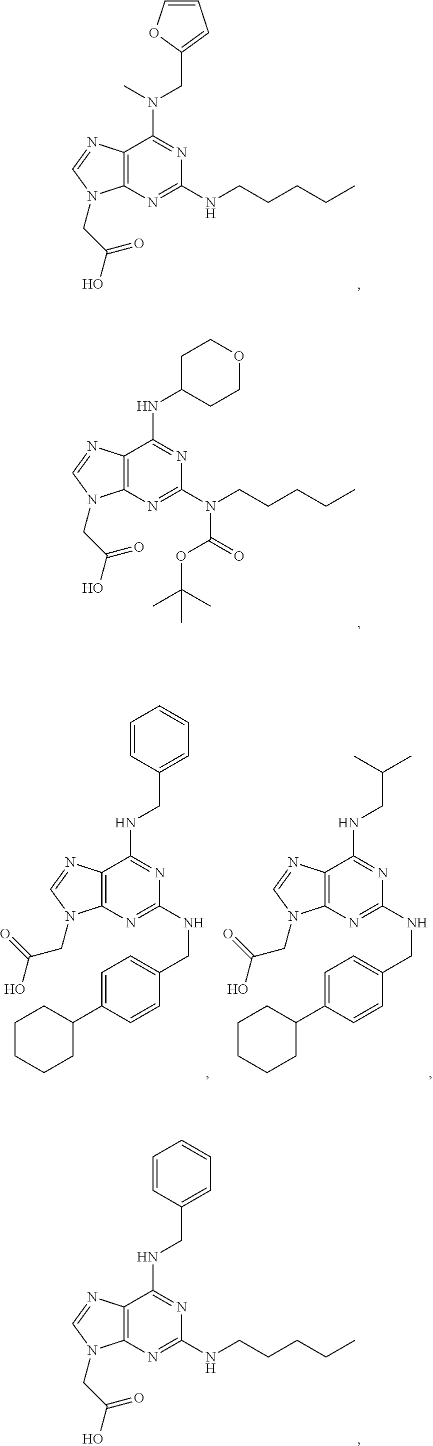

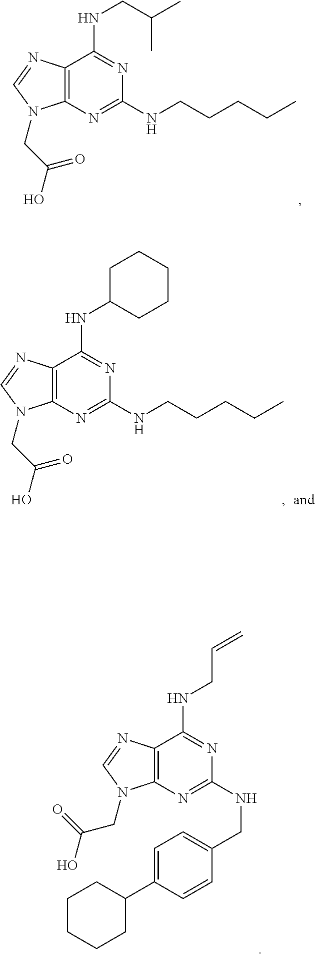 [SMILES: CCCCCNc1nc(NCC(C)C)c2ncn(CC(=O)O)c2n1 ; CCCCCNc1nc(NC2CCCCC2)c2ncn(CC(=O)O)c2n1 ; C=CCNc1nc(NCc2ccc(C3CCCCC3)cc2)nc2c1ncn2CC(=O)O]

In one aspect, a compound can be present as one or more of the following structures:

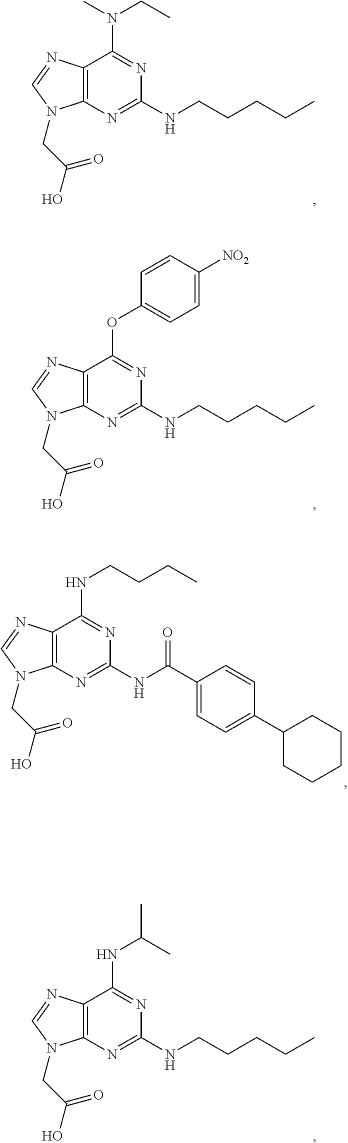 [SMILES: CCCCCNc1nc(N(C)CC)c2ncn(CC(=O)O)c2n1 ; CCCCCNc1nc(Oc2ccc([N+](=O)[O-])cc2)c2ncn(CC(=O)O)c2n1 ; CCCCNc1nc(NC(=O)c2ccc(C3CCCCC3)cc2)nc2c1ncn2CC(=O)O ; CCCCCNc1nc(NC(C)C)c2ncn(CC(=O)O)c2n1]

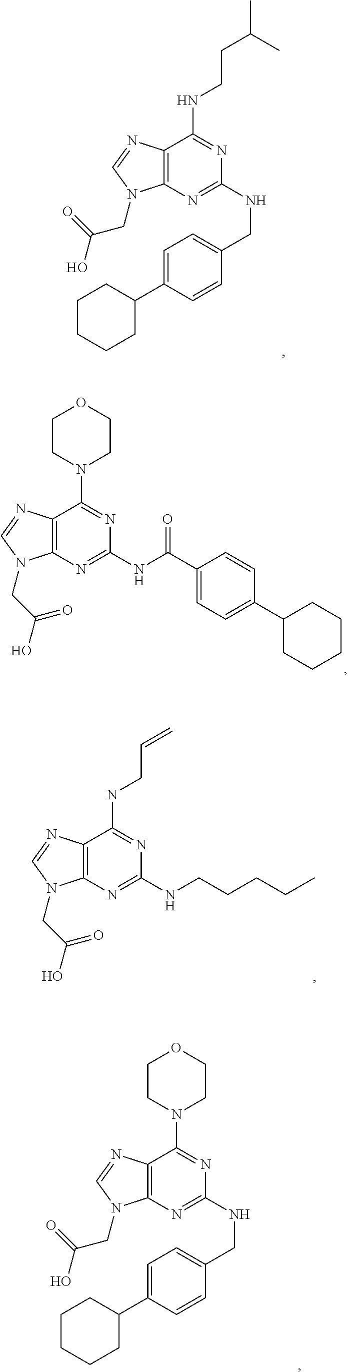 [SMILES: CC(C)CCNc1nc(NCc2ccc(C3CCCCC3)cc2)nc2c1ncn2CC(=O)O ; O=C(O)Cn1cnc2c(N3CCOCC3)nc(NC(=O)c3ccc(C4CCCCC4)cc3)nc21 ; C=CCNc1nc(NCCCCC)nc2c1ncn2CC(=O)O ; O=C(O)Cn1cnc2c(N3CCOCC3)nc(NCc3ccc(C4CCCCC4)cc3)nc21]

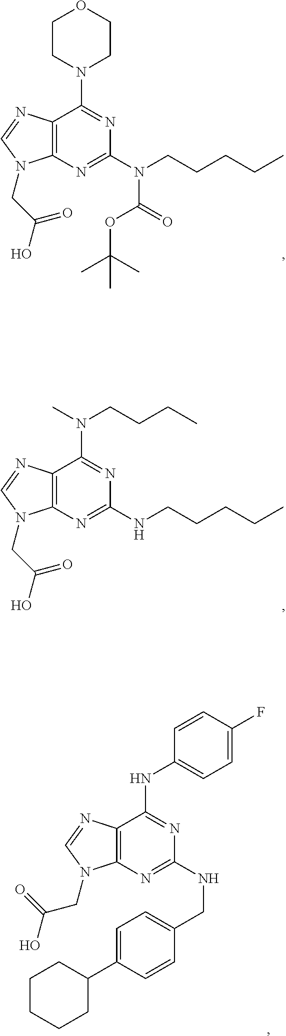 [SMILES: CCCCCN(C(=O)OC(C)(C)C)c1nc(N2CCOCC2)c2ncn(CC(=O)O)c2n1 ; CCCCCNc1nc(N(C)CCCC)c2ncn(CC(=O)O)c2n1 ; O=C(O)Cn1cnc2c(Nc3ccc(F)cc3)nc(NCc3ccc(C4CCCCC4)cc3)nc21]

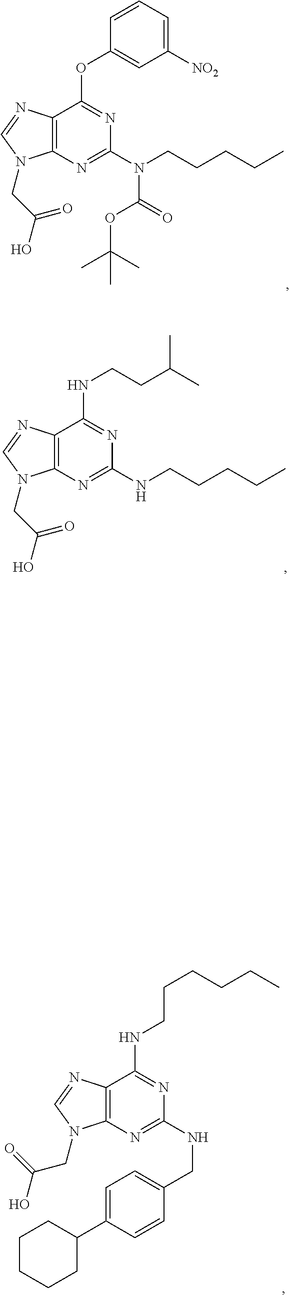 [SMILES: CCCCCN(C(=O)OC(C)(C)C)c1nc(Oc2cccc([N+](=O)[O-])c2)c2ncn(CC(=O)O)c2n1 ; CCCCCNc1nc(NCCC(C)C)c2ncn(CC(=O)O)c2n1 ; CCCCCCNc1nc(NCc2ccc(C3CCCCC3)cc2)nc2c1ncn2CC(=O)O]

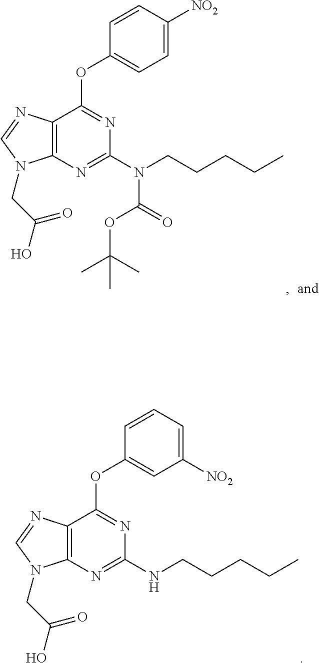 [SMILES: CCCCCN(C(=O)OC(C)(C)C)c1nc(Oc2ccc([N+](=O)[O-])cc2)c2ncn(CC(=O)O)c2n1 ; CCCCCNc1nc(Oc2cccc([N+](=O)[O-])c2)c2ncn(CC(=O)O)c2n1]

In one aspect, a compound can be present as one or more of the following structures:

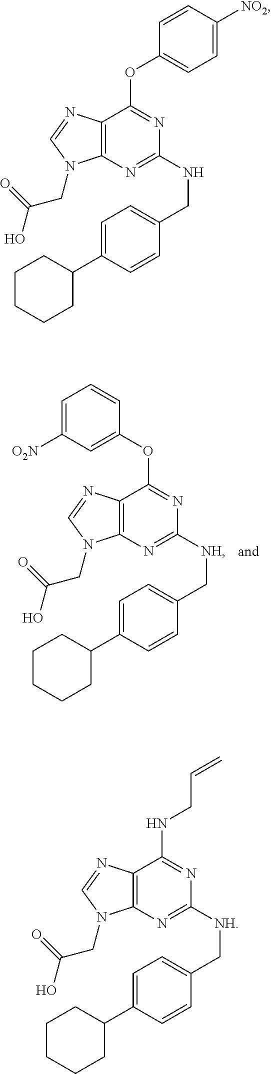 [SMILES: O=C(O)Cn1cnc2c(Oc3ccc([N+](=O)[O-])cc3)nc(NCc3ccc(C4CCCCC4)cc3)nc21 ; O=C(O)Cn1cnc2c(Oc3cccc([N+](=O)[O-])c3)nc(NCc3ccc(C4CCCCC4)cc3)nc21 ; "and" ; C=CCNc1nc(NCc2ccc(C3CCCCC3)cc2)nc2c1ncn2CC(=O)O]

In one aspect, a compound can be present as one or more of the following structures:

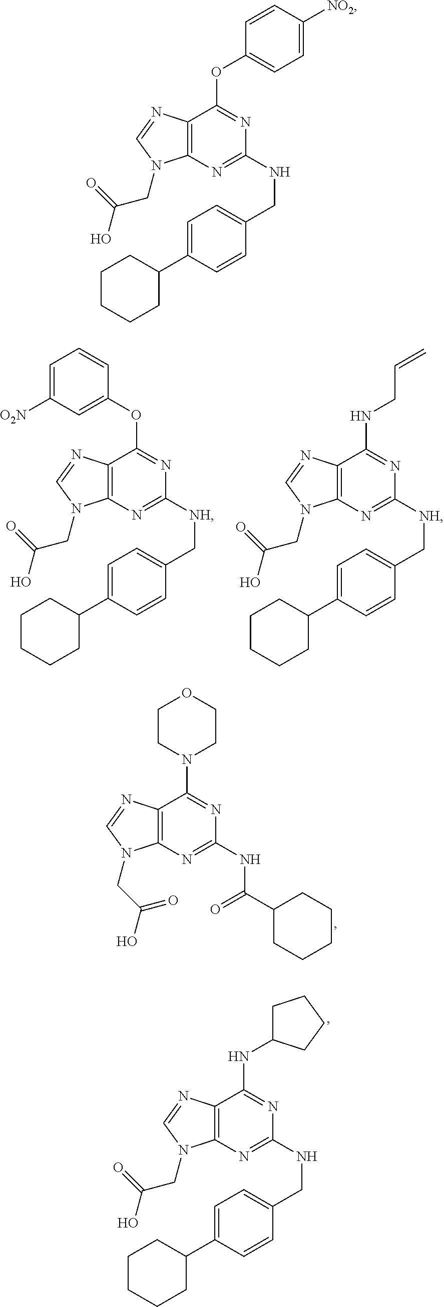

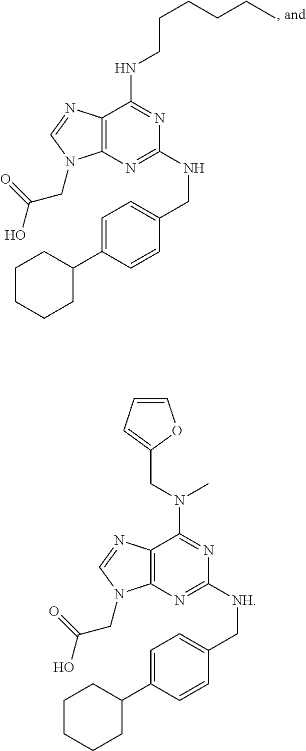 [SMILES: CCCCCCNc1nc(NCc2ccc(C3CCCCC3)cc2)nc2c1ncn2CC(=O)O ; CN(Cc1ccco1)c1nc(NCc2ccc(C3CCCCC3)cc2)nc2c1ncn2CC(=O)O]

It is understood that the disclosed compounds can be used in connection with the disclosed methods, compositions, kits, and uses.

The pharmaceutical acceptable derivatives of the compounds can include any suitable derivative, such as pharmaceutically acceptable salts as discussed below, isomers, radiolabeled analogs, tautomers, and the like.

In a further aspect, the disclosed compounds are inhibitors of STAT protein activity. In a still further aspect, the STAT protein is STAT3.

It is contemplated that one or more compounds can optionally be omitted from the disclosed invention.

3. Modulation of STAT3 Activity

In one aspect, the disclosed compounds exhibit inhibition STAT protein activity. In a further aspect, the disclosed compounds exhibit inhibition of STAT3 protein activity. In a yet further aspect, the disclosed compounds exhibit selective inhibition of STAT3 protein activity. In a still further aspect, the disclosed compounds exhibit prevention of STAT3 protein dimerization. In a yet further aspect, the disclosed compounds exhibit disruption of preformed or existing STAT3 dimers. In a still further aspect, the disclosed compounds exhibit binding to the SH2 domain of STAT3.

Inhibition of STAT3 activity can be determined by a variety of both in vitro and in vivo methods known to one skilled in the art. For example, inhibition of STAT protein activity can be determined using a electrophoretic mobility shift assay (“EMSA”). In one aspect, the disclosed compounds exhibit inhibition of STAT protein activity with an IC₅₀ in an EMSA assay of less than about 100 μM, less than about 50 μM, less than about 10 μM, less than about 1 μM, less than about 500 nM, or of less than about 100 nM. In a further aspect, the IC₅₀ in the EMSA assay is for STAT3 activity.

In one aspect, the disclosed compounds are selective for STAT3. In a further aspect, selective inhibition of STAT3 activity is shown in an EMSA assay. In various further aspects, the compound inhibits STAT3 activity in a EMSA assay with an IC₅₀ less than the IC₅₀ for one or more of STAT1, STAT2, STAT4, STAT5a, STAT5b, or STATE. That is, a disclosed compound can have selectivity for the STAT3 protein vis-à-vis one or more of STAT1, STAT2, STAT4, STAT5a, STAT5b, or STAT6 proteins. For example, in one aspect, a disclosed compound can inhibit STAT3 with an IC₅₀ of about 5-fold less than that for STAT1, of about 10-fold less than that for STAT1, of about 20-fold less than that for STAT1, of about 30-fold less than that for STAT1, or of about 50-fold less than that for STAT1. In a further aspect, a disclosed compound can inhibit STAT3 with an IC₅₀ of about 5-fold less than that for STAT2, of about 10-fold less than that for STAT2, of about 20-fold less than that for STAT2, of about 30-fold less than that for STAT2, or of about 50-fold less than that for STAT2. In a still further aspect, a disclosed compound can inhibit STAT3 with an IC₅₀ of about 5-fold less than that for STAT4, of about 10-fold less than that for STAT4, of about 20-fold less than that for STAT4, of about 30-fold less than that for STAT4, or of about 50-fold less than that for STAT4. In a yet further aspect, a disclosed compound can inhibit STAT3 with an IC₅₀ of about 5-fold less than that for STAT5a, of about 10-fold less than that for STAT5a, of about 20-fold less than that for STAT5a, of about 30-fold less than that for STAT5a, or of about 50-fold less than that for STAT5a. In an even further aspect, a disclosed compound can inhibit STAT3 with an IC₅₀ of about 5-fold less than that for STAT5b, of about 10-fold less than that for STAT5b, of about 20-fold less than that for STAT5b, of about 30-fold less than that for STAT5b, or of about 50-fold less than that for STAT5b. In a still further aspect, a disclosed compound can inhibit STAT3 with an IC₅₀ of about 5-fold less than that for STAT6, of about 10-fold less than that for STAT6, of about 20-fold less than that for STAT6, of about 30-fold less than that for STAT6, or of about 50-fold less than that for STAT6.

Alternatively, the inhibition of STAT protein activity can be determined in a cell-based assay. There are a variety of cell-based assays that are suitable for determination of inhibition of STAT protein activity known to one skilled in the art. For example, cell growth inhibition or cell arrest can be determined using a cell, either a permanent cell-line or a primary cell culture that has a STAT protein with dysfunction activity. In a further aspect, the STAT protein is STAT3. In a yet further aspect, the STAT protein dysfunction is one wherein the STAT protein is has acquired a gain of function mutation. Alternatively, the STAT protein has a phenotype of persistent or constitutive activity. In a further aspect, the STAT protein is overexpressed. In a further aspect, inhibition of STAT protein activity, e.g. STAT3 protein activity, is determined in a cell-line with a persistently active STAT3 protein, e.g. a breast cancer, prostate cancer, or pancreatic cancer cell-line. In yet further aspect, the activity of the disclosed compound is determined in a cell-line selected from MDA-MB-231, Panc-1, and DU 145. In a still further aspect, the activity of the disclosed compound is determined in a cell-line expressing v-Src. In an even further aspect, the cell-line is transformed with v-Src. In a yet further aspect, cell-line which is transformed in the NIH3T3 cell-line.

In one aspect, the disclosed compounds exhibit inhibition of cell growth. In a further aspect, the disclosed compounds inhibit cell growth in a cell with a persistently active STAT protein. In a yet further aspect, the disclosed compounds inhibit cell growth in a cell with a persistently active STAT3 protein. For example, a compound can exhibit inhibition of cell growth with an IC₅₀ of less than about 500 μM, of less than about 250 μM, of less than about 100 μM, of less than about 50 μM, less than about 10 μM, less than about 1 μM, less than about 500 nM, or of less than about 100 nM.

In various aspects, the disclosed compounds exhibit binding to a STAT protein. In a further aspect, the disclosed compounds exhibit binding to the SH2 domain of a STAT protein. In a still further aspect, the disclosed compounds exhibit binding to STAT3 protein. In a yet further aspect, the disclosed compounds exhibit binding to the SH2 domain of STAT3. The binding affinity of a disclosed compound for a STAT protein, e.g. STAT3 protein, can be determined by various methods known to one skilled in the art. For example, inhibition of STAT protein activity can be determined using a surface plasmon resonance (“SPR”) assay. In one aspect, the disclosed compounds exhibit binding to STAT protein with a K_{D} of less than about 50 μM, less than about 10 μM, less than about 1 μM, less than about 500 nM, or of less than about 100 nM. In a further aspect, the K_{D} is determined using an SPR method. In a still further aspect, the binding is determined using STAT3 protein.

In various further aspects, the binding to STAT3 is selective. In a further aspect, the disclosed compounds exhibit a K_{D} for STAT3 binding less than the K_{D} for one or more of STAT1, STAT2, STAT4, STAT5a, STAT5b, or STAT6. That is, a disclosed compound can have selectivity for the STAT3 protein vis-à-vis one or more of STAT1, STAT2, STAT4, STAT5a, STAT5b, or STAT6 proteins. For example, in one aspect, a disclosed compound can inhibit STAT3 with a K_{D} of about 5-fold less than that for STAT1, of about 10-fold less than that for STAT1, of about 20-fold less than that for STAT1, of about 30-fold less than that for STAT1, or of about 50-fold less than that for STAT1. In a further aspect, a disclosed compound can inhibit STAT3 with a K_{D} of about 5-fold less than that for STAT2, of about 10-fold less than that for STAT2, of about 20-fold less than that for STAT2, of about 30-fold less than that for STAT2, or of about 50-fold less than that for STAT2. In a still further aspect, a disclosed compound can inhibit STAT3 with a K_{D} of about 5-fold less than that for STAT4, of about 10-fold less than that for STAT4, of about 20-fold less than that for STAT4, of about 30-fold less than that for STAT4, or of about 50-fold less than that for STAT4. In a yet further aspect, a disclosed compound can inhibit STAT3 with a K_{D} of about 5-fold less than that for STAT5a, of about 10-fold less than that for STAT5a, of about 20-fold less than that for STAT5a, of about 30-fold less than that for STAT5a, or of about 50-fold less than that for STAT5a. In an even further aspect, a disclosed compound can inhibit STAT3 with a K_{D} of about 5-fold less than that for STAT5b, of about 10-fold less than that for STAT5b, of about 20-fold less than that for STAT5b, of about 30-fold less than that for STAT5b, or of about 50-fold less than that for STAT5b. In a still further aspect, a disclosed compound can inhibit STAT3 with a K_{D} of about 5-fold less than that for STAT6, of about 10-fold less than that for STAT6, of about 20-fold less than that for STAT6, of about 30-fold less than that for STAT6, or of about 50-fold less than that for STAT6.

### C. METHODS OF MAKING THE COMPOUNDS

In one aspect, the invention relates to methods of making compounds useful as inhibitors of STAT. In a further aspect, the products of disclosed methods of making are modulators of STAT activity. In a yet further aspect, the products of disclosed methods of making bind to a STAT protein and negatively modulate STAT activity. The compounds can, in one aspect, exhibit subtype selectivity. In a still further aspect, the products of disclosed methods of making exhibit selectivity for the STAT3 member of the STAT protein family.

The compounds of this invention can be prepared by employing reactions as shown in the following schemes, in addition to other standard manipulations that are known in the literature, exemplified in the experimental sections or clear to one skilled in the art. For clarity, examples having a single substituent are shown where multiple substituents are allowed under the definitions disclosed herein.

Reactions used to generate the compounds of this invention are prepared by employing reactions as shown in the following Reaction Schemes, in addition to other standard manipulations known in the literature or to one skilled in the art. The following examples are provided so that the invention might be more fully understood, are illustrative only, and should not be construed as limiting.

In one aspect, the disclosed compounds comprise the products of the synthetic methods described herein. In a further aspect, the disclosed compounds comprise a compound produced by a synthetic method described herein. In a still further aspect, the invention comprises a pharmaceutical composition comprising a therapeutically effective amount of the product of the disclosed methods and a pharmaceutically acceptable carrier. In a still further aspect, the invention comprises a method for manufacturing a medicament comprising combining at least one compound of any of disclosed compounds or at least one product of the disclosed methods with a pharmaceutically acceptable carrier or diluent.

In a further aspect, the compound produced is useful in the treatment of a disorder of uncontrolled cellular proliferation associated with STAT dysfunction and other diseases in which a STAT protein is involved, as further described herein. In a further aspect, the STAT protein is STAT3.

1. Route 1

In one aspect, substituted 2-(9H-purin-9-yl)acetic acid analogs of the present invention can be prepared generically by the synthetic scheme as shown below.

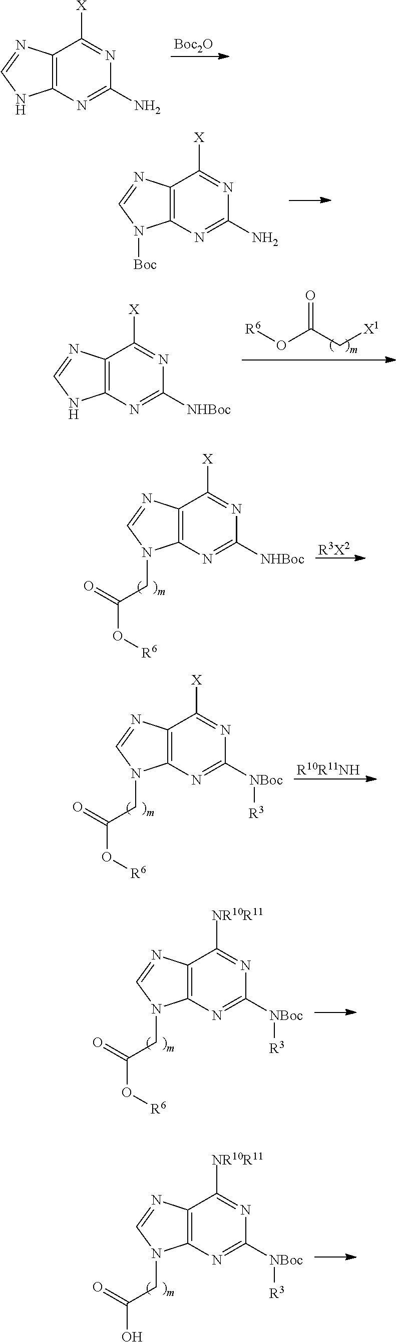

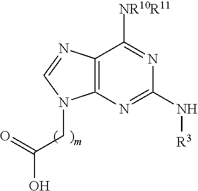

Compounds are represented in generic form, with substituents as noted in compound descriptions elsewhere herein. Route I can generically begin with protection of the amine group of an 6-halo-2-aminopurine. In one aspect, the purine ring nitrogen can be first protected with, for example, a alkyloxycarbonyl group. It is contemplated that alternative amine protecting groups can be instead used. The amine group of the protected purine can then be subsequently mono-protected with a second amine protecting group, for example another alkyloxycarbonyl group, (again, it is contemplated that alternative amine protecting groups can be instead used) followed by selective deprotection of the purine ring nitrogen.

With the purine ring nitrogen available, the compound can then be reacted with a leaving group-substituted ester—for example, methyl 2-chloroacetate, ethyl 3-bromopropanoate, or homolog thereof—to provide carboxyl functionality at the purine ring nitrogen position. In various aspects, the leaving group X¹ can be halogen, pseudohalogen, or hydroxyl under Misunobu reaction conditions (e.g., in the presence of Ph₃P and DIAD). In one aspect, the leaving group-substituted ester can be an alkyl 2-hydroxyacetate, or homolog thereof, and the reaction is carried out under Misunobu reaction conditions.

The mono-protected amine group of the purine can then be substituted by reaction with R₃X². In various aspects, the leaving group X² can be halogen, pseudohalogen, or hydroxyl under Misunobu reaction conditions (e.g., in the presence of Ph₃P and DIAD). In one aspect, R₃X² can be R₃OH, and the reaction is carried out under Misunobu reaction conditions. The resulting 6-halo-2-aminopurine can then be reacted with a suitable nucleophile, for example R¹⁰R¹¹NH, to provide a 2-(6-amino-2-(protected)aminopurinyl)ester. As would be appreciated by one of ordinary skill in the art, subsequent reactions can, if desired, be used to deprotect the various groups to yield the carboxylic acid functionality and/or the amine functionality.

A more specific example is set forth below.

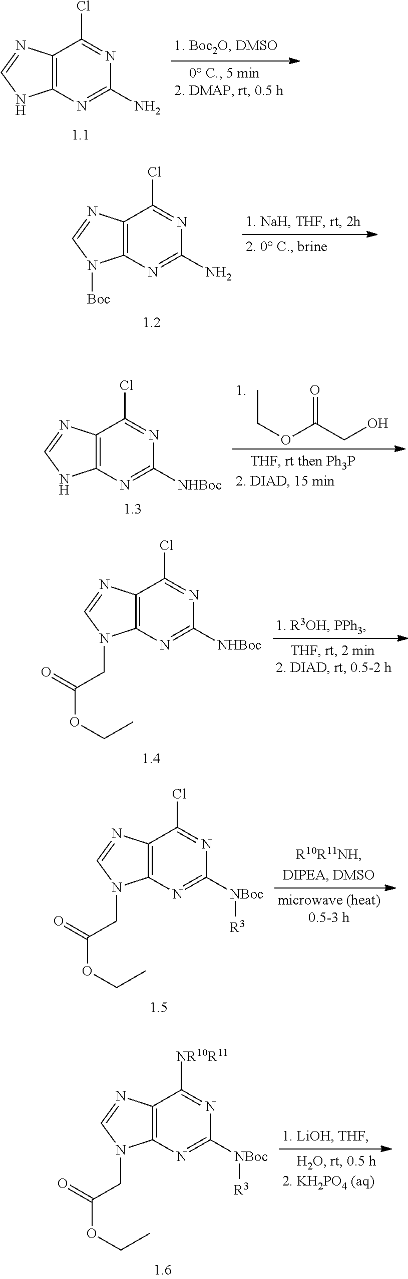

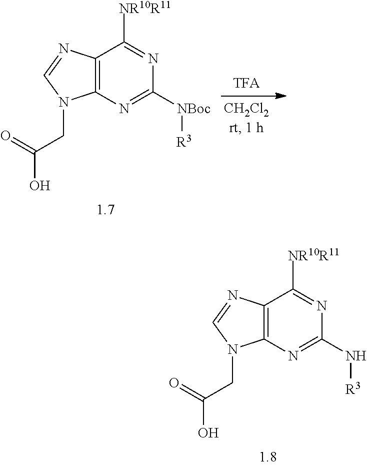

In one aspect, Route I begins with a suitable substituted 2-amino-6-chloropurine (1.1) and di-tert-butyldicarbonate. Suitable purines are commercially available or can be readily prepared by one skilled in the art. The reaction of the purine and di-tert-butyl dicarbonate derivative is typically carried out a suitable solvent such as DMSO. To the reaction a suitable catalytic base is added, e.g., DMAP, and the reaction is maintained at a temperature, e.g., about 0° C. The reaction is then warmed to about room temperature and the reaction is carried out for a time sufficient to complete the reaction, e.g., about two hours. The product, a compound of type 1.2, is isolated and then dissolved in an appropriate solvent, e.g., THF and a base, e.g., NaH is added at room temperature for a time sufficient to complete the reaction, e.g., about 2 hours, to provide compounds of type 1.3 as shown above. The product, a compound of type 1.3, is isolated and then dissolved in an appropriate solvent, e.g., THF and ethyl glycolate (or other suitable glycolate ester derivative) is added. followed by triphenylphosphine. A suitable coupling agent, e.g., DIAD, is added at room temperature for a time sufficient to complete the reaction, e.g., about 15 minutes, to provide compounds of type 1.4 as shown above. The product, a compound of type 1.4, is isolated by methods known to one skilled in the art (e.g., extraction, washing, drying, and concentration under a vacuum; followed by purification, e.g., chromatography, if necessary).

In one aspect, compounds of type 1.5 can be prepared by reaction of compounds of type 1.4 with a suitable aminoalkylating reagent, e.g., an alcohol (R³OH). In a typical reaction, a compound of type 1.4 is dissolved in a suitable solvent, e.g., THF, followed by the addition of triphenylphosphine. A suitable coupling agent, e.g., DIAD, is added at room temperature for a time sufficient to complete the reaction, e.g., about 0.5 to 2 hours, to provide compounds of type 1.5 as shown above. The product, a compound of type 1.5, is isolated by methods known to one skilled in the art (e.g., extraction, washing, drying, and concentration under a vacuum; followed by purification, e.g., chromatography, if necessary).

In one aspect, amination of compounds of type 1.5 can be provide compounds of type 1.6. For example, a mixture of a suitable chloropurine (1.5), a suitable amine or aniline derivative (R¹⁰R¹¹NH), and DIEA are dissolved in a suitable solvent, e.g., DMSO, and the mixture is heated using a microwave reactor at suitable temperature, e.g. about 135° C., for a suitable time, e.g., about 0.5 to 3 hours. The product (1.6) is isolated by methods known to one skilled in the art (e.g., extraction, washing, drying, and concentration under a vacuum; followed by purification, e.g., chromatography).

In one aspect, compounds of type 1.7 can be prepared by the hydrolysis of an ester of compound type 1.6 to the corresponding carboxylic acid. For example, a reaction of this type is commonly carried out by dissolving or suspending the ester (1.6) in a suitable solvent, e.g., THF/H₂O (3:1 ratio), to which is added a suitable base, e.g., lithium hydroxide, and the mixture stirred at an appropriate temperature, e.g., ambient room temperature, for a time sufficient, e.g., about 0.5 hour, to complete the reaction. The mixture is then acidified using a suitable acid, e.g, KH₂PO₄. The product (1.7) is isolated by methods known to one skilled in the art (e.g., extraction, washing, drying, and concentration under a vacuum; followed by purification, e.g., chromatography, and then lyophilized).

In one aspect, compounds of type 1.8 can be prepared by conversion of an N-Boc purine of compound type 1.7 to the corresponding purine (1.8). For example, a reaction of this type is commonly carried out by dissolving the N-Boc purine (1.7) in TFA:CH₂Cl₂ (1:1), and the mixture is stirred for a time sufficient, e.g., about one hour, at ambient room temperature to complete the reaction. The product (1.8) is isolated by methods known to one skilled in the art (e.g., concentration under a vacuum; followed by purification, e.g., chromatography, and then lyophilized).

2. Route II

In one aspect, substituted 2-(9H-purin-9-yl)acetic acid analogs of the present invention can be prepared generically by the synthetic scheme as shown below.

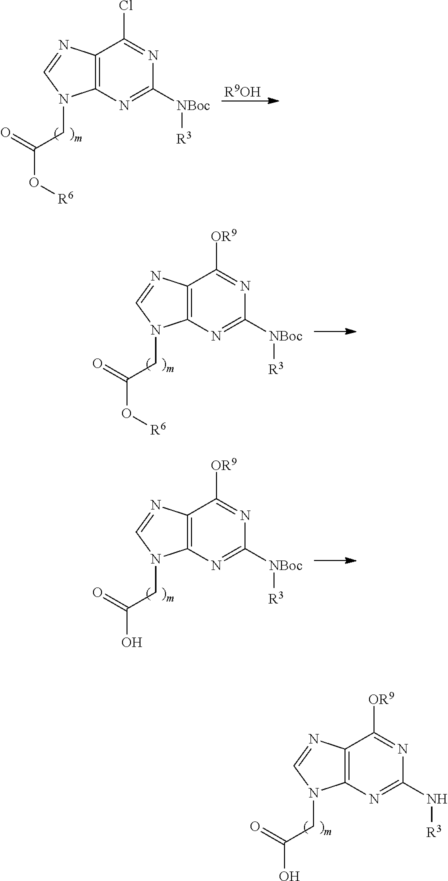

Compounds are represented in generic form, with substituents as noted in compound descriptions elsewhere herein. Route II can generically begin with a 2-(6-amino-2-(protected)aminopurinyl)ester, which can be provided, for example, as described in Route I. The compound can be reacted with a suitable nucleophile, for example R⁹OH, wherein R⁹ is alkyl or aryl, to provide a 2-(6-alkoxyl-2-(protected)aminopurinyl)ester or 2-(6-aryloxyl-2-(protected)aminopurinyl)ester. As would be appreciated by one of ordinary skill in the art, subsequent reactions can, if desired, be used to deprotect the various groups to yield the carboxylic acid functionality and/or the amine functionality.

A more specific example is set forth below.

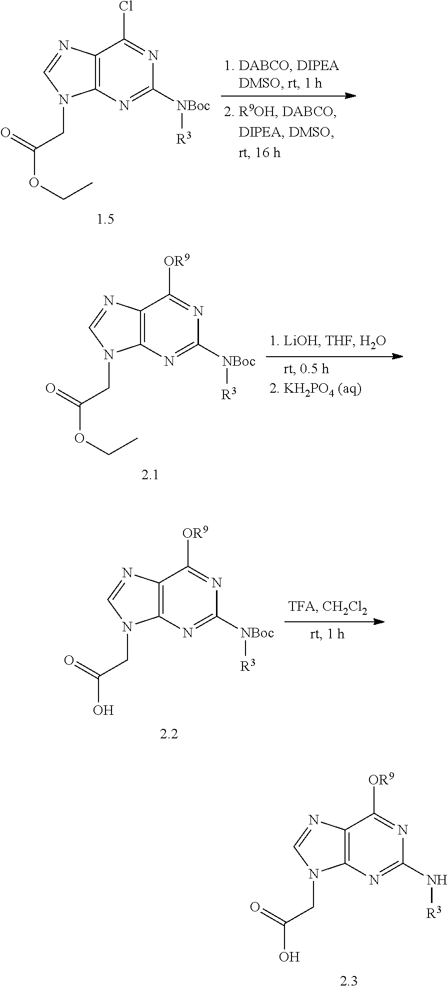

In one aspect, Route II begins with a suitable substituted 6-chloropurine (1.5). Suitable purines can be readily prepared using the method outlined in Route I by one skilled in the art. The reaction of the purine derivative (1.5) is typically carried out a suitable solvent such as DMSO. To the reaction a suitable base is added, e.g., DABCO and DIPEA, and the reaction is stirred at ambient room temperature for a time sufficient to complete the reaction, e.g., about one hour. Then, a solution of a phenol derivative (R⁹OH), a suitable base combination, e.g., DABCO and DIPEA, are added. The reaction is carried out for a time sufficient to complete the reaction, e.g., about sixteen hours. The product, a compound of type 2.1, is isolated by methods known to one skilled in the art (e.g., extraction, washing, drying, and concentration under a vacuum followed by purification, e.g., chromatography).

In one aspect, compounds of type 2.2 can be prepared by hydrolysis of an ester of compound type 2.1 to the corresponding carboxylic acid. For example, a reaction of this type is commonly carried out by dissolving or suspending the ester (2.1) in a suitable solvent, e.g., THF/H₂O (3:1 ratio), to which is added a suitable base, e.g., lithium hydroxide, and the mixture stirred at room temperature for a time sufficient, e.g., about 0.5 hour, to complete the reaction. The mixture is then acidified using a suitable acid, e.g, KH₂PO₄. The product (2.2) is isolated by methods known to one skilled in the art (e.g., extraction, washing, drying, and concentration under a vacuum; followed by purification, e.g., chromatography, and then lyophilized).

In one aspect, compounds of type 2.3 can be prepared by conversion of an N-Boc purine of compound type 2.2 to the corresponding purine (2.3). For example, a reaction of this type is commonly carried out by dissolving the N-Boc purine in TFA:CH₂Cl₂ (1:1), and the mixture is stirred at room temperature for a time sufficient, e.g., about one hour, to complete the reaction. The product (2.3) is isolated by methods known to one skilled in the art (e.g., concentration under a vacuum; followed by purification, e.g., chromatography, and then lyophilized).

3. Route III

In one aspect, substituted 2-(9H-purin-9-yl)acetic acid analogs of the present invention can be prepared generically by the synthetic scheme as shown below.

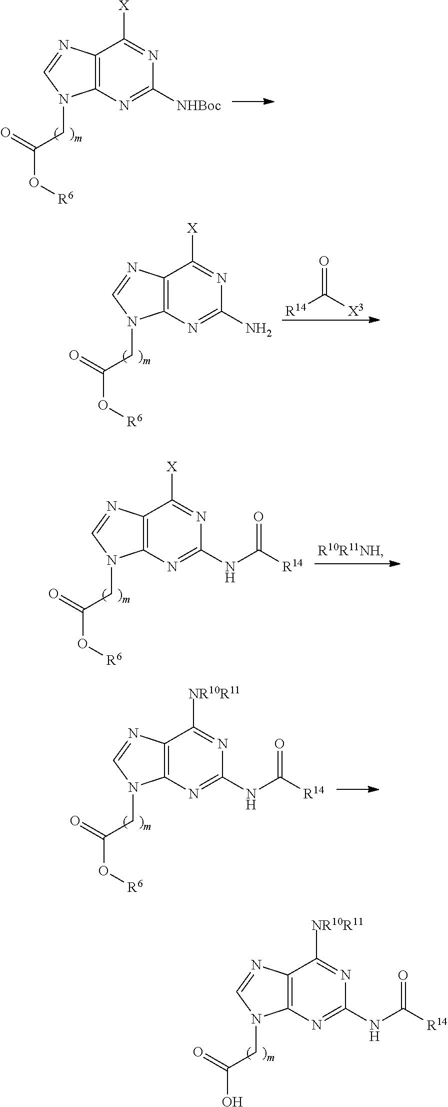

Compounds are represented in generic form, with substituents as noted in compound descriptions elsewhere herein.

Route III can generically begin with a 2-(6-amino-2-(protected)aminopurinyl)ester, which can be provided, for example, as described in Route I. The mono-protected amine group of the purine can then be deprotected and reacted with an acylation reagent, R¹⁴C(O)X³, wherein X³ is a leaving group such as halogen. It is also contemplated that acylation can also be effected by reaction with the corresponding anhydride (e.g., R¹⁴C(O)OC(O)R¹⁴) or corresponding ester (e.g., R¹⁴C(O)R, wherein R is alkyl or aryl). The resulting 6-halo-2-amidopurine can then be reacted with a suitable nucleophile, for example R¹⁰R¹¹NH, to provide a 2-(6-amino-2-amidopurinyl)ester. As would be appreciated by one of ordinary skill in the art, subsequent reaction can, if desired, be used to deprotect ester to yield the carboxylic acid functionality.

It is also contemplated that, in place of R¹⁰R¹¹NH, an oxygen-based nucleophile, for example R⁹OH, wherein R⁹ is alkyl or aryl, can also be used to provide a 2-(6-alkoxyl-2-amidopurinyl)ester or 2-(6-aryloxyl-2-amidopurinyl)ester. Again, subsequent reaction can be used to deprotect the ester to yield the carboxylic acid functionality.

A more specific example is set forth below.

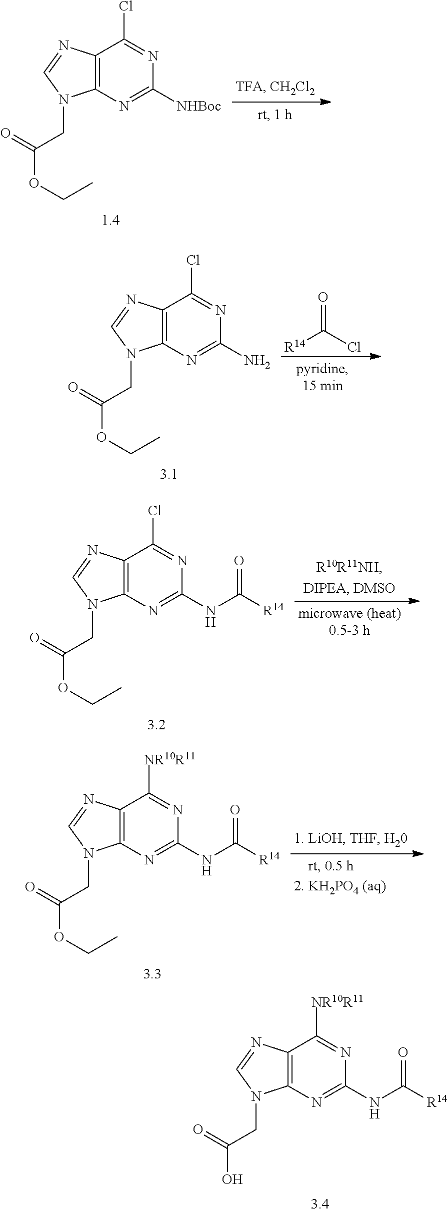

In one aspect, Route III begins with a suitable substituted 6-chloropurine (1.4). Suitable purines can be readily prepared using the method outlined in Route I by one skilled in the art. Compounds of type 3.1 can be prepared by conversion of the N-Boc purine of compound type 1.4 to the corresponding purine (3.1). For example, a reaction of this type is commonly carried out by dissolving the N-Boc purine in TFA:CH₂Cl₂ (1:1), and the mixture is stirred at room temperature for a time sufficient, e.g., about one hour, to complete the reaction. The product (3.1) is isolated by methods known to one skilled in the art (e.g., concentration under a vacuum; followed by purification, e.g., chromatography, and then lyophilized).

In one aspect, compounds of type 3.2 can be prepared by reaction of compounds of type 3.1 with a suitable acylating reagent, e.g., an acid chloride (halide) or a suitable anhydride. In a typical reaction, a compound of type 3.1 is dissolved in a suitable solvent, e.g., pyridine, followed by the addition of the acid halide. After stirring for a time sufficient to complete the reaction, e.g., about 15 minutes, to provide compounds of type 3.2 as shown above after adding water and adjusting the pH. The product, a compound of type 3.2, is isolated by methods known to one skilled in the art (e.g., extraction, washing, drying, and concentration under a vacuum; followed by purification, e.g., chromatography).

In one aspect, amination of compounds of type 3.2 can be provide compounds of type 3.3. For example, a mixture of a suitable chloropurine (3.2), a suitable amine derivative (R¹⁰R¹¹NH), and DIEA are dissolved in a suitable solvent, e.g., DMSO, and the mixture is heated using a microwave reactor at suitable temperature, e.g., about 135° C., for a suitable time, e.g., about 0.5 to 3 hours. The product (3.3) is isolated by methods known to one skilled in the art (e.g. extraction, washing, drying, and concentration under a vacuum; followed by purification, e.g., chromatography).

In one aspect, compounds of type 3.4 can be prepared by hydrolysis of an ester of compound type 3.3 to the corresponding carboxylic acid. For example, a reaction of this type is commonly carried out by dissolving or suspending the ester (3.3) in a suitable solvent, e.g., THF/H₂O (3:1 ratio), to which is added a suitable base, e.g., lithium hydroxide, and the mixture stirred at room temperature for a time sufficient, e.g., about 0.5 hour, to complete the reaction. The mixture is then acidified using a suitable acid, e.g, KH₂PO₄. The product (3.4) is isolated by methods known to one skilled in the art (e.g., extraction, washing, drying, and concentration under a vacuum; followed by purification, e.g., chromatography, and then lyophilized).

Thus, in one aspect, the invention relates to a method of making a compound comprising the steps of: (a) providing a compound having a structure represented by a formula:

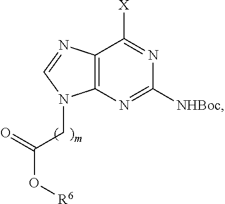

wherein X is a halogen; wherein m is an integer from 0-3; wherein R⁶ is C1-C8 alkyl; and, (b) reacting the compound with a R³OH, wherein R³ is selected from hydrogen and an optionally substituted group selected from C1-C8 alkyl, (CH₂)_{q}R¹⁴, and C═O(CH₂)_{q}R¹⁴; wherein q is an integer from 0-3; wherein R¹⁴ is selected from hydrogen and an optionally substituted group selected from C1-C8 alkyl, C3-C6 cycloalkyl, C3-C6 heterocycloalkyl, and Ar³; wherein Ar³ is selected from an optionally substituted monocyclic aryl and monocyclic heteroaryl; wherein R⁴ is selected from hydrogen and C═OOR¹⁵; wherein R¹⁵ is selected from hydrogen and optionally substituted C1-C8 alkyl; to yield a compound represented by the formula:

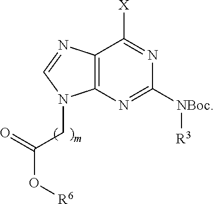

In a further aspect, the halogen is chloro. In a still further aspect, m is 1.

In a further aspect, reaction with R³OH is carried out in the presence of a azodicarboxylate and triarylphosphine. In a yet further aspect, the azodicarboxylate is selected from diisopropyl azodicarboxylate (DIAD), diethyl azodicarboxylate (DEAD), di-tert-butyl azodicarboxylate, and di-p-chlorobenzyl azodicarboxylate (DCAD). In an even further aspect, the triarylphosphine is selected from triphenylphosphine, 1,2-bis(diphenylphosphino)ethane (DPPE). p-dimethylaminophenyl)-diphenylphosphine (DAP-DP), diphenyl(2-pyridyl)phosphine (Ph2P-Py), tris(dimethylamino)phosphine (tris-DAP), and diphenyl(2-pyridyl)phosphine DAP).

In a further aspect, the reaction further comprises the step of reaction of the compound formed, represented by the formula:

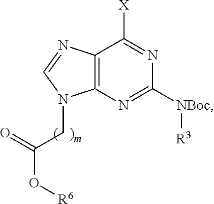

with NHR¹⁰N¹¹, wherein R¹⁰ is selected from hydrogen and an optionally substituted group selected from C1-C8 alkyl, C3-C6 cycloalkyl, C3-C6 heterocycloalkyl, (CH₂)ₚR¹³, and Ar²; wherein n is an integer from 0-3; wherein R¹³ is an optionally substituted group selected from Ar², C3-C6 cycloalkyl and C3-C6 heterocycloalkyl; wherein Ar² is selected from an optionally substituted monocyclic aryl and monocyclic heteroaryl; wherein R¹¹ is selected from hydrogen and C1-C8 alkyl; in the presence of base to yield a compound represented by the formula:

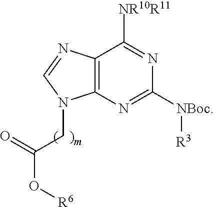

In a yet further aspect, the base is selected from carbonate salt, triethylamine (TEA), N,N-iisopropylethylamine (DIPEA), and 1,8-Diazabicycloundec-7-ene (DBU). In an even further aspect, the base is N,N-diisopropylethylamine (DIPEA). In a still further aspect, the carbonate salt is Cs₂CO₃ or K₂CO₃.

In a further aspect, the product formed in the preceding reaction, represented by the formula:

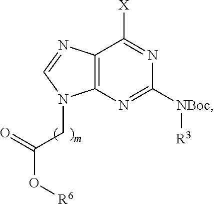

is treated with base to yield the product represented by the formula:

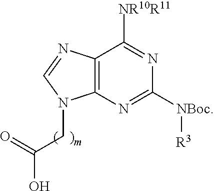

In a further aspect, the base is selected from NaOH, KOH, and LiOH.

In a further aspect, the product formed is treated with an acid to, yielding a compound represented by the formula:

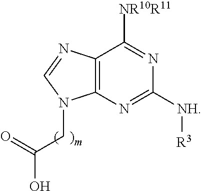

In a yet further aspect, the acid is in the reaction is selected from H₃PO₄, HCl, and trifluoroacetic acid (TFA).

In a further aspect, the compound produced exhibits inhibition of STAT protein activity. In a still further aspect, the compound produced exhibits inhibition of STAT3 protein activity.

In a further aspect, the compound produced exhibits of STAT protein activity with an IC₅₀ in an EMSA assay of less than about 100 μM, less than about 50 μM, less than about 10 μM, less than about 1 μM, less than about 500 nM, or of less than about 100 nM. In a further aspect, the IC₅₀ in the EMSA assay is for STAT3 activity.

In a further aspect, the compound produced exhibits inhibition of cell growth. In a further aspect, the disclosed compounds inhibit cell growth in a cell with a persistently active STAT protein. In a yet further aspect, the disclosed compounds inhibit cell growth in a cell with a persistently active STAT3 protein. For example, a compound can exhibit inhibition of cell growth with an IC₅₀ of less than about 500 μM, of less than about 250 μM, of less than about 100 μM, of less than about 50 μM, less than about 10 μM, less than about 1 μM, less than about 500 nM, or of less than about 100 nM.

In a further aspect, the compound produced exhibits binding to a STAT protein. In a still further aspect, the compound produced binds the SH2 domain of a STAT protein. In a yet further aspect, the disclosed compounds exhibit binding to STAT3 protein. In an even further aspect, the compound produced binds to the SH2 domain of STAT3. In a still further aspect, the compound produced exhibits binding to STAT protein with a K_{D} of less than about 50 μM, less than about 10 μM, less than about 1 μM, less than about 500 nM, or of less than about 100 nM. In a further aspect, the K_{D} is determined using an SPR method. In a still further aspect, the binding is determined using STAT3 protein.

It is contemplated that each disclosed methods can further comprise additional steps, manipulations, and/or components. It is also contemplated that any one or more step, manipulation, and/or component can be optionally omitted from the invention. It is understood that a disclosed methods can be used to provide the disclosed compounds. It is also understood that the products of the disclosed methods can be employed in the disclosed methods of using.

### D. PHARMACEUTICAL COMPOSITIONS

In one aspect, the invention relates to pharmaceutical compositions comprising the disclosed compounds. That is, a pharmaceutical composition can be provided comprising a therapeutically effective amount of at least one disclosed compound or at least one product of a disclosed method and a pharmaceutically acceptable carrier.

In one aspect, the invention relates to a pharmaceutical composition comprising a pharmaceutically acceptable carrier and an effective amount of a compound represented by a formula:

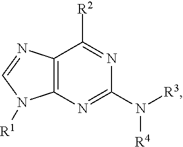

wherein R¹ is selected from H and (CH₂)ₘC═OR⁵, wherein m is an integer from 0-3; wherein R⁵ is selected from OR⁶ and NR⁷R⁸; wherein R⁶ is selected from hydrogen and C1-C8 alkyl; wherein each of R⁷ and R⁸ is independently selected from hydrogen and C1-C8 alkyl; wherein R² is selected from halogen, OR⁹, and NR¹⁰R¹¹; wherein R⁹ is an optionally substituted group selected from C1-C8 alkyl, C3-C6 cycloalkyl, C3-C6 heterocycloalkyl, (CH₂)ₙR¹², and Ar¹; wherein n is an integer from 0-3; wherein R¹² is an optionally substituted group selected from Ar¹, C3-C6 cycloalkyl and C3-C6 heterocycloalkyl; wherein Ar¹ is selected from an optionally substituted monocyclic aryl and monocyclic heteroaryl; wherein R¹⁰ is selected from hydrogen and an optionally substituted group selected from C1-C8 alkyl, C3-C6 cycloalkyl, C3-C6 heterocycloalkyl, (CH₂)ₚR¹³, and Ar²; wherein p is an integer from 0-3; wherein R¹³ is an optionally substituted group selected from Ar², C3-C6 cycloalkyl and C3-C6 heterocycloalkyl; wherein Ar² is selected from an optionally substituted monocyclic aryl and monocyclic heteroaryl; wherein R¹¹ is selected from hydrogen and C1-C8 alkyl; wherein R³ is selected from hydrogen and an optionally substituted group selected from C1-C8 alkyl, (CH₂)_{q}R¹⁴, and C═O(CH₂)_{q}R¹⁴; wherein q is an integer from 0-3; wherein R¹⁴ is selected from hydrogen and an optionally substituted group selected from C1-C8 alkyl, C3-C6 cycloalkyl, C3-C6 heterocycloalkyl, and Ar³; wherein Ar³ is selected from an optionally substituted monocyclic aryl and monocyclic heteroaryl; wherein R⁴ is selected from hydrogen and C═OOR¹⁵; wherein R¹⁵ is selected from hydrogen and optionally substituted C1-C8 alkyl; wherein R³ and R⁴ are optionally covalently bonded and, together with the intermediate carbon, comprise an optionally substituted 3- to 7-membered heterocycloalkyl; and wherein R¹⁰ and R¹¹ are optionally covalently bonded and, together with the intermediate carbon, comprise an optionally substituted 3- to 7-membered heterocycloalkyl; or a pharmaceutically acceptable salt, hydrate, solvate, or polymorph thereof.

In a further aspect, the compound is any of the disclosed compounds or at least one product of the disclosed methods of making. In a yet further aspect, the pharmaceutical composition comprises one or more of any of the disclosed compounds or at least one product of the disclosed methods of making.

In a further aspect, the compound inhibits STAT protein activity with an IC₅₀ in an EMSA assay of less than about 100 μM, less than about 50 μM, less than about 10 μM, less than about 1 μM, less than about 500 nM, or of less than about 100 nM. In a further aspect, the IC₅₀ for inhibition of STAT3 activity.

In a further aspect, the acompound that inhibits cell growth. In a still further aspect, the compound inhibits cell growth with an IC₅₀ of less than about 500 μM. In a yet further aspect, the compound inhibits cell growth with an IC₅₀ of less than about 250 μM. In an even further aspect, the compound inhibits cell growth with an IC₅₀ of less than about 100 μM. In a still further aspect, the compound inhibits cell growth with an IC₅₀ of less than about 50 μM. In a yet further aspect, the compound inhibits cell growth with an IC₅₀ of less than about 10 μM. In an even further aspect, the compound inhibits cell growth with an IC₅₀ of less than about 1 μM. In a still further aspect, the IC₅₀ for inhibition of cell growth is determined in a cell line with a constitutively active STAT protein. In a yet further aspect, the IC₅₀ for inhibition of cell growth is determined in a cell line with a persistently active STAT protein. In an even further aspect, the IC₅₀ for inhibition of cell growth is determined in a cell line derived from a cancer selected from breast cancer, pancreatic cancer, and prostate cancer. In a yet further aspect, the IC₅₀ for inhibition of cell growth is determined in a cell line derived from a cancer with a STAT protein activity dysfunction. In a still further aspect, the IC₅₀ for inhibition of cell growth is determined in a cell line selected from MDA-MB-231, Panc-1, and DU 145. In a yet further aspect, the IC₅₀ for inhibition of cell growth is determined in a cell line transformed with v-Src. In an even further aspect, the IC₅₀ for inhibition of cell growth is determined in the NIH3T3 cell-line transformed with v-Src.

In a further aspect, the pharmaceutical composition treats a disorder of uncontrolled cellular proliferation. In a yet further aspect, the disorder of uncontrolled cellular proliferation is cancer. In a still further aspect, the cancer is selected from a cancer of the head, neck, pancreas, brain, ovary, kidney, prostate, breast, lung, colon, and liver.

In a further aspect, the cancer is a hematological cancer. In a still further aspect, the hematological cancer is selected from acute myeloid leukemia (AML), acute lymphoblastic leukemia (ALL), chronic myeloid leukemia (CML), chronic lymphocytic leukemia (CLL), hairy cell leukemia, chronic myelomonocytic leukemia (CMML), juvenile myelomonocytic leukemia (JMML), Hodgkin lymphoma, Non-Hodgkin lymphoma, multiple myeloma, solitary myeloma, localized myeloma, and extramedullary myeloma.

In a further aspect, the cancer is a cancer of the brain. In a still further aspect, the cancer of the brain is selected from a glioma, medulloblastoma, primitive neuroectodermal tumor (PNET), acoustic neuroma, glioma, meningioma, pituitary adenoma, schwannoma, CNS lymphoma, primitive neuroectodermal tumor, craniopharyngioma, chordoma, medulloblastoma, cerebral neuroblastoma, central neurocytoma, pineocytoma, pineoblastoma, atypical teratoid rhabdoid tumor, chondrosarcoma, chondroma, choroid plexus carcinoma, choroid plexus papilloma, craniopharyngioma, dysembryoplastic neuroepithelial tumor, gangliocytoma, germinoma, hemangioblastoma, hemangiopercytoma, and metastatic brain tumor. In a yet further aspect, the glioma is selected from ependymoma, astrocytoma, oligodendroglioma, and oligoastrocytoma. In an even further aspect, the glioma is selected from juvenile pilocytic astrocytoma, subependymal giant cell astrocytoma, ganglioglioma, subependymoma, pleomorphic xanthoastrocytom, anaplastic astrocytoma, glioblastoma multiforme, brain stem glioma, oligodendroglioma, ependymoma, oligoastrocytoma, cerebellar astrocytoma, desmoplastic infantile astrocytoma, subependymal giant cell astrocytoma, diffuse astrocytoma, mixed glioma, optic glioma, gliomatosis cerebri, multifocal gliomatous tumor, multicentric glioblastoma multiforme tumor, paraganglioma, and ganglioglioma.

In a further aspect, the cancer is selected from a cancer of the breast, ovary, prostate, head, neck, and kidney. In a still further aspect, the cancer is breast cancer. In a yet further aspect, the cancer is pancreatic cancer.

In certain aspects, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

As used herein, the term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (-ic and -ous), ferric, ferrous, lithium, magnesium, manganese (-ic and -ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

As used herein, the term “pharmaceutically acceptable non-toxic acids”, includes inorganic acids, organic acids, and salts prepared therefrom, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids.

In practice, the compounds of the invention, or pharmaceutically acceptable salts thereof, of this invention can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention comprise a compound of the invention (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in moulds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

In the treatment conditions which require modulation of STAT protein activity an appropriate dosage level will generally be about 0.01 to 500 mg per kg patient body weight per day and can be administered in single or multiple doses. Preferably, the dosage level will be about 0.1 to about 250 mg/kg per day; more preferably 0.5 to 100 mg/kg per day. A suitable dosage level can be about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the from of tablets containing 1.0 to 1000 milligrams of the active ingredient, particularly 1.0, 5.0, 10, 15, 20, 25, 50, 75, 100, 150, 200, 250, 300, 400, 500, 600, 750, 800, 900 and 1000 milligrams of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

It is understood, however, that the specific dose level for any particular patient will depend upon a variety of factors. Such factors include the age, body weight, general health, sex, and diet of the patient. Other factors include the time and route of administration, rate of excretion, drug combination, and the type and severity of the particular disease undergoing therapy.

The present invention is further directed to a method for the manufacture of a medicament for modulating STAT protein activity (e.g., treatment of one or more disorders of uncontrolled cellular proliferation associated with a STAT3 protein activity dysfunction) in mammals (e.g., humans) comprising combining one or more disclosed compounds, products, or compositions with a pharmaceutically acceptable carrier or diluent. Thus, in one aspect, the invention relates to a method for manufacturing a medicament comprising combining at least one disclosed compound or at least one disclosed product with a pharmaceutically acceptable carrier or diluent.

The disclosed pharmaceutical compositions can further comprise other therapeutically active compounds, which are usually applied in the treatment of the above mentioned pathological conditions.

It is understood that the disclosed compositions can be prepared from the disclosed compounds. It is also understood that the disclosed compositions can be employed in the disclosed methods of using.

### E. METHODS OF USING THE COMPOUNDS AND COMPOSITIONS

The disclosed compounds can be used as single agents or in combination with one or more other drugs in the treatment, prevention, control, amelioration or reduction of risk of the aforementioned diseases, disorders and conditions for which compounds of formula I or the other drugs have utility, where the combination of drugs together are safer or more effective than either drug alone. The other drug(s) can be administered by a route and in an amount commonly used therefore, contemporaneously or sequentially with a disclosed compound. When a disclosed compound is used contemporaneously with one or more other drugs, a pharmaceutical composition in unit dosage form containing such drugs and the disclosed compound is preferred. However, the combination therapy can also be administered on overlapping schedules. It is also envisioned that the combination of one or more active ingredients and a disclosed compound will be more efficacious than either as a single agent.

In one aspect, the subject compounds can be coadministered with 13-cis-Retinoic Acid, 2-CdA, 2-Chlorodeoxyadenosine, 5-Azacitidine, 5-Fluorouracil, 5-FU, 6-Mercaptopurine, 6-MP, 6-TG, 6-Thioguanine, Abraxane, Accutane®, Actinomycin-D, Adriamycin®, Adrucil®, Afinitor®, Agrylin®, Ala-Cort®, Aldesleukin, Alemtuzumab, ALIMTA, Alitretinoin, Alkaban-AQ®, Alkeran®, All-transretinoic Acid, Alpha Interferon, Altretamine, Amethopterin, Amifostine, Aminoglutethimide, Anagrelide, Anandron®, Anastrozole, Arabinosylcytosine, Ara-C, Aranesp®, Aredia®, Arimidex®, Aromasin®, Arranon®, Arsenic Trioxide, Arzerra™, Asparaginase, ATRA, Avastin®, Azacitidine, BCG, BCNU, Bendamustine, Bevacizumab, Bexarotene, BEXXAR®, Bicalutamide, BiCNU, Blenoxane®, Bleomycin, Bortezomib, Busulfan, Busulfex®, C225Calcium Leucovorin, Campath®, Camptosar®, Camptothecin-11, Capecitabine, Carac™, Carboplatin, Carmustine, Carmustine Wafer, Casodex®, CC-5013, CCI-779, CCNU, CDDP, CeeNU, Cerubidine®, Cetuximab, Chlorambucil, Cisplatin, Citrovorum Factor, Cladribine, Cortisone, Cosmegen®, CPT-11, Cyclophosphamide, Cytadren®, Cytarabine, Cytarabine Liposomal, Cytosar-U®, Cytoxan®, Dacarbazine, Dacogen, Dactinomycin, Darbepoetin Alfa, Dasatinib, Daunomycin, Daunorubicin, Daunorubicin Hydrochloride, Daunorubicin Liposomal, DaunoXome®, Decadron, Decitabine, Delta-Cortef®, Deltasone®, Denileukin Diftitox, DepoCyt™, Dexamethasone, Dexamethasone AcetateDexamethasone Sodium PhosphateDexasone, Dexrazoxane, DHAD, DIC, Diodex, Docetaxel, Doxil®, Doxorubicin, Doxorubicin Liposomal, Droxia™, DTIC, DTIC-Dome®, Duralone®, Efudex®, Eligard™, Ellence™, Eloxatin™, Elspar®, Emcyt®, Epirubicin, Epoetin Alfa, Erbitux, Erlotinib, Erwinia L-asparaginase, Estramustine, EthyolEtopophos®, Etoposide, Etoposide Phosphate, Eulexin®, Everolimus, Evista®, Exemestane, Fareston®, Faslodex®, Femara®, Filgrastim, Floxuridine, Fludara®, Fludarabine, Fluoroplex®, Fluorouracil, Fluorouracil (cream), Fluoxymesterone, Flutamide, Folinic Acid, FUDR®, Fulvestrant, G-CSF, Gefitinib, Gemcitabine, Gemtuzumab ozogamicin, GemzarGleevec™, Gliadel® Wafer, GM-CSF, Goserelin, Granulocyte—Colony Stimulating Factor, Granulocyte Macrophage Colony Stimulating Factor, Halotestin®, Herceptin®, Hexadrol, Hexylen®, Hexamethylmelamine, HMM, Hycamtin®, Hydrea®, Hydrocort Acetate®, Hydrocortisone, Hydrocortisone Sodium Phosphate, Hydrocortisone Sodium Succinate, Hydrocortone Phosphate, Hydroxyurea, Ibritumomab, Ibritumomab Tiuxetanldamycin®, Idarubicin, Ifex®, IFN-alphafosfamide, IL-11IL-2Imatinib mesylate, Imidazole CarboxamideInterferon alfa, Interferon Alfa-2b (PEG Conjugate), Interleukin-2, Interleukin-11, Intron A® (interferon alfa-2b)Iressa®, Irinotecan, Isotretinoin, Ixabepilone, Ixempra™, K, Kidrolase (t), L, Lanacort®, Lapatinib, L-asparaginase, LCR, Lenalidomide, Letrozole, Leucovorin, Leukeran, Leukine™, Leuprolide, Leurocristine, Leustatin™, Liposomal Ara-C, Liquid Pred®, Lomustine, L-PAM, L-Sarcolysin, Lupron®, Lupron Depot®, M, Matulane®, Maxidex, Mechlorethamine, Mechlorethamine Hydrochloride, Medralone®, Medrol®, Megace®, Megestrol, Megestrol Acetate, Melphalan, Mercaptopurine, Mesna, Mesnex™, Methotrexate, Methotrexate Sodium, Methylprednisolone, Meticorten®, Mitomycin, Mitomycin-C, Mitoxantrone, M-Prednisol®, MTC, MTX, Mustargen®, MustineMutamycin ®, Myleran®, Mylocel™, Mylotarg®, N, Navelbine®, Nelarabine, Neosar®, Neulasta™, Neumega®, Neupogen®, Nexavar®, Nilandron®, Nilotinib, Nilutamide, Nipent®, Nitrogen Mustard, Novaldex®, Novantrone®, Nplate, O, Octreotide, Octreotide acetate, Ofatumumab, Oncospar®, Oncovin®, Ontak®, Onxal™, Oprelvekin, Orapred®, Orasone ®, Oxaliplatin, P, Paclitaxel, Paclitaxel Protein-bound, Pamidronate, Panitumumab, Panretin ®, Paraplatin®, Pazopanib, Pediapred®, PEG Interferon, Pegaspargase, Pegfilgrastim, PEG-INTRON™, PEG-L-asparaginase, PEMETREXED, Pentostatin, Phenylalanine Mustard, Platinol®, Platinol-AQ®, Prednisolone, Prednisone, Prelone®, Procarbazine, PROCRIT®, Proleukin®, Prolifeprospan 20 with Carmustine Implant, Purinethol®, R, Raloxifene, Revlimid®, Rheumatrex®, Rituxan®, Rituximab, Roferon-A® (Interferon Alfa-2a)Romiplostim, Rubex®, Rubidomycin hydrochloride, S, Sandostatin®, Sandostatin LAR®, Sargramostim, Solu-Cortef®, Solu-Medrol®, Sorafenib, SPRYCEL™, STI-571, Streptozocin, SU11248, Sunitinib, Sutent®, T, Tamoxifen, Tarceva®, Targretin®, Tasigna ®, Taxol®, Taxotere®, Temodar®, Temozolomide, Temsirolimus, Teniposide, TESPA, Thalidomide, Thalomid®, TheraCys®, Thioguanine, Thioguanine Tabloid®, Thiophosphoamide, Thioplex®, Thiotepa, TICE®, Toposar®, Topotecan, Toremifene, Torisel®, Tositumomab, Trastuzumab, Treanda®, Tretinoin, Trexall™, Trisenox®, TSPA, TYKERB®, V, VCR, Vectibix™, Velban®, Velcade®, VePesid®, Vesanoid®, Viadur™, Vidaza®, Vinblastine, Vinblastine Sulfate, Vincasar Pfs®, Vincristine, Vinorelbine, Vinorelbine tartrate, VLB, VM-26, Vorinostat, Votrient, VP-16, Vumon®, X, Xeloda®, Z, Zanosar®, Zevalin™, Zinecard®, Zoladex®, Zoledronic acid, Zolinza, Zometa®.

In another aspect, the subject compounds can be administered in combination with 13-cis-Retinoic Acid, 2-CdA, 2-Chlorodeoxyadenosine, 5-Azacitidine, 5-Fluorouracil, 5-FU, 6-Mercaptopurine, 6-MP, 6-TG, 6-Thioguanine, Abraxane, Accutane®, Actinomycin-D, Adriamycin®, Adrucil®, Afinitor®, Agrylin®, Ala-Cort®, Aldesleukin, Alemtuzumab, ALIMTA, Alitretinoin, Alkaban-AQ®, Alkeran®, All-transretinoic Acid, Alpha Interferon, Altretamine, Amethopterin, Amifostine, Aminoglutethimide, Anagrelide, Anandron®, Anastrozole, Arabinosylcytosine, Ara-C, Aranesp®, Aredia®, Arimidex®, Aromasin®, Arranon®, Arsenic Trioxide, Arzerra™, Asparaginase, ATRA, Avastin®, Azacitidine, BCG, BCNU, Bendamustine, Bevacizumab, Bexarotene, BEXXAR®, Bicalutamide, BiCNU, Blenoxane®, Bleomycin, Bortezomib, Busulfan, Busulfex®, C225Calcium Leucovorin, Campath®, Camptosar®, Camptothecin-11, Capecitabine, Carac™, Carboplatin, Carmustine, Carmustine Wafer, Casodex®, CC-5013, CCI-779, CCNU, CDDP, CeeNU, Cerubidine®, Cetuximab, Chlorambucil, Cisplatin, Citrovorum Factor, Cladribine, Cortisone, Cosmegen®, CPT-11, Cyclophosphamide, Cytadren®, Cytarabine, Cytarabine Liposomal, Cytosar-U®, Cytoxan®, Dacarbazine, Dacogen, Dactinomycin, Darbepoetin Alfa, Dasatinib, Daunomycin, Daunorubicin, Daunorubicin Hydrochloride, Daunorubicin Liposomal, DaunoXome®, Decadron, Decitabine, Delta-Cortef®, Deltasone®, Denileukin Diftitox, DepoCyt™, Dexamethasone, Dexamethasone AcetateDexamethasone Sodium PhosphateDexasone, Dexrazoxane, DHAD, DIC, Diodex, Docetaxel, Doxil®, Doxorubicin, Doxorubicin Liposomal, Droxia™, DTIC, DTIC-Dome®, Duralone®, Efudex®, Eligard™, Ellence™, Eloxatin™, Elspar®, Emcyt®, Epirubicin, Epoetin Alfa, Erbitux, Erlotinib, Erwinia L-asparaginase, Estramustine, EthyolEtopophos®, Etoposide, Etoposide Phosphate, Eulexin®, Everolimus, Evista®, Exemestane, Fareston®, Faslodex®, Femara®, Filgrastim, Floxuridine, Fludara®, Fludarabine, Fluoroplex®, Fluorouracil, Fluorouracil (cream), Fluoxymesterone, Flutamide, Folinic Acid, FUDR®, Fulvestrant, G-CSF, Gefitinib, Gemcitabine, Gemtuzumab ozogamicin, GemzarGleevec™, Gliadel® Wafer, GM-CSF, Goserelin, Granulocyte—Colony Stimulating Factor, Granulocyte Macrophage Colony Stimulating Factor, Halotestin®, Herceptin®, Hexadrol, Hexylen®, Hexamethylmelamine, HMM, Hycamtin®, Hydrea®, Hydrocort Acetate®, Hydrocortisone, Hydrocortisone Sodium Phosphate, Hydrocortisone Sodium Succinate, Hydrocortone Phosphate, Hydroxyurea, Ibritumomab, Ibritumomab Tiuxetanldamycin®, Idarubicin, Ifex®, IFN-alphafosfamide, IL-11IL-2Imatinib mesylate, Imidazole CarboxamideInterferon alfa, Interferon Alfa-2b (PEG Conjugate), Interleukin-2, Interleukin-11, Intron A® (interferon alfa-2b)Iressa®, Irinotecan, Isotretinoin, Ixabepilone, Ixempra™, K, Kidrolase (t), L, Lanacort®, Lapatinib, L-asparaginase, LCR, Lenalidomide, Letrozole, Leucovorin, Leukeran, Leukine™, Leuprolide, Leurocristine, Leustatin™, Liposomal Ara-C, Liquid Pred ®, Lomustine, L-PAM, L-Sarcolysin, Lupron®, Lupron Depot®, M, Matulane®, Maxidex, Mechlorethamine, Mechlorethamine Hydrochloride, Medralone®, Medrol®, Megace®, Megestrol, Megestrol Acetate, Melphalan, Mercaptopurine, Mesna, Mesnex™, Methotrexate, Methotrexate Sodium, Methylprednisolone, Meticorten®, Mitomycin, Mitomycin-C, Mitoxantrone, M-Prednisol®, MTC, MTX, Mustargen®, MustineMutamycin ®, Myleran®, Mylocel™, Mylotarg®, N, Navelbine®, Nelarabine, Neosar®, Neulasta™, Neumega®, Neupogen®, Nexavar®, Nilandron®, Nilotinib, Nilutamide, Nipent®, Nitrogen Mustard, Novaldex®, Novantrone®, Nplate, O, Octreotide, Octreotide acetate, Ofatumumab, Oncospar®, Oncovin®, Ontak®, Onxal™, Oprelvekin, Orapred®, Orasone ®, Oxaliplatin, P, Paclitaxel, Paclitaxel Protein-bound, Pamidronate, Panitumumab, Panretin ®, Paraplatin®, Pazopanib, Pediapred®, PEG Interferon, Pegaspargase, Pegfilgrastim, PEG-INTRON™, PEG-L-asparaginase, PEMETREXED, Pentostatin, Phenylalanine Mustard, Platinol®, Platinol-AQ®, Prednisolone, Prednisone, Prelone®, Procarbazine, PROCRIT®, Proleukin®, Prolifeprospan 20 with Carmustine Implant, Purinethol®, R, Raloxifene, Revlimid®, Rheumatrex®, Rituxan®, Rituximab, Roferon-A® (Interferon Alfa-2a)Romiplostim, Rubex®, Rubidomycin hydrochloride, S, Sandostatin®, Sandostatin LAR®, Sargramostim, Solu-Cortef®, Solu-Medrol®, Sorafenib, SPRYCEL™, STI-571, Streptozocin, SU11248, Sunitinib, Sutent®, T, Tamoxifen, Tarceva®, Targretin®, Tasigna ®, Taxol®, Taxotere®, Temodar®, Temozolomide, Temsirolimus, Teniposide, TESPA, Thalidomide, Thalomid®, TheraCys®, Thioguanine, Thioguanine Tabloid®, Thiophosphoamide, Thioplex®, Thiotepa, TICE®, Toposar®, Topotecan, Toremifene, Torisel®, Tositumomab, Trastuzumab, Treanda®, Tretinoin, Trexall™, Trisenox®, TSPA, TYKERB®, V, VCR, Vectibix™, Velban®, Velcade®, VePesid®, Vesanoid®, Viadur™, Vidaza®, Vinblastine, Vinblastine Sulfate, Vincasar Pfs®, Vincristine, Vinorelbine, Vinorelbine tartrate, VLB, VM-26, Vorinostat, Votrient, VP-16, Vumon®, X, Xeloda®, Z, Zanosar®, Zevalin™, Zinecard®, Zoladex®, Zoledronic acid, Zolinza, Zometa®

In another aspect, the subject compound can be used in combination with 13-cis-Retinoic Acid, 2-CdA, 2-Chlorodeoxyadenosine, 5-Azacitidine, 5-Fluorouracil, 5-FU, 6-Mercaptopurine, 6-MP, 6-TG, 6-Thioguanine, Abraxane, Accutane®, Actinomycin-D, Adriamycin®, Adrucil®, Afinitor®, Agrylin®, Ala-Cort®, Aldesleukin, Alemtuzumab, ALIMTA, Alitretinoin, Alkaban-AQ®, Alkeran®, All-transretinoic Acid, Alpha Interferon, Altretamine, Amethopterin, Amifostine, Aminoglutethimide, Anagrelide, Anandron®, Anastrozole, Arabinosylcytosine, Ara-C, Aranesp®, Aredia®, Arimidex®, Aromasin®, Arranon®, Arsenic Trioxide, Arzerra™, Asparaginase, ATRA, Avastin®, Azacitidine, BCG, BCNU, Bendamustine, Bevacizumab, Bexarotene, BEXXAR®, Bicalutamide, BiCNU, Blenoxane®, Bleomycin, Bortezomib, Busulfan, Busulfex®, C225Calcium Leucovorin, Campath®, Camptosar®, Camptothecin-11, Capecitabine, Carac™, Carboplatin, Carmustine, Carmustine Wafer, Casodex®, CC-5013, CCI-779, CCNU, CDDP, CeeNU, Cerubidine®, Cetuximab, Chlorambucil, Cisplatin, Citrovorum Factor, Cladribine, Cortisone, Cosmegen®, CPT-11, Cyclophosphamide, Cytadren®, Cytarabine, Cytarabine Liposomal, Cytosar-U®, Cytoxan®, Dacarbazine, Dacogen, Dactinomycin, Darbepoetin Alfa, Dasatinib, Daunomycin, Daunorubicin, Daunorubicin Hydrochloride, Daunorubicin Liposomal, DaunoXome®, Decadron, Decitabine, Delta-Cortef®, Deltasone®, Denileukin Diftitox, DepoCyt™, Dexamethasone, Dexamethasone AcetateDexamethasone Sodium PhosphateDexasone, Dexrazoxane, DHAD, DIC, Diodex, Docetaxel, Doxil®, Doxorubicin, Doxorubicin Liposomal, Droxia™, DTIC, DTIC-Dome®, Duralone®, Efudex®, Eligard™, Ellence™, Eloxatin™, Elspar®, Emcyt®, Epirubicin, Epoetin Alfa, Erbitux, Erlotinib, Erwinia L-asparaginase, Estramustine, EthyolEtopophos®, Etoposide, Etoposide Phosphate, Eulexin®, Everolimus, Evista®, Exemestane, Fareston®, Faslodex®, Femara®, Filgrastim, Floxuridine, Fludara®, Fludarabine, Fluoroplex®, Fluorouracil, Fluorouracil (cream), Fluoxymesterone, Flutamide, Folinic Acid, FUDR®, Fulvestrant, G-CSF, Gefitinib, Gemcitabine, Gemtuzumab ozogamicin, GemzarGleevec™, Gliadel® Wafer, GM-CSF, Goserelin, Granulocyte—Colony Stimulating Factor, Granulocyte Macrophage Colony Stimulating Factor, Halotestin®, Herceptin®, Hexadrol, Hexylen®, Hexamethylmelamine, HMM, Hycamtin®, Hydrea®, Hydrocort Acetate®, Hydrocortisone, Hydrocortisone Sodium Phosphate, Hydrocortisone Sodium Succinate, Hydrocortone Phosphate, Hydroxyurea, Ibritumomab, Ibritumomab Tiuxetanldamycin®, Idarubicin, Ifex®, IFN-alphafosfamide, IL-11IL-2Imatinib mesylate, Imidazole CarboxamideInterferon alfa, Interferon Alfa-2b (PEG Conjugate), Interleukin-2, Interleukin-11, Intron A® (interferon alfa-2b)Iressa®, Irinotecan, Isotretinoin, Ixabepilone, Ixempra™, K, Kidrolase (t), L, Lanacort®, Lapatinib, L-asparaginase, LCR, Lenalidomide, Letrozole, Leucovorin, Leukeran, Leukine™, Leuprolide, Leurocristine, Leustatin™, Liposomal Ara-C, Liquid Pred®, Lomustine, L-PAM, L-Sarcolysin, Lupron®, Lupron Depot®, M, Matulane®, Maxidex, Mechlorethamine, Mechlorethamine Hydrochloride, Medralone®, Medrol®, Megace®, Megestrol, Megestrol Acetate, Melphalan, Mercaptopurine, Mesna, Mesnex™, Methotrexate, Methotrexate Sodium, Methylprednisolone, Meticorten®, Mitomycin, Mitomycin-C, Mitoxantrone, M-Prednisol®, MTC, MTX, Mustargen®, MustineMutamycin®, Myleran®, Mylocel™, Mylotarg®, N, Navelbine®, Nelarabine, Neosar®, Neulasta™, Neumega®, Neupogen®, Nexavar®, Nilandron®, Nilotinib, Nilutamide, Nipent®, Nitrogen Mustard, Novaldex®, Novantrone®, Nplate, O, Octreotide, Octreotide acetate, Ofatumumab, Oncospar®, Oncovin®, Ontak®, Onxal™, Oprelvekin, Orapred®, Orasone ®, Oxaliplatin, P, Paclitaxel, Paclitaxel Protein-bound, Pamidronate, Panitumumab, Panretin ®, Paraplatin®, Pazopanib, Pediapred®, PEG Interferon, Pegaspargase, Pegfilgrastim, PEG-INTRON™, PEG-L-asparaginase, PEMETREXED, Pentostatin, Phenylalanine Mustard, Platinol®, Platinol-AQ®, Prednisolone, Prednisone, Prelone®, Procarbazine, PROCRIT®, Proleukin®, Prolifeprospan 20 with Carmustine Implant, Purinethol®, R, Raloxifene, Revlimid®, Rheumatrex®, Rituxan®, Rituximab, Roferon-A® (Interferon Alfa-2a)Romiplostim, Rubex®, Rubidomycin hydrochloride, S, Sandostatin®, Sandostatin LAR ®, Sargramostim, Solu-Cortef®, Solu-Medrol®, Sorafenib, SPRYCEL™, STI-571, Streptozocin, SU11248, Sunitinib, Sutent®, T, Tamoxifen, Tarceva®, Targretin®, Tasigna ®, Taxol®, Taxotere®, Temodar®, Temozolomide, Temsirolimus, Teniposide, TESPA, Thalidomide, Thalomid®, TheraCys®, Thioguanine, Thioguanine Tabloid®, Thiophosphoamide, Thioplex®, Thiotepa, TICE®, Toposar®, Topotecan, Toremifene, Torisel®, Tositumomab, Trastuzumab, Treanda®, Tretinoin, Trexall™, Trisenox®, TSPA, TYKERB®, V, VCR, Vectibix™, Velban®, Velcade®, VePesid®, Vesanoid®, Viadur™, Vidaza®, Vinblastine, Vinblastine Sulfate, Vincasar Pfs®, Vincristine, Vinorelbine, Vinorelbine tartrate, VLB, VM-26, Vorinostat, Votrient, VP-16, Vumon®, X, Xeloda®, Z, Zanosar®, Zevalin™, Zinecard®, Zoladex®, Zoledronic acid, Zolinza, Zometa®

The pharmaceutical compositions and methods of the present invention can further comprise other therapeutically active compounds as noted herein which are usually applied in the treatment of the above mentioned pathological conditions.

1. Treatment Methods

The compounds disclosed herein are useful for treating, preventing, ameliorating, controlling or reducing the risk of a variety of disorders of uncontrolled cellular proliferation associated with a STAT protein activity dysfunction. In a yet further aspect, the disorder of uncontrolled cellular proliferation is a cancer. In a still further aspect, the STAT protein activity dysfunction is that the STAT protein is persistently active. In a yet further aspect, the STAT protein is constitutively active. In an even further aspect, the STAT protein is overexpressed. In a still further aspect, the STAT protein is STATS.

In one aspect, the invention relates to a method for the treatment of a disorder associated with a STAT protein activity dysfunction in a mammal comprising the step of administering to the mammal at least one disclosed compound or at least one disclosed product in a dosage and amount effective to treat the disorder in the mammal. In a further aspect, the mammal is a human. In a further aspect, the mammal has been diagnosed with a need for treatment of the disorder prior to the administering step. In a further aspect, the method further comprises the step of identifying a mammal in need of treatment of the disorder.

It is understood that cancer refer to or describe the physiological condition in mammals that is typically characterized by unregulated cell growth. The cancer may be multi-drug resistant (MDR) or drug-sensitive. Examples of cancer include but are not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, gastrointestinal cancer, pancreatic cancer, cervical cancer, ovarian cancer, peritoneal cancer, liver cancer, e.g., hepatic carcinoma, bladder cancer, colorectal cancer, endometrial carcinoma, kidney cancer, and thyroid cancer.

In various aspects, further examples of cancers are basal cell carcinoma, biliary tract cancer; bone cancer; brain and CNS cancer; choriocarcinoma; connective tissue cancer; esophageal cancer; eye cancer; cancer of the head and neck; gastric cancer; intra-epithelial neoplasm; larynx cancer; lymphoma including Hodgkin's and Non-Hodgkin's lymphoma; melanoma; myeloma; neuroblastoma; oral cavity cancer (e.g., lip, tongue, mouth, and pharynx); retinoblastoma; rhabdomyosarcoma; rectal cancer; cancer of the respiratory system; sarcoma; skin cancer; stomach cancer; testicular cancer; uterine cancer; cancer of the urinary system, as well as other carcinomas and sarcomas

Examples of disorders such as a cancer associated with STAT protein activity dysfunction, e.g. a STAT3 activity dysfunction, include: Acute Lymphoblastic Leukemia, Adult Hairy Cell Leukemia, Acute Lymphoblastic Leukemia, Head and Neck Cancer, Childhood Hepatocellular (Liver) Cancer, Adult, Acute Myeloid Leukemia, Adult (Primary), Acute Myeloid Leukemia, Childhood Hepatocellular (Liver) Cancer, Childhood, Adrenocortical Carcinoma (Primary), Adrenocortical Carcinoma, Childhood Hodgkin's Lymphoma, AdultAIDS-Related Cancers Hodgkin's Lymphoma, ChildhoodAlDS-Related Lymphoma Hodgkin's Lymphoma During PregnancyAnal Cancer Hypopharyngeal CancerAstrocytoma, Childhood Cerebellar Hypothalamic and Visual Pathway Glioma, Astrocytoma, Childhood Cerebral ChildhoodBasal Cell Carcinoma Intraocular MelanomaBile Duct Cancer, Extrahepatic Islet Cell Carcinoma (Endocrine Pancreas)Bladder Cancer Kaposi's SarcomaBladder Cancer, Childhood Kidney (Renal Cell) CancerBone Cancer, Osteosarcoma/Malignant Kidney Cancer, ChildhoodFibrous Histiocytoma Laryngeal CancerBrain Stem Glioma, Childhood Laryngeal Cancer, ChildhoodBrain Tumor, Adult Leukemia, Acute Lymphoblastic, AdultBrain Tumor, Brain Stem Glioma, Leukemia, Acute Lymphoblastic, ChildhoodChildhood Leukemia, Acute Myeloid, AdultBrain Tumor, Cerebellar Astrocytoma, Leukemia, Acute Myeloid, ChildhoodChildhood Leukemia, Chronic LymphocyticBrain Tumor, Cerebral Leukemia, Chronic MyelogenousAstrocytoma/Malignant Glioma, Leukemia, Hairy CellChildhood Lip and Oral Cavity CancerBrain Tumor, Ependymoma, Childhood Liver Cancer, Adult (Primary)Brain Tumor, Medulloblastoma, Liver Cancer, Childhood (Primary)Childhood Lung Cancer, Non-Small CellBrain Tumor, Supratentorial Primitive Lung Cancer, Small CellNeuroectodermal Tumors, Childhood Lymphoma, AIDS-RelatedBrain Tumor, Visual Pathway and Lymphoma, Burkitt'sHypothalamic Glioma, Childhood Lymphoma, Cutaneous T-Cell, see MycosisBrain Tumor, Childhood Fungoides and Sézary SyndromeBreast Cancer Lymphoma, Hodgkin's, AdultBreast Cancer, Childhood Lymphoma, Hodgkin's, ChildhoodBreast Cancer, Male Lymphoma, Hodgkin's During Pregnancyl)ronchial Adenomas/Carcinoids, Lymphoma, Non-Hodgkin's, AdultChildhood Lymphoma, Non-Hodgkin's, ChildhoodBurkitt's Lymphoma Lymphoma, Non-Hodgkin's DuringCarcinoid Tumor, Childhood PregnancyCarcinoid Tumor, Gastrointestinal Lymphoma, Primary Central Nervous SystemCarcinoma of Unknown Primary Macroglobulinemia, Waldenström'sCentral Nervous System Lymphoma, Malignant Fibrous Histiocytoma of Primary Bone/OsteosarcomaCerebellar Astrocytoma, Childhood Medulloblastoma, ChildhoodCerebral Astrocytoma/Malignant MelanomaGlio ma, Childhood Melanoma, Intraocular (Eye)Cervical Cancer Merkel Cell CarcinomaChildhood Cancers Mesothelioma, Adult MalignantChronic Lymphocytic Leukemia Mesothelioma, ChildhoodChronic Myelogenous Leukemia Metastatic Squamous Neck Cancer withChronic Myeloproliferative Disorders Occult PrimaryColon Cancer Multiple Endocrine Neoplasia Syndrome, Colorectal Cancer, Childhood ChildhoodCutaneous T-Cell Lymphoma, see Multiple Myeloma/Plasma Cell NeoplasmMycosis Fungoides and Sézary Mycosis FungoidesSyndrome Myelodysplastic SyndromesEndometrial Cancer Myelodysplastic/Myeloproliferative DiseasesEpendymoma, Childhood Myelogenous Leukemia, ChronicEsophageal Cancer Myeloid Leukemia, Adult AcuteEsophageal Cancer, Childhood Myeloid Leukemia, Childhood AcuteEwing's Family of Tumors Myeloma, MultipleExtracranial Germ Cell Tumor, Myeloproliferative Disorders, ChronicChildhood Nasal Cavity and Paranasal Sinus CancerExtragonadal Germ Cell Tumor Nasopharyngeal CancerExtrahepatic Bile Duct Cancer Nasopharyngeal Cancer, ChildhoodEye Cancer, Intraocular Melanoma NeuroblastomaEye Cancer, Retinoblastoma Non-Hodgkin's Lymphoma, AdultGallbladder Cancer Non-Hodgkin's Lymphoma, ChildhoodGastric (Stomach) Cancer Non-Hodgkin's Lymphoma During PregnancyGastric (Stomach) Cancer, Childhood Non-Small Cell Lung CancerGastrointestinal Carcinoid Tumor Oral Cancer, ChildhoodGerm Cell Tumor, Extracranial, Oral Cavity Cancer, Lip and Childhood Oropharyngeal CancerGerm Cell Tumor, Extragonadal Osteosarcoma/Malignant FibrousGerm Cell Tumor, Ovarian Histiocytoma of BoneGestational Trophoblastic Tumor Ovarian Cancer, ChildhoodGlioma, Adult Ovarian Epithelial CancerGlioma, Childhood Brain Stem Ovarian Germ Cell TumorGlioma, Childhood Cerebral Ovarian Low Malignant Potential TumorAstrocytoma Pancreatic CancerGlioma, Childhood Visual Pathway and Pancreatic Cancer, ChildhoodHypothalamic Pancreatic Cancer, Islet CellSkin Cancer (Melanoma) Paranasal Sinus and Nasal Cavity CancerSkin Carcinoma, Merkel Cell Parathyroid CancerSmall Cell Lung Cancer Penile CancerSmall Intestine Cancer PheochromocytomaSoft Tissue Sarcoma, Adult Pineoblastoma and Supratentorial PrimitiveSoft Tissue Sarcoma, Childhood Neuroectodermal Tumors, ChildhoodSquamous Cell Carcinoma, see Skin Pituitary Tumor Cancer (non-Melanoma) Plasma Cell Neoplasm/Multiple MyelomaSquamous Neck Cancer with Occult Pleuropulmonary BlastomaPrimary, Metastatic Pregnancy and Breast CancerStomach (Gastric) Cancer Pregnancy and Hodgkin's LymphomaStomach (Gastric) Cancer, Childhood Pregnancy and Non-Hodgkin's LymphomaSupratentorial Primitive Primary Central Nervous System LymphomaNeuroectodermal Tumors, Childhood Prostate CancerT-Cell Lymphoma, Cutaneous, see Rectal CancerMycosis Fungoides and Sézary Renal Cell (Kidney) CancerSyndrome Renal Cell (Kidney) Cancer, ChildhoodTesticular Cancer Renal Pelvis and Ureter, Transitional CellThymoma, Childhood CancerThymoma and Thymic Carcinoma RetinoblastomaThyroid Cancer Rhabdomyosarcoma, ChildhoodThyroid Cancer, Childhood Salivary Gland CancerTransitional Cell Cancer of the Renal Salivary Gland Cancer, ChildhoodPelvis and Ureter Sarcoma, Ewing's Family of TumorsTrophoblastic Tumor, Gestational Sarcoma, Kaposi'sUnknown Primary Site, Carcinoma of, Sarcoma, Soft Tissue, AdultAdult Sarcoma, Soft Tissue, ChildhoodUnknown Primary Site, Cancer of, Sarcoma, UterineChildhood Sezary SyndromeUnusual Cancers of Childhood Skin Cancer (non-Melanoma)Ureter and Renal Pelvis, Transitional Skin Cancer, ChildhoodCell CancerUrethral CancerUterine Cancer, EndometrialUterine SarcomaVaginal CancerVisual Pathway and HypothalamicGlioma, ChildhoodVulvar CancerWaldenstrom's MacroglobulinemiaWilms' Tumor.

The disorders of uncontrolled cellular proliferation, e.g. a cancer, that can be treated or prevented by the compositions disclosed herein include.

Thus, provided is a method for treating or preventing a disorder of uncontrolled cellular proliferation, comprising: administering to a subject at least one disclosed compound; at least one disclosed pharmaceutical composition; and/or at least one disclosed product in a dosage and amount effective to treat the disorder in the subject.

a. Treatment of a Disorder Associated with STAT Activity Dysfunction

In one aspect, the invention relates to a method for the treatment of a disorder associated with STAT activity dysfunction in a mammal comprising the step of administering to the mammal a therapeutically effective amount of a compound having a structure represented by a formula:

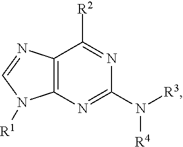

wherein R¹ is selected from H and (CH₂)ₘC═OR⁵, wherein m is an integer from 0-3; wherein R⁵ is selected from OR⁶ and NR⁷R⁸; wherein R⁶ is selected from hydrogen and C1-C8 alkyl; wherein each of R⁷ and R⁸ is independently selected from hydrogen and C1-C8 alkyl; wherein R² is selected from halogen, OR⁹, and NR¹⁰R¹¹; wherein R⁹ is an optionally substituted group selected from C1-C8 alkyl, C3-C6 cycloalkyl, C3-C6 heterocycloalkyl, (CH₂)ₙR¹², and Ar¹; wherein n is an integer from 0-3; wherein R¹² is an optionally substituted group selected from Ar¹, C3-C6 cycloalkyl and C3-C6 heterocycloalkyl; wherein Ar¹ is selected from an optionally substituted monocyclic aryl and monocyclic heteroaryl; wherein R¹⁰ is selected from hydrogen and an optionally substituted group selected from C1-C8 alkyl, C3-C6 cycloalkyl, C3-C6 heterocycloalkyl, (CH₂)ₚR¹³, and Ar²; wherein p is an integer from 0-3; wherein R¹³ is an optionally substituted group selected from Ar², C3-C6 cycloalkyl and C3-C6 heterocycloalkyl; wherein Ar² is selected from an optionally substituted monocyclic aryl and monocyclic heteroaryl; wherein R¹¹ is selected from hydrogen and C1-C8 alkyl; wherein R³ is selected from hydrogen and an optionally substituted group selected from C1-C8 alkyl, (CH₂)_{q}R¹⁴, and C═O(CH₂)_{q}R¹⁴; wherein q is an integer from 0-3; wherein R¹⁴ is selected from hydrogen and an optionally substituted group selected from C1-C8 alkyl, C3-C6 cycloalkyl, C3-C6 heterocycloalkyl, and Ar³; wherein Ar³ is selected from an optionally substituted monocyclic aryl and monocyclic heteroaryl; wherein R⁴ is selected from hydrogen and C═OOR¹⁵; wherein R¹⁵ is selected from hydrogen and optionally substituted C1-C8 alkyl; wherein R³ and R⁴ are optionally covalently bonded and, together with the intermediate carbon, comprise an optionally substituted 3- to 7-membered heterocycloalkyl; and wherein R¹⁰ and R¹¹ are optionally covalently bonded and, together with the intermediate carbon, comprise an optionally substituted 3- to 7-membered heterocycloalkyl; or a pharmaceutically acceptable salt, hydrate, solvate, or polymorph thereof.

In a further aspect, the compound administered is any of the disclosed compounds or at least one product of the disclosed methods of making.

In a further aspect, the mammal is a human. In a yet further aspect, the mammal has been diagnosed with a need for treatment of the disorder prior to the administering step. In a still further aspect, the method further comprises the step of identifying a mammal in need of treatment of the disorder.

In a further aspect, the compound administered inhibits STAT protein activity. In a further aspect, the compound administered inhibits STAT3 protein activity. In a yet further aspect, the compound administered inhibits STAT3 protein activity. In a still further aspect, the compound administered prevents STAT3 protein dimerization. In a yet further aspect, the compound administered disrupts preformed or existing STAT3 dimers. In a still further aspect, the compound administered binds to the SH2 domain of STAT3.

In a further aspect, the compound administered inhibits STAT protein activity with an IC₅₀ in an EMSA assay of less than about 100 μM, less than about 50 μM, less than about 10 μM, less than about 1 μM, less than about 500 nM, or of less than about 100 nM. In a further aspect, the IC₅₀ for inhibition of STAT3 activity.

In a further aspect, the compound administered inhibits cell growth. In a still further aspect, the compound inhibits cell growth with an IC₅₀ of less than about 500 μM. In a yet further aspect, the compound inhibits cell growth with an IC₅₀ of less than about 250 μM. In an even further aspect, the compound inhibits cell growth with an IC₅₀ of less than about 100 μM. In a still further aspect, the compound inhibits cell growth with an IC₅₀ of less than about 50 μM. In a yet further aspect, the compound inhibits cell growth with an IC₅₀ of less than about 10 μM. In an even further aspect, the compound inhibits cell growth with an IC₅₀ of less than about 1 μM. In a still further aspect, the IC₅₀ for inhibition of cell growth is determined in a cell line with a constitutively active STAT protein. In a yet further aspect, the IC₅₀ for inhibition of cell growth is determined in a cell line with a persistently active STAT protein. In an even further aspect, the IC₅₀ for inhibition of cell growth is determined in a cell line derived from a cancer selected from breast cancer, pancreatic cancer, and prostate cancer. In a yet further aspect, the IC₅₀ for inhibition of cell growth is determined in a cell line derived from a cancer with a STAT protein activity dysfunction. In a still further aspect, the IC₅₀ for inhibition of cell growth is determined in a cell line selected from MDA-MB-231, Panc-1, and DU 145. In a yet further aspect, the IC₅₀ for inhibition of cell growth is determined in a cell line transformed with v-Src. In an even further aspect, the IC₅₀ for inhibition of cell growth is determined in the NIH3T3 cell-line transformed with v-Src.

In a further aspect, the compound administered treats a disorder is associated with constitutively active STAT3.

In a further aspect, the compound administered treats a disorder selected from psoriasis and pulmonary arterial hypertension.

In a further aspect, the compound administered treats a disorder of uncontrolled cellular proliferation. In a yet further aspect, the disorder of uncontrolled cellular proliferation is cancer. In a still further aspect, the cancer is selected from a cancer of the head, neck, pancreas, brain, ovary, kidney, prostate, breast, lung, colon, and liver.

In a further aspect, the cancer is a hematological cancer. In a still further aspect, the hematological cancer is selected from acute myeloid leukemia (AML), acute lymphoblastic leukemia (ALL), chronic myeloid leukemia (CML), chronic lymphocytic leukemia (CLL), hairy cell leukemia, chronic myelomonocytic leukemia (CMML), juvenile myelomonocytic leukemia (JMML), Hodgkin lymphoma, Non-Hodgkin lymphoma, multiple myeloma, solitary myeloma, localized myeloma, and extramedullary myeloma.

In a further aspect, the cancer is a cancer of the brain. In a still further aspect, the cancer of the brain is selected from a glioma, medulloblastoma, primitive neuroectodermal tumor (PNET), acoustic neuroma, glioma, meningioma, pituitary adenoma, schwannoma, CNS lymphoma, primitive neuroectodermal tumor, craniopharyngioma, chordoma, medulloblastoma, cerebral neuroblastoma, central neurocytoma, pineocytoma, pineoblastoma, atypical teratoid rhabdoid tumor, chondrosarcoma, chondroma, choroid plexus carcinoma, choroid plexus papilloma, craniopharyngioma, dysembryoplastic neuroepithelial tumor, gangliocytoma, germinoma, hemangioblastoma, hemangiopercytoma, and metastatic brain tumor. In a yet further aspect, the glioma is selected from ependymoma, astrocytoma, oligodendroglioma, and oligoastrocytoma. In an even further aspect, the glioma is selected from juvenile pilocytic astrocytoma, subependymal giant cell astrocytoma, ganglioglioma, subependymoma, pleomorphic xanthoastrocytom, anaplastic astrocytoma, glioblastoma multiforme, brain stem glioma, oligodendroglioma, ependymoma, oligoastrocytoma, cerebellar astrocytoma, desmoplastic infantile astrocytoma, subependymal giant cell astrocytoma, diffuse astrocytoma, mixed glioma, optic glioma, gliomatosis cerebri, multifocal gliomatous tumor, multicentric glioblastoma multiforme tumor, paraganglioma, and ganglioglioma.

In a further aspect, the cancer is selected from a cancer of the breast, ovary, prostate, head, neck, and kidney. In a still further aspect, the cancer is breast cancer. In a yet further aspect, the cancer is pancreatic cancer.

b. Inhibition of STAT Activity in a Mammal

In one aspect, the invention relates to a method for inhibition of STAT activity in a mammal comprising the step of administering to the mammal a therapeutically effective amount of least one compound having a structure represented by a formula:

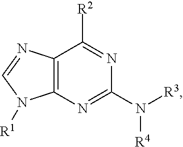

wherein R¹ is selected from H and (CH₂)ₘC═OR⁵, wherein m is an integer from 0-3; wherein R⁵ is selected from OR⁶ and NR⁷R⁸; wherein R⁶ is selected from hydrogen and C1-C8 alkyl; wherein each of R⁷ and R⁸ is independently selected from hydrogen and C1-C8 alkyl; wherein R² is selected from halogen, OR⁹, and NR¹⁰R¹¹; wherein R⁹ is an optionally substituted group selected from C1-C8 alkyl, C3-C6 cycloalkyl, C3-C6 heterocycloalkyl, (CH₂)ₙR¹², and Ar¹; wherein n is an integer from 0-3; wherein R¹² is an optionally substituted group selected from Ar¹, C3-C6 cycloalkyl and C3-C6 heterocycloalkyl; wherein Ar¹ is selected from an optionally substituted monocyclic aryl and monocyclic heteroaryl; wherein R¹⁰ is selected from hydrogen and an optionally substituted group selected from C1-C8 alkyl, C3-C6 cycloalkyl, C3-C6 heterocycloalkyl, (CH₂)ₚR¹³, and Ar²; wherein p is an integer from 0-3; wherein R¹³ is an optionally substituted group selected from Ar², C3-C6 cycloalkyl and C3-C6 heterocycloalkyl; wherein Ar² is selected from an optionally substituted monocyclic aryl and monocyclic heteroaryl; wherein R¹¹ is selected from hydrogen and C1-C8 alkyl; wherein R³ is selected from hydrogen and an optionally substituted group selected from C1-C8 alkyl, (CH₂)_{q}R¹⁴, and C═O(CH₂)_{q}R¹⁴; wherein q is an integer from 0-3; wherein R¹⁴ is selected from hydrogen and an optionally substituted group selected from C1-C8 alkyl, C3-C6 cycloalkyl, C3-C6 heterocycloalkyl, and Ar³; wherein Ar³ is selected from an optionally substituted monocyclic aryl and monocyclic heteroaryl; wherein R⁴ is selected from hydrogen and C═OOR¹⁵; wherein R¹⁵ is selected from hydrogen and optionally substituted C1-C8 alkyl; wherein R³ and R⁴ are optionally covalently bonded and, together with the intermediate carbon, comprise an optionally substituted 3- to 7-membered heterocycloalkyl; and wherein R¹⁰ and R¹¹ are optionally covalently bonded and, together with the intermediate carbon, comprise an optionally substituted 3- to 7-membered heterocycloalkyl; or a pharmaceutically acceptable salt, hydrate, solvate, or polymorph thereof.

In a further aspect, the compound administered is any of the disclosed compounds or at least one product of the disclosed methods of making.

In a further aspect, the mammal is a human. In a yet further aspect, the mammal has been diagnosed with a need for treatment of the disorder prior to the administering step. In a still further aspect, the method further comprises the step of identifying a mammal in need of treatment of the disorder.

In a further aspect, the compound administered inhibits STAT protein activity. In a further aspect, the compound administered inhibits STAT3 protein activity. In a yet further aspect, the compound administered inhibits STAT3 protein activity. In a still further aspect, the compound administered prevents STAT3 protein dimerization. In a yet further aspect, the compound administered disrupts preformed or existing STAT3 dimers. In a still further aspect, the compound administered binds to the SH2 domain of STAT3.

In a further aspect, the compound administered inhibits STAT protein activity with an IC₅₀ in an EMSA assay of less than about 100 μM, less than about 50 μM, less than about 10 μM, less than about 1 μM, less than about 500 nM, or of less than about 100 nM. In a further aspect, the IC₅₀ for inhibition of STAT3 activity.

In a further aspect, the compound administered inhibits cell growth. In a still further aspect, the compound inhibits cell growth with an IC₅₀ of less than about 500 μM. In a yet further aspect, the compound inhibits cell growth with an IC₅₀ of less than about 250 μM. In an even further aspect, the compound inhibits cell growth with an IC₅₀ of less than about 100 μM. In a still further aspect, the compound inhibits cell growth with an IC₅₀ of less than about 50 μM. In a yet further aspect, the compound inhibits cell growth with an IC₅₀ of less than about 10 μM. In an even further aspect, the compound inhibits cell growth with an IC₅₀ of less than about 1 μM. In a still further aspect, the IC₅₀ for inhibition of cell growth is determined in a cell line with a constitutively active STAT protein. In a yet further aspect, the IC₅₀ for inhibition of cell growth is determined in a cell line with a persistently active STAT protein. In an even further aspect, the IC₅₀ for inhibition of cell growth is determined in a cell line derived from a cancer selected from breast cancer, pancreatic cancer, and prostate cancer. In a yet further aspect, the IC₅₀ for inhibition of cell growth is determined in a cell line derived from a cancer with a STAT protein activity dysfunction. In a still further aspect, the IC₅₀ for inhibition of cell growth is determined in a cell line selected from MDA-MB-231, Panc-1, and DU 145. In a yet further aspect, the IC₅₀ for inhibition of cell growth is determined in a cell line transformed with v-Src. In an even further aspect, the IC₅₀ for inhibition of cell growth is determined in the NIH3T3 cell-line transformed with v-Src.

In a further aspect, the compound administered treats a disorder is associated with constitutively active STAT3.

In a further aspect, the compound administered treats a disorder selected from psoriasis and pulmonary arterial hypertension.

In a further aspect, the compound administered treats a disorder of uncontrolled cellular proliferation. In a yet further aspect, the disorder of uncontrolled cellular proliferation is cancer. In a still further aspect, the cancer is selected from a cancer of the head, neck, pancreas, brain, ovary, kidney, prostate, breast, lung, colon, and liver.

In a further aspect, the cancer is a hematological cancer. In a still further aspect, the hematological cancer is selected from acute myeloid leukemia (AML), acute lymphoblastic leukemia (ALL), chronic myeloid leukemia (CML), chronic lymphocytic leukemia (CLL), hairy cell leukemia, chronic myelomonocytic leukemia (CMML), juvenile myelomonocytic leukemia (JMML), Hodgkin lymphoma, Non-Hodgkin lymphoma, multiple myeloma, solitary myeloma, localized myeloma, and extramedullary myeloma.

In a further aspect, the cancer is a cancer of the brain. In a still further aspect, the cancer of the brain is selected from a glioma, medulloblastoma, primitive neuroectodermal tumor (PNET), acoustic neuroma, glioma, meningioma, pituitary adenoma, schwannoma, CNS lymphoma, primitive neuroectodermal tumor, craniopharyngioma, chordoma, medulloblastoma, cerebral neuroblastoma, central neurocytoma, pineocytoma, pineoblastoma, atypical teratoid rhabdoid tumor, chondrosarcoma, chondroma, choroid plexus carcinoma, choroid plexus papilloma, craniopharyngioma, dysembryoplastic neuroepithelial tumor, gangliocytoma, germinoma, hemangioblastoma, hemangiopercytoma, and metastatic brain tumor. In a yet further aspect, the glioma is selected from ependymoma, astrocytoma, oligodendroglioma, and oligoastrocytoma. In an even further aspect, the glioma is selected from juvenile pilocytic astrocytoma, subependymal giant cell astrocytoma, ganglioglioma, subependymoma, pleomorphic xanthoastrocytom, anaplastic astrocytoma, glioblastoma multiforme, brain stem glioma, oligodendroglioma, ependymoma, oligoastrocytoma, cerebellar astrocytoma, desmoplastic infantile astrocytoma, subependymal giant cell astrocytoma, diffuse astrocytoma, mixed glioma, optic glioma, gliomatosis cerebri, multifocal gliomatous tumor, multicentric glioblastoma multiforme tumor, paraganglioma, and ganglioglioma.

In a further aspect, the cancer is selected from a cancer of the breast, ovary, prostate, head, neck, and kidney. In a still further aspect, the cancer is breast cancer. In a yet further.

c. Inhibiting STAT Activity in Cells

In one aspect, the invention relates to a method for inhibiting STAT activity in at least one cell, comprising the step of contacting the at least one cell with an effective amount of least one compound having a structure represented by a formula:

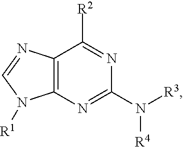

wherein R¹ is selected from H and (CH₂)ₘC═OR⁵, wherein m is an integer from 0-3; wherein R⁵ is selected from OR⁶ and NR⁷R⁸; wherein R⁶ is selected from hydrogen and C1-C8 alkyl; wherein each of R⁷ and R⁸ is independently selected from hydrogen and C1-C8 alkyl; wherein R² is selected from halogen, OR⁹, and NR¹⁰R¹¹; wherein R⁹ is an optionally substituted group selected from C1-C8 alkyl, C3-C6 cycloalkyl, C3-C6 heterocycloalkyl, (CH₂)ₙR¹², and Ar¹; wherein n is an integer from 0-3; wherein R¹² is an optionally substituted group selected from Ar¹, C3-C6 cycloalkyl and C3-C6 heterocycloalkyl; wherein Ar¹ is selected from an optionally substituted monocyclic aryl and monocyclic heteroaryl; wherein R¹⁰ is selected from hydrogen and an optionally substituted group selected from C1-C8 alkyl, C3-C6 cycloalkyl, C3-C6 heterocycloalkyl, (CH₂)ₚR¹³, and Ar²; wherein p is an integer from 0-3; wherein R¹³ is an optionally substituted group selected from Ar², C3-C6 cycloalkyl and C3-C6 heterocycloalkyl; wherein Ar² is selected from an optionally substituted monocyclic aryl and monocyclic heteroaryl; wherein R¹¹ is selected from hydrogen and C1-C8 alkyl; wherein R³ is selected from hydrogen and an optionally substituted group selected from C1-C8 alkyl, (CH₂)_{q}R¹⁴, and C═O(CH₂)_{q}R¹⁴; wherein q is an integer from 0-3; wherein R¹⁴ is selected from hydrogen and an optionally substituted group selected from C1-C8 alkyl, C3-C6 cycloalkyl, C3-C6 heterocycloalkyl, and Ar³; wherein Ar³ is selected from an optionally substituted monocyclic aryl and monocyclic heteroaryl; wherein R⁴ is selected from hydrogen and C═OOR¹⁵; wherein R¹⁵ is selected from hydrogen and optionally substituted C1-C8 alkyl; wherein R³ and R⁴ are optionally covalently bonded and, together with the intermediate carbon, comprise an optionally substituted 3- to 7-membered heterocycloalkyl; and wherein R¹⁰ and R¹¹ are optionally covalently bonded and, together with the intermediate carbon, comprise an optionally substituted 3- to 7-membered heterocycloalkyl; or a pharmaceutically acceptable salt, hydrate, solvate, or polymorph thereof.

In a further aspect, the compound administered is any of the disclosed compounds or at least one product of the disclosed methods of making.

In a further aspect, the at least one cell is in a mammal. In a still further aspect, the method further comprises administering to the mammal the compound in an amount sufficient to contact at least one cell in the mammal. In yet further aspect, the cell is mammalian. In an even further aspect, the cell is human. In a further aspect, the cell has been isolated from a mammal prior to the contacting step.

In a further aspect, contacting is via administration to a mammal. In a further aspect, the mammal has been diagnosed with a need for modulating STAT protein activity prior to the administering step. In a further aspect, the mammal has been diagnosed with a need for treatment of a disorder related to a STAT protein activity dysfunction prior to the administering step. In a still further aspect, the STAT protein is STAT3.

In a further aspect, the at least one cell is in a human. In a yet further aspect, the mammal has been diagnosed with a need for treatment of the disorder prior to the contacting step. In a still further aspect, the method further comprises the step of identifying a mammal in need of treatment of the disorder.

In a further aspect, the compound contacting the cell inhibits STAT protein activity. In a further aspect, the compound contacting the cell inhibits STAT3 protein activity. In a still further aspect, the compound contacting the cell prevents STAT3 protein dimerization. In a yet further aspect the compound contacting the cell disrupts preformed or existing STAT3 dimers. In a still further aspect, the compound contacting the cell binds to the SH2 domain of STAT3.

In a further aspect, the compound contacting the cell inhibits STAT protein activity with an IC₅₀ in an EMSA assay of less than about 100 μM, less than about 50 μM, less than about 10 μM, less than about 1 μM, less than about 500 nM, or of less than about 100 nM. In a further aspect, the IC₅₀ for inhibition of STAT3 activity.

In a further aspect, the compound contacting the cell inhibits cell growth. In a still further aspect, the compound inhibits cell growth with an IC₅₀ of less than about 500 μM. In a yet further aspect, the compound inhibits cell growth with an IC₅₀ of less than about 250 μM. In an even further aspect, the compound inhibits cell growth with an IC₅₀ of less than about 100 μM. In a still further aspect, the compound inhibits cell growth with an IC₅₀ of less than about 50 μM. In a yet further aspect, the compound inhibits cell growth with an IC₅₀ of less than about 10 μM. In an even further aspect, the compound inhibits cell growth with an IC₅₀ of less than about 1 μM. In a still further aspect, the IC₅₀ for inhibition of cell growth is determined in a cell line with a constitutively active STAT protein. In a yet further aspect, the IC₅₀ for inhibition of cell growth is determined in a cell line with a persistently active STAT protein. In an even further aspect, the IC₅₀ for inhibition of cell growth is determined in a cell line derived from a cancer selected from breast cancer, pancreatic cancer, and prostate cancer. In a yet further aspect, the IC₅₀ for inhibition of cell growth is determined in a cell line derived from a cancer with a STAT protein activity dysfunction. In a still further aspect, the IC₅₀ for inhibition of cell growth is determined in a cell line selected from MDA-MB-231, Panc-1, and DU 145. In a yet further aspect, the IC₅₀ for inhibition of cell growth is determined in a cell line transformed with v-Src. In an even further aspect, the IC₅₀ for inhibition of cell growth is determined in the NIH3T3 cell-line transformed with v-Src.

In a further aspect, the compound contacting the cell treats a disorder is associated with constitutively active STAT3.

In a further aspect, the compound contacting the cell treats a disorder selected from psoriasis and pulmonary arterial hypertension.

In a further aspect, the compound contacting the cell treats a disorder of uncontrolled cellular proliferation. In a yet further aspect, the disorder of uncontrolled cellular proliferation is cancer. In a still further aspect, the cancer is selected from a cancer of the head, neck, pancreas, brain, ovary, kidney, prostate, breast, lung, colon, and liver.

In a further aspect, the cancer is a hematological cancer. In a still further aspect, the hematological cancer is selected from acute myeloid leukemia (AML), acute lymphoblastic leukemia (ALL), chronic myeloid leukemia (CML), chronic lymphocytic leukemia (CLL), hairy cell leukemia, chronic myelomonocytic leukemia (CMML), juvenile myelomonocytic leukemia (JMML), Hodgkin lymphoma, Non-Hodgkin lymphoma, multiple myeloma, solitary myeloma, localized myeloma, and extramedullary myeloma.

In a further aspect, the cancer is a cancer of the brain. In a still further aspect, the cancer of the brain is selected from a glioma, medulloblastoma, primitive neuroectodermal tumor (PNET), acoustic neuroma, glioma, meningioma, pituitary adenoma, schwannoma, CNS lymphoma, primitive neuroectodermal tumor, craniopharyngioma, chordoma, medulloblastoma, cerebral neuroblastoma, central neurocytoma, pineocytoma, pineoblastoma, atypical teratoid rhabdoid tumor, chondrosarcoma, chondroma, choroid plexus carcinoma, choroid plexus papilloma, craniopharyngioma, dysembryoplastic neuroepithelial tumor, gangliocytoma, germinoma, hemangioblastoma, hemangiopercytoma, and metastatic brain tumor. In a yet further aspect, the glioma is selected from ependymoma, astrocytoma, oligodendroglioma, and oligoastrocytoma. In an even further aspect, the glioma is selected from juvenile pilocytic astrocytoma, subependymal giant cell astrocytoma, ganglioglioma, subependymoma, pleomorphic xanthoastrocytom, anaplastic astrocytoma, glioblastoma multiforme, brain stem glioma, oligodendroglioma, ependymoma, oligoastrocytoma, cerebellar astrocytoma, desmoplastic infantile astrocytoma, subependymal giant cell astrocytoma, diffuse astrocytoma, mixed glioma, optic glioma, gliomatosis cerebri, multifocal gliomatous tumor, multicentric glioblastoma multiforme tumor, paraganglioma, and ganglioglioma.

In a further aspect, the cancer is selected from a cancer of the breast, ovary, prostate, head, neck, and kidney. In a still further aspect, the cancer is breast cancer. In a yet further.

2. Manufacture of a Medicament

In one aspect, the invention relates to a method for the manufacture of a medicament for uncontrolled cellular proliferation activity in a mammal comprising combining a therapeutically effective amount of a disclosed compound or product of a disclosed method with a pharmaceutically acceptable carrier or diluent.

3. Use of Compounds

In one aspect, the invention relates to the use of a compound having a structure represented by a formula:

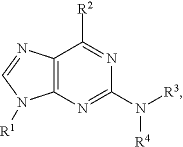

wherein R¹ is selected from H and (CH₂)ₘC═OR⁵, wherein m is an integer from 0-3; wherein R⁵ is selected from OR⁶ and NR⁷R⁸; wherein R⁶ is selected from hydrogen and C1-C8 alkyl; wherein each of R⁷ and R⁸ is independently selected from hydrogen and C1-C8 alkyl; wherein R² is selected from halogen, OR⁹, and NR¹⁰R¹¹; wherein R⁹ is an optionally substituted group selected from C1-C8 alkyl, C3-C6 cycloalkyl, C3-C6 heterocycloalkyl, (CH₂)ₙR¹², and Ar¹; wherein n is an integer from 0-3; wherein R¹² is an optionally substituted group selected from Ar¹, C3-C6 cycloalkyl and C3-C6 heterocycloalkyl; wherein Ar¹ is selected from an optionally substituted monocyclic aryl and monocyclic heteroaryl; wherein R¹⁰ is selected from hydrogen and an optionally substituted group selected from C1-C8 alkyl, C3-C6 cycloalkyl, C3-C6 heterocycloalkyl, (CH₂)ₚR¹³, and Ar²; wherein p is an integer from 0-3; wherein R¹³ is an optionally substituted group selected from Ar², C3-C6 cycloalkyl and C3-C6 heterocycloalkyl; wherein Ar² is selected from an optionally substituted monocyclic aryl and monocyclic heteroaryl; wherein R¹¹ is selected from hydrogen and C1-C8 alkyl; wherein R³ is selected from hydrogen and an optionally substituted group selected from C1-C8 alkyl, (CH₂)_{q}R¹⁴, and C═O(CH₂)_{q}R¹⁴; wherein q is an integer from 0-3; wherein R¹⁴ is selected from hydrogen and an optionally substituted group selected from C1-C8 alkyl, C3-C6 cycloalkyl, C3-C6 heterocycloalkyl, and Ar³; wherein Ar³ is selected from an optionally substituted monocyclic aryl and monocyclic heteroaryl; wherein R⁴ is selected from hydrogen and C═OOR¹⁵; wherein R¹⁵ is selected from hydrogen and optionally substituted C1-C8 alkyl; wherein R³ and R⁴ are optionally covalently bonded and, together with the intermediate carbon, comprise an optionally substituted 3- to 7-membered heterocycloalkyl; and wherein R¹⁰ and R¹¹ are optionally covalently bonded and, together with the intermediate carbon, comprise an optionally substituted 3- to 7-membered heterocycloalkyl; or a pharmaceutically acceptable salt, hydrate, solvate, or polymorph thereof.

In a further aspect, the compound is a disclosed compound or a product of a disclosed method.

In a further aspect, the use relates to inhibition of STAT protein activity. In a further aspect, the use relates to inhibition of STAT3 protein activity. In a still further aspect, the use relates to prevention of STAT3 protein dimerization. In a yet further aspect, the use relates to disruption of preformed or existing STAT3 dimers. In a still further aspect, the use relates to binding to the SH2 domain of STAT3.

In a further aspect, the use relates to inhibition of STAT protein activity with an IC₅₀ in an EMSA assay of less than about 100 μM, less than about 50 μM, less than about 10 μM, less than about 1 μM, less than about 500 nM, or of less than about 100 nM. In a further aspect, the IC₅₀ for inhibition of STAT3 activity.

In a further aspect, the use relates to inhibition of cell growth. In a still further aspect, the use relates to inhibition of cell growth with an IC₅₀ of less than about 500 μM. In a yet further aspect, the use relates to inhibition of cell growth with an IC₅₀ of less than about 250 μM. In an even further aspect, the use relates to inhibition of cell growth with an IC₅₀ of less than about 100 μM. In a still further aspect, the use relates to inhibition of cell growth with an IC₅₀ of less than about 50 μM. In a yet further aspect, the use relates to inhibition of inhibits cell growth with an IC₅₀ of less than about 10 μM. In an even further aspect, the use relates to inhibition of cell growth with an IC₅₀ of less than about 1 μM. In a still further aspect, the IC₅₀ for inhibition of cell growth is determined in a cell line with a constitutively active STAT protein. In a yet further aspect, the IC₅₀ for inhibition of cell growth is determined in a cell line with a persistently active STAT protein. In an even further aspect, the IC₅₀ for inhibition of cell growth is determined in a cell line derived from a cancer selected from breast cancer, pancreatic cancer, and prostate cancer. In a yet further aspect, the IC₅₀ for inhibition of cell growth is determined in a cell line derived from a cancer with a STAT protein activity dysfunction. In a still further aspect, the IC₅₀ for inhibition of cell growth is determined in a cell line selected from MDA-MB-231, Panc-1, and DU 145. In a yet further aspect, the IC₅₀ for inhibition of cell growth is determined in a cell line transformed with v-Src. In an even further aspect, the IC₅₀ for inhibition of cell growth is determined in the NIH3T3 cell-line transformed with v-Src.

In a further aspect, the use treats a disorder is associated with constitutively active STAT3.

In a further aspect, the use treats a disorder selected from psoriasis and pulmonary arterial hypertension.

In a further aspect, the use treats a disorder of uncontrolled cellular proliferation. In a yet further aspect, the disorder of uncontrolled cellular proliferation is cancer. In a still further aspect, the cancer is selected from a cancer of the head, neck, pancreas, brain, ovary, kidney, prostate, breast, lung, colon, and liver.

In a further aspect, the use relates to the manufacture of a medicament for the treatment of a disorder associated with STAT protein activity dysfunction in a mammal. In a further aspect, the disorder is a disorder uncontrolled cellular proliferation. In a further aspect, the disorder is a disease of uncontrolled cellular proliferation. In a further aspect, a use relates to treatment of a disorder of controlled cellular proliferation associated with a STAT protein activity dysfunction in a mammal.

In one aspect, a use is associated with the treatment of a disorder associated with uncontrolled cellular proliferation. In a further aspect, the disorder is cancer. In a still further aspect, the cancer is selected from a cancer of the head, neck, pancreas, brain, ovary, kidney, prostate, breast, lung, colon, and liver.

In a further aspect, the cancer is a hematological cancer. In a still further aspect, the hematological cancer is selected from acute myeloid leukemia (AML), acute lymphoblastic leukemia (ALL), chronic myeloid leukemia (CML), chronic lymphocytic leukemia (CLL), hairy cell leukemia, chronic myelomonocytic leukemia (CMML), juvenile myelomonocytic leukemia (JMML), Hodgkin lymphoma, Non-Hodgkin lymphoma, multiple myeloma, solitary myeloma, localized myeloma, and extramedullary myeloma.

In a further aspect, the cancer is a cancer of the brain. In a still further aspect, the cancer of the brain is selected from a glioma, medulloblastoma, primitive neuroectodermal tumor (PNET), acoustic neuroma, glioma, meningioma, pituitary adenoma, schwannoma, CNS lymphoma, primitive neuroectodermal tumor, craniopharyngioma, chordoma, medulloblastoma, cerebral neuroblastoma, central neurocytoma, pineocytoma, pineoblastoma, atypical teratoid rhabdoid tumor, chondrosarcoma, chondroma, choroid plexus carcinoma, choroid plexus papilloma, craniopharyngioma, dysembryoplastic neuroepithelial tumor, gangliocytoma, germinoma, hemangioblastoma, hemangiopercytoma, and metastatic brain tumor. In a yet further aspect, the glioma is selected from ependymoma, astrocytoma, oligodendroglioma, and oligoastrocytoma. In an even further aspect, the glioma is selected from juvenile pilocytic astrocytoma, subependymal giant cell astrocytoma, ganglioglioma, subependymoma, pleomorphic xanthoastrocytom, anaplastic astrocytoma, glioblastoma multiforme, brain stem glioma, oligodendroglioma, ependymoma, oligoastrocytoma, cerebellar astrocytoma, desmoplastic infantile astrocytoma, subependymal giant cell astrocytoma, diffuse astrocytoma, mixed glioma, optic glioma, gliomatosis cerebri, multifocal gliomatous tumor, multicentric glioblastoma multiforme tumor, paraganglioma, and ganglioglioma.

In a further aspect, the cancer is selected from a cancer of the breast, ovary, prostate, head, neck, and kidney. In a still further aspect, the cancer is breast cancer. In a yet further

In a further aspect, the disorder is selected from psoriasis and pulmonary arterial hypertension.

4. Kits

In one aspect, the invention relates to a kit comprising at least one compound having a structure represented by a formula:

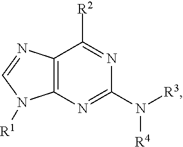

wherein R¹ is selected from H and (CH₂)ₘC═OR⁵, wherein m is an integer from 0-3; wherein R⁵ is selected from OR⁶ and NR⁷R⁸; wherein R⁶ is selected from hydrogen and C1-C8 alkyl; wherein each of R⁷ and R⁸ is independently selected from hydrogen and C1-C8 alkyl; wherein R² is selected from halogen, OR⁹, and NR¹⁰R¹¹; wherein R⁹ is an optionally substituted group selected from C1-C8 alkyl, C3-C6 cycloalkyl, C3-C6 heterocycloalkyl, (CH₂)ₙR¹², and Ar¹; wherein n is an integer from 0-3; wherein R¹² is an optionally substituted group selected from Ar¹, C3-C6 cycloalkyl and C3-C6 heterocycloalkyl; wherein Ar¹ is selected from an optionally substituted monocyclic aryl and monocyclic heteroaryl; wherein R¹⁰ is selected from hydrogen and an optionally substituted group selected from C1-C8 alkyl, C3-C6 cycloalkyl, C3-C6 heterocycloalkyl, (CH₂)ₚR¹³, and Ar²; wherein p is an integer from 0-3; wherein R¹³ is an optionally substituted group selected from Ar², C3-C6 cycloalkyl and C3-C6 heterocycloalkyl; wherein Ar² is selected from an optionally substituted monocyclic aryl and monocyclic heteroaryl; wherein R¹¹ is selected from hydrogen and C1-C8 alkyl; wherein R³ is selected from hydrogen and an optionally substituted group selected from C1-C8 alkyl, (CH₂)_{q}R¹⁴, and C═O(CH₂)_{q}R¹⁴; wherein q is an integer from 0-3; wherein R¹⁴ is selected from hydrogen and an optionally substituted group selected from C1-C8 alkyl, C3-C6 cycloalkyl, C3-C6 heterocycloalkyl, and Ar³; wherein Ar³ is selected from an optionally substituted monocyclic aryl and monocyclic heteroaryl; wherein R⁴ is selected from hydrogen and C═OOR¹⁵; wherein R¹⁵ is selected from hydrogen and optionally substituted C1-C8 alkyl; wherein R³ and R⁴ are optionally covalently bonded and, together with the intermediate carbon, comprise an optionally substituted 3- to 7-membered heterocycloalkyl; and wherein R¹⁰ and R¹¹ are optionally covalently bonded and, together with the intermediate carbon, comprise an optionally substituted 3- to 7-membered heterocycloalkyl; or a pharmaceutically acceptable salt, hydrate, solvate, or polymorph thereof, and one or more of: (a) at least one agent known to decrease STAT activity; (b) at least one agent known to increase STAT activity; (c) at least one agent know to treat a disease of uncontrolled cellular proliferation; (d) at least one agent known to treat psoriasis; (e) at least one agent known to treat pulmonary arterial hypertension; or (f) instructions for treating a disorder associated with STAT dysfunction.

In a further aspect, the kit comprises a disclosed compound or a product of a disclosed method.

In a further aspect, the at least one compound and the at least one agent are co-formulated. In a still further aspect, the at least one compound and the at least one agent are co-packaged.

In a further aspect, the at least one agent know to decrease STAT activity decreases STAT3 activity. In a still further aspect, the at least one agent know to increase STAT activity increases STAT3 activity. In a yet further aspect, the instructions for treating a disorder with STAT dysfunction provide instructions for treating a STAT3 dysfunction.

In a further aspect, the at least one compound in the kit exhibits inhibition of a STAT protein. In a yet further aspect, the compound in the kit inhibits the STAT protein is STAT3.

In a further aspect, the at least one compound in the kit inhibits STAT protein activity with an IC₅₀ in an EMSA assay of less than about 100 μM, less than about 50 μM, less than about 10 μM, less than about 1 μM, less than about 500 nM, or of less than about 100 nM. In a further aspect, the IC₅₀ for inhibition of STAT3 activity.

In a further aspect, the at least one compound in the kit inhibits cell growth. In a still further aspect, the compound inhibits cell growth with an IC₅₀ of less than about 500 μM. In a yet further aspect, the compound inhibits cell growth with an IC₅₀ of less than about 250 μM. In an even further aspect, the compound inhibits cell growth with an IC₅₀ of less than about 100 μM. In a still further aspect, the compound inhibits cell growth with an IC₅₀ of less than about 50 μM. In a yet further aspect, the compound inhibits cell growth with an IC₅₀ of less than about 10 μM. In an even further aspect, the compound inhibits cell growth with an IC₅₀ of less than about 1 μM. In a still further aspect, the IC₅₀ for inhibition of cell growth is determined in a cell line with a constitutively active STAT protein. In a yet further aspect, the IC₅₀ for inhibition of cell growth is determined in a cell line with a persistently active STAT protein. In an even further aspect, the IC₅₀ for inhibition of cell growth is determined in a cell line derived from a cancer selected from breast cancer, pancreatic cancer, and prostate cancer. In a yet further aspect, the IC₅₀ for inhibition of cell growth is determined in a cell line derived from a cancer with a STAT protein activity dysfunction. In a still further aspect, the IC₅₀ for inhibition of cell growth is determined in a cell line selected from MDA-MB-231, Panc-1, and DU 145. In a yet further aspect, the IC₅₀ for inhibition of cell growth is determined in a cell line transformed with v-Src. In an even further aspect, the IC₅₀ for inhibition of cell growth is determined in the NIH3T3 cell-line transformed with v-Src.

In a further aspect, the at least one compound in the kit treats a disorder is associated with constitutively active STATS.

In a further aspect, the at least one agent is a hormone therapy agent. In a still further aspect, the hormone therapy agent is selected from one or more of the group consisting of leuprolide, tamoxifen, raloxifene, megestrol, fulvestrant, triptorelin, medroxyprogesterone, letrozole, anastrozole, exemestane, bicalutamide, goserelin, histrelin, fluoxymesterone, estramustine, flutamide, toremifene, degarelix, nilutamide, abarelix, and testolactone, or a pharmaceutically acceptable salt, hydrate, solvate, or polymorph thereof.

In a further aspect, the at least one agent is a chemotherapeutic agent. In a yet further aspect, the chemotherapeutic agent is selected from one or more of the group consisting of an alkylating agent, an antimetabolite agent, an antineoplastic antibiotic agent, a mitotic inhibitor agent, a mTor inhibitor agent or other chemotherapeutic agent. In a still further aspect, the antineoplastic antibiotic agent is selected from one or more of the group consisting of doxorubicin, mitoxantrone, bleomycin, daunorubicin, dactinomycin, epirubicin, idarubicin, plicamycin, mitomycin, pentostatin, and valrubicin, or a pharmaceutically acceptable salt, hydrate, solvate, or polymorph thereof. In an even further aspect, the antimetabolite agent is selected from one or more of the group consisting of gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt, hydrate, solvate, or polymorph thereof. In a still further aspect, the alkylating agent is selected from one or more of the group consisting of carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt, hydrate, solvate, or polymorph thereof. In a yet further aspect, the mitotic inhibitor agent is selected from one or more of the group consisting of irinotecan, topotecan, rubitecan, cabazitaxel, docetaxel, paclitaxel, etopside, vincristine, ixabepilone, vinorelbine, vinblastine, and teniposide, or a pharmaceutically acceptable salt, hydrate, solvate, or polymorph thereof. In an even further aspect, the mTor inhibitor agent is selected from one or more of the group consisting of everolimus, siroliumus, and temsirolimus, or a pharmaceutically acceptable salt, hydrate, solvate, or polymorph thereof.

In a further aspect, the at least one compound and the at least one agent are co-packaged. In a still further aspect, the at least one agent that is co-packaged with the at least one compound is one of the agents described herein.

In a further aspect, the at least one compound and the at least one agent are co-formulated. In a still further aspect, the at least one agent that is co-formulated with the at least one compound is one of the agents described herein.

In a further aspect, the instructions further comprise providing the compound in connection surgery. In a still further aspect, the instructions provide that surgery is performed prior to the administering of at least one compound. In a yet further aspect, the instructions provide that surgery is performed after the administering of at least one compound. In an even further aspect, the instructions provide that the administering of at least one compound is to effect presurgical debulking of a tumor. In a yet further aspect, the instructions provide that surgery is performed at about the same time as the administering of at least one compound.

In a further aspect, the instructions further comprise providing the compound in connection with radiotherapy. In a yet further aspect, the instructions provide that radiotherapy is performed prior to the administering of at least one compound. In a still further aspect, the instructions provide that radiotherapy is performed after the step of the administering of at least one compound. In an even further aspect, the instructions provide that radiotherapy is performed at about the same time as the step of the administering of at least one compound. In a still further aspect, the instructions further comprise providing the compound in connection with at least one agent that is a chemotherapeutic agent.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is contemplated that the disclosed kits can be used in connection with the disclosed methods of making, the disclosed methods of using, and/or the disclosed compositions.

5. Non-Medical Uses

Also provided are the uses of the disclosed compounds and products as pharmacological tools in the development and standardization of in vitro and in vivo test systems for the evaluation of the effects of inhibitors of STAT protein related activity in laboratory animals such as cats, dogs, rabbits, monkeys, rats and mice, as part of the search for new therapeutic agents targeting STAT protein. Also provided are the uses of the disclosed compounds and products as pharmacological tools in the development and standardization of in vitro and in vivo test systems for the evaluation of the effects of inhibitors of STAT protein related activity in laboratory animals such as cats, dogs, rabbits, monkeys, rats and mice, as part of the search for new therapeutic agents targeting STAT3 protein.

### F. REFERENCES

(1) Darnell, J. E., Jr. Transcription factors as targets for cancer therapy. Nat. Rev. Cancer 2002, 2, 740-749.
   (2) Turkson, J.; Jove, R. STAT proteins: novel molecular targets for cancer drug discovery. Oncogene 2000, 19, 6613-6626.
   (3) Turkson, J. STAT proteins as novel targets for cancer drug discovery. Expert Opin. Ther. Targets 2004, 8, 409-422.
   (4) Yue, P.; Turkson, J. Targeting STAT3 in cancer: how successful are we? Expert Opin. Invest. Drugs 2009, 18, 45-56.
   (5) Turkson, J.; Kim, J. S.; Zhang, S.; Yuan, J.; Huang, M.; Glenn, M.; Haura, E.; Sebti, S.; Hamilton, A. D.; Jove, R. Novel peptidomimetic inhibitors of signal transducer and activator of transcription 3 dimerization and biological activity. Mol. Cancer. Ther. 2004, 3, 261-269.
   (6) Turkson, J.; Ryan, D.; Kim, J. S.; Zhang, Y.; Chen, Z.; Haura, E.; Laudano, A.; Sebti, S.; Hamilton, A. D.; Jove, R. Phosphotyrosyl peptides block STAT3-mediated DNA-binding activity, gene regulation and cell transformation. J. Biol. Chem. 2001, 276, 45443-45455.
   (7) Siddiquee, K.; Zhang, S.; Guida, W. C.; Blaskovich, M. A.; Greedy, B.; Lawrence, H. R.; Yip, M. L.; Jove, R.; McLaughlin, M. M.; Lawrence, N. J.; Sebti, S. M.; Turkson, J. Selective chemical probe inhibitor of STAT3, identified through structure-based virtual screening, induces antitumor activity. Proc. Natl. Acad. Sci. U.S.A. 2007, 104, 7391-7396.
   (8) Siddiquee, K. A. Z.; Gunning, P. T.; Glenn, M.; Katt, W. P.; Zhang, S.; Schroeck, C.; Sebti, S. M.; Jove, R.; Hamilton, A. D.; Turkson, J. An Oxazole-Based Small-Molecule STAT3 Inhibitor Modulates STAT3 Stability and Processing and Induces Antitumor Cell Effects. ACS Chem. Biol. 2007, 2, 787-798.
   (9) Chen, J.; Bai, L.; Bernard, D.; Nikolovska-Coleska, Z.; Gomez, C.; Zhang, J.; Yi, H.; Wang, S. Structure-Based Design of Conformationally Constrained, Cell-Permeable STAT3 Inhibitors. ACS Med. Chem. Lett. 2010, 1, 85-89.
   (10) Lin, L.; Hutzen, B.; Zuo, M.; Ball, S.; Deangelis, S.; Foust, E.; Pandit, B.; Ihnat, M. A.; Shenoy, S. S.; Kulp, S.; Li, P. K.; Li, C.; Fuchs, J.; Lin, J. Novel STAT3 phosphorylation inhibitors exhibit potent growth-suppressive activity in pancreatic and breast cancer cells. Cancer Res. 2010, 70, 2445-2454.
   (11) Bhasin, D.; Cisek, K.; Pandharkar, T.; Regan, N.; Li, C.; Pandit, B.; Lin, J.; Li, P. K. Design, synthesis, and studies of small molecule STAT3 inhibitors. Bioorg. Med. Chem. Lett. 2008, 18, 391-395.
   (12) Song, H.; Wang, R.; Wang, S.; Lin, J. A low-molecular-weight compound discovered through virtual database screening inhibits STAT3 function in breast cancer cells. Proc. Natl. Acad. Sci. U.S.A. 2005, 102, 4700-4705.
   (13) Schust, J.; Sperl, B.; Hollis, A.; Mayer, T. U.; Berg, T. STATtic: a small-molecule inhibitor of STAT3 activation and dimerization. Chem. Biol. 2006, 13, 1235-1242.
   (14) Ren, Z.; Cabell, L. A.; Schaefer, T. S.; McMurray, J. S. Identification of a high-affinity phosphopeptide inhibitor of stat3. Bioorg. Med. Chem. Lett. 2003, 13, 633-636.
   (15) McMurray, J. S.; Mandal, P. K.; Liao, W. S.; Ren, Z.; Chen, X. Inhibition of STAT3 by cell-permeable peptidomimetic prodrugs targeted to its SH2 domain. Adv. Exp. Med. Biol. 2009, 611, 545-546.
   (16) Zhang, X.; Yue, P.; Fletcher, S.; Zhao, W.; Gunning, P. T.; Turkson, J. A novel small-molecule disrupts STAT3 SH2 domain-phosphotyrosine interactions and STAT3-dependent tumor processes. Biochem. Pharmacol. 2010, 79, 1398-1409.
   (17) Gunning, P. T.; Glenn, M. P.; Siddiquee, K. A.; Katt, W. P.; Masson, E.; Sebti, S. M.; Turkson, J.; Hamilton, A. D. Targeting protein-protein interactions: suppression of STAT3 dimerization with rationally designed small-molecule, nonpeptidic SH2 domain binders. ChemBioChem 2008, 9, 2800-2803.
   (18) Gunning, P. T.; Katt, W. P.; Glenn, M.; Siddiquee, K.; Kim, J. S.; Jove, R.; Sebti, S. M.; Turkson, J.; Hamilton, A. D. Isoform selective inhibition of STAT1 or STAT3 homo-dimerization via peptidomimetic probes: structural recognition of STAT SH2 domains. Bioorg. Med. Chem. Lett. 2007, 17, 1875-1878.
   (19) Turkson, J.; Zhang, S.; Palmer, J.; Kay, H.; Stanko, J.; Mora, L. B.; Sebti, S.; Yu, H.; Jove, R. Inhibition of constitutive signal transducer and activator of transcription 3 activation by novel platinum complexes with potent anti-tumor activity. Mol. Cancer. Ther. 2004, 3, 1533-1542.
   (20) Becker, S.; Groner, B.; Muller, C. W. Three-dimensional structure of the STAT3beta homodimer bound to DNA. Nature 1998, 394, 145-151.
   (21) Jones, G.; Willett, P.; Glen, R. C.; Leach, A. R.; Taylor, R. Development and validation of a genetic algorithm for flexible docking. J. Mol. Biol. 1997, 267, 727-748.
   (22) Fletcher, S.; Turkson, J.; Gunning, P. T. Molecular approaches towards the inhibition of the signal transducer and activator of transcription 3 (STAT3) protein. ChemMedChem 2008, 3, 1159-1168.
   (23) Fletcher, S.; Shahani, V. M.; Gunning, P. T. Facile and efficient access to 2,6,9-tri-substituted purines through sequential N9, N2 Mitsunobu reactions. Tetrahedron Lett. 2009, 50 (29), 4258-4261.
   (24) Fletcher, S.; Shahani, V. M.; Lough, A. J.; Gunning, P. T. Concise access to N⁹-mono-, N²-mono- and N²,N⁹-di-substituted guanines via efficient Mitsunobu reactions Tetrahedron 2010, 66:4621-32.
   (25) Turkson, J.; Zhang, S.; Mora, L. B.; Burns, A.; Sebti, S.; Jove, R. A novel platinum compound inhibits constitutive STAT3 signaling and induces cell cycle arrest and apoptosis of malignant cells. J. Biol. Chem. 2005, 280, 32979-32988.
   (26) Coleman, D. R.; Ren, Z.; Mandal, P. K.; Cameron, A. G.; Dyer, G. A.; Muranjan, S.; Campbell, M.; Chen, X.; McMurray, J. S. Investigation of the binding determinants of phosphopeptides targeted to the SRC homology 2 domain of the signal transducer and activator of transcription 3. Development of a high-affinity peptide inhibitor. J. Med. Chem. 2005, 48, 6661-6670.
   (27) Mandal, P. K.; Limbrick, D.; Coleman, D. R.; Dyer, G. A.; Ren, Z.; Birtwistle, J. S.; Xiong, C.; Chen, X.; Briggs, J. M.; McMurray, J. S. Conformationally constrained peptidomimetic inhibitors of signal transducerand activator of transcription 3: evaluation andmolecular modeling. J. Med. Chem. 2009, 52, 2429-2442.
   (28) Galmarini, C. M.; Mackey, J. R.; Dumontet, C. Nucleoside analogues and nucleobases in cancer treatment. Lancet Oncol. 2002, 3, 415-424.
   (29) Parker, W. B.; Secrist, J. A., 3rd; Waud, W. R. Purine nucleoside antimetabolites in development for the treatment of cancer. Curr. Opin. Invest. Drugs 2004, 5, 592-596.
   (30) Darnell, J. E., Jr. (1996) The JAK-STAT pathway: summary of initial studies and recent advances. Recent Prog. Norm. Res. 51, 391-403.
   (31) Darnell, J. E., Jr. (1997) STATs and gene regulation. Science 277, 1630-1635.
   (32) Darnell. J. E. Validating STAT3 in cancer therapy (2005) Nat. Med. 11, 595-596.
   (33) Bowman, T., Garcia, R., Turkson. J., and Jove, R. (2000) STATs in oncogenesis. Oncogene 19, 2474-2488.
   (34) Buettner, R., Mora, L. B., and Jove, R. (2002) Activated STAT signaling in human tumors provides novel molecular targets for therapeutic intervention. Clin. Cancer Res. 8, 945-954.
   (35) Yu, H. and Jove. R. (2004) The STATS of Cancer-New molecular targets come of age. Nat. Rev. Cancer 4, 97-105.
   (36) Haura, E. B., Turkson, J., and Jove, R. (2005) Mechanisms of disease: Insights into the emerging role of signal transducers and activators of transcription in cancer. Nat. Clin. Pract. Oncol. 2, 315-324.
   (37) Fletcher, S.; Singh, J.; Zhang, X.; Yue, P.; Page, B. D.; Sharmeen, S.; Shahani, V. M.; Zhao, W.; Schimmer, A. D.; Turkson, J.; Gunning, P. T. Disruption of transcriptionally active STAT3 dimers with non-phosphorylated, salicylic acid-based small molecules: potent in vitro and tumor cell activities. Chembiochem 2009, 10, 1959-64.
   (38) Turkson, J.; Bowman, T.; Garcia, R.; Caldenhoven, E.; De Groot, R. P.; Jove, R. STAT3 activation by Src induces specific gene regulation and is required for cell transformation. Mol. Cell. Biol. 1998, 18, 2545-2552.
   (39) Wagner, M.; Kleeff, J.; Friess, H.; Buchler, M. W.; Korc, M. Enhanced expression of the type II transforming growth factor-beta receptor is associated with decreased survival in human pancreatic cancer. Pancreas. 1999, 19, 370-6.
   (40) Morton C L, Houghton P J. 2007. Establishment of human tumor xenografts in immunodeficient mice. Nat. Protoc. 2, 247-250.

### G. EXPERIMENTAL

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

Several methods for preparing the compounds of this invention are illustrated in the following Examples. Starting materials and the requisite intermediates are in some cases commercially available, or can be prepared according to literature procedures or as illustrated herein.

The following exemplary compounds of the invention were synthesized. The Examples are provided herein to illustrate the invention, and should not be construed as limiting the invention in any way. The Examples are typically depicted in free base form, according to the IUPAC naming convention. However, some of the Examples were obtained or isolated in salt form.

As indicated, some of the Examples were obtained as racemic mixtures of one or more enantiomers or diastereomers. The compounds may be separated by one skilled in the art to isolate individual enantiomers. Separation can be carried out by the coupling of a racemic mixture of compounds to an enantiomerically pure compound to form a diastereomeric mixture, followed by separation of the individual diastereomers by standard methods, such as fractional crystallization or chromatography. A racemic or diastereomeric mixture of the compounds can also be separated directly by chromatographic methods using chiral stationary phases.

### 1. General Methods

Anhydrous solvents methanol, DMSO, CH₂Cl₂, THF and DMF were purchased from Sigma Aldrich and used directly from Sure-Seal bottles. Molecular sieves were activated by heating to 300° C. under vacuum overnight. All reactions were performed under an atmosphere of dry nitrogen in oven-dried glassware and were monitored for completeness by thin-layer chromatography (TLC) using silica gel (visualized by UV light, or developed by treatment with KMnO₄ stain or phosphomolybdic acid stain). ¹H and ¹³C NMR spectra were recorded on Bruker 400 MHz and a Varian 500 MHz spectrometers in either CDCl₃, CD₃OD or d₆-DMSO. Chemical shifts (δ) are reported in parts per million after calibration to residual isotopic solvent. Coupling constants (J) are reported in Hz. Before biological testing, inhibitor purity was evaluated by reversed-phase HPLC (rpHPLC). Analysis by rpHPLC was performed using a Microsorb-MV 300 A C18 250 mm×4.6 mm column run at 1 mL/min, and using gradient mixtures of (A) water with 0.1M CH₃COONH₄ and (B) methanol. Ligand purity was confirmed using linear gradients from 75% A and 25% B to 100% B after an initial 2 minute period of 100% A. The linear gradient consisted of a changing solvent composition of either (I) 4.7% per minute and UV detection at 254 nm or (II) 1.4% per minute and detection at 254 nm, each ending with 5 min of 100% B. For reporting HPLC data, percentage purity is given in parentheses after the retention time for each condition. All biologically evaluated compounds are >95% chemical purity as measured by HPLC. The HPLC traces for all tested compounds are provided in supporting information.

### 2. Molecular Modeling, Quantitative Structure Activity Relationship, and Pharmacophore Modeling

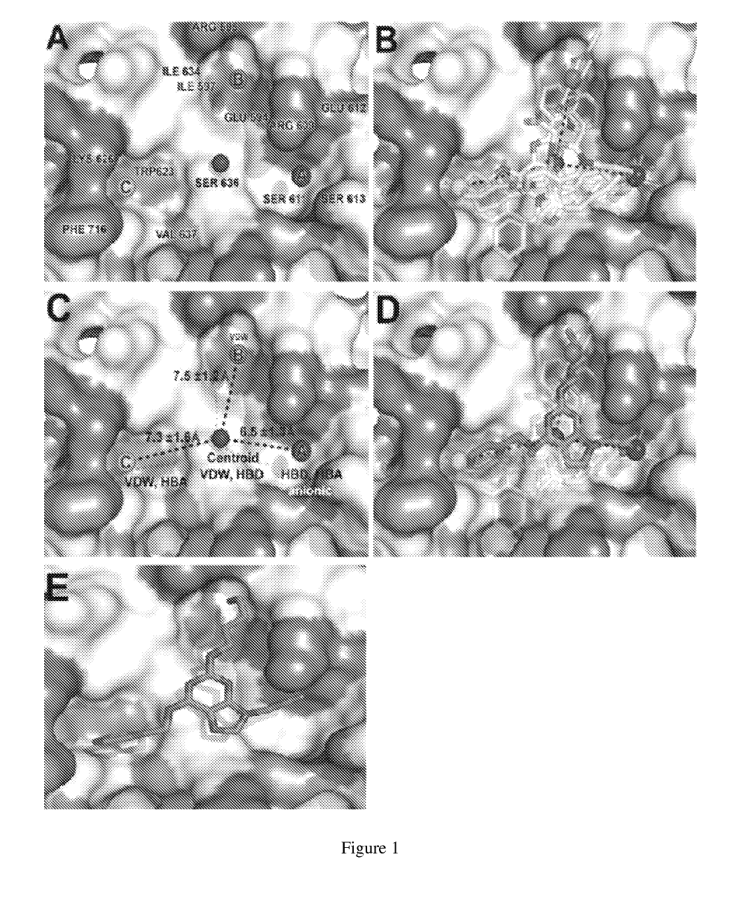

The disclosed compounds were designed using a computational approach. Briefly, STAT3 dimerization-disrupting small molecule compounds were subjected to computational (genetically optimized ligand docking, GOLD) analysis (21) for deriving 3D quantitative structure activity relationships (QSAR) was carried out to derive a pharmacophore model predictive of STAT3 inhibitors. The crystal structure of the STAT3:STAT3-DNA ternary complex (20) provided the structural composition and topology of the SH2 domain binding ‘hotspot,’ identifying three subpockets on the SH2 domain protein surface that are solvent-accessible, labeled A, B and C (see FIG. 1). The GOLD analysis (21) of all known STAT3 inhibitors within the STAT3 SH2 domain yielded homologous binding configurations (FIG. 1B), with inhibitors consistently occupying at least two of the three main subpockets (FIG. 1B). Most notably, all compounds interacted with subpocket A, which hosts the key pTyr705 group, and is broadly composed of the polar residues Lys591, Ser611, Ser613 and Arg609,22 andwere found to engage these residues through predominantly hydrogen bond donor (“HBD”) or hydrogen bond acceptor (“HBA”) groups.

Without wishing to be bound by a particular theory, interaction of residues of sub-pocket A with the pharmacophore can incorporate an anionic functional group or a concentrated array of hydrogen bond donor (“HBD”) and hydrogen bond acceptor (“HBA”) groups to engage the cationic side chains or the numerous HBA and HBD residues present. STAT3 inhibitors of prior research achieved this interaction with the use of tetrazole, phosphate, phosphonate, salicylic acid or malonate functionality (6, 16, 17, 18, 27). In contrast to A, sub-pockets B and C are predominantly non-polar and hydrophobic. Sub-pocket B is derived from the tetramethylene portion of the side chains of Lys592, Arg595, Ile597 and Ile634. Sub-pocket C is composed of Trp623, Val637, Ile659, Phe716 and Lys626. Sub-pocket B has been generally accessed by lipophilic, hydrophobic moieties, such as tosylates, phenyl rings, alkyl groups and heterocycles (22). Similarly, sub-pocket C is predominantly hydrophobic in nature (with the exception of a polar Lys626 residue) and has been previously engaged with isopropyl, hexyl, benzyl and cyclohexylbenzyl substituents (22). Without wishing to be bound by a particular theory, a predominantly hydrophobic appendage can bind optimally to this binding cleft, but that a terminally situated HBA group or carboxylate might be advantageously employed to interact with Lys626.

STAT3's SH2 domain phosphopeptide binding interface is relatively planar with the notable exception of pocket A, which is moderately more cavernous when compared to both B and C, presumably to accommodate the bulky pTyr moiety of the cognate phosphopeptide binding sequence. Without wishing to be bound by a particular theory, given the planarity of the protein surface, a central scaffold with limited flexibility can facilitate suitably situated binding groups to access all three sub-pockets. Based upon the pharmacophore plot disclosed herein, new classes of STAT3 inhibitors can be designed to incorporate the key binding functionality at the desired coordinates.

To effectively target sub-pocket A and replicate the pTyr moiety, all purine scaffolds in this study were regioselectively furnished with a carboxylate appendage on N9 using previously reported facile Mitsunobu conditions (23, 24). The synthetic approach disclosed herein and disclosed compounds provide for the incorporation of binding groups at both the exogenous N2 amino group (position R²) and C6 carbon atom (position NR³R⁴) of the purine core to afford optimal spatial access to sub-pockets B and C, respectively). In most cases, published small-molecule STAT3 inhibitors have been evaluated in dimerization assays, which assess the degree of disruption of STAT3 binding to a high-affinity pTyr peptide probe, or in a STAT3 DNA-binding assay. Herein, the use of Surface Plasmon Resonance (SPR) analysis is utilized, to study the interactions of the disclosed compounds with STAT3 in terms of the association and dissociation characteristics as a method to evaluate the affinity of the compounds for STAT3.

The disclosed compounds comprise a lipophilic pentyl chain at N2 to afford hydrophobic interactions with the alkyl side chains of Ile634 and Ile597, and the tetramethylene portion of the side-chain of Lys592. To the R² position when keeping NR³R⁴=pentyl, the compounds incorporated a focused set of aliphatic and aromatic amine substituents to probe sub-pocket C. Aromatic inhibitors compound Baa (see examples below for compound ID; R²═NH(CH₂)C₆H₆, NR³R⁴═NH(CH₂)₄CH₃), and Sac (R²═NHC₆H₆, NR³R⁴═NH(CH₂)₄CH₃) had K_{D} values of 2.2 and 2.5 μM, respectively. Initial incorporation of aliphatic primary and secondary amines at C6 also yielded active compounds (Table 1, SPR, entries 5-12: K_{D} 6-7 μM for select compounds). Comparative GOLD docking studies revealed that the benzene moiety in the three aromatic compounds displayed an additional edge to face π-π stacking interaction with the side chain of Trp623, possibly accounting for the differences in observed affinity between aromatic and aliphatic substituents. In addition, compounds were prepared with an amphiphilic morpholine group to C6 (Table 1, SPR, entry 13) in an effort to improve water solubility and make additional hydrogen bonds via the terminal HBA oxygen atom, which improved binding affinity (8 am: K_{D}=4.2 μM, Table 1, SPR). Without wishing to be bound by a particular theory, the enhanced affinity can be due to an additional hydrogen bond between the ether group and an SH2 domain backbone NH or due to a different pharmacophore binding pattern.

Generally, for the disclosed compounds, the STAT3 binding affinity improved with the incorporation a larger hydrophobic, cyclohexylbenzyl unit at position NR³R⁴ (Table 1, SPR, entries 16-36) to access sub-pocket B (8, 17). Compounds were prepared that coupled a similar set of privileged binding groups to the R² position and evaluated the relative binding potencies of the inhibitors. Overall, when NR³R⁴═NH(cyclohexybenzyl), equipotent or moderate increases in affinity for the different aromatic R² substituents was observed (8bb (NR³R⁴═NH(cyclohexylbenzyl), R²═NCH₃(benzyl)): K_{D}=4.0 μM cf. 8ab (NR³R⁴═NH(CH₂)₄CH₃, R²═NCH₃(benzyl)): K_{D}=38.4 μM). As illustrated in FIG. 1E, the cyclohexylbenzyl group beneficially orientates the purine skeleton to optimally project both the Z functionality and the carboxylate group into sub-pockets C and A, respectively. Most significantly, of the twenty cyclohexylbenzyl analogs synthesized, over 75% showed promising affinity for the SH2 domain, as assessed by SPR. Moreover, computational docking showed that lead inhibitors, 8bl, 8bm, 8bu, 8bn, 8bv, 8bw, 8bx and 8be elegantly projected the binding groups within the proposed pharmacophore plot. Introduction of an amide linkage to increase structural rigidity (NR³R⁴═NH(cyclohexylbenzamide), Table 1, entries 36-38) conferred minimal benefits. Finally, to further probe sub-pocket B's apparent tolerance for bulky hydrophobic moieties, the cyclohexylbenzyl group was replaced with both a cyclohexylamide (16b) and an N-(Boc)pentyl substituent (Table 1., SPR, entries 40-47). With the exception of lac (K_{D}=0.9 μM) and 7 am (K_{D}=2.0 μM), which showed moderate increases in binding activity, similar or decreased affinities were observed. Overall, nanomolar to low micromolar affinities were observed for the disclosed substituted 2-(9H-purin-9-yl)acetic acid analogs.

In summary, in developing the pharmacophore model disclosed herein from known active compounds, only the functional groups pertinent to effective interaction with the three main binding centers were considered. A simple pharmacophore plot identifying the optimal positioning of functionality crucial for binding to the STAT3-SH2 domain was prepared (FIG. 1C). The centroid unit, shown as a blue sphere, represents the potential positioning of the scaffold core from which functionality can be optimally appended to access A, B and C. The pharmacophore model disclosed herein describes an essentially planar, hetero-trisubstituted scaffold decorated with optimal recognition elements and confined within the specified pharmacophore plot (FIG. 1C).

### 3. General Synthetic Procedures

a. General Synthetic Procedure A—Alkylation of N2 Using Mitsunobu Conditions

To a stirring solution of purine 4 (1.0 equiv) in THF (0.1M) at room temperature the desired alcohol (1.2 equiv) was added and triphenylphosphine (PPh3, 1.3 equiv). After ˜2 min, diisopropylazodicarboxylate (DIAD, 1.3 equiv) was added dropwise (over ˜30 s-1 min). The reaction mixture stirred for 0.5-2 h before the THF was removed under reduced pressure. The resulting residue was chromatographed on a Biotage Isolera system using a gradient of EtOAc and hexanes.

b. General Synthetic Procedure B—Nucleophilic Aromatic Substitution at C6 with Amines

To a solution of the appropriate chloro-purine (1.0 equiv) in DMSO (0.15M), the desired amine (2.0 equiv) and DIPEA (3.0 equiv) were added. The resulting mixture was sealed in a tube vessel and irradiated in a Biotage Initiator microwave reactor (30 min, 135° C.). After cooling, reaction was diluted with water and repeatedly extracted with EtOAc. The combined organics were washed with water and brine, dried over anhydrous Na₂SO₄, filtered and concentrated under reduced pressure. The resulting residue was adsorbed onto silica gel from CH₂Cl₂ and chromatographed using a Biotage Isolera system with a gradient of EtOAc and hexanes.

C. General Synthetic Procedure C—Nucleophilic Aromatic Substitution at C6 with Anilines

To a solution of di-substituted chloro-purine (1.0 equiv) in DMSO (0.2M), the appropriate aniline (3.0 equiv) and DIPEA (3.0 equiv) were added. The resulting mixture was sealed in a tube vessel and irradiated in a Biotage Initiator microwave reactor (3 h, 135° C.). After cooling, the reaction was diluted with water and repeatedly extracted into EtOAc. The combined organics were washed with water and brine, dried over anhydrous Na₂SO₄, filtered and concentrated under reduced pressure. The resulting residue was dry-loaded onto silica gel from CH₂Cl₂ and chromatographed using a Biotage Isolera system with a gradient of EtOAc and hexanes.

d. General Synthetic Procedure D—Nucleophilic Aromatic Substitution at C6 with Phenols

To a solution of the desired chloro-purine (1.0 equiv) in DMSO (0.2M), DABCO (1.1 equiv) and DIPEA (1.5 equiv) were added and stirred. The solution was allowed to stir for 1 h at room temperature before it was deemed complete, at which point a pre-made solution of the appropriate phenol (2.0 equiv) and DIPEA (1.5 equiv) in DMSO was combined with the chloro-purine to make a 0.1M solution. The reaction was left at room temperature for 16 h, then diluted with water and repeatedly extracted with EtOAc. The combined organics were washed with water and brine, dried over anhydrous Na₂SO₄, filtered and concentrated under reduced pressure. The resulting residue was chromatographed using a Biotage Isolera system with a gradient of EtOAc and hexanes.

e. General Synthetic Procedure E—Ester Hydrolysis with LiOH

x LiOH (1.1 equiv) was added at room temperature to a stirring solution (0.1M) of the appropriate purine (1.0 equiv) in THF:H₂O (3:1). The reaction was deemed complete after 30 min, then diluted with water acidified (pH-5.5) by KH₂PO₄, and continuously extracted into EtOAc. Organic layers were washed with brine, dried over anhydrous Na₂SO₄ and concentrated under reduced pressure. The reaction was purified by flash column chromatography using an isocratic solvent system (35:7:1 CH₂Cl₂:CH₃OH:H₂O) on the Biotage Isolera system. The dried product was suspended in a mixture of millicule water:acetonitrile (6:1) and lyophilized.

f. General Synthetic Procedure F. Boc Deprotection

The appropriate purine (1.0 equiv) was dissolved in TFA:CH₂Cl₂ (1:1) (0.1M solution). The reaction was stirred for one hour at room temperature, co-evaporated with MeOH to near dryness, and dry-loaded onto silica and purified using a Biotage Isolera flash chromatography system using an isocratic system (65:25:4 CH₂Cl₂:CH₃OH:H₂O). The pure product was suspended in a mixture of milicule water:acetonitrile (6:1) and lyophilized.

G. General Synthetic Procedure G—Acylation of N⁶

To a stirring solution of the required purine (1.0 equiv) in pyridine (0.1M) was the appropriate acid chloride added (1.1 equiv). The reaction complete within 15 min, diluted with water acidified by 1M HCl (pH ˜2), and repeatedly extracted into EtOAc. The combined organics were washed with several times with acidified water (pH ˜2) and brine, dried over anhydrous Na₂SO₄ and concentrated under reduced pressure. The resulting residue was chromatographed using the Biotage Isolera system with a gradient of CH₂Cl₂ and (92:7:1 CH₂Cl₂:CH₃OH:NH₄OH) and dried under reduced pressure.

### 4. Preparation of N⁹-Boc-2-Amino-6-chloropurine (Compound ID: 2)

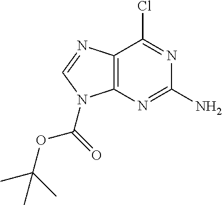 [SMILES: CC(C)(C)OC(=O)n1cnc2c(Cl)nc(N)nc21]

A rapidly stirred solution of 2-amino-6-chloropurine (1 equiv) and di-tert-butyl dicarbonate (Boc₂O; 1 equiv) in anhydrous DMSO (0.3M) was briefly cooled over ice under an N₂ atmosphere. After 5 min (or sooner if the DMSO begins to freeze), the reaction flask was removed from the ice bath and catalytic DMAP (0.05 equiv) was added. The septum was then immediately equipped with a venting needle. After stirring for 30 min at room temperature, TLC indicated the reaction was complete. The reaction mixture was diluted with water and repetitively extracted into EtOAc. The EtOAc layers were combined and washed with water, dried on anhydrous Na₂SO₄, filtered and concentrated to afford N⁹-Boc-2-amino-6-chloropurine (2) as a white solid (75%): δ_{H} (400 MHz, d₆-DMSO) 1.60 (s, 9H, (CH₃)₃), 7.19 (br s, 2H, NH₂), 8.38 (s, 1H, H-8); δC (100 MHz, CDCl₃) 27.9, 87.1, 125.4, 140.1, 145.5, 152.3, 153.3, 160.4; LRMS (ES-MS) calcd for C₁₀H₁₂ClN₅O₂Na[M+Na⁺] m/z=292.06, obsd 291.96.

### 5. Preparation of tert-butyl (6-chloro-9H-purin-2-yl)carbamate (Compound ID: 3)

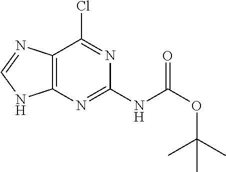 [SMILES: CC(C)(C)OC(=O)Nc1nc(Cl)c2nc[nH]c2n1]

To a stirred solution of purine 2 (1.00 equiv) in anhydrous THF (0.1M) at room temperature was carefully added NaH (60% dispersion in mineral oil; 2.25 equiv) in one portion under an N₂ atmosphere. After 2 h, the Boc transfer reaction was complete. The reaction mixture was cooled to 0° C. then quenched with brine dropwise. The solvent was concentrated down and then poured into a separatory funnel containing saturated aqueous NaHCO₃ solution. The organics were extracted into EtOAc, dried on anhydrous Na₂SO₄, filtered and concentrated. The residue was dry-loaded onto silica gel from CH₂Cl₂, then purified by flash column chromatography (92:7:1 CH₂Cl₂:MeOH:NH₄OH) to afford product as a white powder (95%): δ_{H} (400 MHz, DMSO-d₆) 1.47 (s, 9H, (CH₃)₃), 8.46 (s, 1H, H-8), 10.22 (s, 1H, NHBoc), 13.60 (br s, 1H, H-9); δC (100 MHz, CDCl₃) 28.0, 82.2, 127.8, 145.3, 150.8, 151.1, 151.5, 153.0; LRMS (ES-MS) calcd for C₁₀H₁₂ClN₅O₂Na [M+Na⁺] m/z=292.06, obsd 291.90.

### 6. Preparation of ethyl 2-(2-((tert-butoxycarbonyl)amino)-6-chloro-9H-purin-9-yl)acetate (Compound ID: 4)

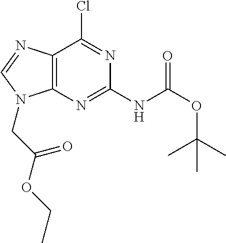 [SMILES: CCOC(=O)Cn1cnc2c(Cl)nc(NC(=O)OC(C)(C)C)nc21]

To a stirred solution of purine 3 (1 equiv) in THF (0.1M) at room temperature was added ethyl glycolate (1.1 equiv) followed by triphenylphosphine (1.1 equiv) under an N₂ atmosphere. To the homogenous solution, DIAD (1 equiv) was added dropwise (over 30 s). TLC indicated the reaction was complete after 15 min and the solvent was removed in vacuo, then the residue was dry-loaded onto silica gel from CH₂C₁₂, and purified by flash column chromatography (2:1 EtOAc:hexanes) to furnish 4 as an off-white foam (83%); mp 129-136° C.; IR (KBr, cm⁻¹) 3462, 3249, 3166, 3106, 2988, 2948, 2362, 1751, 1693, 1612, 1572, 1523, 1499, 1447, 1421; δ_{H} (400 MHz, DMSO-d₆) 1.22 (t, J=7.1 Hz, 3H, CH₃), 1.46 (s, 9H, C(CH₃)₃), 4.18 (q, J=7.1 Hz, 2H, CH₂CH₃), 5.11 (s, 2H, CH₂CO₂Et), 8.46 (s, 1H, H-8), 10.33 (s, 1H, NHBoc); δC (100 MHz, DMSO-d₆) 13.9, 27.8, 44.2, 61.6, 79.7, 126.3, 146.4, 149.0, 150.8, 152.6, 152.9, 167.3; HRMS (ESI⁺) calcd for C₁₄H₁₈ClN₅O₄Na [M+Na⁺] m/z=378.0939, obsd 378.0945.

### 7. Preparation of ethyl 2-(2-((tert-butoxycarbonyl)(pentyl)amino)-6-chloro-9H-purin-9-yl)acetate (Compound ID: 5a)

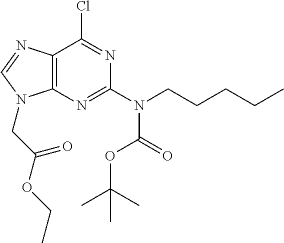 [SMILES: CCCCCN(C(=O)OC(C)(C)C)c1nc(Cl)c2ncn(CC(=O)OCC)c2n1]

Purine 4 was treated according to general procedure A, where ROH was 1-pentanol, to yield final product 5a as a white solid (82%): IR (KBr, cm⁻¹) 3479, 3104, 2960, 2934, 2872, 1754, 1713, 1611, 1563, 1511, 1452, 1407, 1273, 1213, 1136, 1061, 1024; δ_{H} (400 MHz, CDCl₃) 0.88 (t, J=6.9 Hz, 3H, (CH2)₄CH₃), 1.25-1.34 (m, 7H, CO₂CH₂CH₃ and (CH₂)₂CH₂CH₂CH₃), 1.50 (s, 9H, C(CH₃)₃), 1.65 (p, J=7.4 Hz, 2H, CH₂CH₂(CH₂)₂CH₃), 3.89-3.93 (m, 2H, CH₂(CH₂)₃CH₃), 4.27 (q, J=7.1 Hz, 2H, CO₂CH₂CH₃), 4.96 (s, 2H, CH₂CO₂Et), 8.07 (s, 1H, CH(H-8)); LRMS (MS-ES) calcd for C₁₉H₂₉ClN₅O₄ [M+H] m/z=426.18, found 426.43.

### 8. Preparation of ethyl 2-(2-((tert-butoxycarbonyl) (3-cyclohexylbenzyl)amino)-6-chloro-9H-purin-9-yl) acetate (Compound ID: 5b)

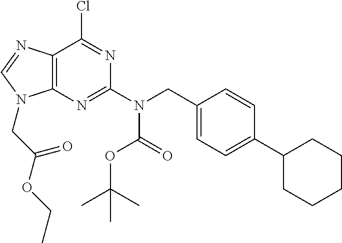 [SMILES: CCOC(=O)Cn1cnc2c(Cl)nc(N(Cc3ccc(C4CCCCC4)cc3)C(=O)OC(C)(C)C)nc21]

Purine 4 was treated according to general procedure A, where ROH was 4-cyclohexyl-benzyl alcohol, to yield final product 5a as a white solid (74%): m.p.=66-71; IR (KBr, cm⁻¹) 2981, 2927, 2852, 1752, 1713, 1564, 1514, 1448, 1405, 1368, 1295, 1278, 1220, 1158, 1109; δ_{H} (400 MHz, CDCl₃) 1.20-1.38 (m, 8H, 5H (cyclohexyl) and CO₂CH₂CH₃), 1.46 (s, 9H, C(CH₃)₃), 1.70-1.83 (m, 5H (cyclohexyl)), 2.43-2.46 (m, 1H, CH), 4.25 (q, J=7.2 Hz, 2H, CO₂CH₂CH₃), 4.93 (s, 2H, CH₂Ar), 5.15 (s, 2H, CH₂CO₂Et), 7.10 (d, J=7.9 Hz, 2H, 2CH(Ar)), 7.28 (d, J=7.9 Hz, 2H, 2CH(Ar)), 8.05 (s, 1H, CH(H-8)); LRMS (MS-ES) calcd for C₂₇H₃₅ClN₅O₄ [M+H] m/z=528.23, found 528.32.

### 9. Preparation of ethyl 2-(6-(benzylamino)-2-((tert-butoxycarbonyl)(pentyl)amino)-9H-purin-9-yl)acetate (Compound ID: 6aa)

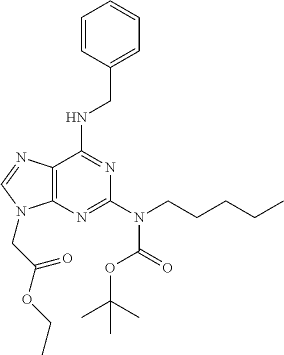 [SMILES: CCCCCN(C(=O)OC(C)(C)C)c1nc(NCc2ccccc2)c2ncn(CC(=O)OCC)c2n1]

Purine 5a was treated with benzylamine according to general procedure B, yielding the final product 6aa as a white solid (52%): m.p.=106-112° C.; IR (KBr, cm⁻¹) 3425, 3275, 2980, 2940, 2868, 1761, 1705, 1625, 1495, 1390, 1270, 1208; δ_{H} (400 MHz, CDCl₃) 0.85 (t, J=7.0 Hz, 3H, (CH₂)₄CH₃), 1.29-1.33 (m, 7H, CO₂CH₂CH₃ and (CH₂)₂CH₂CH₂CH₃), 1.48 (s, 9H, C(CH₃)₃), 1.58-1.65 (m, 2H, (CH₂)₃CH₂CH₃), 3.77-3.81 (m, 2H, CH₂(CH₂)₃CH₃), 4.25 (q, J=7.1 Hz, 2H, CO₂CH₂CH₃), 4.83 (bs, 2H, CH₂Ar), 4.90 (s, 2H, CH₂CO₂Et), 6.05 (bs, 1H, NH), 7.09 (t, J=7.5 Hz, 1H, CH(Ar)), 7.27-7.38 (m, 4H, CH(Ar)), 7.75 (s, 1H, CH(H-8)); LRMS (MS-ES) calcd for C₂₆H₃₇N₆O₄ [M+H] m/z=497.28, found 497.27.

### 10. Preparation of ethyl 2-(6-(benzyl(methyl)amino)-2-((tert-butoxycarbonyl)(pentyl)amino)-9H-purin-9yl)acetate (Compound ID: 6ab)

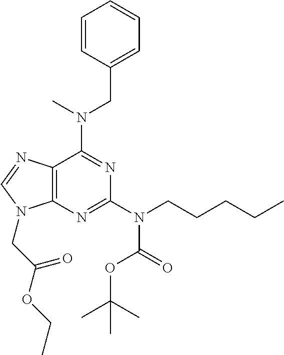 [SMILES: CCCCCN(C(=O)OC(C)(C)C)c1nc(N(C)Cc2ccccc2)c2ncn(CC(=O)OCC)c2n1]

Purine 5a was treated with N-methylbenzylamine according to general procedure B, yielding the final product 6ab as a clear viscous oil (88%): IR (KBr, cm⁻¹) 2958, 2931, 1755, 1701, 1488, 1453, 1385, 1276, 1212, 1145; δ_{H} (400 MHz, CDCl₃) 0.81-0.85 (t, J=7.0 Hz, 3H, (CH₂)₄CH₃), 1.24-1.32 (m, 7H, CO₂CH₂CH₃ and (CH₂)2CH₂CH₂CH₃), 1.46 (s, 9H, C(CH₃)₃), 1.57-1.67 (m, 2H, (CH₂)₃CH₂CH₃), 3.12-3.69 (bm, 3H, NCH₃), 3.76-3.80 (m, 2H, CH₂(CH₂)₃CH₃), 4.25 (q, J=7.1 Hz, 2H, CO₂CH₂CH₃), 4.91 (s, 2H, CH₂CO₂Et), 4.99-5.63 (bm, 2H, CH₂Ar), 7.23-7.33 (m, 5H, CH(Ar)), 7.75 (s, 1H, CH(H-8)); LRMS (MS-ES) calcd for C₂₇H₃₉N₆O₄ [M+H] m/z=511.30, found 511.39.

### 11. Preparation of ethyl 2-(2-((tert-butoxycarbonyl)(pentyl)amino)-6-(phenylamino)-9H-purin-9-yl)acetate (Compound ID: 6ac)

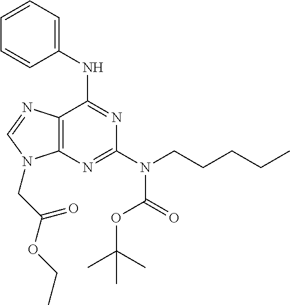 [SMILES: CCCCCN(C(=O)OC(C)(C)C)c1nc(Nc2ccccc2)c2ncn(CC(=O)OCC)c2n1]

Purine 5a was treated with aniline according to general procedure C, yielding the final product 6ac as a clear viscous oil (63%): (KBr, cm⁻¹) 3234, 2932, 1753, 1584, 1499, 1459, 1385, 1274, 1213, 1136; δ_{H} (400 MHz, CDCl₃) 0.88 (t, J=7.0 Hz, 3H, (CH₂)₄CH₃), 1.29-1.33 (m, 7H, CO₂CH₂CH₃ and (CH₂)₂CH₂CH₂CH₃), 1.48 (s, 9H, C(CH₃)₃), 1.66-1.73 (m, 2H, (CH₂)₃CH₂CH₃), 3.86-3.90 (m, 2H, CH₂(CH₂)₃CH₃), 4.27 (q, J=7.1 Hz, 2H, CO₂CH₂CH₃), 4.94 (s, 2H, CH₂CO₂Et), 7.09 (t, J=7.5 Hz, 1H, CH(Ar)), 7.35 (t, J=8.0 Hz, 2H, 2CH(Ar)), 7.66 (bs, 1H, NH), 7.83 (d, J=7.7 Hz, 2H, 2CH(Ar)), 7.85 (s, 1H, CH(H-8)); LRMS (MS-ES) calcd for C₂₅H₃₅N₆O₄ [M+H] m/z=483.26, found 483.31.

### 12. Preparation of ethyl 2-(2-((tert-butoxycarbonyl)(pentyl)amino)-6-((furan-2-ylmethyl)(methyl)amino)-9Hpurin-9-yl)acetate (Compound ID: 6ad)

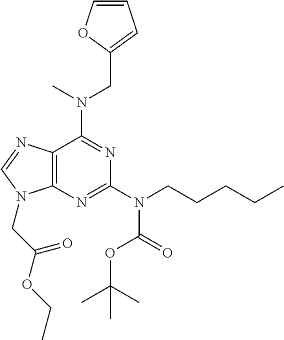 [SMILES: CCCCCN(C(=O)OC(C)(C)C)c1nc(N(C)Cc2ccco2)c2ncn(CC(=O)OCC)c2n1]

Purine 5a was treated with N-methylfurfurylamine according to general procedure B, yielding the final product 6ad as a clear viscous oil (91%): IR (KBr, cm⁻¹) 3538, 3475, 3400, 3225, 2925, 2860, 1750, 1700, 1600, 1435, 1380, 1210; δ_{H} (400 MHz, CDCl₃) 0.86 (t, J=6.9 Hz, 3H, (CH₂)₄CH₃), 1.25-1.34 (m, 7H, CO₂CH₂CH₃ and (CH₂)₂CH₂CH₂CH₃), 1.48 (s, 9H, C(CH₃)₃), 1.65 (p, 2H, CH₂CH₂(CH₂)₂CH₃), 3.56 (vbs, 3H, CH₃(furfuryl)), 3.81 (t, J=7.6 Hz, 2H, CH₂(CH₂)₃CH₃), 4.25 (q, J=7.1 Hz, 2H, CO₂CH₂CH₃), 4.93 (s, 2H, CH₂CO₂Et), 5.35 (vbs, 2H, CH2 (furfuryl)), 6.28-6.31 (m, 2H, CH (furfuryl)), 7.34-7.35 (m, 1H, (furfuryl)) 7.78 (s, 1H, CH(H-8)); LRMS (MS-ES), calcd for C₂₅H₃₇N₆O₅ [M+H] m/z=501.27, found 501.30.

### 13. Preparation of ethyl 2-(2-((tert-butoxycarbonyl)(pentyl)amino)-6-(cyclopentylamino)-9H-purin-9-yl) acetate (Compound ID: 6ae)

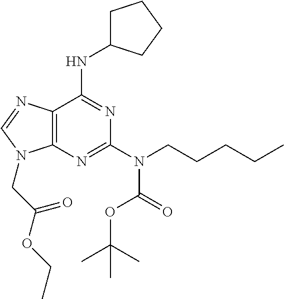 [SMILES: CCCCCN(C(=O)OC(C)(C)C)c1nc(NC2CCCC2)c2ncn(CC(=O)OCC)c2n1]

Purine 5a was treated with cyclopentanamine according to general procedure B, yielding the final product 6ae as a clear viscous oil (83%): IR (KBr, cm⁻¹) 3546, 3475, 3410, 3230, 2950, 2865, 1760, 1710, 1625, 1480, 1400, 1270; δ_{H} (400 MHz, CDCl₃) 0.87 (t, J=7.0 Hz, 3H, (CH₂)₄CH₃), 1.25-1.34 (m, 7H, CO₂CH₂CH₃ and (CH₂)₂CH₂CH₂CH₃), 1.48 (s, 9H, C(CH₃)₃), 1.54-1.84 (m, 8H, CH₂CH₂(CH₂)₂CH₃ and 3CH₂ (cyclopentyl)), 2.11 (m, 2H, CH₂ (cyclopentyl)), 3.81 (t, J=7.6 Hz, 2H, CH₂(CH₂)₃CH₃), 4.25 (q, J=7.0 Hz, 2H, CO₂CH₂CH₃), 4.53 (bs, 1H, CH (cyclopentyl)), 4.89 (s, 2H, CH₂CO₂Et), 5.68 (bs, 1H, NH), 7.76 (s, 1H, CH(H-8)); LRMS (MS-ES), calcd for C₂₄H₃₉N₆O₄ [M+H] m/z=475.30, found 475.37

### 14. Preparation of ethyl 2-(2-((tert-butoxycarbonyl)(pentyl)amino)-6-(cyclohexylamino)-9H-purin-9-yl)acetate (Compound ID: 6af)

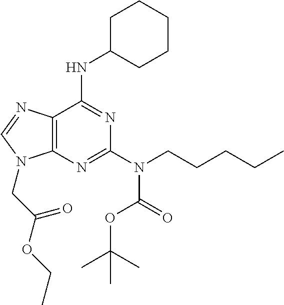 [SMILES: CCCCCN(C(=O)OC(C)(C)C)c1nc(NC2CCCCC2)c2ncn(CC(=O)OCC)c2n1]

Purine 5a was treated with cyclohexanamine according to general procedure B, yielding the final product 6af as a clear viscous oil (70%): IR (KBr, cm⁻¹) 2940, 2586, 1760, 1390, 1150; δ_{H} (400 MHz, CDCl₃) 0.88 (t, J=6.9 Hz, 3H, (CH₂)₄CH₃), 1.25-1.44 (m, 13H, CO₂CH₂CH₃, (CH₂)₂CH₂CH₂CH₃ and 3CH₂ (cyclohexyl)), 1.49 (s, 9H, C(CH₃)₃), 1.62-1.71 (m, 2H, CH₂CH₂(CH₂)₂CH₃), 1.76-1.84 (m, 2H, CH₂ (cyclohexyl)), 2.06-2.13 (m, 2H, CH₂ (cyclohexyl)), 3.80 (t, J=7.7 Hz, 2H, CH₂(CH₂)₃CH₃), 4.10 (bs, 1H, CH (cyclohexyl)), 4.25 (q, J=7.1 Hz, 2H, CO₂CH₂CH₃), 4.88 (s, 2H, CH₂CO₂Et), 5.62 (bs, 1H, NH), 7.76 (s, 1H, CH(H8)); LRMS (MS-ES), calcd for C₂₅H₄₁N₆O₄ [M+H] m/z=489.31, found 489.34.

### 15. Preparation of ethyl 2-(2-((tert-butoxycarbonyl)(pentyl)amino)-6-(ethyl(methyl)amino)-9H-purin-9-yl)acetate (Compound ID: 6ag)

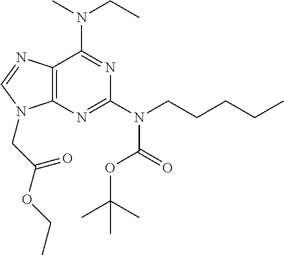 [SMILES: CCCCCN(C(=O)OC(C)(C)C)c1nc(N(C)CC)c2ncn(CC(=O)OCC)c2n1]

Purine 5a was treated with N-methylethylamine according to general procedure B, yielding the final product 6ag as a clear viscous oil (84%): IR (KBr, cm⁻¹) 3530, 3475, 3413, 2970, 2950, 2880, 1760, 1700, 1600, 1475, 1440, 1390; δ_{H} (400 MHz, CDCl₃) 0.87 (t, J=7.0 Hz, 3H, (CH₂)₄CH₃), 1.24-1.33 (m, 10H, CO₂CH₂CH₃, (CH₂)₂CH₂CH₂CH₃ and NCH₂CH₃), 1.47 (s, 9H, C(CH₃)₃), 1.67 (p, 2H, CH₂CH₂(CH₂)₂CH₃), 3.42 (bm, 3H, NCH), 3.79 (t, J=7.7 Hz, 2H, CH₂(CH₂)₃CH₃), 4.04 (bm, 2H, NCH₂CH₃), 4.24 (q, J=7.1 Hz, 2H, CO₂CH₂CH₃), 4.89 (s, 2H, CH₂CO₂Et), 7.74 (s, 1H, CH(H-8)); LRMS (MS-ES), calcd for C₂₂H₃₇N₆O₄ [M+H] m/z=449.28, found 449.44.

### 16. Preparation of ethyl 2-(2-((tert-butoxycarbonyl)(pentyl)amino)-6-(isopropylamino)-9H-purin-9-yl)acetate (Compound ID: 6ah)

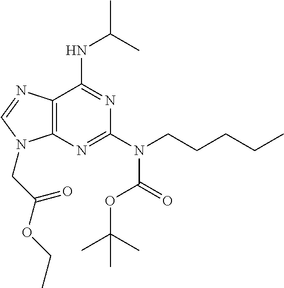 [SMILES: CCCCCN(C(=O)OC(C)(C)C)c1nc(NC(C)C)c2ncn(CC(=O)OCC)c2n1]

Purine 5a was treated with isopropylamine according to general procedure B, yielding the final product bah as a clear viscous oil (75%): IR (KBr, cm⁻¹) 3546, 3475, 3410, 2975, 2925, 1775, 1700, 1615, 1475, 1380, 1370, 1225; δ_{H} (400 MHz, CDCl₃) 0.87 (t, J=7.0 Hz, 3H, (CH₂)₄CH₃), 1.24-1.33 (m, 13H, CO₂CH₂CH₃, (CH₂)₂CH₂CH₂CH₃ and CH(CH₃)₂), 1.48 (s, 9H, C(CH₃)₃), 1.66 (p, 2H, CH₂CH₂(CH₂)₂CH₃), 3.80 (t, J=7.6 Hz, 2H, CH₂(CH₂)₃CH₃), 4.25 (q, J=7.1 Hz, 2H, CO₂CH₂CH₃), 4.45 (bs, 1H, CH(CH₃)₂), 4.89 (s, 2H, CH₂CO₂Et), 5.56 (bs, 1H, NH), 7.76 (s, 1H, CH(H-8)); LRMS (MS-ES), calcd for C₂₂H₃₇N₆O₄ [M+H] m/z=449.28, found 449.38.

### 17. Preparation of ethyl 2-(6-(allylamino)-2-((tert-butoxycarbonyl)(pentyl)amino)-9H-purin-9-yl)acetate (Compound ID: 6ai)

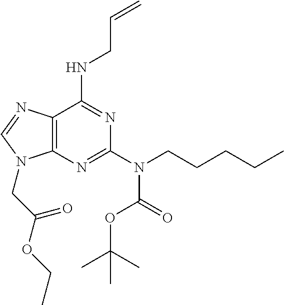 [SMILES: C=CCNc1nc(N(CCCCC)C(=O)OC(C)(C)C)nc2c1ncn2CC(=O)OCC]

Purine 5a was treated with allylamine according to general procedure B, yielding the final product 6ai as a white solid (72%): m.p.=67-78° C.; IR (KBr, cm⁻¹) 3546, 3476, 3413, 3276, 2940, 1760, 1710, 1680, 1625, 1490, 1380, 1200; δ_{H} (400 MHz, CDCl₃) 0.87 (t, J=6.8 Hz, 3H, (CH₂)₄CH₃), 1.25-1.33 (m, 7H, CO₂CH₂CH₃ and (CH₂)₂CH₂CH₂CH₃), 1.49 (s, 9H, C(CH₃)₃), 1.65 (p, 2H, CH₂CH₂(CH₂)₂CH₃), 3.82 (t, J=7.6 Hz, 2H, CH₂(CH₂)₃CH₃), 4.25 (q, J=7.1 Hz, 2H, CO₂CH₂CH₃), 4.27 (vbs, 2H, CH₂CHCH₂), 4.90 (s, 2H, CH₂CO₂Et), 5.18 (d, J=9.9 Hz, 1H, CH₂CHCH₂), 5.31 (d, J=17.4 Hz, 1H, CH₂CHCH₂), 5.85 (bs, 1H, NH), 5.94-6.04 (m, 1H, CH₂CHCH₂), 7.80 (s, 1H, CH(H-8)); LRMS (MS-ES), calcd for C₂₂H₃₅N₆O₄ [M+H] m/z=447.26, found 447.36.

### 18. Preparation of ethyl 2-(2-((tert-butoxycarbonyl)(pentyl)amino)-6-(isobutylamino)-9H-purin-9-yl)acetate (Compound ID: 6aj)

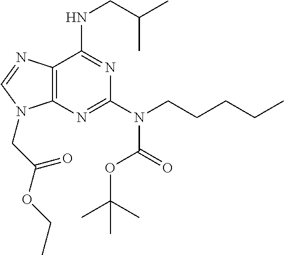 [SMILES: CCCCCN(C(=O)OC(C)(C)C)c1nc(NCC(C)C)c2ncn(CC(=O)OCC)c2n1]

Purine 5a was treated with isobutylamine according to general procedure B, yielding the final product 6aj as a white solid (83%): m.p.=89-93° C.; IR (KBr, cm⁻¹) 3425, 3290, 2960, 2925, 2885, 1760, 1670, 1630, 1580, 1380, 1249, 1200; δ_{H} (400 MHz, CDCl₃) 0.87 (t, J=6.8 Hz, 3H, (CH₂)₄CH₃), 0.99 (s, 3H, CH(CH₃)₂), 1.00 (s, 3H, CH(CH₃)2), 1.25-1.34 (m, 7H, CO₂CH₂CH₃ and (CH₂)₂CH₂CH₂CH₃), 1.48 (s, 9H, C(CH₃)₃), 1.66 (p, 2H, CH₂CH₂(CH₂)₂CH₃), 1.97 (septet, J=6.6 Hz, 1H, CH(CH₃)₂), 3.43 (bs, 2H, CH₂CH(CH₃)₂), 3.81 (t, J=7.6 Hz, 2H, CH₂(CH₂)₃CH₃), 4.25 (q, J=7.1 Hz, 2H, CO₂CH₂CH₃), 4.89 (s, 2H, CH₂CO₂Et), 5.79 (bs, 1H, NH), 7.76 (s, 1H, CH(H-8)); LRMS (MS-ES), calcd for C₂₃H₃₉N₆O₄ [M+H] m/z=463.30, found 463.41.

### 19. Preparation of ethyl 2-(2-((tert-butoxycarbonyl)(pentyl)amino)-6-(butyl(methyl)amino)-9H-purin-9-yl)acetate (Compound ID: 6ak)

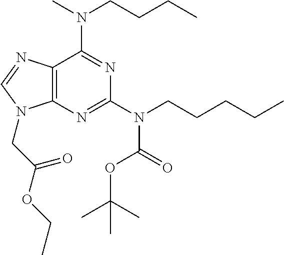 [SMILES: CCCCCN(C(=O)OC(C)(C)C)c1nc(N(C)CCCC)c2ncn(CC(=O)OCC)c2n1]

Purine 5a was treated with N-butylmethylamine according to general procedure B, yielding the final product 6ak as a clear viscous oil (63%): IR (KBr, cm⁻¹) 3550, 3460, 3410, 2950, 2925, 2860, 1760, 1700, 1600, 1440, 1400, 1200; δ_{H} (400 MHz, CDCl₃) 0.88 (t, J=7.0 Hz, 3H, (CH₂)₄CH₃), 0.96 (t, J=7.3 Hz, 3H, (CH₂)₃CH₃), 1.26-1.45 (m, 9H, CO₂CH₂CH₃, CH₂CH₂CH₂CH₃ and (CH₂)₂CH₂CH₂CH₃), 1.49 (s, 9H, C(CH₃)₃), 1.62-1.73 (4H, CH₂CH₂CH₂CH₃ and CH₂CH₂(CH₂)₂CH₃), 3.20-4.24 (m, 5H, CH₂(CH₂)₂CH₃ and CH₂(CH₂)₃CH₃), 3.79 (t, J=7.6 Hz, 2H, CH₂(CH₂)₃CH₃), 4.25 (q, J=7.1 Hz, 2H, CO₂CH₂CH₃), 4.91 (s, 2H, CH₂CO₂Et), 7.74 (s, 1H, CH(H-8)); LRMS (MS-ES), calcd for C₂₄H₄₁N₆O₄ [M+H] m/z=477.31, found 477.38.

### 20. Preparation of ethyl 2-(2-((tert-butoxycarbonyl)(pentyl)amino)-6-(isopentylamino)-9H-purin-9-yl)acetate (Compound ID: 6al)

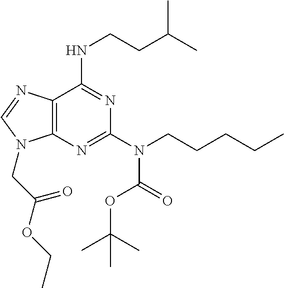 [SMILES: CCCCCN(C(=O)OC(C)(C)C)c1nc(NCCC(C)C)c2ncn(CC(=O)OCC)c2n1]

Purine 5a was treated with isoamylamine according to general procedure B, yielding the final product 6al as a white solid (70%): m.p.=70-91° C.; IR (KBr, cm⁻¹) 3546, 3475, 3410, 2960, 2925, 2860, 1760, 1700, 1608, 1380, 1250, 1213; δ_{H} (400 MHz, CDCl₃) 0.87 (t, J=6.9 Hz, 3H, (CH₂)₄CH₃), 0.95 (s, 3H, (CH₂)₂CH(CH₃)₂), 0.96 (s, 3H, (CH₂)₂CH(CH₃)₂), 1.25-1.34 (m, 7H, CO₂CH₂CH₃, and (CH₂)₂CH₂CH₂CH₃), 1.49 (s, 9H, C(CH₃)₃), 1.53-1.78 (m, 5H, CH₂CH₂(CH₂)₂CH₃ and CH₂CH₂CH(CH₃)₂), 3.62 (bs, 2H, CH₂(CH₂)₂(CH₃)₂), 3.81 (t, J=7.6 Hz, 2H, CH₂(CH₂)₃CH₃), 4.25 (q, J=7.1 Hz, 2H, CO₂CH₂CH₃), 4.89 (s, 2H, CH₂CO₂Et) 5.76 (bs, 1H, NH), 7.77 (s, 1H, CH(H-8)); LRMS (MS-ES), calcd for C₂₄H₄₁N₆O₄ [M+H] m/z=477.3, fnd 477.32.

### 21. Preparation of ethyl 2-(2-((tert-butoxycarbonyl)(pentyl)amino)-6-morpholino-9H-purin-9-yl)acetate (Compound ID: 6am)

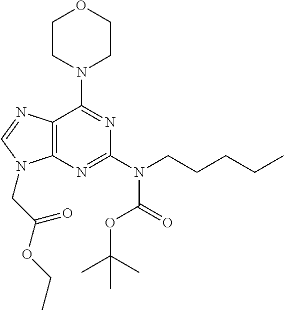 [SMILES: CCCCCN(C(=O)OC(C)(C)C)c1nc(N2CCOCC2)c2ncn(CC(=O)OCC)c2n1]

Purine 5a was treated with morpholine according to general procedure B, yielding the final product 6 am as a clear viscous oil (83%): IR (KBr, cm⁻¹) 2960, 2931, 2858, 1755, 1712, 1589, 1478, 1444, 1386, 1365, 1220, 1146, 1117; δ_{H} (400 MHz, CDCl₃) 0.87 (t, J=6.9 Hz, 3H, (CH₂)₄CH₃), 1.25-1.31 (m, 7H, CO₂CH₂CH₃ and (CH₂)₂CH₂CH₂CH₃), 1.47 (s, 9H, C(CH₃)₃), 1.65 (p, J=7.4 Hz, 2H, CH₂CH₂(CH₂)₂CH₃), 3.78-3.84 (m, 6H, CH₂(CH₂)₃CH₃ and 2CH₂ (morpholine)), 4.25 (q, J=7.2 Hz, 2H, CO₂CH₂CH₃), 4.26 (bs, 4H, 2CH₂ (morpholine)), 4.89 (s, 2H, CH₂CO₂Et), 7.75 (s, 1H, CH(H-8)); LRMS (MS-ES), calcd for C₂₃H₃₆N₆O₄Na [M+Na] m/z=499.27, fnd 499.43.

### 22. Preparation of ethyl 2-(2-((tert-butoxycarbonyl)(pentyl)amino)-6-(3-nitrophenoxy)-9H-purin-9-yl)acetate (Compound ID: 6an)

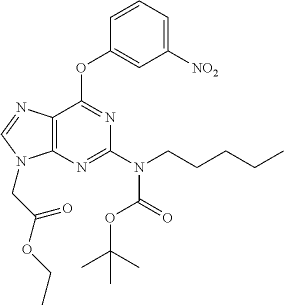 [SMILES: CCCCCN(C(=O)OC(C)(C)C)c1nc(Oc2cccc([N+](=O)[O-])c2)c2ncn(CC(=O)OCC)c2n1]

Purine 5a was treated with 3-nitrophenol according to general procedure D, yielding the final product 6an as a clear viscous oil (73%): IR (KBr, cm⁻¹) 2959, 2931, 1752, 1713, 1578, 1533, 1448, 1407, 1354, 1276, 1222, 1149; δ_{H} (400 MHz, CDCl₃) 0.82 (t, J=7.3 Hz, 3H, (CH₂)₄CH₃), 1.04-1.21 (m, 4H, (CH₂)₂CH₂CH₂CH₃), 1.32 (t, J=7.2 Hz, 3H, CO₂CH₂CH₃), 1.40 (s, 9H, C(CH₃)₃), 1.44-1.52 (m, 2H, CH₂CH₂(CH₂)₂CH₃), 3.65-3.69 (m, 2H, CH₂(CH₂)₃CH₃), 4.28 (q, J=7.2 Hz, 2H, CO₂CH₂CH₃), 4.98 (s, 2H, CH₂CO₂Et), 7.60 (t, J=8.2 Hz, 1H, CH(Ar)), 7.68 (d, J=8.2 Hz, 1H, CH(Ar)), 8.02 (s, 1H, CH(H-8)), 8.14 (d, J=8.2 Hz, 1H, 1 CH(Ar)), 8.23 (d, J=2.2 Hz, 1H, 1 CH(Ar)); LRMS (MS-ES), calcd for C₂₄H₃₃N₆O₇ [M+H] m/z=529.23, fnd 529.45.

### 23. Preparation of ethyl 2-(2-((tert-butoxycarbonyl)(pentyl)amino)-6-(4-nitrophenoxy)-9H-purin-9-yl)acetate (Compound ID: 6ao)

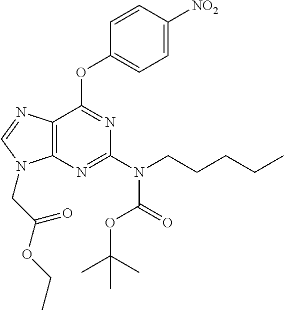 [SMILES: CCCCCN(C(=O)OC(C)(C)C)c1nc(Oc2ccc([N+](=O)[O-])cc2)c2ncn(CC(=O)OCC)c2n1]

Purine 5a was treated with 4-nitrophenol according to general procedure D, yielding the final product 6ao as a white solid (68%): m.p.>99-110° C.; IR (KBr, cm⁻¹) 3100, 3080, 2940, 2870, 1760, 1725, 1608, 1570, 1530, 1345, 1250, 1230; δ_{H} (400 MHz, CDCl₃) 0.82 (t, J=7.1 Hz, 3H, (CH₂)₄CH₃), 1.01-1.26 (m, 4H, (CH₂)₂CH₂CH₂CH₃), 1.32 (t, J=7.2 Hz, 3H, CO₂CH₂CH₃), 1.43 (s, 9H, C(CH₃)₃), 1.51 (p, J=7.6 Hz, 2H, CH₂CH₂(CH₂)₂CH₃), 3.68-3.72 (m, 2H, CH₂(CH₂)₃CH₃), 4.28 (q, J=7.6 Hz, 2H, CO₂CH₂CH₃), 4.98 (s, 2H, CH₂CO₂Et), 7.54 (d, J=9.1 Hz, 2H, 2CH(Ar)), 8.02 (s, 1H, CH(H-8)), 8.31 (d, J=9.1 Hz, 2H, 2CH(Ar)); LRMS (MS-ES), calcd for C₂₅H₃₂N₆O₇Na [M+Na] m/z=528.23, found 551.27.

### 24. Preparation of ethyl 2-(2-((tert-butoxycarbonyl)(pentyl)amino)-6-((tetrahydro-2H-pyran-4-yl)amino)-9H-purin-9-yl)acetate (Compound ID: 6ay)

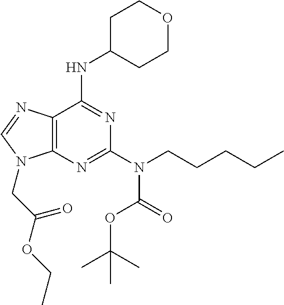 [SMILES: CCCCCN(C(=O)OC(C)(C)C)c1nc(NC2CCOCC2)c2ncn(CC(=O)OCC)c2n1]

Purine 5a was treated with tetrahydro-2H-pyran-4-amine according to general procedure B, yielding the final product 6ay as a white solid (86%): m.p.>183° C. (dec); IR (KBr, cm⁻¹) 2953, 2850, 1760, 1683, 1472, 1441, 1400, 1383, 1366, 1298, 1277, 1208, 1140; 6_{H} (400 MHz, CDCl₃) 0.87 (t, J=6.9 Hz, 3H, (CH₂)₄CH₃), 1.25-1.34 (m, 7H, CO₂CH₂CH₃, and (CH₂)₂CH₂CH₂CH₃), 1.48 (s, 9H, C(CH₃)₃), 1.58-1.73 (m, 4H, 2H, CH₂, (tetrahydropyran) and CH₂CH₂(CH₂)₂CH₃)), 2.04-2.08 (m, 2H, CH₂, (tetrahydropyran)), 3.48-3.60 (m, 2H, CH₂, (tetrahydropyran)), 3.79 (t, J=7.6 Hz, 2H, CH₂(CH₂)₃CH₃), 3.95-4.07 (m, 2H, CH₂, (tetrahydropyran)), 4.25 (q, J=7.1 Hz, 2H, CO₂CH₂CH₃), 4.33 (bs, 1H, CH), 4.89 (s, 2H, CH₂CO₂Et) 6.01 (bs, 1H, NH), 7.77 (s, 1H, CH(H-8)); LRMS (MS-ES), calcd for C₂₄H₃₈N₆O₅Na [M+Na] m/z=513.29, found 513.44.

### 25. Preparation of ethyl 2-(6-(benzylamino)-2-((tert-butoxycarbonyl)(4-cyclohexylbenzyl)amino)-9H-purin-9yl)acetate (Compound ID: 6ba)

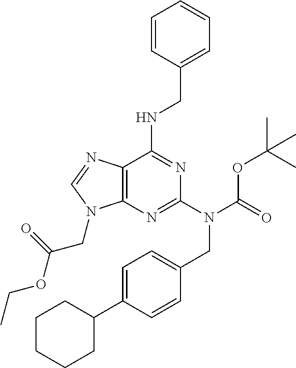 [SMILES: CCOC(=O)Cn1cnc2c(NCc3ccccc3)nc(N(Cc3ccc(C4CCCCC4)cc3)C(=O)OC(C)(C)C)nc21]

Purine 5b was treated with benzylamine according to general procedure B, yielding the final product 6ba as a white solid (85%): m.p.>116° C. (dec); IR (KBr, cm⁻¹) 3325, 3140, 2990, 2925, 2850, 1750, 1697, 1625, 1600, 1390, 1370, 1225; δ_{H} (400 MHz, CDCl₃) 1.21-1.40 (m, 8H, 5H (cyclohexyl) and CO₂CH₂CH₃), 1.46 (s, 9H, C(CH₃)₃), 1.71-1.85 (m, 5H (cyclohexyl)), 2.41-2.46 (m, 1H, CH), 4.25 (q, J=7.2 Hz, 2H, CO₂CH₂CH₃), 4.75 (bs, 2H, HNCH₂), 4.90 (bs, 2H, CH₂Ar), 5.06 (s, 2H, CH₂CO₂Et), 7.08 (m, 2H, 2CH (Ar)), 7.21-7.32 (m, 7H, 7 CH(Ar)), 7.43 (bs, 1H, NH), 7.87 (s, 1H, CH(H-8)); LRMS (MS-ES), calcd for C₃₄H₄₃N₆O₄ [M+H] m/z=599.33, found 599.49.

### 26. Preparation of ethyl 2-(6-(benzyl(methyl)amino)-2-((tert-butoxycarbonyl)(4-cyclohexylbenzyl)amino)-9Hpurin-9-yl)acetate (Compound ID: 6bb)

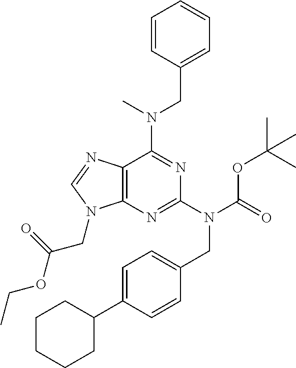 [SMILES: CCOC(=O)Cn1cnc2c(N(C)Cc3ccccc3)nc(N(Cc3ccc(C4CCCCC4)cc3)C(=O)OC(C)(C)C)nc21]

Purine 5b was treated with N-methylbenzylamine according to general procedure B, yielding the final product Ebb as a white solid (72%): m.p.=115-121° C.; IR (KBr, cm⁻¹) 3419, 2979, 2925, 2851, 1755, 1698, 1594, 1558, 1488, 1454, 1418, 1377, 1204, 1152, 1107; δ_{H} (400 MHz, CDCl₃) 1.29 (t, J=7.2 Hz, 3H, CO₂CH₂CH₃), 1.33-1.41 (m, 5H, (cyclohexyl)), 1.41 (s, 9H, C(CH₃)₃), 1.71-1.82 (m, 5H (cyclohexyl)), 2.40-2.47 (m, 1H, CH), 3.06-3.71 (bm, 3H, NCH₃), 4.24 (q, J=7.2 Hz, 2H, CO₂CH₂CH₃), 4.89 (s, 2H, CH₂Ar), 5.03 (bs, 2H, CH₂CO₂Et), 5.17-5.61 (bm, 2H, CH₃NCH₂), 7.03-7.05 (m, 2H, CH(Ar)), 7.23-7.31 (m, H, 7 CH(Ar)), 7.73 (s, 1H, CH(H-8)); LRMS (MS-ES), calcd for C₃₅H₄₅N₆O₄ [M+H] m/z=613.34, found 613.50

### 27. Preparation of ethyl 2-(2-((tert-butoxycarbonyl)(4-cyclohexylbenzyl)amino)-6-((furan-2-ylmethyl)(methyl)amino)-9H-purin-9-yl)acetate (Compound ID: 6bd)

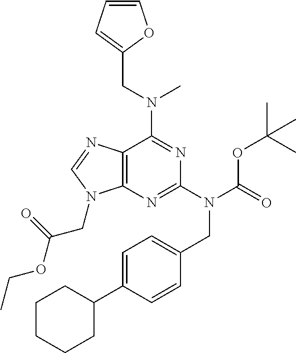 [SMILES: CCOC(=O)Cn1cnc2c(N(C)Cc3ccco3)nc(N(Cc3ccc(C4CCCCC4)cc3)C(=O)OC(C)(C)C)nc21]

Purine 5b was treated with N-methylfurfurylamine according to general procedure B, yielding the final product bbd as a white solid (67%): m.p.>120° C. (dec); IR (KBr, cm⁻¹) 1158, 1213, 1377, 1447, 1591, 1699, 1755, 2850, 2900, 2945; δ_{H} (400 MHz, CDCl₃) 1.26-1.39 (m, 8H, 5H (cyclohexyl) and CO₂CH₂CH₃), 1.41 (s, 9H, C(CH₃)₃), 1.71-1.83 (m, 5H (cyclohexyl)), 2.40-2.46 (m, 1H, CH), 3.14-3.75 (vbs, 3H, NCH), 4.23 (q, J=7.2 Hz, 2H, CO₂CH₂CH₃), 4.88 (s, 2H, CH₂Ar), 5.05 (s, 2H, CH₂CO₂Et), 5.17 (vbs, 2H, CH₂ (furfuryl)), 6.17-6.23 (m, 1H, CH (furfuryl)), 6.28-6.29 (m, 1H, CH (furfuryl)), 7.07 (d, J=8.1 Hz, 2H, 2 CH(Ar)), 7.27 (d, J=8.2 Hz, 2H, 2CH(Ar)), 7.32-7.33 (m, 1H, CH (furfuryl)), 7.75 (s, 1H, CH(H-8)); LRMS (MS-ES), calcd for C₃₃H₄₂N₆O₅Na [M+Na] m/z=625.32, found 625.49.

### 28. Preparation of ethyl 2-(2-((tert-butoxycarbonyl)(4-cyclohexylbenzyl)amino)-6-(cyclopentylamino)-9Hpurin-9-yl)acetate (Compound ID: 6be)

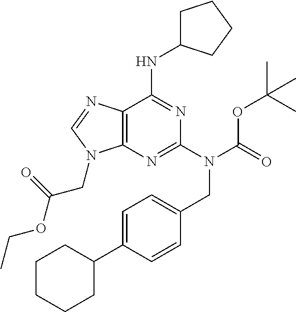 [SMILES: CCOC(=O)Cn1cnc2c(NC3CCCC3)nc(N(Cc3ccc(C4CCCCC4)cc3)C(=O)OC(C)(C)C)nc21]

Purine 5b was treated with cyclopentanamine according to general procedure B, yielding the final product 6be as a white solid (81%): m.p.>133° C. (dec); IR (KBr, cm⁻¹) 3549, 2978, 2926, 2851, 1752, 1702, 1541, 1515, 1481, 1438, 1391, 1238, 1212, 1158, 1110, 1022; δ_{H} (400 MHz, CDCl₃) 1.18-1.46 (m, 14H, 5H (cyclohexyl) and C(CH₃)₃), 1.28 (t, J=7.2 Hz, 3H, CO₂CH₂CH₃), 1.46-1.54 (m, 4H (cyclopentyl)), 1.71-1.82 (m, 7H, 5H (cyclohexyl) and 2H (cyclopentyl)), 2.03 (bs, 2H (cyclopentyl)), 2.41-2.47 (m, 1H, CH), 4.23 (q, J=7.2 Hz, 2H, CO₂CH₂CH₃), 4.44 (bs, 1H, NCH), 4.87 (s, 2H, CH₂Ar), 5.05 (s, 2H, CH₂CO₂Et), 5.76 (bs, 1H, NH), 7.09 (d, J=8.1 Hz, 2H, 2CH(Ar)), 7.30 (d, J=8.1 Hz, 2H, 2CH(Ar)), 7.74 (s, 1H, CH(H-8)); LRMS (MS-ES), calcd for C₃₂H₄₅N₆O₄ [M+H] m/z=577.34, found 577.46.

### 29. Preparation of ethyl 2-(2-((tert-butoxycarbonyl)(4-cyclohexylbenzyl)amino)-6-(cyclohexylamino)-9Hpurin-9-yl)acetate (Compound ID: 6bf)

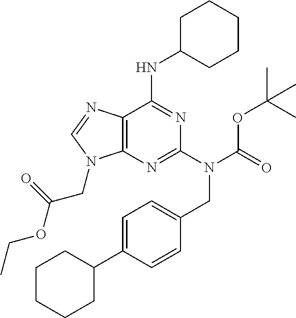 [SMILES: CCOC(=O)Cn1cnc2c(NC3CCCCC3)nc(N(Cc3ccc(C4CCCCC4)cc3)C(=O)OC(C)(C)C)nc21]

Purine 5b was treated with cyclohexanamine according to general procedure B, yielding the final product 6bf as a white solid (88%): m.p.=75-84° C.; IR (KBr, cm⁻¹) 3413, 2913, 2850, 1712, 1475, 1357, 1237, 1213, 1150; δ_{H} (400 MHz, CDCl₃) 1.15-1.40 (m, 13H, 5H, (cyclohexyl)), 5H, (NH-cyclohexyl) and CO₂CH₂CH₃)), 1.42 (s, 9H, C(CH₃)₃), 1.57-1.86 (m, 10H, 5H, (cyclohexyl) and 5H, (NH-cyclohexyl)), 2.38-2.48 (m, 1H, CH), 4.02 (bs, 1H, HNCH), 4.24 (q, J=7.2 Hz, 2H, CO₂CH₂CH₃), 4.87 (s, 2H, CH₂Ar), 5.03 (s, 2H, CH₂CO₂Et), 5.58 (bs, 1H, NH), 7.09 (d, J=8.1 Hz, 2H, 2CH(Ar)), 7.30 (d, J=8.1 Hz, 2H, 2 CH(Ar)), 7.73 (s, 1H, CH(H-8)); LRMS (MS-ES), calcd for C₃₃H₄₇N₆O₄ [M+H] m/z=591.36, found 591.54.

### 30. Preparation of ethyl 2-(6-(allylamino)-2-((tert-butoxycarbonyl)(4-cyclohexylbenzyl)amino)-9H-purin-9-yl)acetate (Compound ID: 6bi)

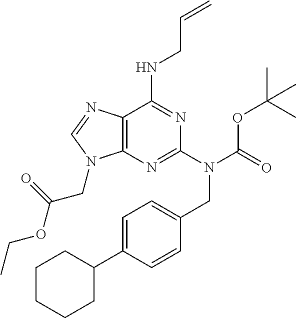 [SMILES: C=CCNc1nc(N(Cc2ccc(C3CCCCC3)cc2)C(=O)OC(C)(C)C)nc2c1ncn2CC(=O)OCC]

Purine 5b was treated with allylamine according to general procedure B, yielding the final product 6bi as a white solid (82%): m.p.=125-134° C.; IR (KBr, cm⁻¹) 3559, 3475, 3410, 3245, 2930, 2858, 1755, 1700, 1630, 1615, 1480, 1408; δ_{H} (400 MHz, CDCl₃) 1.25-1.40 (m, 8H, 5H (cyclohexyl) and CO₂CH₂CH₃), 1.41 (s, 9H, C(CH₃)₃), 1.70-1.86 (m, 5H (cyclohexyl)), 2.40-2.48 (m, 1H, CH), 4.25 (q, J=7.2 Hz, 2H, CO₂CH₂CH₃), 4.26 (bs, 2H, CH₂CHCH₂), 4.88 (s, 2H, CH₂Ar), 5.05 (s, 2H, CH₂CO₂Et), 5.15 (dd, J=10.3 and 1.5 Hz, 1H, CH₂CHCH₂), 5.25 (dd, J=17.1 and 1.5 Hz, 1H, CH₂CHCH₂), 5.70 (bs, 1H, NH), 5.89-5.99 (m, 1H, CH₂CHCH₂), 7.09 (d, J=8.1 Hz, 2H, 2CH(Ar)), 7.29 (d, J=8.3 Hz, 2H, 2CH (Ar)), 7.75 (s, 1H, CH(H-8)); LRMS (MS-ES), calcd for C₃₀H₄₁N₆O₄ [M+H] m/z=549.31, found 549.45.

### 31. Preparation of ethyl 2-(2-((tert-butoxycarbonyl)(4-cyclohexylbenzyl)amino)-6-(isobutylamino)-9H-purin-9-yl)acetate (Compound ID: 6bj)

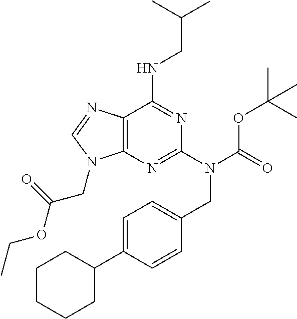 [SMILES: CCOC(=O)Cn1cnc2c(NCC(C)C)nc(N(Cc3ccc(C4CCCCC4)cc3)C(=O)OC(C)(C)C)nc21]

Purine 5b was treated with isobutylamine according to general procedure B, yielding the final product 6bj as a white solid (77%): m.p.=70-85° C.; IR (KBr, cm⁻¹) 2926, 1755, 1532, 1479, 1448, 1385, 1352, 1240, 1210, 1152; δ_{H} (400 MHz, CDCl₃) 0.93 (s, 3H, CH₂CH(CH₃)₂), 0.95 (s, 3H, CH₂CH(CH₃)₂), 1.21-1.40 (m, 8H, 5H (cyclohexyl) and CO₂CH₂CH₃), 1.42 (s, 9H, C(CH₃)₃), 1.67-1.84 (m, 5H (cyclohexyl)), 1.86-1.96 (m, 1H, CH₂CH(CH₃)₂), 2.40-2.47 (m, 1H, CH(CH₃)₂), 3.37 (bs, 2H, CH₂CH(CH₃)₂), 4.24 (q, J=7.2 Hz, 2H, CO₂CH₂CH₃), 4.88 (s, 2H, CH₂Ar), 5.04 (s, 2H, CH₂CO₂Et), 5.75 (bs, 1H, NH), 7.08 (d, J=8.1 Hz, 2H, 2CH(Ar)), 7.30 (d, J=8.1 Hz, 2H, 2CH(Ar)), 7.74 (s, 1H, CH(H-8)); LRMS (MS-ES), calcd for C₃₁H₄₄N₆O₄Na [M+Na] m/z=587.34, found 587.51.

### 32. Preparation of ethyl 2-(2-((tert-butoxycarbonyl)(4-cyclohexylbenzyl)amino)-6-(isopentylamino)-9H-purin-9-yl)acetate (Compound ID: 6bl)

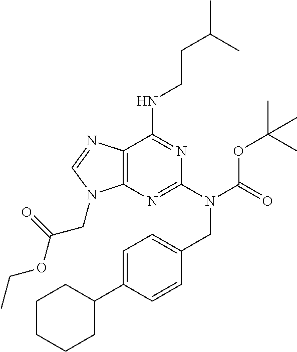 [SMILES: CCOC(=O)Cn1cnc2c(NCCC(C)C)nc(N(Cc3ccc(C4CCCCC4)cc3)C(=O)OC(C)(C)C)nc21]

Purine 5b was treated with isoamylamine according to general procedure B, yielding the final product 6bl as a clear viscous oil (88%): IR (KBr, cm⁻¹) 2924, 2851, 1755, 1704, 1514, 1434, 1385, 1244, 1160, 1023; δ_{H} (400 MHz, CDCl₃) 0.91 (s, 3H, (CH₂)₂CH(CH₃)₂), 0.93 (s, 3H, (CH₂)₂CH(CH₃)₂), 1.25-1.39 (m, 8H, 5H (cyclohexyl) and CO₂CH₂CH₃), 1.41 (s, 9H, C(CH₃)₃), 1.49-1.55 (m, 1H, (CH₂)₂CH(CH₃)₂), 1.65-1.83 (m, 7H, CH₂CH₂CH(CH₃)₂ and 5H (cyclohexyl)), 2.40-2.47 (m, 1H, CH), 3.58 (bs, 2H, CH₂CH₂CH(CH₃)₂), 4.24 (q, J=7.2 Hz, 2H, CO₂CH₂CH₃), 4.87 (s, 2H, CH₂Ar), 5.06 (s, 2H, CH₂CO₂Et), 5.58 (bs, 1H, NH), 7.08 (d, J=8.1 Hz, 2H, 2CH(Ar)), 7.30 (d, J=8.1 Hz, 2H, 2CH(Ar)), 7.73 (s, 1H, CH(H-8)); LRMS (MS-ES), calcd for C₃₂H₄₇N₆O₄ [M+H] m/z=579.36, found 579.48

### 33. Preparation of ethyl 2-(2-((tert-butoxycarbonyl)(4-cyclohexylbenzyl)amino)-6-morpholino-9H-purin-9yl)acetate (Compound ID: 6bm)

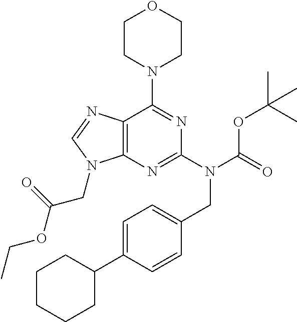 [SMILES: CCOC(=O)Cn1cnc2c(N3CCOCC3)nc(N(Cc3ccc(C4CCCCC4)cc3)C(=O)OC(C)(C)C)nc21]

Purine 5b was treated with morpholine according to general procedure B, yielding the final product 6bm as a white solid (81%): m.p.=166-167° C.; IR (KBr, cm⁻¹) 2925, 2852, 1755, 1698, 1590, 1479, 1440, 1384, 1305, 1240, 1209, 1154, 1116; δ_{H} (400 MHz, CDCl₃) 1.21-1.40 (m, 8H, 5H (cyclohexyl) and CO₂CH₂CH₃), 1.41 (s, 9H, C(CH₃)₃), 1.75-1.84 (m, 5H (cyclohexyl)), 2.40-2.45 (m, 1H, CH), 3.77 (t, J=4.7 Hz, 4H, 2CH₂, (morpholine)), 4.19 (bs, 4H, 2CH₂, (morpholine)), 4.24 (q, J=7.1 Hz, 2H, CO₂CH₂CH₃), 4.88 (s, 2H, CH₂Ar), 5.03 (s, 2H, CH₂CO₂Et), 7.08 (d, J=8.1 Hz, 2H, 2CH(Ar)), 7.27 (d, J=7.5 Hz, 2H, 2CH (Ar)), 7.73 (s, 1H, CH(H-8)); LRMS (MS-ES), calcd for C₃₁H₄₂N₆O₅Na [M+Na] m/z=601.32, found 601.49.

### 34. Preparation of ethyl 2-(2-((tert-butoxycarbonyl)(4-cyclohexylbenzyl)amino)-6-(3-nitrophenoxy)-9H-purin-9-yl)acetate (Compound ID: 6bn)

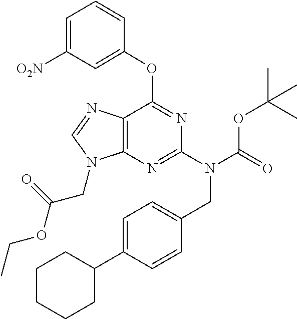 [SMILES: CCOC(=O)Cn1cnc2c(Oc3cccc([N+](=O)[O-])c3)nc(N(Cc3ccc(C4CCCCC4)cc3)C(=O)OC(C)(C)C)nc21]

Purine 5b was treated with 3-nitrophenol according to general procedure D, yielding the final product 6bn as a white solid (82%): m.p.=57.8-79.3° C.; IR (KBr, cm⁻¹) 3546, 3480, 3425, 2930, 2846, 1750, 1708, 1625, 1580, 1545, 1455, 1360; δ_{H} (400 MHz, CDCl₃) 1.19-1.41 (m, 8H, 5H (cyclohexyl) and CO₂CH₂CH₃), 1.33 (s, 9H, C(CH₃)₃), 1.7-1.84 (m, 5H (cyclohexyl)), 2.40-2.45 (m, 1H, CH), 4.27 (q, J=7.1 Hz, 2H, CO₂CH₂CH₃), 4.92 (s, 2H, CH₂Ar), 4.97 (s, 2H, CH₂CO₂Et), 6.98-7.09 (m, 4H, 4 CH(Ar)), 7.52 (t, J=8.2 Hz, 1H, CH(Ar)), 7.61 (d, J=8.1 Hz, 1H, CH(Ar)), 8.01 (s, 1H, CH, (H-8)), 8.09 (d, J=8.1 Hz, 1H, CH(Ar)), 8.2 (t, J=2.2 Hz, 1H, CH(Ar)); LRMS (MS-ES), calcd for C₃₃H₃₈N₆O₇Na [M+Na] m/z=653.28, found 653.39

### 35. Preparation of ethyl 2-(2-((tert-butoxycarbonyl)(4-cyclohexylbenzyl)amino)-6-(4-nitrophenoxy)-9H-purin-9-yl)acetate (Compound ID: 6bo)

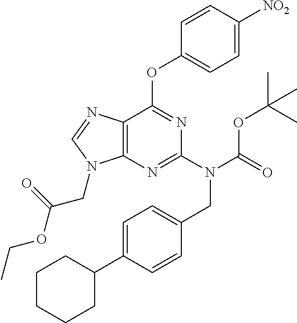 [SMILES: CCOC(=O)Cn1cnc2c(Oc3ccc([N+](=O)[O-])cc3)nc(N(Cc3ccc(C4CCCCC4)cc3)C(=O)OC(C)(C)C)nc21]

Purine 5b was treated with 4-nitrophenol according to general procedure B, yielding the final product 6bo as a clear viscous oil (79%): IR (KBr, cm⁻¹) 3530, 3480, 3425, 2925, 2850, 1770, 1725, 1640, 1625, 1575, 1540, 1350; δ_{H} (400 MHz, CDCl₃) 1.20-1.33 (m, 8H, 5H (cyclohexyl) and CO₂CH₂CH₃), 1.36 (s, 9H, C(CH₃)₃), 1.7-1.83 (m, 5H (cyclohexyl)), 2.43-2.46 (m, 1H, CH), 4.25 (q, J=7.1 Hz, 2H, CO₂CH₂CH₃), 4.94 (s, 2H, CH₂Ar), 4.99 (s, 2H, CH₂CO₂Et), 7.06 (s, 4H, 4 CH(Ar)), 7.45 (d, J=9.0 Hz, 2H, 2CH(Ar)), 8.13 (s, 1H, CH(H-8)), 8.22 (d, J=9.2 Hz, 2H, 2CH(Ar)); LRMS (MS-ES), calcd for C₃₃H₃₈N₆O₇Na [M+Na] m/z=653.28, found 653.30.

### 36. Preparation of ethyl 2-(2-((tert-butoxycarbonyl)(4-cyclohexylbenzyl)amino)-6-((4-fluorophenyl)amino)-9H-purin-9-yl)acetate (Compound ID: 6bp)

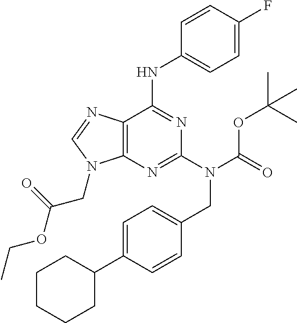 [SMILES: CCOC(=O)Cn1cnc2c(Nc3ccc(F)cc3)nc(N(Cc3ccc(C4CCCCC4)cc3)C(=O)OC(C)(C)C)nc21]

Purine 5b was treated with 4-fluoroaniline according to general procedure C, yielding the final product 6bp as a white solid (56%): m.p.>125° C. (dec); IR (KBr, cm⁻¹) 2926, 2852, 1707, 1593, 1389, 1229, 1157; δ_{H} (400 MHz, CDCl₃) 1.20-1.38 (m, 8H, 5H (cyclohexyl) and CO₂CH₂CH₃), 1.41 (s, 9H, C(CH₃)₃), 1.70-1.85 (m, 5H (cyclohexyl)), 2.43-2.48 (m, 1H, CH), 4.25 (q, J=7.1 Hz, 2H, CO₂CH₂CH₃), 4.91 (s, 2H, CH₂Ar), 5.10 (s, 2H, CH₂CO₂Et), 6.91-6.96 (m, 2H, 2CH(Ar)), 7.11 (d, J=8.0 Hz, 2H, 2CH(Ar)), 7.27 (d, J=8.0 Hz, 2H, 2CH(Ar)), 7.57 (bs, 1H, CH(Ar)), 7.67-7.72 (m, 2H, 2CH(Ar)), 7.83 (s, 1H, CH(H-8)); LRMS (MS-ES), calcd for C₃₃H₃₉FN₆O₄Na [M+Na] m/z=625.30, found 625.43.

### 37. Preparation of ethyl 2-(2-((tert-butoxycarbonyl)(4-cyclohexylbenzyl)amino)-6-((furan-2-ylmethyl)amino)-9H-purin-9-yl)acetate (Compound ID: 6bq)

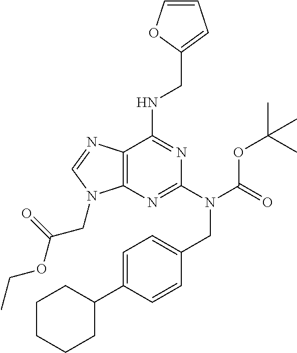 [SMILES: CCOC(=O)Cn1cnc2c(NCc3ccco3)nc(N(Cc3ccc(C4CCCCC4)cc3)C(=O)OC(C)(C)C)nc21]

Purine 5b was treated with furfurylamine according to general procedure B, yielding the final product 6bq as a white solid (87%): m.p.>120 (dec)° C.; IR (KBr, cm⁻¹) 2925, 2851, 1755, 1703, 1481, 1438, 1390, 1237, 1156, 1109; δ_{H} (400 MHz, CDCl₃) 1.25-1.40 (m, 8H, 5H (cyclohexyl) and CO₂CH₂CH₃), 1.41 (s, 9H, C(CH₃)₃), 1.71-1.82 (m, 5H (cyclohexyl)), 2.41-2.47 (m, 1H, CH), 4.23 (q, J=7.1 Hz, 2H, CO₂CH₂CH₃), 4.76 (bs, 2H, CH₂ (furfuryl)), 4.88 (s, 2H, CH₂Ar), 5.07 (s, 2H, CH₂CO₂Et), 6.01 (bs, 1H, NH (furfuryl)), 6.19-6.20 (m, 1H, CH (furfuryl)), 6.29-6.30 (m, 1H, CH (furfuryl)), 7.09 (d, J=8.1 Hz, 2H, 2 CH(Ar)), 7.29 (d, J=8.0 Hz, 2H, 2CH(Ar)), 7.34-7.35 (m, 1H, CH (furfuryl)), 7.75 (s, 1H, CH(H-8)); LRMS (MS-ES), calcd for C₃₂H₄₁N₆O₅ [M+H] m/z=589.31, found 589.43.

### 38. Preparation of ethyl 2-(2-((tert-butoxycarbonyl)(4-cyclohexylbenzyl)amino)-6-(propylamino)-9H-purin-9-yl)acetate (Compound ID: 6bs)

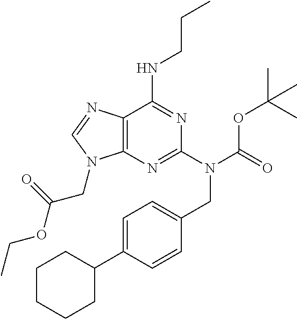 [SMILES: CCCNc1nc(N(Cc2ccc(C3CCCCC3)cc2)C(=O)OC(C)(C)C)nc2c1ncn2CC(=O)OCC]

Purine 5b was treated with n-propylamine according to general procedure B, yielding the final product 6bs as a clear viscous oil (77%): IR (KBr, cm⁻¹) 2926, 1703, 1384, 1213, 1156; δ_{H} (400 MHz, CDCl₃) 0.95 (t, J=7.4 Hz, 3H, NHCH₂CH₂CH₃), 1.20-1.33 (m, 8H, 5H (cyclohexyl) and CO₂CH₂CH₃), 1.42 (s, 9H, C(CH₃)₃), 1.64 (m, 2H, NHCH₂CH₂CH₃) 1.7-1.83 (m, 5H (cyclohexyl)), 2.40-2.46 (m, 1H, CH), 3.52 (bs, 2H, NHCH₂CH₂CH₃), 4.24 (q, J=7.1 Hz, 2H, CO₂CH₂CH₃), 4.87 (s, 2H, CH2Ar), 5.05 (s, 2H, CH₂CO₂Et), 5.70 (bs, 1H, NH), 7.08 (d, J=8.1 Hz, 2H, 2CH(Ar)), 7.30 (d, J=8.0 Hz, 2H, 2CH(Ar)), 7.73 (s, 1H, CH(H-8)); LRMS (MSES), calcd for C₃₀H₄₃N₆O₄ [M+H] m/z=551.33, found 551.54.

### 39. Preparation of ethyl 2-(2-((tert-butoxycarbonyl)(4-cyclohexylbenzyl)amino)-6-(hexylamino)-9H-purin-9-yl)acetate (Compound ID: 6bt)

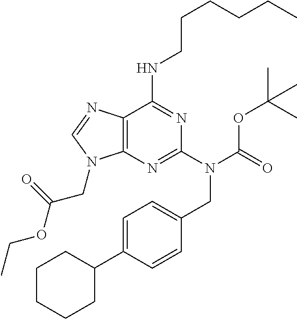 [SMILES: CCCCCCNc1nc(N(Cc2ccc(C3CCCCC3)cc2)C(=O)OC(C)(C)C)nc2c1ncn2CC(=O)OCC]

Purine 5b was treated with n-hexylamine according to general procedure B, yielding the final product 6bt as a white solid (81%): m.p.=115-121° C.; IR (KBr, cm⁻¹) 3546, 3490, 3425, 2925, 2860, 1770, 1700, 1625, 1530, 1440, 1360, 1246; δ_{H} (400 MHz, CDCl₃) 0.88 (t, J=7.2 Hz, 3H, NH(CH₂)₄CH₃), 1.16-1.34 (m, 14H, 5H (cyclohexyl) and 6H NH(CH₂)₂CH₂CH₂CH₂CH₃ and CO₂CH₂CH₃), 1.42 (s, 9H, C(CH₃)₃), 1.51-1.76 (m, 7H, 5H (cyclohexyl) and NHCH₂CH₂(CH₂)₃CH3)), 2.40-2.46 (m, 1H, CH), 3.54 (bs, 2H, NHCH₂(CH₂)₄CH₃), 4.24 (q, J=7.1 Hz, 2H, CO₂CH₂CH₃), 4.87 (s, 2H, CH₂Ar), 5.05 (s, 2H, CH₂CO₂Et), 5.93 (bs, 1H, NH), 7.08 (d, J=8.1 Hz, 2H, 2CH(Ar)), 7.30 (d, J=8.0 Hz, 2H, 2CH(Ar)), 7.74 (s, 1H, CH(H-8)); LRMS (MS-ES), calcd for C₃₃H₄₉N₆O₄ [M+H] m/z=593.37, found 593.51.

### 40. Preparation of ethyl 2-(6-(3-bromophenoxy)-2-((tert-butoxycarbonyl)(4-cyclohexylbenzyl)amino)-9H-purin-9-yl)acetate (Compound ID: 6bu)

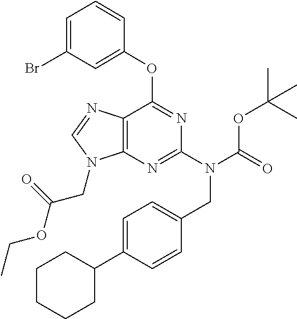 [SMILES: CCOC(=O)Cn1cnc2c(Oc3cccc(Br)c3)nc(N(Cc3ccc(C4CCCCC4)cc3)C(=O)OC(C)(C)C)nc21]

Purine 5b was treated with 3-bromophenol according to general procedure D, yielding the final product 6bu as a clear viscous oil (76%): IR (KBr, cm⁻¹) 3546, 3480, 3425, 3230, 2930, 2840, 1750, 1710, 1625, 1580, 1470, 1400; δ_{H} (400 MHz, CDCl₃) 1.14-1.34 (m, 8H, 5H (cyclohexyl) and CO₂CH₂CH₃), 1.35 (s, 9H, C(CH₃)₃), 1.67-1.86 (m, 5H, (cyclohexyl)), 2.38-2.49 (m, 1H, CH), 4.26 (q, J=7.1 Hz, 2H, CO₂CH₂CH₃), 4.93 (s, 2H, CH₂Ar), 4.95 (s, 2H, CH₂CO₂Et), 7.12-7.07 (m, 4H, 4 CH(Ar)), 7.17-7.21 (m, 1H, CH(Ar)), 7.23-7.28 (m, 1H, CH(Ar)), 7.35-7.41 (m, 1H, CH(Ar)), 7.49 (t, J=2.0 Hz, CH, (Ar)), 7.98 (s, 1H, CH, (H-8)); LRMS (MS-ES), calcd for C₃₃H₃₉BrN₅O₅ [M+H] m/z=664.21, found 664.28.

### 41. Preparation of ethyl 2-(2-((tert-butoxycarbonyl)(4-cyclohexylbenzyl)amino)-6-(4-fluorophenoxy)-9H-purin-9-yl)acetate (Compound ID: 6bv)

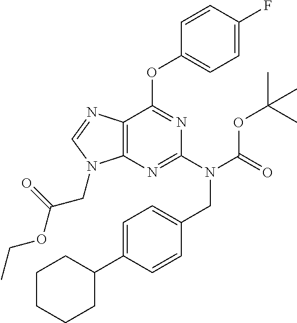 [SMILES: CCOC(=O)Cn1cnc2c(Oc3ccc(F)cc3)nc(N(Cc3ccc(C4CCCCC4)cc3)C(=O)OC(C)(C)C)nc21]

Purine 5b was treated with 4-fluorophenol according to general procedure D, yielding the final product 6bv as a white solid (67%): m.p.=93-97° C.; IR (KBr, cm⁻¹) 3546, 3470, 3408, 3230, 2925, 2846, 1760, 1700, 1625, 1500, 1440, 1400; δ_{H} (400 MHz, CDCl₃) 1.14-1.32 (m, 8H, 5H (cyclohexyl) and CO₂CH₂CH₃), 1.35 (s, 9H, C(CH₃)₃), 1.70-1.84 (m, 5H (cyclohexyl)), 2.40-2.47 (m, 1H, CH), 4.26 (q, J=7.1 Hz, 2H, CO₂CH₂CH₃), 4.90 (s, 2H, CH₂Ar), 4.95 (s, 2H, CH₂CO₂Et), 7.02-7.07 (m, 6H, 6 CH(Ar)), 7.18-7.21 (m, 2H, 2CH(Ar)), 7.98 (s, 1H, CH, (H-8)); LRMS (MS-ES), calcd for C₃₃H₃₉FN₅O₅ [M+H] m/z=604.29, found 604.37.

### 42. Preparation of ethyl 2-(2-((tert-butoxycarbonyl)(4-cyclohexylbenzyl)amino)-6-(perfluorophenoxy)-9H-purin-9-yl)acetate (Compound ID: 6bw)

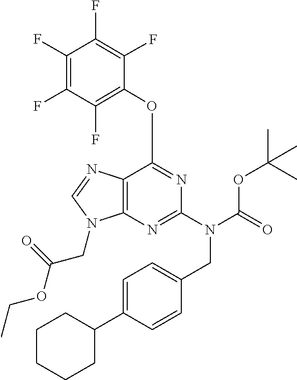 [SMILES: CCOC(=O)Cn1cnc2c(Oc3c(F)c(F)c(F)c(F)c3F)nc(N(Cc3ccc(C4CCCCC4)cc3)C(=O)OC(C)(C)C)nc21]

Purine 5b was treated with pentafluorophenol according to general procedure D, yielding the final product 6bw as a white solid (75%): m.p.=91-110° C.; IR (KBr, cm⁻¹) 3546, 3475, 3425, 2905, 2860, 1760, 1730, 1630, 1560, 1400, 1370, 1230; δ_{H} (400 MHz, CDCl₃) 1.21-1.34 (m, 8H, 5H (cyclohexyl) and CO₂CH₂CH₃), 1.37 (s, 9H, C(CH₃)₃), 1.71-1.84 (m, 5H (cyclohexyl)), 2.40-2.47 (m, 1H, CH), 4.28 (q, J=7.1 Hz, 2H, CO₂CH₂CH₃), 4.88 (s, 2H, CH₂Ar), 4.98 (s, 2H, CH₂CO₂Et), 6.98 (d, J=8.2 Hz, 2H, 2CH(Ar)), 7.04 (d, J=8.2 Hz, 2H, 2CH(Ar)), 8.04 (s, 1H, CH, (H-8)); LRMS (MS-ES), calcd for C₃₃H₃₄F₅N₅O₅Na [M+Na] m/z=698.25, found 698.34

### 43. Preparation of ethyl 2-(2-((tert-butoxycarbonyl)(4-cyclohexylbenzyl)amino)-6-phenoxy-9H-purin-9-yl)acetate (Compound ID: 6bx)

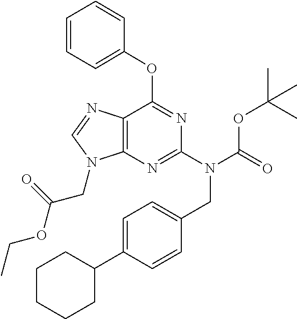 [SMILES: CCOC(=O)Cn1cnc2c(Oc3ccccc3)nc(N(Cc3ccc(C4CCCCC4)cc3)C(=O)OC(C)(C)C)nc21]

Purine 5b was treated with phenol according to general procedure D, yielding the final product 6bx as a white solid (79%): m.p.=104-110° C.; IR (KBr, cm⁻¹) 3546, 3470, 3425, 2940, 2850, 1750, 1700, 1630, 1570, 1490, 1395, 1230; δ_{H} (400 MHz, CDCl₃) 1.11-1.40 (m, 8H, 5H (cyclohexyl) and CO₂CH₂CH₃), 1.34 (s, 9H, C(CH₃)₃), 1.70-1.83 (m, 5H (cyclohexyl)), 2.40-2.47 (m, 1H, CH), 4.26 (q, J=7.1 Hz, 2H, CO₂CH₂CH₃), 4.91 (s, 2H, CH₂Ar), 4.95 (s, 2H, CH₂CO₂Et), 7.01-7.07 (m, 4H, 4 CH(Ar)), 7.22-7.26 (m, 3H, 3CH (Ar)), 7.37-7.42 (m, 2H, 2CH(Ar)), 7.97 (s, 1H, CH(H-8)); LRMS (MS-ES), calcd for C₃₃H₄₀N₅O₅ [M+H] m/z=586.30, found 586.43.

### 44. Preparation of 2-(6-(benzylamino)-2-((tert-butoxycarbonyl)(pentyl)amino)-9H-purin-9-yl)acetic acid (Compound ID: 7aa)

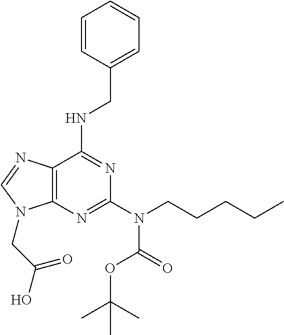 [SMILES: CCCCCN(C(=O)OC(C)(C)C)c1nc(NCc2ccccc2)c2ncn(CC(=O)O)c2n1]

Purine 6aa was treated according to general procedure E, to yield lyophilized product 7aa as a white solid (72%): m.p.>198 (dec)° C.; IR (KBr, cm⁻¹) 3549, 3476, 3414, 2959, 1707, 1624, 1390, 1367, 1355, 1300, 1271, 1217; δ_{H} (400 MHz, DMSO-d₆) 0.77 (t, J=7.0 Hz, 3H, (CH₂)₄CH₃), 1.09-1.23 (m, 4H, (CH₂)₂CH₂CH₂CH₃), 1.35 (s, 9H, C(CH₃)₃), 1.52-1.59 (m, 2H, CH₂CH₂(CH₂)₂H₃), 3.59 (t, J=6.9 Hz, 2H, CH₂(CH₂)₃CH₃), 4.65 (bs, 2H, CH₂Ar), 4.89 (s, 2H, CH₂CO₂H), 7.20 (t, J=7.2 Hz, 1H, CH(Ar)), 7.28 (t, J=7.5 Hz, 2H, 2 CH(Ar)), 7.30-7.35 (m, 2H, 2CH(Ar)), 8.07 (s, 1H, CH(H-8)), 8.43 (m, 1H, NH) 13.26 (vbs, 1H, CH₂CO₂H); δC (100 MHz, DMSO-d₆) 13.8, 21.7, 27.8, 27.9, 28.3, 43.0, 43.7, 47.3, 79.3, 115.7, 126.5, 127.0, 127.1, 128.0, 140.0, 141.3, 149.8, 153.9, 155.2, 169.2; HRMS (MS-ES), calcd for C₂₄H₃₃N₆O₄ [M+H] m/z=469.2562, found 469.2557; rpHPLC t_{R}: condition (I) 14.246 (II) 39.742 min, purity 91.2% and 93.4%.

### 45. Preparation of 2-(6-(benzyl(methyl)amino)-2-((tert-butoxycarbonyl)(pentyl)amino)-9H-purin-9-yl)acetic acid (Compound ID: 7ab)

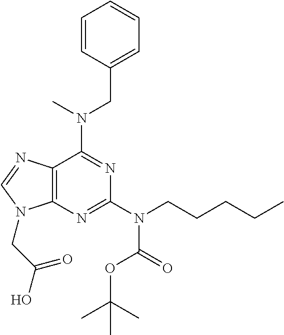 [SMILES: CCCCCN(C(=O)OC(C)(C)C)c1nc(N(C)Cc2ccccc2)c2ncn(CC(=O)O)c2n1]

Purine 6ab was treated according to general procedure E, to yield lyophilized product lab as a white solid (87%): m.p.=116-127° C.; IR (KBr, cm⁻¹) 3294, 2924, 2444, 2356, 1399, 1198; δ_{H} (400 MHz, DMSO-d₆) 0.82 (m, 3H, (CH₂)₄CH₃), 1.21-1.29 (m, 4H, (CH₂)₂CH₂CH₂CH₃), 1.38 (s, 9H, C(CH₃)₃), 1.43-1.58 (m, 2H, (CH₂)₃CH₂CH₃), 3.15-3.60 (bm, 3H, NCH₃), 3.60-3.70 (m, 2H, CH₂(CH₂)₃CH₃), 4.78 (s, 2H, CH₂CO₂H), 4.86-5.55 (bm, 2H, CH₂Ar), 7.24-7.31 (m, 5H, 2CH(Ar)), 7.71 (s, 1H, CH(H-8)), 13.23 (vbs, 1H, CH₂CO₂₁H); HRMS (MS-ES), calcd for C₂₅H₃₅N₆O₄ [M+H] m/z=483.2701, found 483.2714; rpHPLC tR: condition (I) 15.031 (II) 38.982 min, purity 90.0% and 90.4%.

### 46. Preparation of 2-(2((tert-butoxycarbonyl)(pentyl)amino)-6-(phenylamino)-9H-purin-9-yl)acetic acid (Compound ID: 7ac)

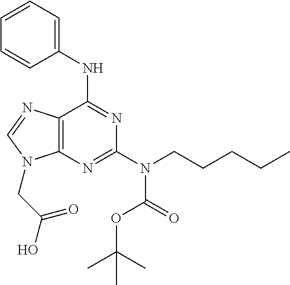 [SMILES: CCCCCN(C(=O)OC(C)(C)C)c1nc(Nc2ccccc2)c2ncn(CC(=O)O)c2n1]

Purine 6ac was treated according to general procedure E, to yield lyophilized product lac as an off-white solid (75%): m.p.>139° C. (dec); IR (KBr, cm⁻¹) 3424, 2958, 1704, 1442, 1364, 1164; δ_{H} (400 MHz, DMSO-d₆) 0.81 (t, J=7.0 Hz, 3H, (CH₂)₄CH₃), 1.23-1.27 (m, 4H, (CH₂)₂CH₂CH₂CH₃), 1.39 (s, 9H, C(CH₃)₃), 1.52-1.59 (m, 2H, (CH₂)₃CH₂CH₃), 3.72 (t, J=7.4 Hz, 2H, CH₂(CH₂)₃CH₃), 4.90 (s, 2H, CH₂CO₂H), 7.03 (t, J=7.3 Hz, 1H, CH (Ar)), 7.29 (t, J=7.9 Hz, 2H, 2CH(Ar)), 7.96 (d, J=7.5 Hz, 2H, 2CH(Ar)), 8.21 (s, 1H, CH(H-8)), 9.93 (s, 1H, NH); δC (100 MHz, DMSO-d₆) 13.8, 21.7, 27.7, 27.9, 28.3, 44.2, 47.5, 79.6, 116.6, 120.4, 122.4, 128.1, 139.5, 142.3, 150.6, 151.5, 153.7, 154.7, 169.1. HRMS (MS-ES), calcd for C₂₃H₃₁N₆O₄ [M+H] m/z=455.2387, found 455.2401; rpHPLC t_{R}: condition (I) 14.988 (II) 38.416 min, purity 93.1% and 98.2%.

### 47. Preparation of 2-(2-((tert-butoxycarbonyl)(pentyl)amino)-6-((furan-2-ylmethyl)(methyl)amino)-9H-purin-9-yl)acetic acid (Compound ID: 7ad)

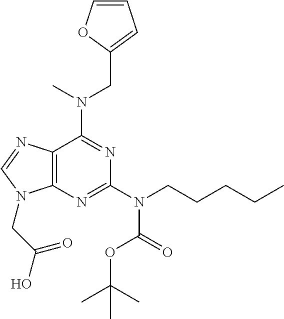 [SMILES: CCCCCN(C(=O)OC(C)(C)C)c1nc(N(C)Cc2ccco2)c2ncn(CC(=O)O)c2n1]

Purine 6ad was treated according to general procedure E, to yield product lad as a clear viscous oil (92%): IR (KBr, cm⁻¹) 3549, 3471, 3415, 3120, 2958, 2925, 2855, 1703, 1637, 1618, 1591, 1460; δ_{H} (400 MHz, CDCl₃) 0.83-0.88 (m, 3H, (CH₂)₄CH₃), 1.25-1.34 (m, 4H, (CH₂)₂CH₂CH₂CH₃), 1.48 (s, 9H, C(CH₃)₃), 1.56-1.69 (m, 2H, CH₂CH₂(CH₂)₂CH₃), 3.50 (vbs, 3H, CH₃(furfuryl)), 3.80 (t, J=7.6 Hz, 2H, CH₂(CH₂)₃CH₃), 4.86 (s, 2H, CH₂CO₂H), 5.22 (vbs, 2H, CH₃(furfuryl)), 6.29-6.33 (m, 2H, CH (furfuryl)), 7.35-7.36 (m, 1H, CH (furfuryl)), 7.81 (s, 1H, CH(H-8)); LRMS (MS-ES), calcd for C₂₃H₃₁N₆O₅ [M−H] m/z=471.24, found 471.25.

### 48. Preparation of 2-(2-((tert-butoxycarbonyl)(pentyl)amino)-6-(cyclopentylamino)-9H-purin-9-yl)acetic acid (Compound ID: 7af)

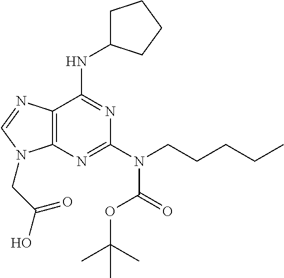 [SMILES: CCCCCN(C(=O)OC(C)(C)C)c1nc(NC2CCCC2)c2ncn(CC(=O)O)c2n1]

Purine 6ae was treated according to general procedure E, to yield product Tae as a white solid (95%): m.p.=140-146° C.; IR (KBr, cm⁻¹) 3551, 3474, 3413, 2959, 2929, 2871, 1713, 1619, 1475, 1387, 1365, 1273; δ_{H} (400 MHz, CDCl₃) 0.82-0.90 (m, 3H, (CH₂)₄CH₃), 1.23-1.32 (m, 4H, (CH₂)₂CH₂CH₂CH₃), 1.49 (s, 9H, C(CH₃)₃), 1.56-1.80 (m, 8H, CH₂CH₂(CH₂)₂CH₃ and 3CH₂ (cyclopentyl)), 2.00-2.11 (m, 2H, CH₂ (cyclopentyl)), 3.80-3.86 (m, 2H, CH₂(CH₂)₃CH₃), 4.45 (bs, 1H, CH (cyclopentyl)), 4.89 (s, 2H, CH₂CO₂H), 7.10 (s, 1H, NH), 7.90 (s, 1H, CH(H-8)); LRMS (MS-ES), calcd for C₂₂H₃₃N₆O₄ [M−H] m/z=445.26, found 445.27.

### 49. Preparation of 2-(2-((tert-butoxycarbonyl)(pentyl)amino)-6-(cyclohexylamino)-9H-purin-9-yl)acetic acid (Compound ID: 7af)

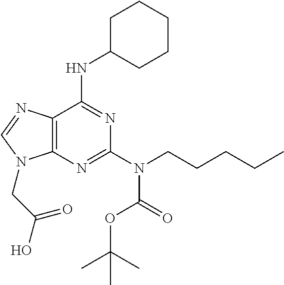 [SMILES: CCCCCN(C(=O)OC(C)(C)C)c1nc(NC2CCCCC2)c2ncn(CC(=O)O)c2n1]

Purine 6af was treated according to general procedure E, to yield product 7af as a white solid (70%): m.p.=140-158° C.; IR (KBr, cm⁻¹) 3550, 3413, 2930, 2855, 1741, 1707, 1618, 1450, 1382, 1366, 1257, 1242; δ_{H} (400 MHz, CDCl₃) 0.82-0.90 (m, 3H, (CH₂)₄CH₃), 1.17-1.42 (m, 10H, (CH₂)₂CH₂CH₂CH₃ and 3CH₂(cyclohexyl)), 1.49 (s, 9H, C(CH₃)₃), 1.60-1.72 (m, 2H, CH₂CH₂(CH₂)₂CH₃), 1.75-1.83 (m, 2H, CH₂ (cyclohexyl)), 2.00-2.07 (m, 2H, CH₂ (cyclohexyl)), 3.78-3.85 (m, 2H, CH₂(CH₂)₃CH₃), 4.03 (bs, 1H, CH (cyclohexyl)), 4.89 (s, 2H, CH₂CO₂H), 6.90 (bs, 1H, NH), 7.89 (s, 1H, CH(H-8)); LRMS (MS-ES), calcd for C₂₃H₃₅N₆O₄ [M−H] m/z=459.28, found 459.35.

### 50. Preparation of 2-(2-((tert-butoxycarbonyl)(pentyl)amino)-6-(ethyl(methyl)amino)-9H-purin-9-yl)acetic acid (Compound ID: 7ag)

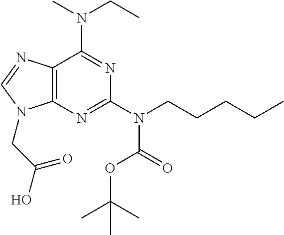 [SMILES: CCCCCN(C(=O)OC(C)(C)C)c1nc(N(C)CC)c2ncn(CC(=O)O)c2n1]

Purine 6ag was treated according to general procedure E, to yield product 7ag as a clear oil (94%): IR (KBr, cm⁻¹) 3414, 2961, 2931, 2859, 1723, 1596, 1492, 1456, 1433, 1418, 1380, 1296; δ_{H} (400 MHz, CDCl₃) 0.89 (t, J=6.9 Hz, 3H, (CH₂)₄CH₃), 1.25-1.36 (m, 7H, (CH₂)₂CH₂CH₂CH₃ and NCH₂CH₃), 1.52 (s, 9H, C(CH₃)₃), 1.68 (p, 7.4 Hz, 2H, CH₂CH₂(CH₂)₂CH₃), 3.21-3.76 (bm, 3H, NCH), 3.85-3.91 (m, 2H, CH₂(CH₂)₃CH₃), 4.28 (bs, 2H, NCH₂CH₃), 5.00 (s, 2H, CH₂CO₂H), 7.74 (s, 1H, CH(H-8)); LRMS (MS-ES), calcd for C₂₀H₃₁N₆O₄ [M−H] m/z=419.25, found 419.36.

### 51. Preparation of 2-(2-((tert-butoxycarbonyl)(pentyl)amino)-6-(isopropylamino)-9H-purin-9-yl)acetic acid (Compound ID: 7ah)

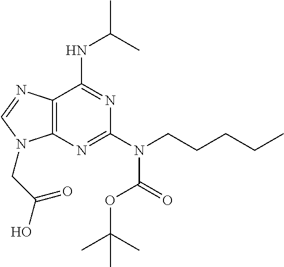 [SMILES: CCCCCN(C(=O)OC(C)(C)C)c1nc(NC(C)C)c2ncn(CC(=O)O)c2n1]

Purine 6ah was treated according to general procedure E, to yield product 7ah as a white solid (98%): m.p.>146° C. (dec); IR (KBr, cm⁻¹) 3413, 3314, 2976, 2929, 1714, 1613, 1468, 1403, 1384, 1367, 1325, 1275; δ_{H} (400 MHz, CDCl₃) 0.82-0.91 (m, 3H, (CH₂)₄CH₃), 1.20-1.41 (m, 10H, (CH₂)₂CH₂CH₂CH₃ and CH(CH₃)₂), 1.52 (s, 9H, C(CH₃)₃), 1.58-1.72 (m, 2H, CH₂CH₂(CH₂)₂CH₃), 3.80-3.90 (m, 2H, CH₂(CH₂)₃CH₃), 4.33 (bs, 1H, CH(CH₃)₂), 4.92 (s, 2H, CH₂CO₂H), 7.26 (bs, 1H, NH), 7.96 (bs, 1H, CH(H-8)); LRMS (MS-ES), calcd for C₂₀H₃₁N₆O₄ [M−H] m/z=419.25, found 419.36.

### 52. Preparation of 2-(6-(allylamino)-2-((tert-butoxycarbonyl)(pentyl)amino)-9H-purin-9-yl)acetic acid (Compound ID: 7ai)

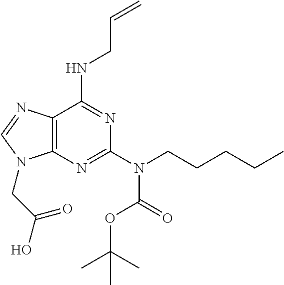 [SMILES: C=CCNc1nc(N(CCCCC)C(=O)OC(C)(C)C)nc2c1ncn2CC(=O)O]

Purine 6ai was treated according to general procedure E, to yield product 7ai as a white solid (96%): m.p.=174-176° C.; IR (KBr, cm⁻¹) 3550, 3475, 3414, 2931, 1711, 1619, 1477, 1445, 1403, 1386, 1365, 1349; δ_{H} (400 MHz, CDCl₃) 0.86 (t, J=6.8 Hz, 3H, (CH₂)₄CH₃), 1.26-1.34 (m, 4H, (CH₂)₂CH₂CH₂CH₃), 1.49 (s, 9H, C(CH₃)₃), 1.64 (p, J=7.3 Hz, 2H, CH₂CH₂(CH₂)₂CH₃), 3.82 (t, J=7.6 Hz, 2H, CH₂(CH₂)₃CH₃), 4.24 (bs, 2H, CH₂CHCH₂), 4.89 (s, 2H, CH₂CO₂H), 5.16 (d, J=10.1 Hz, 1H, CH₂CHCH₂), 5.30 (d, J=17.4 Hz, 1H, CH₂CHCH₂), 5.91-6.03 (m, 1H, CH₂CHCH₂), 7.86 (s, 1H, CH(H-8)); LRMS (MS-ES), calcd for C₂₀H₂₉N₆O₄ [M−H] m/z=417.23, found 417.37.

### 53. Preparation of 2-(2-((tert-butoxycarbonyl)(pentyl)amino)-6-(isobutylamino)-9H-purin-9-yl)acetic acid (Compound ID: 7aj)

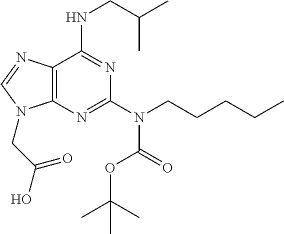 [SMILES: CCCCCN(C(=O)OC(C)(C)C)c1nc(NCC(C)C)c2ncn(CC(=O)O)c2n1]

Purine 6aj was treated according to general procedure E, to yield product Taj as a white solid (96%): m.p.=160-162° C.; IR (KBr, cm⁻¹) 3413, 3315, 2958, 2928, 2872, 1704, 1621, 1597, 1478, 1430, 1404, 1383; δ_{H} (400 MHz, CDCl₃) 0.82-0.91 (m, 3H, (CH₂)₄CH₃), 0.96-1.02 (m, 6H, CH(CH₃)₂), 1.20-1.35 (m, 4H, (CH₂)₂CH₂CH₂CH₃), 1.51 (s, 9H, C(CH₃)₃), 1.61-1.73 (m, 2H, CH₂CH₂(CH₂)₂CH₃), 1.94-2.08 (m, 1H, CH(CH₃)₂), 3.36-3.44 (m, 2H, CH₂CH(CH₃)₂), 3.78-3.92 (m, 2H, CH2(CH₂)₃CH₃), 4.92 (s, 2H, CH₂CO₂H), 7.94 (s, 1H, CH (H-8)); LRMS (MS-ES), calcd for C₂₁H₃₃N₆O₄ [M−H] m/z=433.26, found 433.37.

### 54. Preparation of 2-(2-((tert-butoxycarbonyl)(pentyl)amino)-6-(butyl(methyl)amino)-9H-purin-9-yl)acetic acid (Compound ID: 7ak)

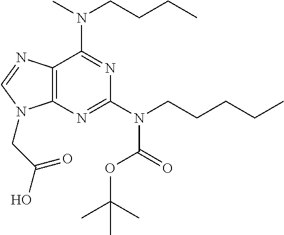 [SMILES: CCCCCN(C(=O)OC(C)(C)C)c1nc(N(C)CCCC)c2ncn(CC(=O)O)c2n1]

Purine 6ak was treated according to general procedure E, to yield product 7ak as a clear viscous oil (89%): IR (KBr, cm⁻¹) 3549, 3476, 3414, 2958, 2926, 1702, 1637, 1618, 1384; δ_{H} (400 MHz, CDCl₃) 0.84-0.89 (m, 3H, (CH₂)₄CH₃), 0.94 (t, J=7.3 Hz, 3H, (CH₂)₃CH₃), 1.25-1.43 (m, 6H, CH₂CH₂CH₂CH₃ and (CH₂)₂CH₂CH₂CH₃), 1.47 (s, 9H, C(CH₃)₃), 1.59-1.70 (m, 4H, CH₂CH₂CH₂CH₃ and CH₂CH₂(CH₂)₂CH₃), 3.14-3.86 (bm, 4H, CH₂(CH₂)₂CH₃ and CH₂(CH₂)₃CH₃), 3.79 (t, J=7.6 Hz, 2H, CH₂(CH₂)₃CH₃), 4.84 (s, 2H, CH₂CO₂H), 7.78 (s, 1H, CH(H-8)); LRMS (MS-ES), calcd for C₂₂H₃₅N₆O₄ [M−H] m/z=447.28, found 447.38

### 55. Preparation of 2-(2-((tert-butoxycarbonyl)(pentyl)amino)-6-(isopentylamino)-9H-purin-9-yl)acetic acid (Compound ID: 7al)

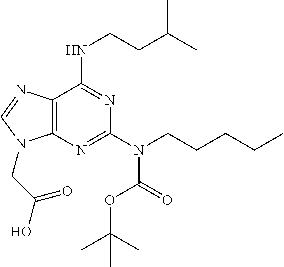 [SMILES: CCCCCN(C(=O)OC(C)(C)C)c1nc(NCCC(C)C)c2ncn(CC(=O)O)c2n1]

Purine 6al was treated according to general procedure E, to yield product 7al as a white solid (67%): m.p.=169-173° C.; IR (KBr, cm⁻¹) 3550, 3414, 3322, 2957, 2930, 2871, 1741, 1708, 1621, 1468, 1383, 1365; δH (400 MHz, CDCl₃) 0.82-0.90 (m, 3H, (CH₂)₄CH₃), 0.92 (s, 3H, (CH₂)₂CH(CH₃)₂), 0.94 (s, 3H, (CH₂)₂CH(CH₃)₂), 1.19-1.35 (m, 4H, (CH₂)₂CH₂CH₂CH₃), 1.50 (s, 9H, C(CH₃)₃), 1.54-1.76 (m, 5H, CH₂CH₂(CH₂)₂CH₃ and CH₂CH₂CH(CH₃)₂), 3.57 (bs, 2H, (CH₂)(CH₂)(CH₃)₂), 3.80-3.89 (m, 2H, (CH₂)(CH₂)₃CH₃), 4.05 (bs, 1H, NH), 4.91 (s, 2H, CH₂)CO₂H), 7.92 (bs, 1H, CH(H-8)); LRMS (MS-ES), calcd for C₂₂H₃₅N₆O₄ [M−H] m/z=447.28, found 447.38.

### 56. Preparation of 2-(2-((tert-butoxycarbonyl)(pentyl)amino)-6-(morpholino)-9H-purin-9-yl)acetic acid (Compound ID: 7am)

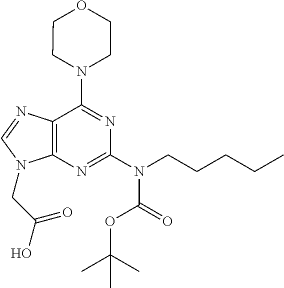 [SMILES: CCCCCN(C(=O)OC(C)(C)C)c1nc(N2CCOCC2)c2ncn(CC(=O)O)c2n1]

Purine 6 am was treated according to general procedure E, to yield product 7 am as a lyophilized white powder (94%): m.p.>143 (dec); IR (KBr, cm⁻¹) 2959, 2929, 2857, 1588, 1478, 1446, 1388, 1304, 1266, 1241, 1137; δ_{H} (400 MHz, DMSO-d₆) 0.87 (t, J=6.9 Hz, 3H, (CH₂)4CH₃), 1.17-1.26 (m, 4H, (CH₂)₂CH₂CH₂CH₃), 1.38 (s, 9H, C(CH₃)₃), 1.52 (p, J=7.3 Hz, 2H, CH₂CH₂(CH₂)₂CH₃), 3.64-3.75 (m, 6H, CH₂(CH₂)₃CH₃ and 2CH₂ (morpholine)), 4.17 (bs, 4H, 2CH₂ (morpholine)), 4.76 (s, 2H, CH₂CO₂H), 8.07 (s, 1H, CH(H-8)); δ_{C} (100 MHz, DMSO-d₆) 13.8, 21.6, 27.8, 27.9, 28.3, 44.6, 45.0, 47.3, 66.1, 79.3, 115.9, 141.0, 151.8, 152.9, 153.8, 154.5, 169.2; HRMS (MS-ES), calcd for C₂₁H₃₃N₆O₅ [M+H] m/z=449.2506, found 449.2497; rpHPLC t_{R}: condition (I) 13.883 (II) 32.404 min, purity 90.8% and 90.9%.

### 57. Preparation of 2-(2-((tert-butoxycarbonyl)(pentyl)amino)-6-(3-nitrophenoxy)-9H-purin-9-yl)acetic acid (Compound ID: 7an)

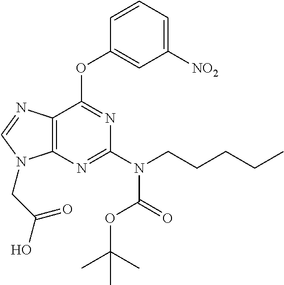 [SMILES: CCCCCN(C(=O)OC(C)(C)C)c1nc(Oc2cccc([N+](=O)[O-])c2)c2ncn(CC(=O)O)c2n1]

Purine 6an was treated according to general procedure E, to yield product 7an as a lyophilized white solid (62%): m.p.>85° C. (dec); IR (KBr, cm⁻¹) 3595, 3385, 3115, 2945, 1533, 1246; δ_{H} (400 MHz, DMSO-d₆) 0.75 (t, J=7.2 Hz, 3H, (CH₂)₄CH₃), 0.97-1.13 (m, 4H, (CH₂)₂CH₂CH₂CH₃), 1.29 (s, 9H, C(CH₃)₃), 1.31-1.38 (m, 2H, (CH₂)₃CH₂CH₃), 3.52 (t, J=7.4 Hz, 2H, CH₂(CH₂)₃CH₃), 5.03 (s, 2H, CH₂CO₂H), 7.78 (t, J=8.1 Hz, 1H, CH(Ar)), 7.85-7.88 (m, 1H, CH(Ar)), 8.18-8.21 (m, 1H, CH(Ar)), 8.28 (t, J=2.2 Hz, 1H, CH(Ar), 8.43 (s, 1H, CH(H-8)), 13.44 (vbs, 1H, CH₂CO₂H); δ_{C} (100 MHz, DMSO-d₆) 13.7, 21.6, 27.7, 28.2, 28.5, 44.3, 47.7, 80.2, 116.8, 117.6, 120.5, 129.1, 130.8, 145.4 148.3, 152.3, 153.2, 154.0, 154.2, 158.2, 168.9; HRMS (MS-ES), calcd for C₂₃H₂₉N₆O₇ [M+H] m/z=501.2095, found 501.2092; rpHPLC t_{R}: condition (I) 14.230 (II) 36.038 min, purity 98.3% and 97.16%.

### 58. Preparation of 2-(2-((tert-butoxycarbonyl)(pentyl)amino)-6-(3-nitrophenoxy)-9H-purin-9-yl)acetic acid (Compound ID: 7ao)

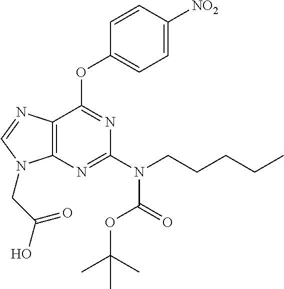 [SMILES: CCCCCN(C(=O)OC(C)(C)C)c1nc(Oc2ccc([N+](=O)[O-])cc2)c2ncn(CC(=O)O)c2n1]

Purine 6ao was treated according to general procedure E, to yield product Tao as a lyophilized white solid (70%): m.p.>194° C. (dec); IR (KBr, cm⁻¹) 3119, 2959, 2931, 2861, 1723, 1579, 1525, 1489, 1407, 1347, 1252, 1209, 1137, 1045; δ_{H} (400 MHz, DMSO-d₆) 0.74 (t, J=7.2 Hz, 3H, (CH₂)₄CH₃), 0.99-1.17 (m, 4H, (CH₂)₂CH₂CH₂CH₃), 1.31 (s, 9H, C(CH₃)₃), 1.34-1.39 (m, 2H, CH₂CH₂(CH₂)2CH₃), 2.81-3.03 (m, 2H, CH₂(CH₂)₃CH₃), 5.04 (s, 2H, CH₂CO₂H), 7.66 (d, J=9.0 Hz, 2H, 2CH(Ar)), 8.43 (s, 1H, CH(H-8)), 8.35 (d, J=9.1 Hz, 2H, 2CH(Ar)); δ_{C} (100 MHz, DMSO-d₆) 13.7, 21.7, 27.6, 27.8, 28.3, 41.0, 44.3, 47.8, 112.9, 116.8, 123.2, 125.2, 144.7, 145.6, 153.1, 154.3, 157.2, 158.4, 168.8; HRMS (MS-ES), calcd for C₂₃H₂₉N₆O₇ [M+H] m/z=501.2110, found 501.2092; rpHPLC t_{R}: condition (I) 14.647 (II) 36.729 min, purity 98.2% and 98.3%.

### 59. Preparation of 2-(2-((tert-butoxycarbonyl)(pentyl)amino)-6-((-tetrahydro)-2H-pyran-4-yl)amino)-9H-purin-9-yl)acetic acid (Compound ID: 7ay)

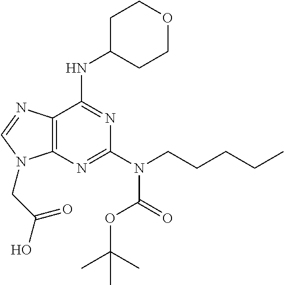 [SMILES: CCCCCN(C(=O)OC(C)(C)C)c1nc(NC2CCOCC2)c2ncn(CC(=O)O)c2n1]

Purine 6ay was treated according to general procedure E, to yield product lay as a lyophilized a white powder (88%): m.p.>112° C. (dec); IR (KBr, cm⁻¹) 3666, 2958, 2927, 2856, 1707, 1475, 1384, 1367, 1275, 1241, 1151; δ_{H} (400 MHz, DMSO-d₆) 0.83 (t, J=6.9 Hz, 3H, (CH₂)₄CH₃), 1.08-1.26 (m, 4H, (CH₂)₂CH₂CH₂CH₃), 1.38 (s, 9H, C(CH₃)₃), 1.47-1.88 (m, 6H, 2CH₂ (tetrahydropyran) and CH₂CH₂(CH₂)₂CH₃), 3.34-3.51 (m, 2H, CH₂ (tetrahyropyran)), 3.64 (t, J=7.1 Hz, 2H, CH₂(CH₂)₃CH₃), 3.85-3.96 (m, 2H, CH₂ (tetrahydropyran)), 4.22 (bs, 1H, CH), 4.77 (s, 2H, CH₂CO₂Et) 7.74 (bs, 1H, NH), 8.02 (s, 1H, CH(H-8)); δ_{C} (100 MHz, DMSO-d₆); 13.9, 21.7, 27.9, 28.0, 28.4, 32.3, 44.3, 46.2, 47.5, 66.3, 79.2, 115.8, 141.3, 150.0, 153.5, 153.9, 155.2, 169.2 HRMS (MS-ES), calcd for C₂₂H₃₅N₆O₅ [M+H] m/z=463.2666, found 463.2663; rpHPLC t_{R}: condition (I) 13.944 (II) 32.497 min, purity 90.8% and 91.6%.

### 60. Preparation of 2-(64(benzylamino)-2-((tert-butoxycarbonyl)(4-cyclohexylbenzyl)amino)-9H-purin-9-yl) acetic acid (Compound ID: 7ba)

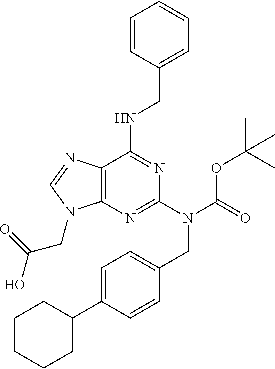 [SMILES: CC(C)(C)OC(=O)N(Cc1ccc(C2CCCCC2)cc1)c1nc(NCc2ccccc2)c2ncn(CC(=O)O)c2n1]

Purine 6ba was treated according to general procedure E, to yield product 7ba as a white solid (63%): m.p.>147° C. (dec); IR (KBr, cm⁻¹) 3552, 3476, 3414, 3261, 2919, 2849, 1741, 1631, 1478, 1446, 1421, 1398; δ_{H} (400 MHz, CDCl₃) 1.20-1.42 (m, 14H, 5H (cyclohexyl) and C(CH₃)₃), 1.69-1.81 (m, 5H (cyclohexyl)), 2.39-2.45 (m, 1H, CH), 4.72 (bs, 2H, HNCH₂), 4.87 (bs, 2H, CH₂Ar), 5.04 (s, 2H, CH₂CO₂H), 6.94-7.27 (m, 10H, NH and 9 CH(Ar)), 7.88 (bs, 1H, CH(H-8)); LRMS (MS-ES), calcd for C₃₂H₃₇N₆O₄ [M−H] m/z=569.30, found 569.40.

### 61. Preparation of 2-(64(benzyl(methyl)amino)-2-((tert-butoxycarbonyl)(4-cyclohexylbenzyl)amino)-9H-purin-9-yl) acetic acid (Compound ID: 7bb)

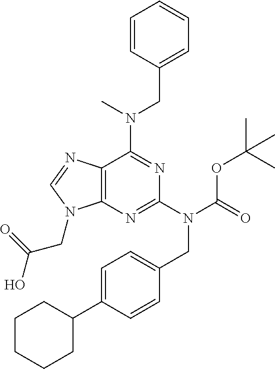 [SMILES: CN(Cc1ccccc1)c1nc(N(Cc2ccc(C3CCCCC3)cc2)C(=O)OC(C)(C)C)nc2c1ncn2CC(=O)O]

Purine 6bb was treated according to general procedure E, to yield product 7bb as a white solid (90%): m.p.>126-131° C.; IR (KBr, cm⁻¹) 3414, 2922, 2850, 1743, 1702, 1655, 1596, 1480, 1445, 1398, 1367, 1282; δ_{H} (400 MHz, CDCl₃) 1.28-1.43 (m, 14H, 5H (cyclohexyl) and C(CH₃)₃), 1.71-1.81 (m, 5H (cyclohexyl)), 2.38-2.42 (m, 1H, CH), 2.97-3.77 (bm, 3H, NCH₃), 4.94 (s, 2H, CH₂Ar), 5.03 (bs, 2H, CH₂CO₂H), 5.39-5.62 (bm, 2H, CH₃NCH₂), 6.98-7.29 (m, 9H, 9 CH(Ar)), 7.73 (s, 1H, CH(H-8)); LRMS (MS-ES), calcd for C₃₃H₃₉N₆O₄ [M−H] m/z=583.31, found 583.38.

### 62. Preparation of 2-(2-((tert-butoxycarbonyl)(4-cyclohexylbenzyl)amino)-6-(furan-2-ylmethyl)(methyl)amino)-9H-purin-9-yl)acetic acid (Compound ID: 7bd)

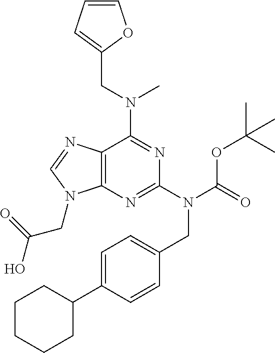 [SMILES: CN(Cc1ccco1)c1nc(N(Cc2ccc(C3CCCCC3)cc2)C(=O)OC(C)(C)C)nc2c1ncn2CC(=O)O]

Purine 6bd was treated according to general procedure E, to yield product 7bd as a white solid (72%): m.p.>130° C. (dec); IR (KBr, cm⁻¹) 2919, 2849, 1741, 1648, 1601, 1445, 1406, 1392, 1367, 1290, 1274, 1245; δH (400 MHz, CDCl₃) 1.20-1.40 (m, 5H, 5H (cyclohexyl)), 1.43 (s, 9H, C(CH₃)₃), 1.71-1.82 (m, 5H (cyclohexyl)), 2.42-2.47 (m, 1H, CH), 3.05-3.81 (m, 5H, CH₂ and CH₃ (furfuryl)), 5.01 (bs, 2H, CH₂Ar), 5.12 (s, 2H, CH₂CO₂H), 6.26-6.38 (m, 2H, 2CH (furfuryl)), 7.10 (d, J=7.7 Hz, 2H, 2CH(Ar)), 7.24 (d, J=8.3 Hz, 2H, 2CH(Ar)), 7.34 (s, 1H, CH (furfuryl)), 7.77 (s, 1H, CH(H-8)); LRMS (MS-ES), calcd for C₃₁H₃₇N₆O₅ [M−H] m/z=573.29, found 573.37.

### 63. Preparation of 2-(2-((tert-butoxycarbonyl)(4-cyclohexylbenzyl)amino)-6-(cyclopentylamino)-9H-purin-9-yl)acetic acid (Compound ID: 7be)

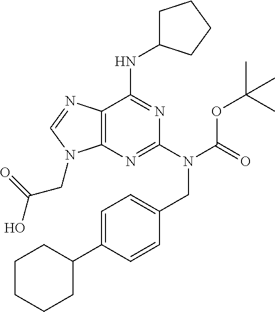 [SMILES: CC(C)(C)OC(=O)N(Cc1ccc(C2CCCCC2)cc1)c1nc(NC2CCCC2)c2ncn(CC(=O)O)c2n1]

Purine 6be was treated according to general procedure E, to yield product 7be as a white solid (68%): m.p.>144° C.; IR (KBr, cm⁻¹) 3550, 3475, 3414, 2925, 2851, 1706, 1618, 1448, 1366, 1241; δ_{H} (400 MHz, CDCl₃) 1.18-1.46 (m, 14H, 5H (cyclohexyl) and C(CH₃)₃), 1.46-1.54 (m, 4H (cyclopentyl)), 1.71-1.82 (m, 7H, 5H (cyclohexyl) and 2H (cyclopentyl)), 1.91-1.99 (bs, 2H (cyclopentyl), 2.42-2.47 (m, 1H, CH), 4.40 (bs, 1H, NCH), 4.86 (s, 2H, CH₂Ar), 5.05 (s, 2H, CH₂CO₂H), 7.00 (bs, 1H, NH), 7.07 (d, J=7.7 Hz, 2H, 2CH(Ar)), 7.28 (d, J=7.9 Hz, 2H, 2CH(Ar)), 7.79 (s, 1H, CH(H-8)); LRMS (MS-ES), calcd for C₃₀H₃₉N₆O₄ [M−H] m/z=547.31, found 547.44.

### 64. Preparation of 2-(2-((tert-butoxycarbonyl)(4-cyclohexylbenzyl)amino)-6-(cyclopentylamino)-9H-purin-9-yl)acetic acid (Compound ID: 7bf)

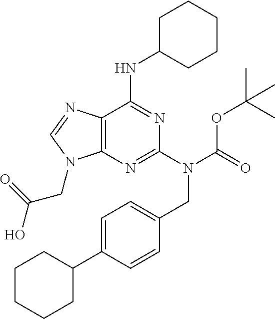 [SMILES: CC(C)(C)OC(=O)N(Cc1ccc(C2CCCCC2)cc1)c1nc(NC2CCCCC2)c2ncn(CC(=O)O)c2n1]

Purine 6bf was treated according to general procedure E, to yield product 7bf as a white solid (89%): m.p.=118-123° C.; IR (KBr, cm⁻¹) 2926, 2852, 1617, 1477, 1449, 1389, 1367, 1245, 1158, 1108; δ_{H} (400 MHz, CDCl₃) 1.16-1.38 (m, 10H, 5H (cyclohexyl) and 5H (NH-cyclohexyl)), 1.35 (s, 9H, C(CH₃)₃), 1.59-1.94 (m, 10H, 5H (cyclohexyl) and 5H(NH-cyclohexyl)), 2.38-2.48 (m, 1H, CH), 3.90 (bs, 1H, HNCH), 4.79 (s, 2H, CH₂Ar), 5.00 (s, 2H, CH₂CO₂H), 6.29 (bs, 1H, NH), 7.08 (d, J=8.1 Hz, 2H, 2CH(Ar)), 7.24 (d, J=7.9 Hz, 2H, 2CH(Ar)), 7.71 (s, 1H, CH(H-8)); LRMS (MS-ES), calcd for C₃₁H₄₁N₆O₄ [M−H] m/z=561.33, found 561.44.

### 65. Preparation of 2-(2-(allylamino)-2-((tert-butoxycarbonyl)(4-cyclohexylbenzyl)amino)-9H-purin-9-yl)acetic acid (Compound ID: 7bi)

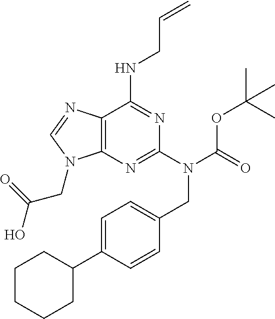 [SMILES: C=CCNc1nc(N(Cc2ccc(C3CCCCC3)cc2)C(=O)OC(C)(C)C)nc2c1ncn2CC(=O)O]

Purine 6bi was treated according to general procedure E, to yield product 7bai as a white solid (88%): m.p.>123° C. (dec); IR (KBr, cm⁻¹) 3549, 3476, 3414, 3275, 2920, 2849, 1745, 1618, 1449, 1404, 1366, 1249; δ_{H} (400 MHz, CDCl₃) 1.17-1.30 (m, 5H, (cyclohexyl)), 1.36 (s, 9H, C(CH₃)₃), 1.68-1.87 (m, 5H (cyclohexyl)), 2.36-2.49 (m, 1H, CH), 4.15 (bs, 2H, CH₂CHCH₂), 4.88 (s, 2H, CH₂Ar), 5.06 (s, 2H, CH₂CO₂H), 5.12 (dd, J=10.6 and 1.5 Hz, 1H, CH₂CHCH₂), 5.21 (dd, J=17.2 and 1.5 Hz, 1H, CH₂CHCH₂), 5.79-5.97 (m, 1H, CH₂CHCH₂), 6.39 (bs, 1H, NH), 7.09 (d, J=7.9 Hz, 2H, 2CH(Ar)), 7.25 (d, J=7.2 Hz, 2H, 2CH(Ar)), 7.74 (s, 1H, CH(H-8)); LRMS (MS-ES), calcd for C₂₈H₃₅N₆O₄ [M−H] m/z=519.28, found 519.30.

### 66. Preparation of 2-(2-((tert-butoxycarbonyl)(4-cyclohexylbenzyl)amino)-6-(isobuytylamino)-9H-purin-9-yl)acetic acid (Compound ID: 7bj)

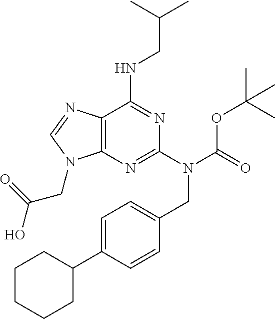 [SMILES: CC(C)CNc1nc(N(Cc2ccc(C3CCCCC3)cc2)C(=O)OC(C)(C)C)nc2c1ncn2CC(=O)O]

Purine 6bj was treated according to general procedure E, to yield product 7bj as a white solid (86%): m.p.>124-126° C.; IR (KBr, cm⁻¹) 3549, 3476, 3414, 3335, 2929, 1759, 1683, 1619, 1591, 1434, 1388, 1343; δ_{H} (400 MHz, CDCl₃) 0.93 (s, 3H, CH₂CH(CH₃)₂), 0.95 (s, 3H, CH₂CH(CH₃)₂), 1.19-1.38 (m, 5H (cyclohexyl), 1.39 (s, 9H, C(CH₃)₃), 1.67-1.79 (m, 5H, (cyclohexyl)), 1.84-1.93 (m, 1H, CH₂CH(CH₃)₂) 2.40-2.47 (m, 1H, CH), 3.37 (bs, 2H, CH₂CH(CH₃)₂), 4.88 (s, 2H, CH₂Ar), 5.04 (s, 2H, CH₂CO₂H), 6.03 (bs, 1H, NH), 7.1 (d, J=8.1 Hz, 2H, 2CH(Ar)), 7.23 (d, J=8.1 Hz, 2H, 2CH(Ar)), 7.74 (s, 1H, CH(H-8)); LRMS (MS-ES), calcd for C₂₉H₃₉N₆O₄ [M−H] m/z=535.31, found 535.35.

### 67. Preparation of 2-(2-((tert-butoxycarbonyl)(4-cyclohexylbenzyl)amino)-6-(isobuytylamino)-9H-purin-9-yl)acetic acid (Compound ID: 7bl)

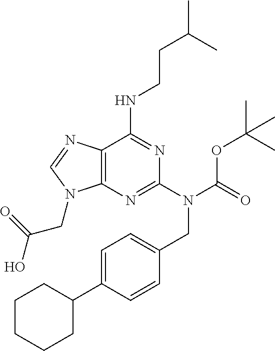 [SMILES: CC(C)CCNc1nc(N(Cc2ccc(C3CCCCC3)cc2)C(=O)OC(C)(C)C)nc2c1ncn2CC(=O)O]

Purine 6bl was treated according to general procedure E, to yield product 7bl as a white solid (93%): m.p.>128° C. (dec); IR (KBr, cm⁻¹) 2925, 2852, 1707, 1485, 1440, 1400, 1379, 1246; δ_{H} (400 MHz, CDCl₃) 0.87 (s, 3H, (CH₂)₂CH(CH₃)₂), 0.88 (s, 3H, (CH₂)₂CH(CH₃)₂), 1.25-1.39 (m, 5H, (cyclohexyl)), 1.39 (s, 9H, C(CH₃)₃), 1.46-1.66 (m, 3H, CH₂CH₂CH(CH₃)₂), 1.67-1.84 (m, 5H, (cyclohexyl)), 2.39-2.47 (m, 1H, CH), 3.50 (bs, 2H, CH₂CH₂CH(CH₃)₂), 4.88 (s, 2H, CH₂Ar), 5.08 (s, 2H, CH₂CO₂H), 6.76 (bs, 1H, NH), 7.07 (d, J=7.9 Hz, 2H, 2CH(Ar)), 7.27 (d, J=7.9 Hz, 2H, 2CH(Ar)), 7.76 (s, 1H, CH(H-8)); LRMS (MS-ES), calcd for C₃₀R₄₁N₆O₄ [M−H] m/z=549.33, found 549.39.

### 68. Preparation of 2-(2-((tert-butoxycarbonyl)(4-cyclohexylbenzyl)amino)-6-morpholino-9H-purin-9-yl)acetic acid (Compound ID: 7bm)

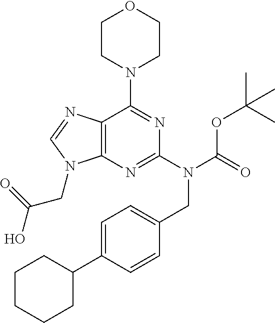 [SMILES: CC(C)(C)OC(=O)N(Cc1ccc(C2CCCCC2)cc1)c1nc(N2CCOCC2)c2ncn(CC(=O)O)c2n1]

Purine 6bm was treated according to general procedure E, to yield product 7bm as a lyophilized white powder (83%): m.p.=166-167° C.; IR (KBr, cm⁻¹) 3666, 2958, 2927, 2856, 1707, 1475, 1385, 1367, 1275, 1242, 1151, 1011; δ_{H} (400 MHz, DMSO-d₆) 1.17-1.36 (m, 5H, (cyclohexyl)), 1.37 (s, 9H, C(CH₃)₃), 1.66-1.77 (m, 5H, (cyclohexyl)), 2.38-2.44 (m, 1H, CH), 3.68 (t, J=4.5 Hz, 4H, 2CH₂, (morpholine)), 4.12 (bs, 4H, 2CH₂, (morpholine)), 4.85 (s, 2H, CH₂Ar), 4.91 (s, 2H, CH₂CO₂H), 7.1 (d, J=8.1 Hz, 2H, 2CH(Ar)), 7.21 (d, J=8.1 Hz, 2H, 2CH(Ar)), 8.07 (s, 1H, CH(H-8)); δ_{C} (100 MHz, DMSO-d₆) 25.5, 26.3, 27.8, 33.9, 43.4, 44.2, 50.3, 66.1, 79.9, 115.8, 126.3, 127.3, 136.5, 140.8, 145.9, 151.8, 152.7, 154.1, 154.5, 169.2; HRMS (MS-ES), calcd for C₂₉H₃₉N₆O_{5 [}M+H] m/z=551.2962, found 551.2976; rpHPLC t_{R}: condition (I) 15.722 (II) 41.975 min, purity 91.8% and 90.7%.

### 69. Preparation of 2-(2-((tert-butoxycarbonyl)(4-cyclohexylbenzyl)amino)-6-(-3-nitrophenoxy)-9H-purin-9-yl)acetic acid (Compound ID: 7bn)

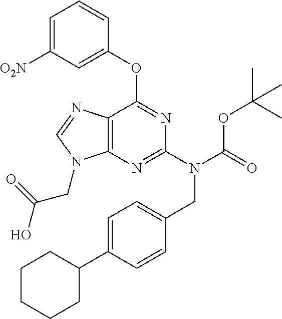 [SMILES: CC(C)(C)OC(=O)N(Cc1ccc(C2CCCCC2)cc1)c1nc(Oc2cccc([N+](=O)[O-])c2)c2ncn(CC(=O)O)c2n1]

Purine 6bn was treated according to general procedure E, to yield product 7bn as a white solid (90%): m.p.=103-107° C.; IR (KBr, cm⁻¹) 2925, 2852, 1578, 1532, 1448, 1402, 1368, 1351, 1275, 1236, 1154; δ_{H} (400 MHz, CDCl₃) 1.19 (s, 9H, C(CH₃)₃), 1.31-1.42 (m, 5H, (cyclohexyl)), 1.72-1.84 (m, 5H, (cyclohexyl)), 2.37-2.44 (m, 1H, CH), 4.79 (s, 2H, CH₂Ar), 4.88 (s, 2H, CH₂CO₂H), 6.92 (d, J=8.1 Hz, 2H, 2CH(Ar)), 6.97 (d, J=8.1 Hz, 2H, 2CH(Ar)), 7.45-7.51 (m, 2H, 2CH(Ar)), 7.99-8.08 (m, 2H, 2CH(Ar)), 8.10 (s, 1H, CH(H-8)); LRMS (MS-ES), calcd for C₃₁H₃₃N₆O₇ [M−H] m/z=601.25, found 601.42

### 70. Preparation of 2-(2-((tert-butoxycarbonyl)(4-cyclohexylbenzyl)amino)-6-(-4-nitrophenoxy)-9H-purin-9-yl)acetic acid (Compound ID: 7bo)

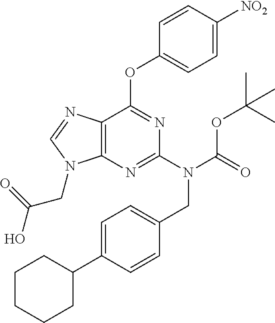 [SMILES: CC(C)(C)OC(=O)N(Cc1ccc(C2CCCCC2)cc1)c1nc(Oc2ccc([N+](=O)[O-])cc2)c2ncn(CC(=O)O)c2n1]

Purine 6bo was treated according to general procedure E, to yield product 7bo as a white solid (83%): m.p.>126° C. (dec); IR (KBr, cm⁻¹) 3550, 3474, 3415, 2924, 2853, 1747, 1638, 1617, 1576, 1524, 1486, 1457; δH (400 MHz, CDCl₃) 1.19-1.28 (m, 5H, (cyclohexyl)), 1.35 (s, 9H, C(CH₃)₃), 1.7-1.83 (m, 5H, (cyclohexyl)), 2.40-2.49 (m, 1H, CH), 4.91 (s, 2H, CH₂Ar), 5.02 (s, 2H, CH₂CO₂H), 6.95-7.12 (m, 4H, 4 CH(Ar)), 7.34-7.41 (m, 2H, 2CH(Ar)), 8.02 (s, 1H, CH(H-8)), 8.17-8.22 (m, 2H, 2CH(Ar)); LRMS (MS-ES), calcd for C₃₁H₃₃N₆O₇ [M−H] m/z=601.25, found 601.31

### 71. Preparation of 2-(2-((tert-butoxycarbonyl)(4-cyclohexylbenzyl)amino)-6-((-4-fluorophenyl)amino)-9H-purin-9-yl)acetic acid (Compound ID: 7bp)

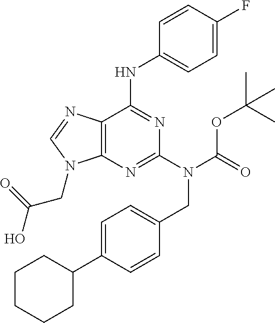 [SMILES: CC(C)(C)OC(=O)N(Cc1ccc(C2CCCCC2)cc1)c1nc(Nc2ccc(F)cc2)c2ncn(CC(=O)O)c2n1]

Purine 6bp was treated according to general procedure E, to yield product 7bp as a white solid (92%): m.p.>124° C. (dec); IR (KBr, cm⁻¹) 3549, 3475, 3415, 3238, 2925, 1710, 1638, 1617, 1509, 1474, 1449, 1408; δ_{H} (400 MHz, CDCl₃) 1.22-1.42 (m, 14H, 5H (cyclohexyl) and C(CH3)-₃), 1.67-1.85 (m, 5H (cyclohexyl)), 2.40-2.45 (m, 1H, CH), 4.99 (s, 2H, CH2Ar), 5.10 (s, 2H, CH₂CO₂H), 6.88-6.92 (m, 2H, 2CH(Ar)), 7.10 (d, J=8.1 Hz, 2H, 2CH(Ar)), 7.25 (d, J=7.9 Hz, 2H, 2CH(Ar)), 7.68-7.72 (m, 2H, 2CH(Ar)), 7.95 (s, 1H, CH(H-8)); LRMS (MS-ES), calcd for C₃₁H₃₄FN₆O₄ [M−H] m/z=573.27, found 573.37

### 72. Preparation of 2-(2-((tert-butoxycarbonyl)(4-cyclohexylbenzyl)amino)-6-(furan-2-ylmethyl)amino)-9H-purin-9-yl)acetic acid (Compound ID: 7bq)

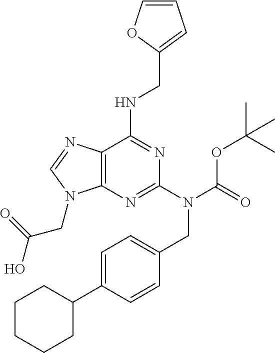 [SMILES: CC(C)(C)OC(=O)N(Cc1ccc(C2CCCCC2)cc1)c1nc(NCc2ccco2)c2ncn(CC(=O)O)c2n1]

Purine 6bq was treated according to general procedure E, to yield product 7bq as a white solid (91%): m.p.>132 (dec)° C.; IR (KBr, cm⁻¹) 2920, 2850, 1744, 1701, 1478, 1445, 1391, 1366, 1301, 1241, 1209, 1161, 1109; δ_{H} (400 MHz, CDCl₃) 1.19-1.38 (m, 5H, 5H (cyclohexyl)), 1.40 (s, 9H, C(CH₃)₃), 1.79-1.81 (m, 5H (cyclohexyl)), 2.39-2.45 (m, 1H, CH), 4.73 (bs, 2H, CH₂ (furfuryl)), 4.85 (s, 2H, CH₂Ar), 5.06 (s, 2H, CH₂CO₂H), 6.16-6.17 (m, 1H, CH (furfuryl)), 6.26 (bs, 1H, NH), 6.26-6.27 (m, 1H, CH (furfuryl)), 7.07 (d, J=7.5 Hz, 2H, 2CH(Ar)), 7.29 (d, J=8.2 Hz, 2H, 2CH(Ar)), 7.30-7.31 (m, 1H, CH (furfuryl)), 7.76 (s, 1H, CH(H-8)); LRMS (MSES), calcd for C₃₀H₃₅FN₆O₅ [M−H] m/z=559.27, found 559.36.

### 73. Preparation of 2-(2-((tert-butoxycarbonyl)(4-cyclohexylbenzyl)amino)-6-(propylamino)-9H-purin-9-yl)acetic acid (Compound ID: 7bs)

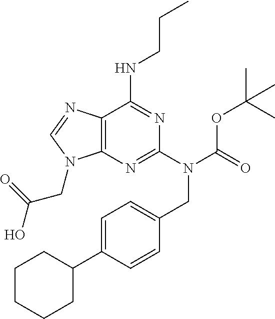 [SMILES: CCCNc1nc(N(Cc2ccc(C3CCCCC3)cc2)C(=O)OC(C)(C)C)nc2c1ncn2CC(=O)O]

Purine 6bs was treated according to general procedure E, to yield product 7bs as a white solid (89%): m.p.>68° C. (dec); IR (KBr, cm⁻¹) 3412, 2926, 2852, 1515, 1482, 1448, 1381, 1244, 1156; δ_{H} (400 MHz, CDCl₃) 0.91 (t, J=7.3 Hz, 3H, NHCH₂CH₂CH₃), 1.21 (s, 9H, C(CH₃)₃), 1.28-1.43 (m, 5H (cyclohexyl)), 1.59 (m, 2H, NHCH₂CH₂CH₃), 1.7-1.83 (m, 5H (cyclohexyl)), 2.40-2.46 (m, 1H, CH), 3.42 (bs, 2H, NHCH₂CH₂CH₃), 4.81 (s, 2H, CH₂Ar), 5.00 (s, 2H, CH₂CO₂H), 6.12 (bs, 1H, NH), 7.08 (d, J=8.1 Hz, 2H, 2CH(Ar)), 7.23 (d, J=8.1 Hz, 2H, 2CH(Ar)), 7.67 (s, 1H, CH, (H-8)); LRMS (MS-ES), calcd for C₂₈H₃₇N₆O₄ [M−H] m/z=521.30, found 521.42.

### 74. Preparation of preparation OF 2-(2-((tert-butoxycarbonyl)(4-cyclohexylbenzyl)amino)-6-(hexylamino)-9H-purin-9-yl)acetic acid (Compound ID: 7bt)

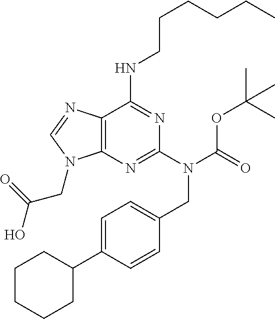 [SMILES: CCCCCCNc1nc(N(Cc2ccc(C3CCCCC3)cc2)C(=O)OC(C)(C)C)nc2c1ncn2CC(=O)O]

Purine 6bt was treated according to general procedure E, to yield product 7bt as a white solid (85%): m.p.>122° C. (dec); IR (KBr, cm⁻¹) 3414, 2956, 2926, 2853, 1707, 1619, 1514, 1449, 1389, 1242; δ_{H} (400 MHz, CDCl₃) 0.88 (t, J=7.2 Hz, 3H, NH(CH₂)₅CH₃), 1.11-1.23 (m, 11H, 5H (cyclohexyl), 6H, NH(CH₂)₂CH₂CH₂CH₂CH₃), 1.25 (s, 9H, C(CH₃)₃), 1.53-1.76 (m, 7H, 5H, (cyclohexyl) and NH(CH₂)₄CH₂CH₃)), 2.36-2.47 (m, 1H, CH), 3.40 (bs, 2H, NHCH₂(CH₂)₄CH₃), 4.53-4.75 (m, 2H, CH₂Ar), 4.95 (s, 2H, CH₂CO₂H), 7.03-7.23 (m, 4H, 4 CH(Ar)), 7.58 (bs, 1H, NH), 7.73 (s, 1H, CH, (H-8)); LRMS (MS-ES), calcd for C₃₁H₄₃N₆O₄ [M−H] m/z=563.34, found 563.43.

### 75. Preparation of 2-(6-(3-bromophenoxy)-2-((tert-butoxycarbonyl)(4-cyclohexylbenzyl)amino)-9H-purin-9-yl)acetic acid (Compound ID: 7bu)

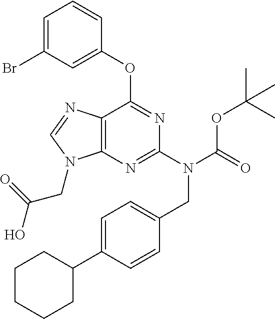 [SMILES: CC(C)(C)OC(=O)N(Cc1ccc(C2CCCCC2)cc1)c1nc(Oc2cccc(Br)c2)c2ncn(CC(=O)O)c2n1]

Purine 6bu was treated according to general procedure E, to yield product 7bu as a white solid (84%): m.p.>127° C. (dec); IR (KBr, cm⁻¹) 3550, 3478, 3415, 2924, 2851, 1721, 1709, 1626, 1602, 1577, 1515, 1473; δ_{H} (400 MHz, CDCl₃) 1.10-1.33 (m, 5H, (cyclohexyl)), 1.37 (s, 9H, C(CH₃)₃), 1.67-1.85 (m, 5H, (cyclohexyl)), 2.37-2.47 (m, 1H, CH), 4.90 (s, 2H, CH₂, (Ar)), 4.99 (s, 2H, CH₂CO₂H), 6.98-7.07 (m, 4H, 4 CH(Ar)), 7.12-7.17 (m, 1H, CH (Ar)), 7.23 (t, J=8.1 Hz, 1H, CH(Ar)), 7.36-7.40 (m, 1H, CH(Ar)), 7.45 (t, J=2.0 Hz, 1H, CH(Ar)), 8.13 (s, 1H, CH, (H-8)); LRMS (MS-ES), calcd for C₃₁H₃₃BrN₅O₅ [M−H] m/z=634.17, found 634.33.

### 76. Preparation of 2-(2-((tert-butoxycarbonyl)(4-cyclohexylbenzyl)amino)-6-(4-fluorophenoxy)-9H-purin-9-yl)acetic acid (Compound ID: 7bv)

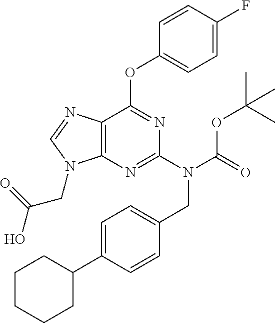 [SMILES: CC(C)(C)OC(=O)N(Cc1ccc(C2CCCCC2)cc1)c1nc(Oc2ccc(F)cc2)c2ncn(CC(=O)O)c2n1]

Purine 6bv was treated according to general procedure E, to yield product 7bv as a white solid (86%): m.p.=119-133° C.; IR (KBr, cm⁻¹) 3550, 3475, 3415, 3236, 2924, 2852, 1707, 1619, 1587, 1503, 1449, 1393; δ_{H} (400 MHz, CDCl₃) 1.11-1.34 (m, 5H, (cyclohexyl)), 1.36 (s, 9H, C(CH₃)₃), 1.70-1.84 (m, 5H (cyclohexyl)), 2.40-2.47 (m, 1H, CH), 4.87 (s, 2H, CH₂Ar), 4.99 (s, 2H, CH₂CO₂H), 6.97-7.04 (m, 6H, 6 CH(Ar)), 7.12-7.15 (m, 2H, 2CH (Ar)), 8.11 (s, 1H, CH, (H-8)); LRMS (MS-ES), calcd for C₃₁H₃₃FN₅O₅ [M−H] m/z=574.25, found 574.36.

### 77. Preparation of 2-(2-((tert-butoxycarbonyl)(4-cyclohexylbenzyl)amino)-6-(perfluorophenoxy)-9H-purin-9-yl)acetic acid (Compound ID: 7bw)

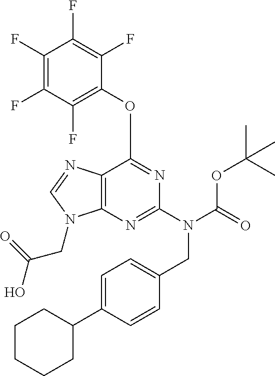 [SMILES: CC(C)(C)OC(=O)N(Cc1ccc(C2CCCCC2)cc1)c1nc(Oc2c(F)c(F)c(F)c(F)c2F)c2ncn(CC(=O)O)c2n1]

Purine 6bw was treated according to general procedure E, to yield product 7bw as a white solid (79%): m.p.>94.1-104° C.; IR (KBr, cm⁻¹) 3414, 2927, 2852, 1743, 1669, 1637, 1618, 1581, 1522, 1452, 1409, 1380; δ_{H} (400 MHz, CDCl₃) 1.18-1.28 (m, 5H (cyclohexyl)), 1.36 (s, 9H, C(CH₃)₃), 1.71-1.85 (m, 5H (cyclohexyl)), 2.40-2.47 (m, 1H, CH), 4.85 (s, 2H, CH₂Ar), 5.06 (s, 2H, CH₂CO₂H) 6.93 (d, J=8.2 Hz, 2H, 2CH(Ar)), 7.03 (d, J=8.1 Hz, 2H, 2CH(Ar)), 8.16 (s, 1H, CH, (H-8)); LRMS (MS-ES), calcd for C₃₁H₂₉F₅N₅O₅ [M−H] m/z=646.22, found 646.35.

### 78. Preparation of 2-(2-((tert-butoxycarbonyl)(4-cyclohexylbenzyl)amino)-6-phenoxy-9H-purin-9-yl)acetic acid (Compound ID: 7bx)

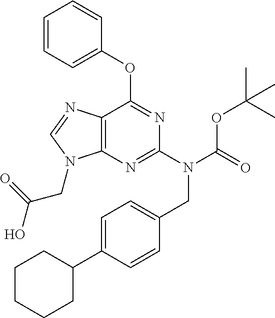 [SMILES: CC(C)(C)OC(=O)N(Cc1ccc(C2CCCCC2)cc1)c1nc(Oc2ccccc2)c2ncn(CC(=O)O)c2n1]

Purine 6bx was treated according to general procedure E, to yield product 7bx as a white solid (83%): m.p.>129° C. (dec); IR (KBr, cm⁻¹) 3549, 3477, 3414, 2923, 2851, 1741, 1618, 1578, 1491, 1446, 1391, 1367; δ_{H} (400 MHz, CDCl₃) 1.11-1.34 (m, 5H, (cyclohexyl)), 1.36 (s, 9H, C(CH₃)₃), 1.70-1.84 (m, 5H (cyclohexyl)), 2.40-2.47 (m, 1H, CH), 4.87 (s, 2H, CH₂Ar), 4.99 (s, 2H, CH₂CO₂H), 6.96-7.02 (m, 4H, 4 CH(Ar)), 7.16-7.26 (m, 3H, 3CH (Ar)), 7.35-7.40 (m, 2H, 2CH(Ar)), 8.04 (s, 1H, CH, (H-8)); LRMS (MS-ES), calcd for C₃₁H₃₄N₅O₅ [M−H] m/z=556.26, found 556.34.

### 79. Preparation of 2-(6-(benzylamino)-2-(pentylamino)-9H-purin-9-yl)acetic acid (Compound ID: 8aa)

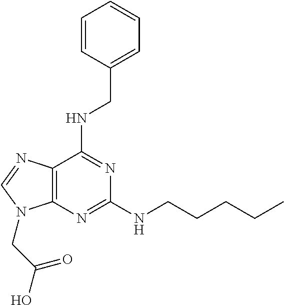 [SMILES: CCCCCNc1nc(NCc2ccccc2)c2ncn(CC(=O)O)c2n1]

Purine 7aa was treated according to general procedure F, to yield final product Baa as an off-white lyophilized powder (85%): m.p.>81° C. (dec); IR (KBr, cm⁻¹) 3504, 3281, 2934, 2485, 1351, 1184; δ_{H} (400 MHz, DMSO-d₆) 0.84 (m, 3H, (CH₂)₄CH₃), 1.19-1.34 (m, 4H, (CH₂)₂CH₂CH₂CH₃), 1.42-1.54 (m, 2H, CH₂CH₂(CH₂)₂CH₃), 3.26 (t, J=6.9 Hz, 2H, CH₂(CH₂)₃CH₃), 4.67 (bs, 2H, CH₂Ar), 4.89 (s, 2H, CH₂CO₂H), 7.22-7.37 (m, 5H, CH(Ar)), 7.31 (bs, 1H, NH), 7.93 (s, 1H, CH(H-8)), 8.89 (bs, 1H, NH); HRMS (MS-ES), calcd for C₁₉H₂₅N₆O₂ [M+H] m/z=369.2035, found 369.2033; rpHPLC tR: condition (I) 13.814 (II) 33.928 min, purity 97.58% and 96.7%.

### 80. Preparation of 2-(6-(benzyl(methyl)amino)-2-(pentylamino)-9H-purin-9-yl)acetic acid (Compound ID: 8ab)

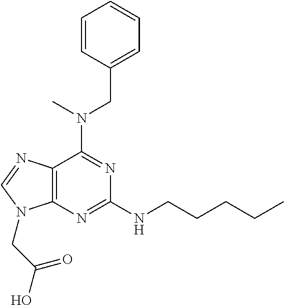 [SMILES: CCCCCNc1nc(N(C)Cc2ccccc2)c2ncn(CC(=O)O)c2n1]

Purine lab was treated according to general procedure F, to yield final product 8ab as a white lyophilized powder (83%): m.p.=134-142° C.; IR (KBr, cm⁻¹) 3466, 3080, 1937, 1419, 1246, 1203, 1140; δ_{H} (400 MHz, DMSO-d₆) 0.82-0.87 (m, 3H, (CH₂)₄CH₃), 1.12-1.30 (m, 4H, CH₂CH₂CH₂CH₂CH₃), 1.49-1.53 (m, 2H, CH₂CH₂CH₂CH₂CH₃), 3.04-3.67 (m, 3H, NCH₃), 3.23-3.31 (m, 2H, CH₂(CH₂)₃CH₃), 4.67-5.59 (bm, 2H, CH₂Ar), 4.87 (s, 2H, CH₂CO₂H), 7.22 (bs, 1H, NH), 7.24-7.35 (m, 5H, CH(Ar)), 7.83 (s, 1H, CH(H-8)); δ_{C} (100 MHz, DMSO-d₆) 13.8, 21.8, 27.8, 28.6, 40.3, 41.0, 44.3, 47.4, 112.5, 126.8, 127.1, 127.2, 128.3, 137.0, 138.5, 154.1, 158.5, 169.1; HRMS (MS-ES), calcd for C₂₀H₂₇N₆O₂ [M+H] m/z=383.2177, found 383.2190; rpHPLC t_{R}: condition (I) 14.619 (II) 36.342 min, purity 97.6% and 94.9%.

### 81. Preparation of 2-(2-(pentylamino)-6-(pentylamino)-9H-purin-9-yl)acetic acid (Compound ID: 8ac)

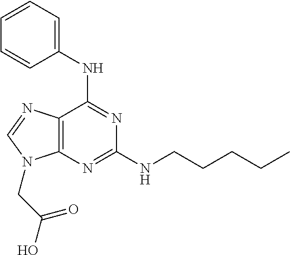 [SMILES: CCCCCNc1nc(Nc2ccccc2)c2ncn(CC(=O)O)c2n1]

Purine 7aa was treated according to general procedure F, to yield final product 8aa as a white lyophilized powder (86%): m.p.>145° C. (dec); IR (KBr, cm⁻¹) 3071, 2962, 2934, 1736, 1554, 1439, 1359, 1245, 1186, 1142; δ_{H} (400 MHz, DMSO-d₆) 0.87 (t, J=7.0 Hz, 3H, (CH₂)₄CH₃), 1.22-1.32 (m, 4H, (CH₂)₂CH₂CH₂CH₃), 1.52-1.59 (m, 2H, (CH₂)₃CH₂CH₃), 3.27 (t, J=7.2 Hz, 2H, CH₂(CH₂)₃CH₃), 4.85 (s, 2H, CH₂CO₂H), 6.95 (bs, 1H, NHCH₂), 7.01 (t, J=7.3 Hz, 1H, CH(Ar)), 7.29 (t, J=7.9 Hz, 2H, 2CH(Ar)), 7.93 (s, 1H, CH(H-8)), 7.97 (d, J=7.7 Hz, 2H, 2CH(Ar)), 9.64 (bs, 1H, ArNH); δ_{C} (100 MHz, DMSO-d₆) 13.9, 21.8, 28.7, 28.3, 41.7, 43.8, 116.6, 120.4, 122.4, 128.1, 139.5, 142.3, 150.6, 151.5, 153.7, 154.7, 169.1; HRMS (MS-ES), calcd for C₁₈H₂₃N₆O₂ [M+H] m/z=355.1870, found 355.1877; rpHPLC t_{R}: condition (I) 13.985 (II) 33.862 min, purity 99.09% and 98.4%.

### 82. Preparation of 2-(6-((furan-2-ylmethyl)(methyl)amino)-2-(pentylamino)-9H-purin-9-yl)acetic acid (Compound ID: 8ad)

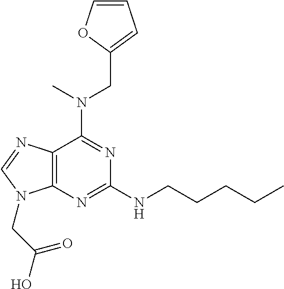 [SMILES: CCCCCNc1nc(N(C)Cc2ccco2)c2ncn(CC(=O)O)c2n1]

Purine lad was treated according to general procedure F, to yield final product Bad as a white lyophilized powder (78%): m.p.>164° C. (dec); IR (KBr, cm⁻¹) 3631, 2925, 1561, 1456, 1384, 1313, 1147; δ_{H} (400 MHz, DMSO-d₆) (0.85, t, J=6.9 Hz, 3H, (CH₂)₄CH₃), 1.22-1.30 (m, 4H, (CH₂)₂CH₂CH₂CH₃), 1.50 (p, J=7.1 Hz, 2H, CH₂CH₂(CH₂)₂CH₃), 3.21 (q, J=6.7 Hz, 2H, CH₂(CH₂)₃CH₃), 3.32 (vbs, 3H, NCH3), 4.55 (s, 2H, CH₂CO₂H), 5.26 (vbs, 2H, CH₂(furfuryl)), 6.27-6.29 (m, 1H, CH (furfuryl)), 6.33 (bs, 1H, NH), 6.37-6.39 (m, 1H, CH (furfuryl)), 7.55-7.57 (m, 1H, CH (furfuryl)), 7.66 (s, 1H, CH (H-8)); δC (100 MHz, DMSO-d₆) 13.9, 21.9, 22.5, 25.3, 28.8, 29.1, 37.7, 38.4, 41.0, 43.6, 112.5, 137.4, 151.0, 154.6, 159.4, 169.7; HRMS (MS-ES), calcd for C₁₈H₂₅N₆O₃ [M+H] m/z=373.1994, found 373.1982; rpHPLC t_{R}: condition (I) 14.074 (II) 33.425 min, purity 99.3% and 94.0%.

### 83. Preparation of 2-(6-(cyclopentylamino)-2-(pentylamino)-9H-purin-9-yl)acetic acid (Compound ID: 8ae)

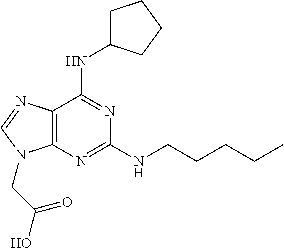 [SMILES: CCCCCNc1nc(NC2CCCC2)c2ncn(CC(=O)O)c2n1]

Purine 7ae was treated according to general procedure F, to yield final product Bae as a white lyophilized powder (92%): m.p.>139° C. (dec); IR (KBr, cm⁻¹) 3233, 3071, 2962, 2934, 1736, 1648, 1554, 1439, 1359, 1245, 1186, 1142; δ_{H} (400 MHz, DMSO-d₆) 0.87 (t, J=6.8 Hz, 3H, (CH₂)₄CH₃), 1.21-1.35 (m, 4H, (CH₂)₂CH₂CH₂CH₃), 1.50-1.77 (m, 8H, CH₂CH₂(CH₂)₂CH₃ and 3CH₂ (cyclopentyl)), 1.92-2.04 (m, 2H, CH₂ (cyclopentyl)), 3.27-3.33 (m, 2H, CH₂(CH₂)₃CH₃), 4.35 (vbs, 1H, CH (cyclopentyl)), 4.88 (s, 2H, CH₂CO₂H), 7.30 (vbs, 1H, NH), 7.96 (bs, 1H, NH), 8.32 (s, 1H, CH(H-8)), 13.34 (br s, 1H, CH₂CO₂H): HRMS (MS-ES), calcd for C₁₇H₂₇N₆O₂ [M+H] m/z=347.2192, found 347.2190; rpHPLC t_{R}: condition (I) 14.582 (II) 34.685 min, purity 90.1% and 97.6%.

### 84. Preparation of 2-(6-(cyclohexylamino)-2-(pentylamino)-9H-purin-9-yl)acetic acid (Compound ID: 8af)

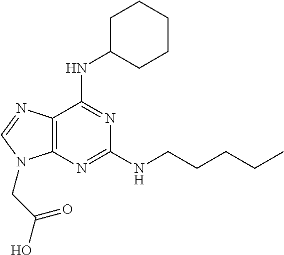 [SMILES: CCCCCNc1nc(NC2CCCCC2)c2ncn(CC(=O)O)c2n1]

Purine 7af was treated according to general procedure F, to yield final product 8af as a white lyophilized powder (97%): m.p.>188° C. (dec); IR (KBr, cm⁻¹) 2929, 2857, 1736, 1439, 1391, 1246, 1194, 1185, 1141; δ_{H} (400 MHz, DMSO-d₆) 0.87 (t, J=6.8 Hz, 3H, (CH₂)4CH₃), 1.10-1.45 (m, 10H, (CH₂)₂CH₂CH₂CH₃ and 3CH₂(cyclohexyl)), 1.53 (p, J=6.8 Hz, 2H, CH₂CH₂(CH₂)₂CH₃), 1.71-1.79 (m, 2H, CH₂ (cyclohexyl)), 1.84-1.99 (m, 2H, CH₂ (cyclohexyl)), 3.26 (t, J=6.6, 2H, CH₂(CH₂)₃CH₃), 3.95 (bs, 1H, CH (cyclohexyl)), 4.84 (s, 2H, CH₂CO₂H), 7.07 (vbs, 1H, NH), 7.86 (bs, 1H, NH), 8.32 (1H, s, CH(H-8)); HRMS (MS-ES), calcd for C₁₈H₂₉N₆O₂ [M+H] m/z=361.2356, found 361.2346; rpHPLC t_{R}: condition (I) 14.966 (II) 37.235 min, purity 94.7% and 91.5%.

### 85. Preparation of 2-(6-(ethyl(methyl)amino)-2-(pentylamino)-9H-purin-9-yl)acetic acid (Compound ID: 8ag)

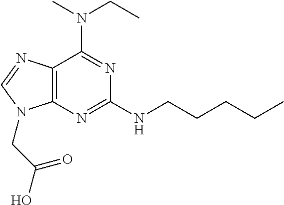 [SMILES: CCCCCNc1nc(N(C)CC)c2ncn(CC(=O)O)c2n1]

Purine 7ag was treated according to general procedure F, to yield final product 8ag as a white lyophilized powder (65%): m.p.>168° C. (dec); IR (KBr, cm⁻¹) 3626, 2958, 2931, 1385, 1326, 1183, 1057; δ_{H} (400 MHz, DMSO-d₆) 0.85 (t, J=6.9 Hz, 3H, (CH₂)₄CH₃), 1.13 (t, 7.0 Hz, 3H, NCH₂CH₃), 1.22-1.33 (m, 4H, (CH₂)₂CH₂CH₂CH₃), 1.49 (p, J=7.0 Hz, 2H, CH₂CH₂(CH₂)₂CH₃), 3.20 (q, J=6.7 Hz, 2H, CH₂(CH₂)₃CH₃), 3.30 (vbs, 3H, NCH₃), 3.97 (vbs, 2H, NCH₂CH₃), 4.59 (s, 2H, CH₂CO₂H), 6.26 (bs, 1H, NH), 7.63 (s, 1H, CH(H-8)); HRMS (MS-ES), calcd for C₁₅H₂₅N₆O₂ [M+H] m/z=321.2034, found 321.2033; rpHPLC t_{R}: condition (I) 13.789 (II) 30.775 min, purity 99.7% and 99.5%.

### 86. Preparation of 2-(6-(isopropylamino)-2-(pentylamino)-9H-purin-9-yl)acetic acid (Compound ID: 8ah)

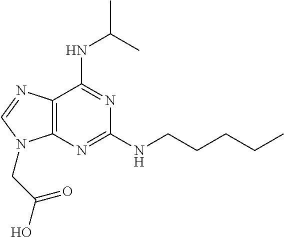 [SMILES: CCCCCNc1nc(NC(C)C)c2ncn(CC(=O)O)c2n1]

Purine 7ah was treated according to general procedure F, to yield final product Bah as a white lyophilized powder (73%): m.p.=173-176° C.; (KBr, cm⁻¹) 3685, 3653, 2926, 2857, 1581, 1420, 1383, 1304, 1202; δ_{H} (400 MHz, DMSO-d₆) 0.86 (t, J=6.7 Hz, 3H, (CH₂)₄CH₃), 1.20 (s, 3H, CH(CH₃)₂), 1.22 (s, 3H, CH(CH₃)₂), 1.25-1.34 (m, 4H, (CH₂)₂CH₂CH₂CH₃), 1.51 (p, J=6.8 Hz, 2H, CH₂CH₂(CH₂)₂CH₃), 3.22-3.28 (m, 2H, CH₂(CH₂)₃CH₃), 4.35 (bs, 1H, CH(CH₃)₂), 4.79 (s, 2H, CH₂CO₂H), 6.65 (bs, 1H, NH), 7.40 (bs, 1H, NH), 7.75 (s, 1H, CH(H-8)) 13.15 (vbs, 1H CH₂CO₂H); HRMS (MS-ES), calcd for C₁₅H₂₅N₆O₂ [M+H] m/z=321.2039, found 321.2033; rpHPLC tR: condition (I) 13.698 (II) 30.922 min, purity 94.6% and 91.0%.

### 87. Preparation of 2-(6-(allylamino)-2-(pentylamino)-9H-purin-9-yl)acetic acid (Compound ID: 8ai)

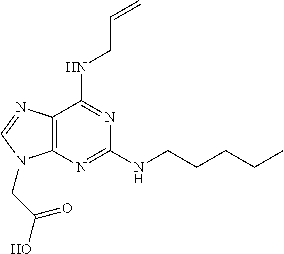 [SMILES: C=CCNc1nc(NCCCCC)nc2c1ncn2CC(=O)O]

Purine 7ai was treated according to general procedure F, to yield final product 8ai as a white lyophilized powder (76%): m.p.>153° C. (dec); IR (KBr, cm⁻¹) 3855, 3630, 1523, 1384, 1142; δ_{H} (400 MHz, DMSO-d₆) 0.85 (t, J=6.9, 3H, (CH₂)₄CH₃), 1.22-1.33 (m, 4H, (CH₂)₂CH₂CH₂CH₃), 1.49 (p, J=7.0 Hz, 2H, CH₂CH₂(CH₂)₂CH₃), 3.20 (q, J=6.6 Hz, 2H, CH₂(CH₂)₃CH₃), 4.07 (bs, 2H, CH₂CHCH₂), 4.47 (s, 2H, CH₂CO₂H), 5.02 (dd, 1H, J=10.3 Hz and 1.7 Hz, CH₂CHCH₂), 5.14 (dd, 1H, J=17.2 Hz and 1.8 Hz, CH₂CHCH₂), 5.88-5.99 (m, 1H, CH₂CHCH₂), 6.20 (bs, 1H, NH), 7.24 (bs, 1H, NH), 7.59 (s, 1H, CH(H-8)); δ_{C} (100 MHz, DMSO-d₆) 14.0, 21.9, 28.8, 29.0, 41.0, 45.0, 45.1, 112.5, 114.6, 136.4, 138.1, 144.5, 154.3, 159.2, 170.6; HRMS (MS-ES), calcd for C₁₅H₂₃N_{602 [}M+H] m/z=319.1869, found 319.1877; rpHPLC tR: condition (I) 13.326 (II) 28.780 min, purity 95.07% and 90.4%.

### 88. Preparation of 2-(6-(isobutylamino)-2-(pentylamino)-9H-purin-9-yl)acetic acid (Compound ID: 8aj)

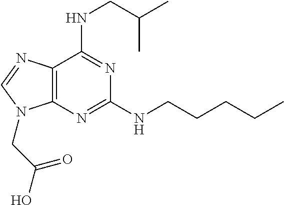 [SMILES: CCCCCNc1nc(NCC(C)C)c2ncn(CC(=O)O)c2n1]

Purine 7aj was treated according to general procedure F, to yield final product 8aj as a white lyophilized powder (75%): m.p.=139.1-147.8° C.; IR (KBr, cm⁻¹) 2956, 2926, 2854, 1467, 1385, 1246, 1186, 1142; δ_{H} (400 MHz, DMSO-d₆) 0.81-0.86 (m, 3H, (CH₂)₄CH₃), 0.87-0.92 (m, 6H, CH(CH₃)₂), 1.13-1.31 (m, 4H, (CH₂)₂CH₂CH₂CH₃), 1.52 (p, J=7.1 Hz, 2H, CH₂CH₂(CH₂)₂CH₃), 1.89-1.98 (m, 1H, CH(CH₃)₂), 3.23-3.31 (m, 4H, CH₂(CH₂)₃CH₃ and CH₂CH(CH₃)₂), 4.76 (s, 2H, CH₂CO₂H), 7.63 (bs, 1H, NH), 7.90 (s, 2H, CH(H-8) and NH); HRMS (MS-ES), calcd for C₁₆H₂₇N₆O₂ [M+H] m/z=335.2201, found 335.2190; rpHPLC t_{R}: condition (I) 14.357 (II) 22.765 min, purity 93.9% and 93.5%.

### 89. Preparation of 2-(6-(butyl(methyl)amino)-2-(pentylamino)-9H-purin-9-yl)acetic acid (Compound ID: 8ak)

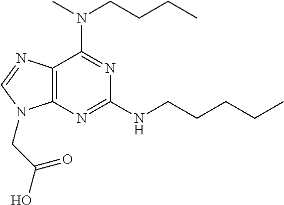 [SMILES: CCCCCNc1nc(N(C)CCCC)c2ncn(CC(=O)O)c2n1]

Purine 7ak was treated according to general procedure F, to yield final product 8ak as a white lyophilized powder (97%): m.p.>74° C. (dec); IR (KBr, cm⁻¹) 2959, 2931, 2859, 1561, 1459, 1396, 1324, 1203, 1137; δ_{H} (400 MHz, DMSO-d₆) 0.87 (t, J=6.7 Hz, 3H, (CH₂)₄CH₃), 0.91 (t, 3H, J=7.3 Hz, (CH₂)₃CH₃), 1.22-1.36 (m, 6H, CH₂CH₂CH₂CH₃ and (CH₂)₂CH₂CH₂CH₃), 1.53 (p, J=6.9 Hz, 2H, CH₂CH₂(CH₂)₂CH₃), 1.61 (p, 2H, CH₂CH₂CH₂CH₃), 3.23-4.17 (bm, 5H, CH₂(CH₂)₂CH₃ and NCH), 3.27 (t, J=7.3 Hz, 2H, CH₂CH₂(CH₂)₂CH₃), 4.84 (s, 2H, CH₂CO₂H), 6.90 (vbs, 1H, NH), 7.80 (s, 1H, CH(H-8)); HRMS (MS-ES), calcd for C₁₇H₂₉N₆O₂ [M+H] m/z=349.2342, found 349.2346; rpHPLC t_{R}: condition (I) 14.902 (II) 36.830 min, purity 97.8% and 95.8%.

### 90. Preparation of 2-(6-(isopentylamino)-2-(pentylamino)-9H-purin-9-yl)acetic acid (Compound ID: 8al)

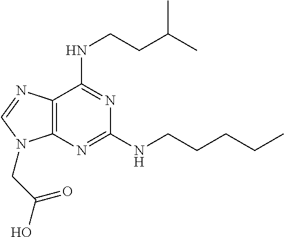 [SMILES: CCCCCNc1nc(NCCC(C)C)c2ncn(CC(=O)O)c2n1]

Purine 7al was treated according to general procedure F, to yield final product 8al as a white lyophilized powder (91%): m.p.>196° C. (dec); IR (KBr, cm⁻¹) 2956, 2928, 2858, 1578, 1470, 1431, 1409, 1367, 1306, 1224; δ_{H} (400 MHz, DMSO-d₆) 0.79-0.86 (m, 3H, (CH₂)₄CH₃), 0.87 (s, 3H, (CH₂)₂CH(CH₃)₂), 0.89 (s, 3H, (CH₂)₂CH(CH₃)₂), 1.10-1.30 (m, 4H, (CH₂)₂CH₂CH₂CH₃), 1.45-1.55 (m, 4H, CH₂CH₂(CH₂)₂CH₃ and CH₂CH₂CH(CH₃)₂), 1.59-1.67 (m, 1H, CH₂CH₂CH(CH₃)₂), 3.25-3.33 (m, 2H, CH₂(CH₂)₃CH₃), 3.41-3.53 (m, 2H, CH₂(CH₂)₃CH₃), 4.77 (s, 2H, CH₂CO₂H), 7.63 (bs, 1H, NH), 7.86 (s, 1H, CH(H-8)), 7.87 (bs, 1H, NH); δ_{C} (100 MHz, DMSO-d₆) 13.9, 21.9, 22.5, 25.3, 28.8, 29.1, 41.0, 43.6, 112.5, 131.0, 137.4, 154.6, 159.4, 169.7; HRMS (MS-ES), calcd for C₁₇H₂₉N₆O₂ [M+H] m/z=349.2339, found 349.2346; rpHPLC t_{R}: condition (I) 14.864 (II) 36.430 min, purity 90.3% and 96.1%.

### 91. Preparation of 2-(6-morpholino-2-(pentylamino)-9H-purin-9-yl)acetic acid (Compound ID: 8am)

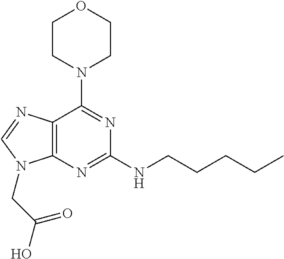 [SMILES: CCCCCNc1nc(N2CCOCC2)c2ncn(CC(=O)O)c2n1]

Purine 7 am was treated according to general procedure F, to yield final product 8 am as a white lyophilized powder (86%): m.p.>162° C. (dec); IR (KBr, cm⁻¹) 2956, 2926, 2855, 1444, 1384, 1120; δ_{H} (400 MHz, DMSO-d₆) 0.85 (t, J=6.9 Hz, 3H, (CH₂)₄CH₃), 1.22-1.31 (m, 4H, (CH₂)₂CH₂CH₂CH₃), 1.49 (p, J=6.9 Hz, 2H, CH₂CH₂(CH₂)₂CH₃), 3.2 (m, 2H, CH₂(CH₂)₃CH₃), 3.63-3.76 (m, 4H, 2CH₂ (morpholine)), 4.11 (bs, 4H, 2CH₂ (morpholine)), 4.69 (s, 2H, CH₂CO₂H), 6.40 (bs, 1H, NH), 7.69 (s, 1H, CH(H-8)); δ_{C} (100 MHz, DMSO-d₆) 13.9, 21.9, 28.7, 28.8, 40.9, 43.9, 44.9, 66.2, 112.7, 137.4, 153.3, 153.4, 158.7, 169.7; HRMS (MS-ES), calcd for C₁₆H₂₆N₆O₃ [M+H] m/z=349.1982, found 349.1974; rpHPLC t_{R}: condition (I) 12.899 (II) 26.385 min, purity 94.2% and 98.1%.

### 92. Preparation of 2-(6-(3-nitrophenoxy)-2-(pentylamino)-9H-purin-9-yl)acetic acid (Compound ID: 8an)

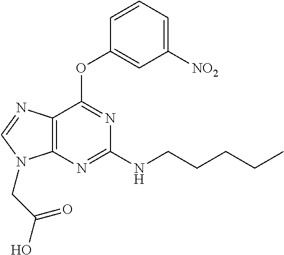 [SMILES: CCCCCNc1nc(Oc2cccc([N+](=O)[O-])c2)c2ncn(CC(=O)O)c2n1]

Purine 7an was treated according to general procedure F, to yield final product Ban as a white lyophilized powder (75%): m.p.>130° C. (dec); IR (KBr, cm⁻¹) 3550, 3407, 3336, 2958, 1352, 1200; δ_{H} (400 MHz, DMSO-d₆) 0.73-0.79 (m, 3H, (CH₂)₄CH₃), 0.96-1.42 (m, 6H, CH₂CH₂CH₂CH₂CH₃), 2.84-3.10 (m, 2H, CH₂(CH₂)₃CH₃), 4.87 (s, 2H, CH₂CO₂H), 7.14 (bm, 1H, NH), 7.75 (t, J=8.1 Hz, 1H, CH(Ar)), 7.78-7.81 (m, 1H, CH(Ar)), 8.00 (s, 1H, CH(H-8)), 8.14-8.20 (m, 2H, 2Ar)); δ_{C} (100 MHz, DMSO-d₆) 13.8, 21.7, 28.2, 28.5, 41.0, 43.8, 112.7, 117.3, 120.1, 128.9, 130.7, 141.6, 148.2, 152.7, 158.0, 158.5, 158.7, 169.2; HRMS (MS-ES), calcd for C₁₈H₂₁N₆O₅ [M+H] m/z=401.1568, found 401.1567; rpHPLC tR: condition (I) 13.772 (II) 31.491 min, purity 92.67% and 92.5%.

### 93. Preparation of 2-(6-(4-nitrophenoxy)-2-(pentylamino)-9H-purin-9-yl)acetic acid (Compound ID: 8ao)

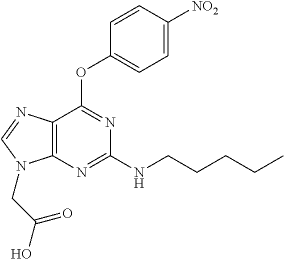 [SMILES: CCCCCNc1nc(Oc2ccc([N+](=O)[O-])cc2)c2ncn(CC(=O)O)c2n1]

Purine 7ao was treated according to general procedure F, to yield final product 8ao as a white lyophilized powder (72%): m.p.>101° C. (dec); IR (KBr, cm⁻¹) 3571, 3100, 2921, 1582, 1342, 1254; δ_{H} (400 MHz, DMSO-d₆) 0.78-0.86 (m, 3H, (CH₂)₄CH₃), 1.00-1.44 (m, 6H, CH₂CH₂CH₂CH₂CH₃), 2.87-2.92 (m, 2H, CH₂(CH₂)₃CH₃), 4.88 (s, 2H, CH₂CO₂H), 7.15 (bs, 1H, NH), 7.57 (d, J=9.2 Hz, 2H, 2CH(Ar)), 8.01 (s, 1H, CH(H-8)), 8.31 (d, J=9.2 Hz, 2H, 2CH(Ar)); δ_{C} (100 MHz, DMSO-d₆) 13.8, 21.7, 28.6, 41.0, 43.7, 43.8, 52.4, 112.9, 122.8, 125.1, 141.7, 144.3, 155.9, 157.8, 158.4, 158.6, 169.2; HRMS (MS-ES), calcd for C₁₈H₂₁N₆O₅ [M+H] m/z=401.1577, found 401.1567; rpHPLC t_{R}: condition (I) 13.586 (II) 30.762 min, purity 97.1% and 95.7%.

### 94. Preparation of 2-(6-(benzylamino)-2-((4-cyclohexylbenzyl)amino)-9H-purin-9-yl)acetic acid (Compound ID: 8ba)

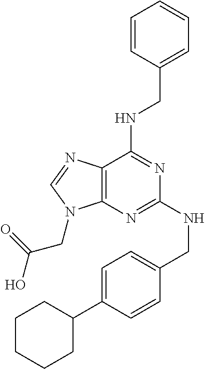 [SMILES: O=C(O)Cn1cnc2c(NCc3ccccc3)nc(NCc3ccc(C4CCCCC4)cc3)nc21]

Purine 7ba was treated according to general procedure F, to yield final product 8ba as a white lyophilized powder (79%): m.p.>182° C. (dec); IR (KBr, cm⁻¹) 3548, 3475, 3414, 2925, 2852, 1733, 1642, 1618, 1425, 1394, 1345, 1244; δ_{H} (400 MHz, DMSO-d₆) 1.28-1.38 (m, 5H (cyclohexyl)), 1.67-1.78 (m, 5H (cyclohexyl)), 2.42-2.45 (m, 1H, CH), 4.44 (s, 2H, HNCH₂), 4.63 (bs, 2H, CH₂Ar), 4.88 (s, 2H, CH₂CO₂H), 7.10-7.29 (m, 9H, 9 CH(Ar)), 7.63 (bs, 1H, NHAr), 7.94 (s, 1H, CH(H-8)), 8.70 (bs, 1H, NHAr); HRMS (MS-ES), calcd for C₂₇H₃₁N₆O₂ [M+H] m/z=471.2514, found 471.2503; rpHPLC t_{R}: condition (I) 18.355 (II) 42.706 min, purity 98.0% and 90.1%.

### 95. Preparation of 2-(6-(benzyl(methyl)amino)-2-((4-cyclohexylbenzyl)amino)-9H-purin-9-yl)acetic acid (Compound ID: 8bb)

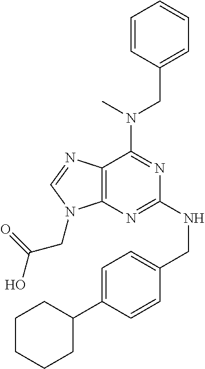 [SMILES: CN(Cc1ccccc1)c1nc(NCc2ccc(C3CCCCC3)cc2)nc2c1ncn2CC(=O)O]

Purine 7bb was treated according to general procedure F, to yield final product 8bb as a white lyophilized powder (82%): m.p.>133° C. (dec); IR (KBr, cm⁻¹) 3318, 2925, 2852, 1735, 1655, 1625, 1558, 1421, 1244, 1199; δ_{H} (400 MHz, DMSO-d₆) 1.29-1.42 (m, 5H, (cyclohexyl)), 1.67-1.77 (m, 5H (cyclohexyl)), 2.40-2.45 (m, 1H, CH), 2.97-3.71 (bm, 3H, NCH₃), 4.43 (s, 2H, CH₂Ar), 4.77-5.63 (br m, 2H, CH₃NCH₂), 4.87 (bs, 2H, CH₂CO₂H), 7.09-7.30 (m, 9H, 9 CH(Ar)), 7.47 (bs, 1H, NH), 7.82 (s, 1H, CH(H-8)); HRMS (MS-ES), calcd for C₂₈H₃₃N₆O₂ [M+H] m/z=485.2676, found 485.2659; rpHPLC t_{R}: condition (I) 18.496 (II) 44.040 min, purity 92.6% and 90.89%.

### 96. Preparation of 2-(2-((4-cyclohexylbenzyl)amino)-6-((furan-2-ylmethyl)(methyl)amino)-9H-purin-9yl)acetic acid (Compound ID: 8bd)

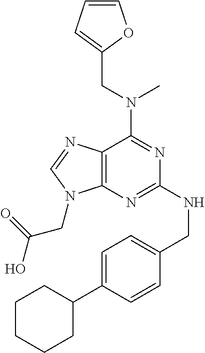 [SMILES: CN(Cc1ccco1)c1nc(NCc2ccc(C3CCCCC3)cc2)nc2c1ncn2CC(=O)O]

Purine 7bc was treated according to general procedure F, to yield final product 8bc as a white lyophilized powder (84%): m.p.>74° C. (dec); IR (KBr, cm⁻¹) 2925, 2851, 1661, 1555, 1402, 1320, 1201, 1138; δ_{H} (400 MHz, DMSO-d₆) 1.29-1.40 (m, 5H, 5H (cyclohexyl)), 1.67-1.77 (m, 5H (cyclohexyl)), 2.40-2.45 (m, 1H, CH), 2.98-3.57 (bm, 3H, CH₃ (furfuryl)), 4.24 (bm, 2H, CH₂ (furfuryl)), 4.43 (s, 2H, CH₂Ar), 4.84 (s, 2H, CH₂CO₂H), 6.21-6.40 (m, 2H, 2CH (furfuryl)), 7.11 (d, J=8.1 Hz, 2H, 2CH(Ar)), 7.24 (d, J=7.9 Hz, 2H, 2CH(Ar)), 7.20 (bs, 1H, NH), 7.54-7.60 (m, 1H, CH (furfuryl)), 7.80 (s, 1H, CH(H-8)); ε_{c} (100 MHz, DMSO-d₆) 25.5, 26.3, 33.9, 41.7, 43.4, 43.9, 44.2, 53.5, 108.0, 110.3, 112.8, 126.2, 127.4, 137.6, 138.1, 142.5, 145.7, 151.3, 153.6, 153.6, 169.3; HRMS (MS-ES), calcd for C₂₆H₃₁N₆O₃ [M+H] m/z=475.2445, found 475.2452; rpHPLC tR: condition (I) 16.862 (II) 42.090 min, purity 91.9% and 90.2%.

### 97. Preparation of 2-(2-((4-cyclohexylbenzyl)amino)-6-(cyclopentylamino)-9H-purin-9-yl)acetic acid (Compound ID: 8be)

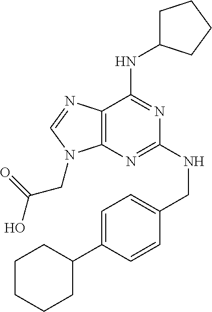 [SMILES: O=C(O)Cn1cnc2c(NC3CCCC3)nc(NCc3ccc(C4CCCCC4)cc3)nc21]

Purine 7be was treated according to general procedure F, to yield final product 8be as a white lyophilized powder (91%): m.p.>140° C. (dec); IR (KBr, cm⁻¹) 3855, 3508, 3294, 2928, 1388, 1202; δ_{H} (400 MHz, DMSO-d₆) 1.28-1.41 (m, 5H, (cyclohexyl)), 1.47-1.59 (m, 4H (cyclopentyl)), 1.65-1.93 (m, 9H, 5H (cyclohexyl) and 4H (cyclopentyl)), 2.40-2.45 (m, 1H, CH), 4.37 (bs, 1H, NCH), 4.41 (bs, 2H, CH₂Ar), 4.81 (s, 2H, CH₂CO₂H), 7.11 (d, J=7.9 Hz, 2H, 2CH(Ar)), 7.22 (bs, 1H, NH), 7.24 (d, J=8.1 Hz, 2H, 2CH(Ar)), 7.56 (br s, 1H, NH), 7.78 (bs, 1H, CH(H-8)); HRMS (MS-ES), calcd for C₂₅H₃₃N₆O₂ [M+H] m/z=449.2680, found 449.2659; rpHPLC t_{R}: condition (I) 17.193 (II) 43.772 min, purity 95.1% and 91.9%.

### 98. Preparation of 2-(6-(cyclohexylamino)-2-((4-cyclohexylbenzyl)amino)-9H-purin-9-yl)acetic acid (Compound ID: 8bf)

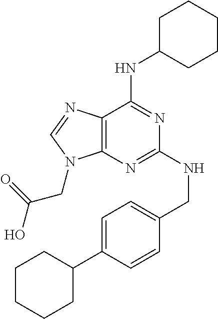 [SMILES: O=C(O)Cn1cnc2c(NC3CCCCC3)nc(NCc3ccc(C4CCCCC4)cc3)nc21]

Purine 7bf was treated according to general procedure F, to yield final product 8bf as a white lyophilized powder (88%): m.p.=172-179° C.; IR (KBr, cm⁻¹) 2927, 2854, 1448, 1388, 1201, 1142; δ_{H} (400 MHz, DMSO-d₆) 1.13-1.38 (m, 10H, 5H (cyclohexyl) and 5H (NH-cyclohexyl)), 1.56-1.81 (m, 10H, 5H (cyclohexyl) and 5H(NH-cyclohexyl)), 2.38-2.48 (m, 1H, CH), 3.89 (bs, 1H, HNCH), 4.44 (s, 2H, CH₂Ar), 4.89 (s, 2H, CH₂CO₂H), 7.14 (d, J=7.7 Hz, 2H, 2CH(Ar)), 7.26 (d, J=7.7 Hz, 2H, 2CH(Ar)), 7.75 (bs, 1H, NH), 7.99 (s, 1H, CH(H-8)); HRMS (MSES), calcd for C₂₆H₃₅N₆O₂ [M+H] m/z=463.2819, found 463.2816; rpHPLC t_{R}: condition (I) 17.233 (II) 44.956 min, purity 95.3% and 92.2%.

### 99. Preparation of 2-(6-(allylamino)-2-((4-cyclohexylbenzyl)amino)-9H-purin-9-yl)acetic acid (Compound ID: 8bi)

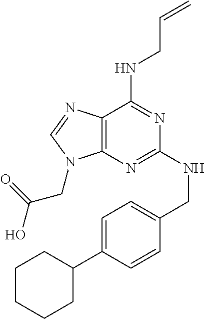 [SMILES: C=CCNc1nc(NCc2ccc(C3CCCCC3)cc2)nc2c1ncn2CC(=O)O]

Purine 7bi was treated according to general procedure F, to yield final product 8bi as a white lyophilized powder (81%): m.p.>170° C. (dec); IR (KBr, cm⁻¹) 3550, 3477, 3414, 2924, 2852, 1638, 1618, 1385, 1201; δ_{H} (400 MHz, DMSO-d₆) 1.27-1.44 (m, 5H (cyclohexyl)), 1.62-1.81 (m, 5H (cyclohexyl)), 2.38-2.48 (m, 1H, CH), 4.04 (bs, 2H, CH₂CHCH₂), 4.43 (s, 2H, CH₂Ar), 4.84 (s, 2H, CH₂CO₂H), 5.05 (dd, J=10.1 and 1.5 Hz, 1H, CH₂CHCH₂), 5.14 (dd, J=17.1 and 1.5 Hz, 1H, CH₂CHCH₂), 5.81-5.97 (m, 1H, CH₂CHCH₂), 7.12 (d, J=8.1 Hz, 2H, 2CH(Ar)), 7.25 (d, J=8.1 Hz, 2H, 2CH(Ar)), 7.36 (bs, 1H, NH), 7.85 (s, 1H, CH(H-8)), 8.04 (bs, 1H, NH); HRMS (MS-ES), calcd for C₂₃H₂₉N₆O₂ [M+H] m/z=421.2349, found 421.2346; rpHPLC tR: condition (I) 15.403 (II) 40.030 min, purity 96.4% and 93.86%.

### 100. Preparation of 2-(2-((4-cyclohexylbenzyl)amino)-6-(isobutylamino)-9H-purin-9-yl)acetic acid (Compound ID: 8bj)

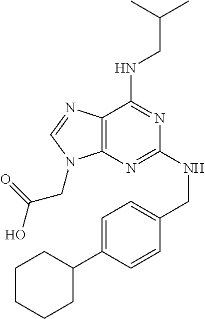 [SMILES: CC(C)CNc1nc(NCc2ccc(C3CCCCC3)cc2)nc2c1ncn2CC(=O)O]

Purine 7bj was treated according to general procedure F, to yield final product 8bj as a white lyophilized powder (73%): m.p.>116° C. (dec); IR (KBr, cm⁻¹) 3549, 3477, 3414, 2920, 1744, 1620, 1449, 1404, 1387, 1367, 1248, 1206; δ_{H} (400 MHz, DMSO-d₆) 0.81 (s, 3H, CH₂CH(CH₃)₂), 0.83 (s, 3H, CH₂CH(CH₃)₂), 1.26-1.42 (m, 5H, (cyclohexyl)), 1.62-1.80 (m, 5H, (cyclohexyl)), 1.79-1.92 (m, 1H, CH₂CH(CH₃)₂) 2.33-2.46 (m, 1H, CH), 3.16 (bs, 2H, CH₂CH(CH₃)₂), 4.30-4.43 (m, 2H, CH₂Ar), 4.69 (s, 2H, CH₂CO₂H), 6.90 (bs, 1H, NH), 7.1 (d, J=7.9 Hz, 2H, 2CH(Ar)), 7.23 (d, J=7.9 Hz, 2H, 2CH(Ar)), 7.27 (bs, 1H, NH), 7.64 (s, 1H, CH(H-8)); δ_{C} (100 MHz, DMSO-d₆) 20.1, 25.5, 26.3, 34.0, 43.4, 43.8, 44.3, 46.9, 112.6, 126.1, 127.3, 137.5, 138.9, 145.4, 151.4, 154.7, 159.1, 169.7; HRMS (MS-ES), calcd for C₂₄H₃₃N₆O₂ [M+H] m/z=437.2663, found 437.2659; rpHPLC t_{R}: condition (I) 16.906 (II) 45.089 min, purity 96.6% and 97.8%.

### 101. Preparation of 2-(2-((tert-butoxycarbonyl)(4-cyclohexylbenzyl)amino)-6-(isopentylamino)-9H-purin-9yl)acetic acid (Compound ID: 8bl)

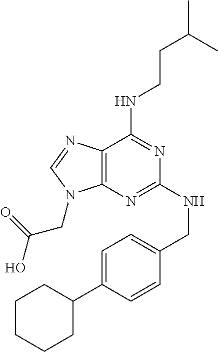 [SMILES: CC(C)CCNc1nc(NCc2ccc(C3CCCCC3)cc2)nc2c1ncn2CC(=O)O]

Purine 7bl was treated according to general procedure F, to yield final product 8bl as a white lyophilized powder (69%): m.p.>153° C. (dec); IR (KBr, cm⁻¹) 2937, 2851, 1736, 1646, 1528, 1432, 1244, 1201; δ_{H} (400 MHz, DMSO-d₆) 0.85 (s, 3H, (CH₂)₂CH(CH₃)₂), 0.86 (s, 3H, (CH₂)₂CH(CH₃)₂), 1.18-1.60 (m, 8H, 5H (cyclohexyl) and (CH₂)₂CH(CH₃)₂ and CH₂CH₂CH(CH₃)₂), 1.67-1.77 (m, 5H, (cyclohexyl)), 2.41-2.47 (m, 1H, CH), 3.41 (bs, 2H, CH₂CH₂CH(CH₃)₂), 4.47 (s, 2H, CH₂Ar), 4.88 (s, 2H, CH₂CO₂H), 7.14 (d, J=7.9 Hz, 2H, 2 CH(Ar)), 7.25 (d, J=7.7 Hz, 2H, 2CH(Ar)), 7.58 (bs, 1H, NH), 7.91 (s, 1H, CH(H-8)), 8.32 (bs, 1H, NH); HRMS (MS-ES), calcd for C₂₅H₃₅N₆O₂ [M+H] m/z=451.2835, found 451.2816; rpHPLC t_{R}: condition (I) 17.061 (II) 44.519 min, purity 91.9% and 94.2%.

### 102. Preparation of 2-(2-((4-cyclohexylbenzyl)amino)-6-morpholino-9H-purin-9-yl)acetic acid (Compound ID: 8bm)

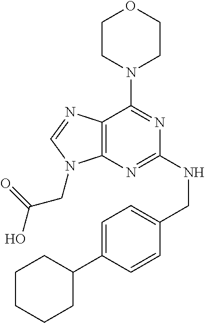 [SMILES: O=C(O)Cn1cnc2c(N3CCOCC3)nc(NCc3ccc(C4CCCCC4)cc3)nc21]

Purine 7bm was treated according to general procedure F, to yield final product 8bm as a white lyophilized powder (73%): m.p.>147° C. (dec); IR (KBr, cm⁻¹) 3422, 2923, 2851, 1603, 1542, 1516, 1446, 1416, 1384, 1314, 1272, 1242, 1207, 1121, 1003; δ_{H} (400 MHz, DMSO-d₆) 1.171.36 (m, 5H, (cyclohexyl)), 1.66-1.77 (m, 5H, (cyclohexyl)), 2.38-2.45 (m, 1H, CH), 3.63 (t, J=4.4 Hz, 4H, 2CH₂, (morpholine)), 4.08 (bs, 4H, 2CH₂, (morpholine)), 4.37 (d, J=5.1 Hz, 2H, CH₂Ar), 4.78 (s, 2H, CH₂CO₂H), 7.03 (bs, 1H, NH), 7.1 (d, J=8.1 Hz, 2H, 2CH(Ar)), 7.23 (d, J=7.9 Hz, 2H, 2CH(Ar)), 7.73 (s, 1H, CH, (H-8)); HRMS (MS-ES), calcd for C₂₄H₃ᵢN₆O₃ [M+H] m/z=451.2463, found 451.2452; rpHPLC t_{R}: condition (I) 14.895 (II) 38.319 min, purity 99.9% and 96.6%.

### 103. Preparation of 2-(2-((4-cyclohexylbenzyl)amino)-6-(3-nitrophenoxy)-9H-purin-9-yl)acetic acid (Compound ID: 8bl)

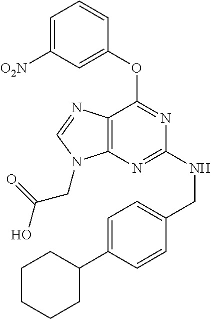 [SMILES: O=C(O)Cn1cnc2c(Oc3cccc([N+](=O)[O-])c3)nc(NCc3ccc(C4CCCCC4)cc3)nc21]

Purine 7bn was treated according to general procedure F, to yield final product 8bl as a white lyophilized powder (86%): m.p.>150° C. (dec); IR (KBr, cm⁻¹) 3434, 2926, 2853, 1587, 1526, 1417, 1352, 1252; δ_{H} (400 MHz, DMSO-d₆) 1.29-1.37 (m, 5H, (cyclohexyl)), 1.66-1.77 (m, 5H, (cyclohexyl)), 2.35-2.41 (m, 1H, CH), 4.17 (m, 2H, CH₂Ar), 4.89 (s, 2H, CH₂CO₂H), 6.81-7.20 (m, 4H, 3CH(Ar) and NH), 7.72-7.75 (m, 3H, 3CH (Ar)), 8.00 (s, 1H, CH, (H-8)), 8.10-8.17 (m, 2H, 2CH(Ar)); δ_{C} (100 MHz, DMSO-d₆) 25.5, 26.3, 33.9, 43.4, 43.9, 44.2, 113.0, 117.3, 120.2, 126.0, 127.6, 129.1, 130.7, 137.5, 141.7, 145.7, 148.3, 152.7, 155.8, 158.3, 158.8, 169.2; HRMS (MS-ES), calcd for C₂₆H₂₇N₆O₅ [M+H] m/z=503.2018, found 503.2037; rpHPLC t_{R}: condition (I) 15.558 (II) 40.643, purity 99.7% and 99.0%

### 104. Preparation of 2-(2-((4-cyclohexylbenzyl)amino)-6-(4-nitrophenoxy)-9H-purin-9-yl)acetic acid (Compound ID: 8bo)

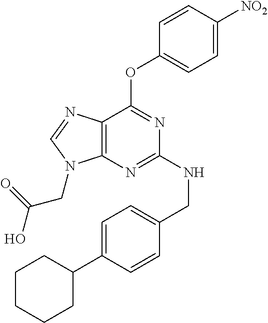 [SMILES: O=C(O)Cn1cnc2c(Oc3ccc([N+](=O)[O-])cc3)nc(NCc3ccc(C4CCCCC4)cc3)nc21]

Purine 7bo was treated according to general procedure F, to yield final product 8bo as a white lyophilized powder (74%): m.p.>170° C. (dec); IR (KBr, cm⁻¹) 3550, 3413, 2924, 2852, 1724, 1636, 1616, 1581, 1552, 1522, 1488, 1449; δ_{H} (400 MHz, DMSO-d₆) 1.31-1.39 (m, 5H, (cyclohexyl)), 1.67-1.78 (m, 5H, (cyclohexyl)), 2.37-2.44 (m, 1H, CH), 4.07-4.36 (m, 2H, CH₂Ar), 4.89 (s, 2H, CH₂CO₂H), 6.92-7.26 (m, 4H, 4 CH(Ar)), 7.44-7.53 (m, 2H, 2CH(Ar)), 7.70 (bs, 1H, NH), 8.01 (s, 1H, CH, (H-8)), 8.23-8.28 (m, 2H, 2CH(Ar)); δ_{C} (100 MHz, DMSOd₆) 25.5, 26.3, 33.9, 43.4, 43.8, 44.2, 113.2, 115.8, 122.6, 125.1, 126.1, 127.4, 127.9, 137.4, 141.9, 144.2, 145.7, 157.6, 158.4, 169.2; HRMS (MS-ES), calcd for C₂₆H₂₇N₆O₅ [M+H] m/z=503.2026, found 503.2037; rpHPLC t_{R}: condition (I) 13.824 (II) 41.102 min, purity 90.4% and 90.2%.

### 105. Preparation of 2-(2-((4-cyclohexylbenzyl)amino)-6-((4-fluorophenyl)amino)-9H-purin-9-yl)acetic acid (Compound ID: 8bp)

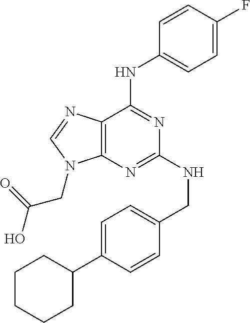 [SMILES: O=C(O)Cn1cnc2c(Nc3ccc(F)cc3)nc(NCc3ccc(C4CCCCC4)cc3)nc21]

Purine 7bp was treated according to general procedure F, to yield final product 8bp as a white lyophilized powder (91%): m.p.>125° C. (dec); IR (KBr, cm⁻¹) 3429, 3226, 2924, 2851, 1682, 1646, 1509, 1206, 1134; δ_{H} (400 MHz, DMSO-d₆) 1.31-1.40 (m, 5H, (cyclohexyl)), 1.66-1.76 (m, 5H (cyclohexyl)), 2.40-2.46 (m, 1H, CH), 4.42 (d, J=6.2 Hz, 2H, CH₂Ar), 4.83 (s, 2H, CH₂CO₂H), 6.99-7.04 (m, 2H, 2CH(Ar)), 7.12 (d, J=8.1 Hz, 2H, 2CH(Ar)), 7.24 (d, J=8.1 Hz, 2H, 2CH(Ar)), 7.29 (bs, 1H, NHAr), 7.75-7.90 (m, 2H, 2 CH(Ar)), 7.83 (s, 1H, CH(H8)), 9.50 (s, 1H, NHAr), 13.21 (bs, 1H, CO₂H); δ_{C} (100 MHz, DMSO-d₆) 25.5, 26.3, 34.0, 41.7, 43.4, 44.3, 113.1, 114.4, 114.6, 121.5, 121.6, 126.2, 136.5, 138.3, 138.6 145.5, 151.8, 156.0, 158.9, 169.5; HRMS (MS-ES), calcd for C₂₆H₂₈N₆O₂F [M+H] m/z=475.2266, found 475.2252; rpHPLC t_{R}: condition (I) 17.250 (II) 43.207 min, purity 99.9% and 95.6%.

### 106. Preparation of 2-(2-((4-cyclohexylbenzyl)amino)-6-((furan-2-ylmethyl)amino)-9H-purin-9-yl)acetic acid (Compound ID: 8bq)

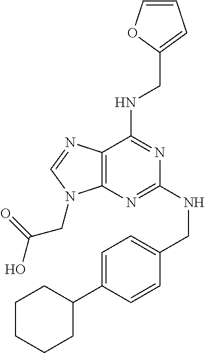 [SMILES: O=C(O)Cn1cnc2c(NCc3ccco3)nc(NCc3ccc(C4CCCCC4)cc3)nc21]

Purine 7bq was treated according to general procedure F, to yield final product 8bq as a white lyophilized powder (88%): m.p.>162 (dec)° C.; IR (KBr, cm⁻¹) 3320, 2920, 2855, 1731, 1574, 1530, 1426, 1246, 1201, 1141; δ_{H} (400 MHz, DMSO-d₆) 1.29-1.42 (m, 5H, 5H (cyclohexyl)), 1.67-1.77 (m, 5H (cyclohexyl)), 2.41-2.47 (m, 1H, CH), 4.46 (s, 2H, CH₂ (furfuryl)), 4.62 (bs, 2H, CH₂Ar), 4.88 (s, 2H, CH₂CO₂H), 6.15-6.26 (m, 1H, CH (furfuryl)), 6.35 (bs, 1H, CH (furfuryl)), 7.12 (d, J=7.9 Hz, 2H, 2CH(Ar)), 7.25 (d, J=7.9 Hz, 2H, 2CH(Ar)), 7.55 (s, 1H, CH (furfuryl)), 7.61 (bs, 1H, NH), 7.96 (bs, 1H, CH(H-8)), 8.33-8.55 (bm, 1H, NH); HRMS (MS-ES), calcd for C₂₅H₂₉N₆O₃ [M+H] m/z=461.2297, found 461.2295; rpHPLC t_{R}: condition (I).

### 107. Preparation of 2-(2-((tert-butoxycarbonyl)(4-cyclohexylbenzyl)amino)-6-(propylamino)-9H-purin-9-yl) acetic acid (Compound ID: 8bs)

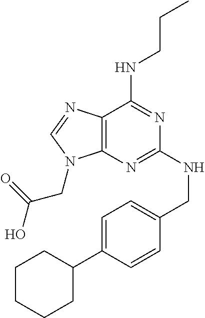 [SMILES: CCCNc1nc(NCc2ccc(C3CCCCC3)cc2)nc2c1ncn2CC(=O)O]

Purine 7bs was treated according to general procedure H, to yield final product 8bs as a white lyophilized powder (78%): m.p.>202° C. (dec); IR (KBr, cm⁻¹) 3677, 3519, 3396, 2922, 1452, 1123; δ_{H} (400 MHz, DMSO-d₆) 0.85 (m, 3H, NHCH₂CH₂CH₃), 1.26-1.41 (m, 5H (cyclohexyl)), 1.53 (m, 2H, NHCH₂CH₂CH₃), 1.65-1.78 (m, 5H (cyclohexyl)), 2.40-2.46 (m, 1H, CH), 3.38 (bs, 2H, NHCH₂CH₂CH₃), 4.41-4.44 (m, 2H, CH₂Ar), 4.85 (s, 2H, CH₂CO₂H), 7.11 (d, J=8.1 Hz, 2H, 2CH(Ar)), 7.23 (d, J=7.9 Hz, 2H, 2CH(Ar)), 7.88 (s, 1H, CH, (H-8)); δ_{C} (100 MHz, DMSO-d₆) 11.2, 25.5, 26.3, 33.9, 43.5, 43.9, 44.1, 44.2, 112.7, 115.7, 118.6, 121.6, 126.3, 127.6, 157.9, 158.2, 158.5, 158.8, 169.1; HRMS (MS-ES), calcd for C₂₃H₃₁N₆O₂ [M+H] m/z=423.2499, found 423.5203; rpHPLC t_{R}: condition (I) 15.644 (II) 41.468 min, purity 90.4% and 90.2%.

### 108. Preparation of 2-(2-((4-cyclohexylbenzyl)amino)-6-(hexylamino)-9H-purin-9-yl)acetic acid (Compound ID: 8bt)

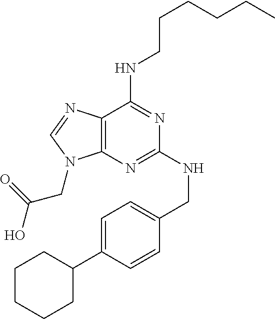 [SMILES: CCCCCCNc1nc(NCc2ccc(C3CCCCC3)cc2)nc2c1ncn2CC(=O)O]

Purine 7bt was treated according to general procedure F, to yield final product 8bt as a white lyophilized powder (85%): m.p.>105° C. (dec); IR (KBr, cm⁻¹) 3549, 3413, 2925, 2853, 1686, 1638, 1618, 1448, 1384, 1303, 1208, 1183; δ_{H} (400 MHz, CDCl₃) 0.84 (t, J=7.1 Hz, 3H, (CH₂)₅CH₃), 1.10-1.39 (m, 11H, 5H (cyclohexyl) and (CH₂)₂CH₂CH₂CH₂CH₃), 1.43-1.57 (m, 2H, CH₂CH₂(CH₂)₃CH₃)), 1.60-1.87 (m, 5H, (cyclohexyl)), 2.37-2.47 (m, 1H, CH), 3.42 (bs, 2H, CH₂(CH₂)₄CH₃), 4.42 (s, 2H, CH₂Ar), 4.81 (s, 2H, CH₂CO₂H), 7.11 (d, J=7.9 Hz, 2H, 2CH(Ar)), 7.24 (d, J=8.1 Hz, 2H, 2CH(Ar)), 7.25 (bs, 1H, NH), 7.76 (bs, 1H, NH), 7.77 (s, 1H, CH, (H-8)); δ_{C} (100 MHz, DMSO-d₆) 13.8, 18.8, 22.1, 25.5, 26.1, 26.3, 28.9, 31.0, 33.9, 43.4, 43.7, 43.8, 44.2, 112.1, 121.9, 126.2, 127.4, 128.6, 131.5, 145.6, 158.1, 169.3; HRMS (MSES), calcd for C₂₆H₃₇N₆O₂ [M+H] m/z=465.2991, found 465.2983; rpHPLC t_{R}: condition (I) 16.366 (II) 30.267 min, purity 92.7% and 95.7%.

### 109. Preparation of 2-(6-(3-bromophenoxy)-2-((4-cyclohexylbenzyl)amino)-9H-purin-9-yl)acetic acid (Compound ID: 8bu)

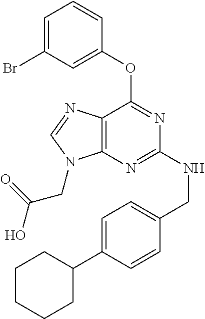 [SMILES: O=C(O)Cn1cnc2c(Oc3cccc(Br)c3)nc(NCc3ccc(C4CCCCC4)cc3)nc21]

Purine 7bu was treated according to general procedure F, to yield final product 8bu as a white lyophilized powder (93%): m.p.>128° C. (dec); IR (KBr, cm⁻¹) 3462, 2921, 2850, 1729, 1626, 1449, 1349, 1237; δ_{H} (400 MHz, DMSO-d₆) 1.22-1.38 (m, 5H, (cyclohexyl)), 1.64-1.82 (m, 5H, (cyclohexyl)), 2.35-2.47 (m, 1H, CH), 4.04-4.25 (m, 2H, CH₂, (Ar)), 4.88 (s, 2H, CH₂CO₂H), 6.71-7.18 (m, 4H, 4 CH(Ar)), 7.22-7.34 (m, 1H, CH (Ar)), 7.42 (t, J=8.1 Hz, 1H, CH(Ar)), 7.47-7.52 (m, 1H, CH(Ar)), 7.54 (t, J=2.02 Hz, 1H, CH(Ar)), 7.69 (bs, 1H, NH), 8.00 (s, 1H, CH, (H-8)); δ_{C} (100 MHz, DMSO-d₆) 25.5, 26.3, 33.9, 43.4, 43.8, 44.2, 112.8, 121.4, 121.5, 125.1, 125.2, 126.2, 127.8, 128.3, 131.1, 137.5, 141.4, 145.7, 153.1, 158.4, 159.1, 169.2; HRMS (MS-ES), calcd for C₂₆H₂₇N₅O₃Br [M+H] m/z=536.1271, found 536.1291; rpHPLC t_{R}: condition (I) 16.049 (II) 43.812 min, purity 99.8% and 97.32%.

### 110. Preparation of 2-(2-((4-cyclohexylbenzyl)amino)-6-(4-fluorophenoxy)-9H-purin-9-yl)acetic acid (Compound ID: 8bv)

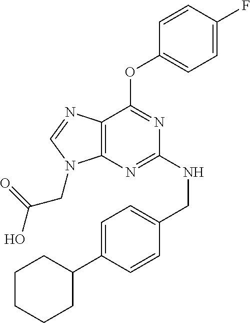 [SMILES: O=C(O)Cn1cnc2c(Oc3ccc(F)cc3)nc(NCc3ccc(C4CCCCC4)cc3)nc21]

Purine 7bv was treated according to general procedure F, to yield final product 8bv as a white lyophilized powder (77%): m.p.>100° C. (dec); IR (KBr, cm⁻¹) 3550, 3414, 3235, 2925, 2852, 1619, 1587, 1504, 1450, 1408, 1349, 1256; δ_{H} (400 MHz, DMSO-d₆) 1.31-1.40 (m, 5H, (cyclohexyl)), 1.67-1.79 (m, 5H, (cyclohexyl)), 2.38-2.44 (m, 1H, CH), 4.01-4.32 (m, 2H, CH₂Ar), 4.88 (s, 2H, CH₂CO₂H), 6.57-7.14 (m, 4H, 4 CH(Ar)), 7.26 (d, J=6.8 Hz, 4H, 4 CH(Ar)), 7.61 (bs, 1H, NH), 8.00 (s, 1H, CH, (H-8)); δ_{C} (100 MHz, DMSO-d₆) 25.5, 26.3, 33.9, 43.4, 43.8, 44.2, 112.8, 115.8, 116.0, 123.6, 123.7, 126.1, 127.8, 137.5, 145.7, 148.2, 148.3, 158.1, 158.4, 159.4, 160.5, 169.2; HRMS (MS-ES), calcd for C₂₆H₂₇FN₅O₃ [M+H] m/z=476.2073, found 476.2092; rpHPLC t_{R}: condition (I) 15.577 (II) 41.341 min, purity 95.7% and 92.1%.

### 111. Preparation of 2-(2-((4-cyclohexylbenzyl)amino)-6-(perfluorophenoxy)-9H-purin-9-yl)acetic acid (Compound ID: 8bw)

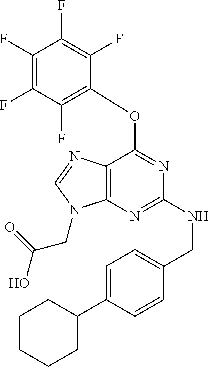 [SMILES: O=C(O)Cn1cnc2c(Oc3c(F)c(F)c(F)c(F)c3F)nc(NCc3ccc(C4CCCCC4)cc3)nc21]

Purine 7bw was treated according to general procedure F, to yield final product 8bw as a white lyophilized powder (84%): m.p.>110° C. (dec); IR (KBr, cm⁻¹) 3550, 3408, 2925, 1637, 1618, 1584, 1558, 1521, 1404, 1227; δ_{H} (400 MHz, DMSO-d₆) 1.29-1.40 (m, 5H, (cyclohexyl)), 1.67-1.80 (m, 5H, (cyclohexyl)), 2.38-2.45 (m, 1H, CH), 4.02-4.41 (m, 2H, CH₂Ar), 4.91 (s, 2H, CH₂CO₂H), 6.74-7.31 (m, 4H, 4 CH(Ar)), 8.01 (bs, 1H, NH), 8.07 (s, 1H, CH(H-8)), 13.3 (vbs, 1H, CO₂H); δC (100 MHz, DMSO-d₆) 25.5, 26.3, 33.9, 43.4, 43.9, 44.4, 112.0, 126.1, 127.1, 132.9, 138.5, 142.5, 142.9, 145.6, 152.3, 156.1, 158.1, 159.2, 169.1; HRMS (MS-ES), calcd for C₂₆H₂₃F₅N₅O₃ [M+H] m/z=548.1704, found 548.1715; rpHPLC t_{R}: condition (I) 16.078 (II) 44.286 min, purity 97.2% and 97.3%.

### 112. Preparation of 2-(2-((4-cyclohexylbenzyl)amino)-6-(perfluorophenoxy)-9H-purin-9-yl)acetic acid (Compound ID: 8bx)

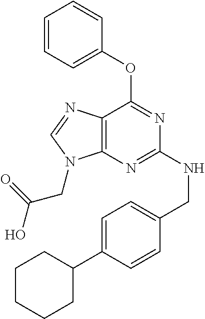 [SMILES: O=C(O)Cn1cnc2c(Oc3ccccc3)nc(NCc3ccc(C4CCCCC4)cc3)nc21]

Purine 7bx was treated according to general procedure F, to yield final product 8bx as a white lyophilized powder (82%): m.p.>129° C. (dec); IR (KBr, cm⁻¹) 3707, 2925, 2851, 1580, 1546, 1401, 1349, 1254; δ_{H} (400 MHz, DMSO-d₆) 1.25-1.38 (m, 5H, (cyclohexyl)), 1.66-1.76 (m, 5H, (cyclohexyl)), 2.37-2.43 (m, 1H, CH), 4.01-4.25 (m, 2H, CH₂Ar), 4.60 (s, 2H, CH₂CO₂H), 7.02 0.05 (m, 3H, 3CH(Ar)), 7.17-7.28 (m, 4H, 4 CH(Ar)), 7.39-7.46 (m, 3H, 2CH(Ar) and 1NH), 0.89 (s, 1H, CH(H-8)); δ_{C} (100 MHz, DMSO-d₆) 25.5, 26.3, 33.9, 43.4, 44.1, 45.4, 113.2, 121.8, 125.0, 126.2, 127.7, 129.4, 137.8, 141.9, 145.6, 152.4, 155.4, 158.3, 159.3, 170.0; HRMS (MSES), calcd for C₂₆H₂₈N₅O₃ [M+H] m/z=458.2180, found 458. 2186; rpHPLC t_{R}: condition (I) 15.570 (II) 40.997 min, purity 97.8% and 97.1%.

### 113. Preparation of methyl 2-(2-amino-6-chloro-9H-purin-9-yl(acetate (Compound ID: 9)

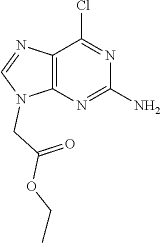 [SMILES: CCOC(=O)Cn1cnc2c(Cl)nc(N)nc21]

Purine 4 was treated according to general procedure F, to yield lyophilized product 9 as an off-white solid (90%): m.p.=148-150° C.; IR (KBr, cm⁻¹) 2982, 1761, 1738, 1522, 1473, 1423, 1441, 1380, 1343, 1310, 1286, 1225, 1173, 1143, 1023, 1002; δ_{H} (400 MHz, CDCl₃) 1.30 (t, J=7.2 Hz, 3H, CO₂CH₂CH₃), 4.26 (q, J=7.2 Hz, 2H, CO₂CH₂CH₃), 4.84 (s, 2H, CH₂CO₂Et), 5.15 (bs, 2H, NH₂), 7.83 (s, 1H, CH(H-8)); LRMS (MS-ES), calcd for C₉H₁₁C₁N₅O₂ [M+H] m/z=256.05, found 256.18.

### 114. Preparation of ethyl 2-(6-chloro-2-(4-cyclohexylbenzamido)-9H-purin-9-yl)acetate (Compound ID: 10)

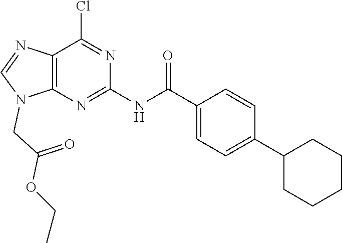 [SMILES: CCOC(=O)Cn1cnc2c(Cl)nc(NC(=O)c3ccc(C4CCCCC4)cc3)nc21]

Purine 9 was treated with 4-cyclohexylbenzoyl chloride according to general procedure G, to yield lyophilized product 10 as a yellow solid (63%): m.p.=90-107° C.; IR (KBr, cm⁻¹) 2924, 2850, 1750, 1576, 1493, 1437, 1402, 1285, 1215, 1172; δ_{H} (400 MHz, CDCl₃) 1.32 (t, J=7.2 Hz, 3H, CO₂CH₂CH₃), 1.38-1.49 (m, 5H, (cyclohexyl), 1.76-1.92 (m, 5H, (cyclohexyl)), 2.56-2.62 (m, 1H, CH), 4.29 (q, J=7.2 Hz, 2H, CO₂CH₂CH₃), 5.06 (s, 2H, CH₂CO₂Et), 7.35 (d, J=8.3 Hz, 2H, 2CH(Ar)), 7.87 (d, J=8.3 Hz, 2H, 2CH(Ar)), 8.12 (s, 1H, CH(H-8)), 8.71 (bs, 1H, NH); LRMS (MS-ES), calcd for C₂₂H₂₄C₁₁N₅O₃Na [M+Na] m/z=464.16, found 464.32.

### 115. Preparation of ethyl 2-(6-(butyl(methyl)amino)-2-(4-cyclohexylbenzamido)-9H-purin-9-yl)acetate (Compound ID: 11a)

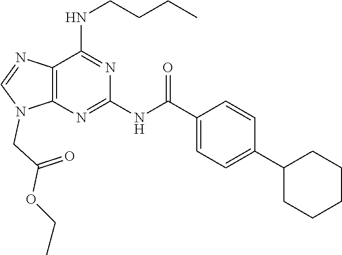 [SMILES: CCCCNc1nc(NC(=O)c2ccc(C3CCCCC3)cc2)nc2c1ncn2CC(=O)OCC]

Purine 10 was treated with N-butylmethylamine according to general procedure B, yielding the final product 11a as a clear viscous oil (69%): IR (KBr, cm⁻¹) 3630, 2931, 1752, 1578, 1533, 1449, 1406, 1353, 1275, 1221, 1149; δ_{H} (400 MHz, CDCl₃) 0.95 (t, J=7.4 Hz, 3H, N(CH₂)₃CH₃), 1.27-1.49 (m, 7H, N(CH₂)₂CH₂CH₃ and 5H (cyclohexyl)), 1.30 (t, J=7.2 Hz, 3H, CO₂CH₂CH₃), 1.63-1.91 (m, 7H, NCH₂CH₂CH₂CH₃ and 5H (cyclohexyl)), 2.54-2.61 (m, 1H, CH), 3.16-4.34 (bm, 5H, CH₃NCH₂(CH₂)₂CH₃), 4.26 (q, J=7.2 Hz, 2H, CO₂CH₂CH₃), 4.92 (s, 2H, CH₂CO₂Et), 7.31 (d, J=8.3 Hz, 2H, 2CH(Ar)), 7.72 (s, 1H, CH (H-8)), 7.82 (d, J=7.9 Hz, 2H, 2CH(Ar)), 8.24 (bs, 1H, NH); LRMS (MS-ES), calcd for C₂₇H₃₇N₆O₃ [M+H] m/z=493.28, found 493.47.

### 116. Preparation of ethyl 2-(6-(benzylamino)-2-(4-cyclohexylbenzamido)-9H-purin-9-yl)acetate (Compound ID: 11b)

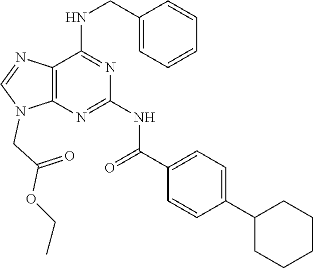 [SMILES: CCOC(=O)Cn1cnc2c(NCc3ccccc3)nc(NC(=O)c3ccc(C4CCCCC4)cc3)nc21]

Purine 10 was treated with benzylamine according to general procedure B, yielding the final product 11b as a off-white solid (83%): m.p.>100-118° C.; IR (KBr, cm⁻¹) 2924, 1449, 1385, 1245; δ_{H} (400 MHz, CDCl₃) 1.31 (t, J=7.2 Hz, 3H, CO₂CH₂CH₃), 1.35-1.50 (m, 5H, (cyclohexyl)), 1.75-1.89 (m, 5H, (cyclohexyl)), 2.53-2.60 (m, 1H, CH), 4.27 (q, J=7.2 Hz, 2H, CO₂CH₂CH₃), 4.83 (bs, 2H, CH₂Ar), 4.97 (s, 2H, CH₂CO₂Et), 6.32 (bs, 1H, HNCH₂Ar), 7.28 7.41 (m, 7H, 2CH(Ar)), 7.79 (s, 1H, CH(H-8)), 7.86 (d, J=7.9 Hz, 2H, 2 CH(Ar)), 8.54 (bs, 1H, NH); LRMS (MS-ES), calcd for C₂₉H₃₃N₆O₃ [M+H] m/z=513.25, found 513.50.

### 117. Preparation of ethyl 2-(2-(4-cyclohexylbenzamido)-6-morpholino-9H-purin-9-yl)acetateacetate (Compound ID: 11c)

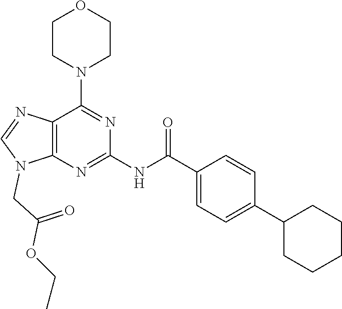 [SMILES: CCOC(=O)Cn1cnc2c(N3CCOCC3)nc(NC(=O)c3ccc(C4CCCCC4)cc3)nc21]

Purine 10 was treated with morpholine according to general procedure B, yielding the final product 11c as a off-white solid (67%); m.p, >70° C. (dec); IR (KBr, cm⁻¹) 2958, 2926, 2856, 1752, 1730, 1590, 1458, 1389, 1305, 1267, 1244, 1146, 1113; δ_{H} (400 MHz, CDCl3) 1.30 (t, J=7.2 Hz, 3H, CO₂CH₂CH₃), 1.36-1.50 (m, 5H, (cyclohexyl)), 1.75-1.90 (m, 5H, (cyclohexyl)), 2.54-2.60 (m, 1H, CH), 3.82 (t, J=4.7 Hz, 4H, 2CH2 (morpholine)), 4.26 (q, J=7.2 Hz, 2H, CO₂CH₂CH₃), 4.29 (bs, 4H, 2CH₂ (morpholine)), 4.92 (s, 2H, CH₂CO₂Et), 7.31 (d, J=8.1 Hz, 2H, 2CH(Ar)), 7.73 (s, 1H, CH(H-8)), 7.82 (d, J=7.9 Hz, 2H, 2CH (Ar)), 8.28 (bs, 1H, NH); LRMS (MS-ES), calcd for C₂₆H₃₃N₆O₄ [M+H] m/z=493.25, found 493.41.

### 118. Preparation of 2-(6-(butyl(methyl)amino)-2-(4-cyclohexylbenzamido)-9H-purin-9-yl)acetic acid (Compound ID: 12a)

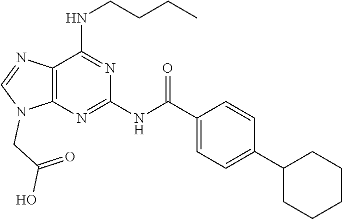 [SMILES: CCCCNc1nc(NC(=O)c2ccc(C3CCCCC3)cc2)nc2c1ncn2CC(=O)O]

Purine 11a was treated according to general procedure E, to yield final product 12a as a white lyophilized powder (85%): m.p.>124° C. (dec); IR (KBr, cm⁻¹) 2925, 2852, 1504, 1463, 1402, 1314, 1256, 1059; δ_{H} (400 MHz, DMSO-d₆) 0.89 (t, J=7.3 Hz, 3H, N(CH₂)₃CH₃), 1.27-1.49 (m, 7H, N(CH₂)₂CH₂CH₃ and 5H (cyclohexyl)), 1.52-1.64 (m, 2H, NCH₂CH₂CH₂CH₃), 1.69-1.80 (m, 5H, (cyclohexyl)), 2.54-2.61 (m, 1H, CH), 2.99-4.34 (bm, 5H, CH₃NCH₂(CH₂)₂CH₃), 4.60 (s, 2H, CH₂CO₂H), 7.29 (d, J=7.9 Hz, 2H, 2CH(Ar)), 7.80 (d, J=7.9 Hz, 2H, 2CH(Ar)), 7.95 (s, 1H, CH(H-8)), 10.24 (s, 1H, NH); δ_{C} (100 MHz, DMSO-d₆) 13.8, 19.3, 25.5, 26.2, 29.2, 33.6, 33.6, 43.6, 46.1, 49.4, 116.0, 126.4, 128.0, 132.6, 140.6, 151.1, 151.5, 153.7, 165.5, 170.3; HRMS (MS-ES), calcd for C₂₅H₃₃N₆O₃ [M+H] m/z=465.2601, found 465.2608; rpHPLC t_{R}: condition (I) 15.259 (II) 39.232 min, purity 95.4% and 96.9%.

### 119. Preparation of 2-(6-(benzylamino)-2-(4-cyclohexylbenzamido)-9H-purin-9-yl)acetic acid (Compound ID: 12b)

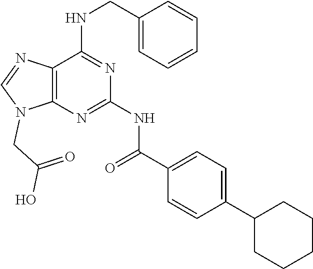 [SMILES: O=C(O)Cn1cnc2c(NCc3ccccc3)nc(NC(=O)c3ccc(C4CCCCC4)cc3)nc21]

Purine 11b was treated according to general procedure E, to yield final product 12b as a white lyophilized powder (78%): m.p.>167° C.; IR (KBr, cm⁻¹) 2926, 2851, 1454, 1386, 1352, 1252, 1126; δ_{H} (400 MHz, DMSO-d₆) 1.32-1.48 (m, 5H, (cyclohexyl)), 1.69-1.84 (m, 5H, (cyclohexyl)), 2.52-2.60 (m, 1H, CH), 4.64 (bs, 4H, HNCH₂ and CH₂CO₂H), 7.18-7.21 (m, 1H, 1 CH(Ar)), 7.26-7.32 (m, 4H, CH(Ar)), 7.40 (d, J=7.3 Hz, 2H, 2CH(Ar)), 7.84 (d, J=8.3 Hz, 2H, 2CH(Ar)), 7.98 (s, 1H, CH(H-8)), 8.21 (bs, 1H, NH), 10.30 (s, 1H, CONH); δC (100 MHz, 1 DMSO-d₆) 25.5, 26.2, 33.6, 42.7, 43.6, 45.4, 116.0, 126.4, 126.6, 127.6, 128.0, 132.5, 140.3, 141.4, 151.3, 152.8, 154.3, 165.5, 169.8; HRMS (MS-ES), calcd for C₂₇H₂₉N₆O₃ [M+H] m/z=485.2286, found 485.2295; rpHPLC t_{R}: condition (I) 14.987 (II) 33.307 min, purity 99.0% and 98.7%

### 120. Preparation of 2-(2-(4-cyclohexylbenzamido)-6-morpholino-9H-purin-9-yl)acetic acid (Compound ID: 12c)

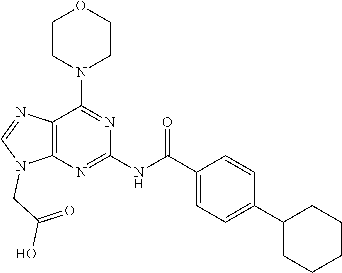 [SMILES: O=C(O)Cn1cnc2c(N3CCOCC3)nc(NC(=O)c3ccc(C4CCCCC4)cc3)nc21]

Purine 11c was treated according to general procedure E, to yield final product 12c as a white lyophilized powder (73%): m.p.>113° C. (dec); IR (KBr, cm⁻¹) 3672, 2925, 2854, 1720, 1523, 1459, 1384, 1266, 1241, 1194; δ_{H} (400 MHz, DMSO-d₆) 1.32-1.52 (m, 5H, (cyclohexyl)), 1.69 1.81 (m, 5H, (cyclohexyl)), 2.53-2.59 (m, 1H, CH), 3.67-3.69 (m, 4H, 2CH2 (morpholine)), 4.14 (bs, 4H, CH2 (morpholine)), 4.77 (s, 2H, CH₂CO₂H), 7.30 (d, J=8.3 Hz, 2H, 2CH(Ar)), 7.81 (d, J=8.1 Hz, 2H, 2CH(Ar)), 8.04 (s, 1H, CH(H-8)), 10.37 (s, 1H, NH); δ_{C} (100 MHz, DMSO-d₆) 25.5, 26.2, 33.6, 43.6, 44.9, 45.0, 66.2, 115.8, 126.4, 128.0, 132.6, 140.6, 151.2, 152.0, 152.3, 152.9, 165.5, 169.3; HRMS (MS-ES), calcd for C₂₄H₂₉N₆O₄ [M+H] m/z=465.2246, found 465.2244; rpHPLC t_{R}: condition (I) 14.199 (II) 33.308 min, purity 96.2% and 99.26%

### 121. Preparation of ethyl 2-(2-((tert-butoxycarbonyl)amino)-6-morpholino-9H-purin-9-yl)acetate (Compound ID: 13)

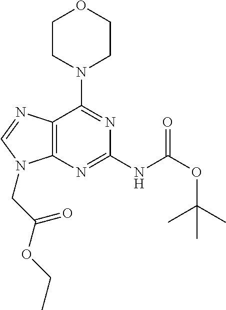 [SMILES: CCOC(=O)Cn1cnc2c(N3CCOCC3)nc(NC(=O)OC(C)(C)C)nc21]

Purine 4 was treated with morpholine according to general procedure B, yielding the final product 13 as an off-white solid (83%): m.p.=69-85° C.; IR (KBr, cm⁻¹) 3689, 2978, 1750, 1583, 1517, 1472, 1367, 1268, 1221, 1151; δ_{H} (400 MHz, CDCl₃) 1.30 (t, J=7.2 Hz, 3H, CO₂CH₂CH₃), 1.52 (s, 9H, C(CH₃)₃), 3.82 (t, J=4.9 Hz, 4H, 2CH₂ (morpholine)), 4.24 (q, J=7.2 Hz, 2H, COCH₂CH₃), 4.27 (bs, 4H, 2CH₂ (morpholine)), 4.89 (s, 2H, CH₂CO₂Et), 7.13 (s, 1H, NH), 7.69 (s, 1H, CH(H-8)); LRMS (MS-ES), calcd for C₁₈H₂₇N₆O₅ [M+H] m/z=407.20, found 407.43

### 122. Preparation of ethyl 2-(2-amino-6-morpholino-9H-purin-9-yl)acetate (Compound ID: 14)

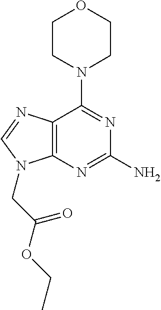 [SMILES: CCOC(=O)Cn1cnc2c(N3CCOCC3)nc(N)nc21]

Purine 13 was treated according to general procedure F, to yield product 14 as an off-white solid (94%): m.p.=93-98° C.; IR (KBr, cm⁻¹) 3672, 2922, 1736, 1540, 1459, 1312, 1182; δ_{H} (400 MHz, CDCl₃) 1.31 (t, J=7.2 Hz, 3H, CO₂CH₂CH₃), 3.82 (t, J=4.9 Hz, 4H, 2 CH₂ (morpholine)), 4.26 (q, J=7.2 Hz, 2H, COCH₂CH₃), 4.29 (bs, 4H, 2CH₂ (morpholine)), 4.92 (s, 2H, CH₂CO₂Et), 7.48 (s, 1H, CH(H-8)); LRMS (MS-ES), calcd for C₁₃H₁₉N₆O₃ [M+H] m/z=307.14, found 307.28.

### 123. Preparation of ethyl 2-(6-morpholino-2-pentanamido-9H-purin-9-yl)acetate (Compound ID: 15a)

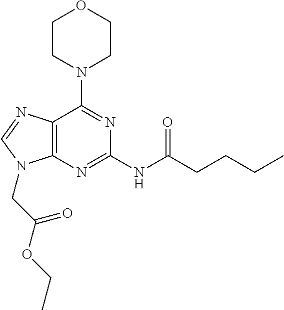 [SMILES: CCCCC(=O)Nc1nc(N2CCOCC2)c2ncn(CC(=O)OCC)c2n1]

Purine 14 was treated with valeryl chloride according to general procedure G, to yield lyophilized product 15a as a white solid (72%): m.p.>141° C. (dec); IR (KBr, cm⁻¹) 3551, 3477, 3414, 3228, 3110, 2956, 2930, 2849, 1751, 1670, 1638, 1608; δH (400 MHz, CDCl₃) 0.94 (t, J=7.3 Hz, 3H, (CH₂)₃CH₃), 1.30 (t, J=7.2 Hz, 3H, CO₂CH₂CH₃), 1.41 (sextet, J=7.4 Hz, 2H, (CH₂)₂CH₂CH₃), 1.71 (p, J=7.5 Hz, 2H, CH₂CH₂CH₂CH₃), 2.78 (m, 2H, CH₂(CH₂)₂CH₃), 3.83 (t, J=4.9 Hz, 4H, 2CH2 (morpholine)), 4.26 (q, J=7.2 Hz, 2H, CO₂CH₂CH₃), 4.28 (bs, 4H, 2CH2 (morpholine)), 4.86 (s, 2H, CH₂CO₂Et), 7.69 (bs, 1H, NH), 7.70 (s, 1H, CH(H-8)); LRMS (MS-ES), calcd for C₁₈H₂₆N₆O₄Na [M+Na] m/z=413.20, found 413.37

### 124. Preparation of ethyl 2-(2-(cyclohexanecarboxamido)-6-morpholino-9H-purin-9-yl)acetate (Compound ID: 15b)

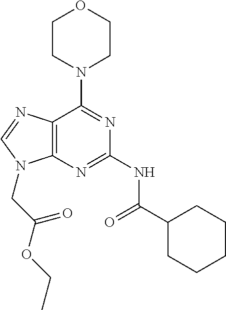 [SMILES: CCOC(=O)Cn1cnc2c(N3CCOCC3)nc(NC(=O)C3CCCCC3)nc21]

Purine 14 was treated with valeryl chloride according to general procedure G, to yield lyophilized product 15b as an off-white solid (74%): m.p.=142-147° C.; IR (KBr, cm⁻¹) 3551, 3415, 3238, 2928, 2852, 1755, 1669, 1604, 1585, 1514, 1448, 1407; δ_{H} (400 MHz, CDCl₃) 1.29 (t, J=7.2 Hz, 3H, CO₂CH₂CH₃), 1.28-1.32 (m, 2H, CH₂ (cyclohexyl)), 1.49 (m, 3H, (cyclohexyl)), 1.70-1.71 (m, 1H, (cyclohexyl)), 1.82 (m, 2H, (cyclohexyl)), 1.96-1.99 (m, 2H, (cyclohexyl)), 2.88 (m, 1H, CH), 3.82 (t, J=4.9 Hz, 4H, 2CH₂ (morpholine)), 4.25 (q, J=7.2 Hz, 2H, CO₂CH₂CH₃), 4.27 (bs, 4H, 2CH₂ (morpholine)), 4.87 (s, 2H, CH₂CO₂Et), 7.69 (bs, 1H, NH), 7.70 (s, 1H, CH(H-8)); LRMS (MS-ES), calcd for C₂₀H₂₉N₆O₄ [M+H] m/z=417.22, fnd 417.40.

### 125. Preparation of 2-(6-morpholino-2-pentanamido-9H-purin-9-yl)acetic acid (Compound ID: 16a)

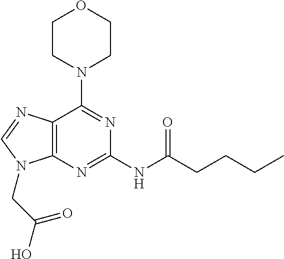 [SMILES: CCCCC(=O)Nc1nc(N2CCOCC2)c2ncn(CC(=O)O)c2n1]

Purine 15a was treated according to general procedure E, to yield final product 16a as a white lyophilized powder (71%): m.p.>138° C. (dec); IR (KBr, cm⁻¹) 3233, 1753, 1516, 1466, 1385, 1311, 1267, 1220, 1114, 1009; δ_{H} (400 MHz, DMSO-d₆) 0.87 (t, J=7.3 Hz, 3H, (CH₂)₃CH₃), 1.29 (sextet, J=7.5 Hz, 2H, (CH₂)₂CH₂CH₃), 1.52 (p, J=7.5 Hz, 2H, CH₂CH₂CH₂CH₃), 2.47 (t, J=7.2 Hz, 2H, CH₂(CH₂)₂CH₃), 3.83 (t, J=4.3 Hz, 4H, 2CH₂ (morpholine)), 4.19 (bs, 4H, 2CH₂ (morpholine)), 4.74 (s, 2H, CH₂CO₂H), 7.99 (s, 1H, CH (H-8)), 9.92 (s, 1H, NH); δ_{C} (100 MHz, DMSO-d₆) 13.7, 21.8, 26.8, 35.9, 44.7, 45.0, 66.1, 115.3, 140.2, 151.9, 152.1, 152.9, 169.2, 171.5; HRMS (MS-ES), calcd for C₁₆H₂₃N₆O₄ [M+H] m/z=363.1775, found 363.1775; rpHPLC t_{R}: condition (I) 10.270 (II) 15.079 min, purity 98.2% and 98.0%.

### 126. Preparation of 2-(2-(cyclohexanecarboxamido)-6-morpholino-9H-purin-9-yl)acetic acid (Compound ID: 16b)

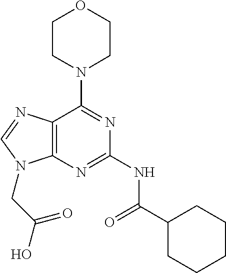 [SMILES: O=C(O)Cn1cnc2c(N3CCOCC3)nc(NC(=O)C3CCCCC3)nc21]

Purine 15b was treated according to general procedure E, to yield final product 16b as a white lyophilized powder (68%): m.p.>122° C. (dec); IR (KBr, cm⁻¹) 3631, 2927, 2856, 1743, 1514, 1466, 1385, 1306, 1265, 1240, 1192, 1116, 1069; δ_{H} (400 MHz, DMSO-d₆) 1.09-1.38 (m, 5H, (cyclohexyl)), 1.61-1.78 (m, 5H, (cyclohexyl)), 2.61-2.75 (m, 1H, (cyclohexyl)), 3.82 (t, J=4.6 Hz, 4H, 2CH₂ (morpholine)), 4.19 (bs, 4H, 2CH₂ (morpholine)), 4.74 (s, 2H, CH₂CO₂H), 7.99 (s, 1H, CH(H-8)), 9.85 (s, 1H, NH); δ_{C} (100 MHz, DMSO-d₆) 25.2, 25.4, 29.0, 43.8, 44.7, 45.0, 66.2, 140.2, 151.9, 152.2, 153.0, 169.3, 174.3; HRMS (MS-ES), calcd for C₁₈H₂₅N₆O₄ [M+H] m/z=389.1919, found 389.1931; rpHPLC t_{R}: condition (I) 10.978 (II) 17.891 min, purity 97.9% and 98.0%.

### 127. Cells and Reagents

Normal mouse fibroblasts (NIH3T3) and counterparts transformed by v-Src (NIH3T3/v-Src) or overexpressing the human epidermal growth factor (EGF) receptor (NIH3T3/hEGFR), the murine thymus epithelial stromal cells, and the human breast cancer (MDA-MB-231) and pancreatic cancer (Panc-1) cells have all been previously reported (6, 16). Antibodies against STAT3, pY705STAT3, Erk1/2, and pErk1/2 are from Cell Signaling Technology (Danvers, Mass.). Recombinant human epidermal growth factor (rhEGF) was obtained from Invitrogen (Carlsbad, Calif.).

### 128. Cloning and Protein Expression

Coding regions for the murine STAT3 protein and STAT3 SH2 domain were amplified by PCR and cloned into vectors pET-44 Ek/LIC (Novagen) and pET SUMO (Invitrogen), respectively. The primers used for amplification were: STAT3 Forward: GACGACGACAAGATGGCTCAGTGGAACCAGCTGC; STAT3 Reverse: GAGGAGAAGCCCGGTTATCACATGGGGGAGGTAGCACACT; STAT3-SH2 Forward: ATGGGTTTCATCAGCAAGGA; STAT3-SH2 Reverse: TCACCTACAGTACTTTCCAAATGC. Clones were sequenced to verify the correct sequences and orientation. His-tagged recombinant proteins were expressed in BL21(DE3) cells, and purified on Ni-ion sepharose column.

### 129. Nuclear Extract Preparation, Gel Shift Assays, and Densitometric Analysis

Nuclear extract preparations and electrophoretic mobility shift assay (EMSA) were carried out as previously described (38). Briefly, nuclear extracts of equal total protein were pre-incubated with increasing concentration of compound for 30 min at room temperature prior to the incubation with the radiolabeled probe for 30 min at 30° C. before subjecting to EMSA analysis. The ³²P-labeled oligonucleotide probe used was hSIE (high affinity sis-inducible element from the c-fos gene, m67 variant, 5′-AGCTTCATTTCCCGTAAATCCCTA) that binds STAT1 and STAT3 (39). Bands corresponding to DNA-binding activities were scanned and quantified for each concentration of compound using ImageQuant and plotted as percent of control (vehicle) against concentration of compound, from which the IC50 values were derived, as previously reported (25).

### 130. Immunoprecipitation and Immunoblotting Assay

Immunoprecipitation, and SDS/PAGE and Western blotting analysis were performed as previously described (6, 16). Primary antibodies used were anti-STAT3, pY705STAT3, pY416Src, Src, pErk1/2, Erk1/2, pSTAT1, STAT1, (Cell Signaling), and antiphosphotyrosine, clone 4G10 (Upstate Biotechnology, Lake Placid, N.Y.). Where appropriate, cells were stimulated for 12 min by 9 ng/μl rhEGF (12 μl into 3 ml culture) prior to preparation of whole-cell lysates for immunoprecipitation and/or immunoblotting analysis.

### 131. Cell Viability and Proliferation Assay

Cells in culture in 6-well or 96-well plates were treated with or without agents for 24-144 h and subjected to CyQuant cell proliferation assay (Invitrogen Corp/Life Technologies Corp, Carlsbad, Calif.). IC₅₀ values shown below were derived from the plot of viability versus drug concentration.

### 132. Surface Plasmon Resonance Analysis

Surface Plasmon resonance analysis was performed to characterize the binding of compounds to STAT3, as previously reported (16). SensiQ and its analysis software Qdat (ICX Technologies, Oklahoma City, Okla.) were used to analyze the interaction between agents and the STAT3 protein and to determine the binding affinity. Purified STAT3 was immobilized on a HisCap Sensor Chip by injecting 50 μg/ml of STAT3 onto the chip. Various concentrations of compounds in running buffer (1×PBS, 0.5% DMSO) were passed over the sensor chip to produce response signals. The association and dissociation rate constants were calculated using the Qdat software. The ratio of the association and dissociation rate constants was determined as the affinity (K_{D}).

### 133. Activity of Substituted Purine Analog Compounds

Substituted purine analogs were synthesized as described above. Activity (K_{D} for binding to STAT3 and IC₅₀ for inhibition of STAT3 activity in an EMSA) was determined as described above, and the data are shown below in Table I. The compound identifier corresponds to the number given parenthetically for the preparation of compounds described above. The compounds in Table I reference for convenience substituents X and Y for the structure given immediately below. In the context of the disclosed compounds as discussed herein, R² corresponds to the X substituent given below and NR³R⁴ is collectively represented by Y below.

TABLE I

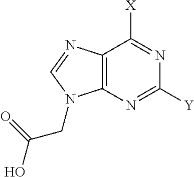

| | Compound | | | K_{D} | IC₅₀ |
|---|---|---|---|---|---|
| No. | ID | Substituent X | Substituent Y | (μM) | (μM) |
| 1 | 8aa | 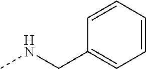 | 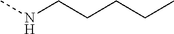 | 2.2 | >100 |
| 2 | 8ab | 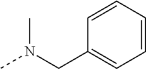 | 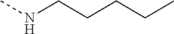 | 38.4 | >100 |
| 3 | 8ac | 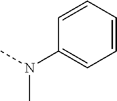 | 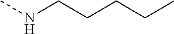 | 2.5 | >100 |
| 4 | 8ad | 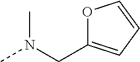 | 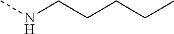 | 38.4 | >100 |
| 5 | 8ae | 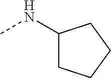 | 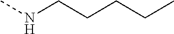 | 6.4 | >100 |
| 6 | 8af | 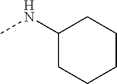 | 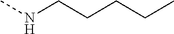 | 6.1 | >100 |
| 7 | 8ag | 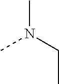 | 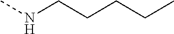 | >50 | >100 |
| 8 | 8ah | 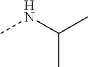 | 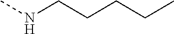 | >50 | >100 |
| 9 | 8ai | 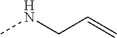 | 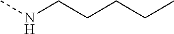 | >50 | >100 |
| 10 | 8aj | 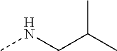 | 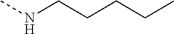 | 6.0 | >100 |
| 11 | 8ak | 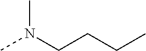 | 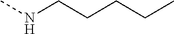 | >50 | >100 |
| 12 | 8al | 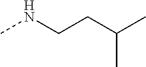 | 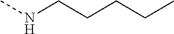 | >50 | >100 |
| 13 | 8am | 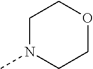 | 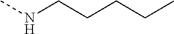 | 4.2 | >100 |
| 14 | 8an | 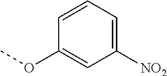 | 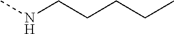 | >50 | >100 |
| 15 | 8ao | 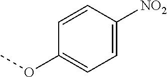 | 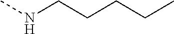 | >50 | >100 |
| 16 | 8ba | 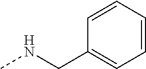 | 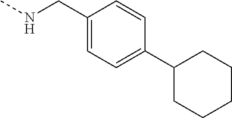 | 5.4 | >100 |
| 17 | 8bb | 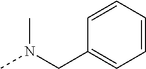 | 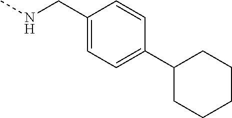 | 4.0 | >100 |
| 18 | 8bd | 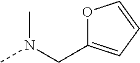 | 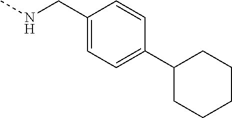 | 2.8 | 83.6 ± 6.1 |
| 19 | 8be | 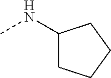 | 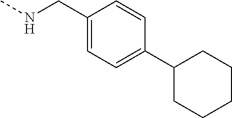 | 16.7 | 74.4 ± 5.4 |
| 20 | 8bf | 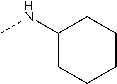 | 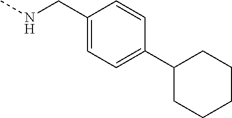 | 11.2 | >100 |
| 21 | 8bi | 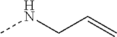 | 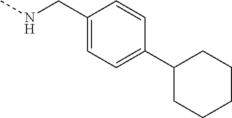 | 2.3 | 57.4 ± 3.9 |
| 22 | 8bj | 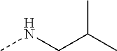 | 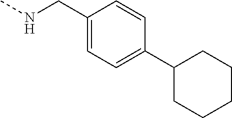 | 39.0 | >100 |
| 23 | 8bl | 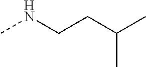 | 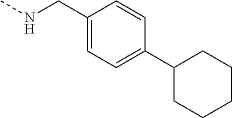 | >50 | >100 |
| 24 | 8bm | 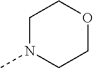 | 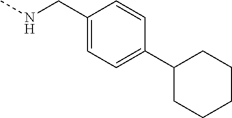 | >50 | >100 |
| 25 | 8bl | 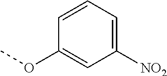 | 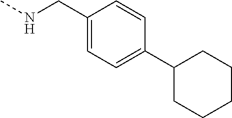 | 7.9 | 53.5 ± 3.8 |
| 26 | 8bo | 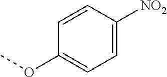 | 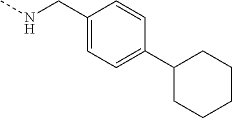 | 1.2 | 27.2 ± 4.3 |
| 27 | 8bp | 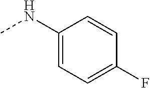 | 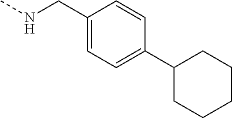 | >50 | >100 |
| 28 | 8bq | 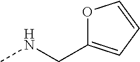 | 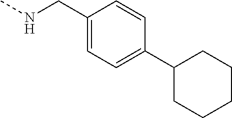 | 1.8 | >100 |
| 29 | 8bs | 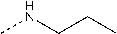 | 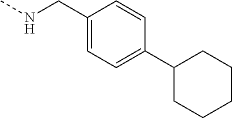 | 3.1 | >100 |
| 30 | 8bt | 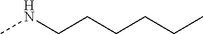 | 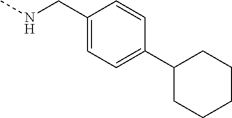 | >50 | 79.7 ± 7.4 |
| 31 | 8bu | 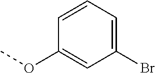 | 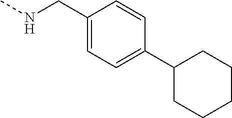 | 0.8 | >100 |
| 32 | 8bv | 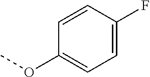 | 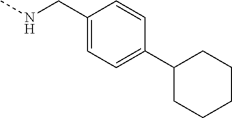 | 0.8 | >100 |
| 33 | 8bw | 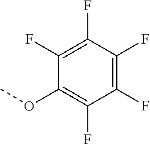 | 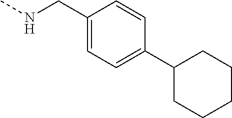 | 1.3 | >100 |
| 34 | 8bx | 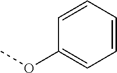 | 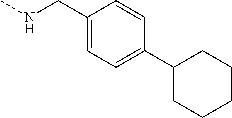 | 1.0 | >100 |
| 35 | 12a | 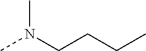 | 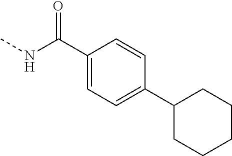 | >50 | n/d |
| 36 | 12b | 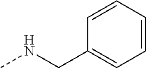 | 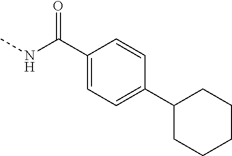 | 2.9 | >100 |
| 37 | 12c | 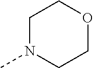 | 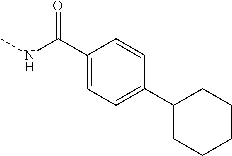 | 50 | >100 |
| 38 | 16a | 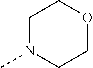 | 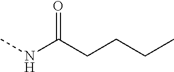 | 3.2 | >100 |
| 39 | 16b | 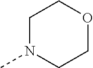 | 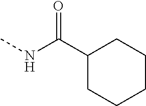 | 1.3 | 64.4 ± 4.8 |
| 40 | 7aa | 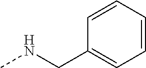 | 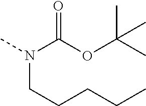 | 27.9 | >100 |
| 41 | 7ab | 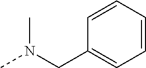 | 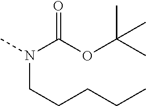 | 14.8 | >100 |
| 42 | 7ac | 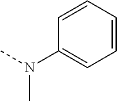 | 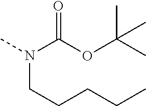 | 0.9 | >100 |
| 43 | 7am | 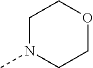 | 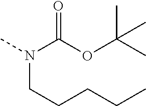 | >50 | >100 |
| 44 | 7an | 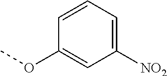 | 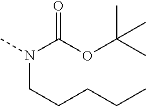 | >50 | >100 |
| 45 | 7ao | 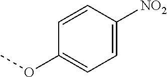 | 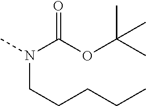 | >50 | >100 |
| 46 | 7ay | 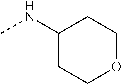 | 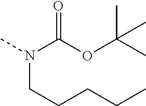 | 2.0 | >100 |
| 47 | 7bm | 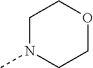 | 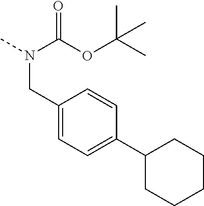 | 4.1 | >100 |

### 134. Effect of Compounds in EMSA Assay

The disclosed compounds, substituted 2-(9H-purin-9-yl)acetic acid analogs, interacted with STAT3. The interaction with STAT3 was expected to disrupt STAT3 binding to its cognate pTyr peptide and prevent STAT3:STAT3 dimerization. (Turkson et al., 2001; Siddiquee et al., 2007; Siddiquee et al., 2007(b); Zhang et al., 2010). Therefore, further evaluation of substituted 2-(9H-purin-9-yl)acetic acid analogs in in vitro STAT3 DNA-binding assay/electrophoretic mobility shift assay (EMSA) analysis was performed. The EMSA analysis demonstrated that the select agents, 8bq, 8bp, 8be, 8bl, 8bj, 17b, and 8bo, all of which showed binding affinity for STAT3 (see, e.g., Table 1, SPR), also inhibited STAT3 activity. These experiments generated IC₅₀ values of 27-84 μM (see Table 1, EMSA).

Some of the data from the EMSA analysis did not show a direct correlation to the SPR analysis. For example, although STAT3 dimerization disruptors have exhibited high potency in inhibiting dimerization, the activities in the DNA binding assay/EMSA analysis have generally been weaker. (Zhang et al., 2010). In these experiments, this discrepancy can be explained by the presence of a large number of protein “targets” in the nuclear extract preparations utilized in the DNA-binding assay and that the disrupting preformed STAT3:STAT3 dimers. (See, e.g., Turkson et al., 2001; Turkson et al., 2007).

### 135. Effect of Compounds on STAT3, Erk^{MAPK}, Src, and STAT1 Activation

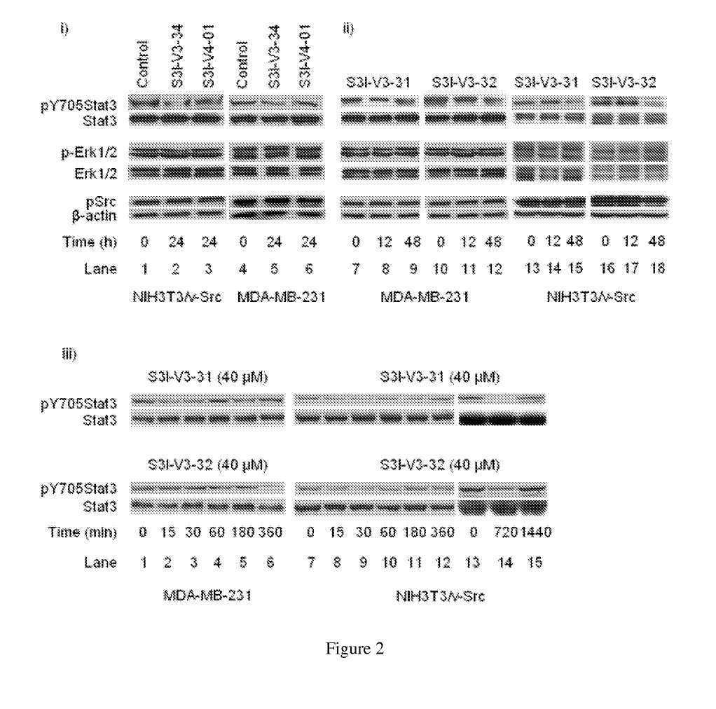

Following treatments with compounds 8be, 8bj, 8bl, and 17b for 12, 24, or 48 h, immunoblotting analysis of whole cell lysates of equal total protein showed inhibition of constitutive STAT3 phosphorylation in v-Src transformed mouse fibroblasts (NIH3T3/v-Src) and in the human breast cancer cell line MDA-MB-231 (both of which harbor aberrant STAT3 activity) (Turkson et al., 2004; Yue et al., 2009). In FIG. 2 (i) and FIG. 2 (ii, the immunoblots were probed with pY705STAT3, STAT3, pErk 1/2, Erk 1/2, pSrc, Src, pSTAT1, STAT1, and β-actin or pTyr. pY705 was the top band in all panels and 8be was represented in lanes 8 and 9 (MDA-MB-231) and in lanes 14 and 15 (NIH3T3/v-Src); 8bl was represented in lanes 11 and 12 (MDA-MB-231) and in lane 18 (NIH3T3/v-Src); 8bj was represented in lane 2 (NIH3T3/v-Src) and in lane 5 (MDA-MB-231); and 17b was represented in lane 3 (NIH3T3/v-Src) and in lane 6 (MDA-MB-231). In FIG. 2A(i) and (ii), the control lanes were lanes 1, 4, 7, 10, 13, and 16 (representing whole-cell lysates from 0.05% DMSO-treated cells).

The same treatments (i.e., 8be, 8bl, 8bj, and 17b) had no effect on the constitutive levels of phospho-ErkMAPK (pErk1/2), which is the third band from the top in all panels of FIG. 2, or on the constitutive levels of Src (pSrc), which is the fifth band from the top in all panels of FIG. 2.

The inhibition of STAT3 phosphorylation showed different kinetics among the purine compounds studied. The difference in kinetics was further explored using additional time-course experiments. In NIH3T3/v-Src fibroblasts, STAT3 activity was inhibited as early as 15-60 min after being treated with 8be or 8bl. (Figure iii), right panels). During prolonged treatment of NIH3T3/v-Src fibroblasts with 8be or 8bl, STAT3 phosphorylation appeared to be temporarily restored (FIG. 2 (iii), right panels). In the MDA-MB-231 cell line, STAT3 phosphorylation was inhibited as early as 15-30 min following 8be treatment. There was evidence of a restoration of STAT3 phosphorylation after 6 h of 8be treatment. (FIG. 2A(iii), left, top). In the MDA-MB-231 cell line, STAT3 phosphorylation remained inhibited after 6 h following 8bl treatment. (FIG. 2 (iii), left, bottom). While these date indicated that purine scaffolds exhibited different kinetics of inhibition of intracellular constitutive STAT3 phosphorylation, the level of STAT3 phosphorylation 48 h following treatment remained low.

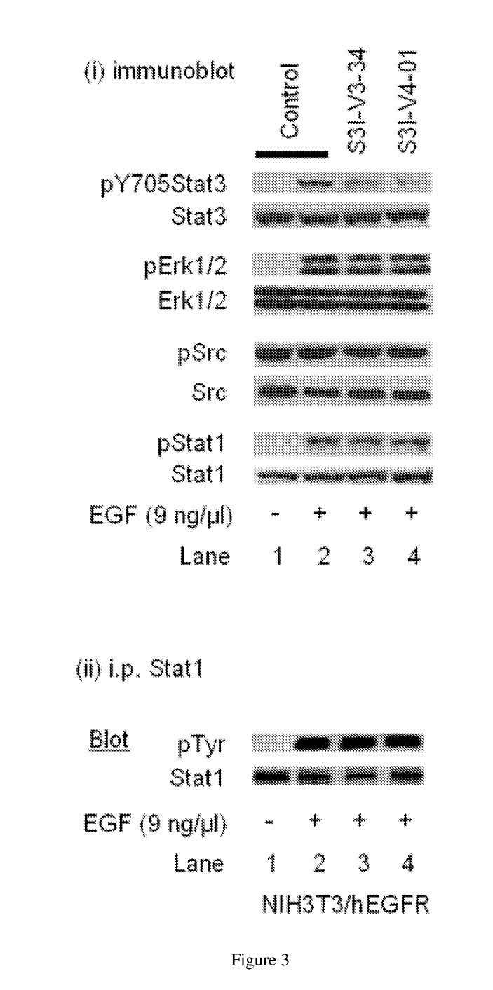

The effect of 8bj and 17b on ligand-stimulated STAT activation was also examined. 8bj and 17b selectively inhibited epidermal growth factor (EGF)-induced STAT3 phosphorylation in mouse fibroblasts overexpressing the human EGF receptor (NIH3T3/hEGFR) (see, e.g., FIG. 3(i), which showed pY705-STAT3 immunoblotting of whole-cell lysates treated with 8bj (lane 3) and 17b (lane 4) as compared to the control (no treatment, lane 2). The blocking of ligand-induced STAT3 phosphorylation by 8bj and 17b indicated that purine agents interacted with the inactive STAT3 monomers, e.g., by the SH2 domain, and occluded STAT3 binding to the EGFR receptor. Furthermore, these purine agents blocked de novo STAT3 phosphorylation, and blocked STAT3:STAT3 dimerization. These findings were consistent with other reports regarding other SH2 domain binding STAT3 inhibitors. (Turkson et al., 2001; Siddiquee et al., 2007; Siddiquee et al., 2007(b)).

As shown in FIG. 3(i), S31-V3-34 and S31-V4-01 did not inhibit the phosphorylation of Erk (pErk1/2) and Src (pSrc) in NIH3T3/hEGFR fibroblasts. S31-V3-34 and S31-V4-01 treatment did not significantly affect EGF-induced phosphorylation of STAT1. (FIG. 2B(i), pSTAT1, second panel from the bottom). The lack of an effect on EGF-induced phosphorylation of STAT1 in NIH3T3/hEGFR fibroblasts by S31-V3-34 and S31-V4-01 treatment was also demonstrated using STAT1 immune complex precipitation, with general pTyr immunoblotting analysis. (See FIG. 3(ii)). The data represented by FIG. 2(A)-(B) were the results of experiments performed in triplicate or quadruplicate.

### 136. Effect of Compounds on Cell Viability

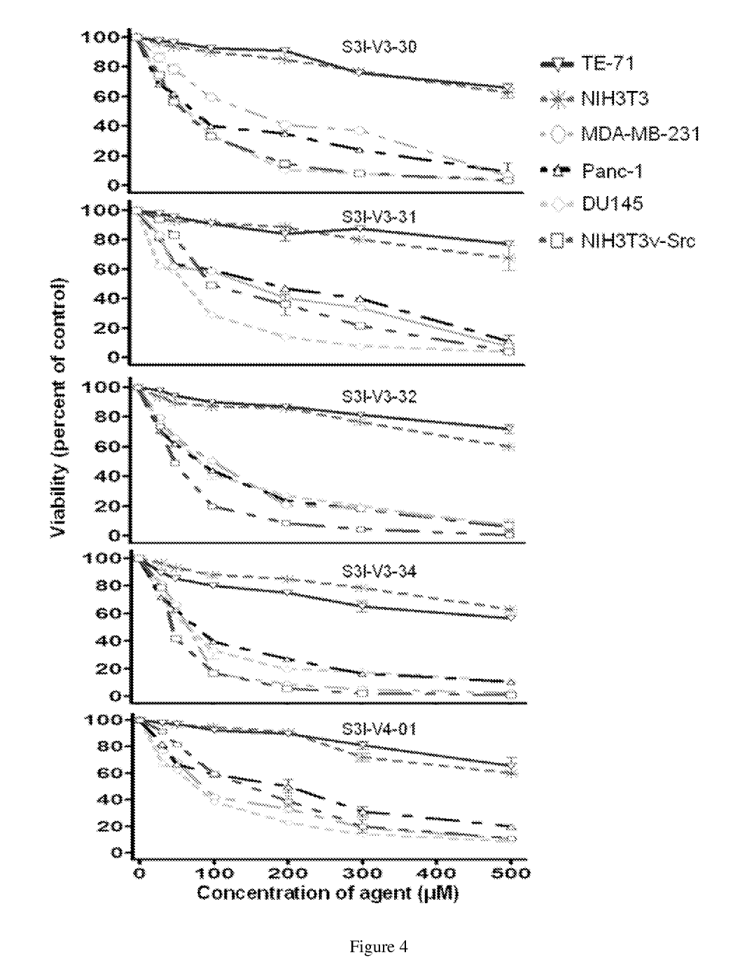

As aberrant STAT3 activity promotes cancer cell growth and survival, and tumor angiogenesis and metastasis (Turkson et al., 2004; Yue et al., 2009; Turkson et al., 2004; Turkson et al., 2001; Siddiquee et al., 2007; Siddiquee et al., 2007(b); Song et al., 2005; Turkson et al., 2005), the sensitivity of various cell lines to the purine scaffold treatment was tested. Following 48 hours of treatment with purine-scaffold small-molecules (30-500 μM), including 531-V3-30, 8be, 8bl, 8bj, and 17b, CyQuant cell proliferation assay showed that the human prostate (DU145), breast (MDA-MB-231), and pancreatic cancer (Panc-1) cell lines and the v-Src-transformed mouse fibroblasts (NIH3T3/v-Src) experienced a decrease in malignant cell viability. For example, FIG. 4 demonstrated those cells that harbor constitutively active STAT3 were sensitive to treatment with purine-scaffold small-molecules, generating IC₅₀ values of 41-80 μM concentrations (see also Table 2). FIG. 4 also showed that those cells that do not harbor aberrant STAT3 activity such as TE-71 and NIH3T3 (Zhang et al., 2010) were not affected by the selected purine-scaffold small-molecules, having IC50 values over 500 μM. (see also Table 2). The data represented by FIG. 4 were the results of experiments performed in triplicate.

These data demonstrated that purine-scaffold small molecules, including 8be, 8bl, 8bj and 17b, selectively inhibited STAT3 activation in malignant cells, and induced the loss of viability of tumor cells that harbored persistently active STAT3. These data indicated that select purine compounds affected STAT3 activity and induced loss of viability of malignant cells that harbored constitutively active STAT3.

TABLE 2

| | | | IC₅₀ (μM) | | | |
|---|---|---|---|---|---|---|
| | | | MDA- | | | NIH3T3/ |
| Compound | NIH3T3 | TE-71 | MB-231 | Panc-1 | DU 145 | v-Src |
| S3I-V3-30 | 420 | >500 | 147.8 ± 5.1 | 74.5 ± 8.4 | 65.1 ± 8.3 | 62.5 ± 3.4 |
| 8be | >500 | >500 | 145.2 ± 4.4 | 175.7 ± 5.3 | 68.6 ± 1.8 | 100.2 ± 2.2 |
| 8bl | >500 | >500 | 100.4 ± 3.2 | 77.7 ± 2.7 | 80.2 ± 5.1 | 45.6 ± 3.1 |
| 8bj | >500 | >500 | 66.1 ± 2.1 | 77.2 ± 1.9 | 65.3 ± 2.3 | 41.1 ± 4.6 |
| 17b | >500 | >500 | 82.4 ± 1.4 | 197.6 ± 2.1 | 75.4 ± 3.4 | 145.6 ± 6.1 |

### 137. Prospective In Vivo Activity

Generally compounds that inhibit STAT protein activity in preclinical animal models of tumor growth and pathology. In vivo effects of the compounds described in the preceding examples are expected to be show activity in various models cancer biology known to the skilled person, such as a mouse subcutaneous xenograft model or, alternatively, the mouse orthotopic xenograft model. These models are typically conducted in an immunocompromised mouse, e.g. athymic nude mice, severely compromised immunodeficient (SCID) mice, or other immunocompromised mice (see reference (40) above), but may be conducted in other animal species as is convenient to the study goals. Alternatively, a genetically engineered mouse (GEM) model can be used to assess the efficacy of the disclosed compounds on inhibiting tumor growth. The genetic profile of GEM mice is altered such that one or several genes thought to be involved in transformation or malignancy are mutated, deleted or overexpressed; subsequently, the effect of altering these genes is studied over time and therapeutic responses to these tumors may be followed in vivo.

The subcutaneous xenograft model is frequently used by one skilled in the art to assess anti-cancer activity. Briefly, the cell-line of choice, e.g. MDA-MB-231, Panc-1, DU 145, or NIT3T3/v-Src cells, are grown in vitro in culture flasks, and then collected with using trypsin (if adherent) or by simple centrifugation (if suspension cultures), and then suspended in PBS at about 6×10⁷ cells/mL. In one experimental approach, about 10⁵ cells are injected in mice subcutaneously on Day 0, and the tumors allowed to develop to about 10⁶ cells (about 7-10 days). Suitable mice strains to use in this include nu/nu nude mice or CAnN.Cg-Foxnlnu/CrlCrlj (nu/nu), and are readily available from suitable commercial sources (e.g. Harlan or Charles River). Drug is administered by a suitable route of administration, e.g. intravenous or intraperitoneally, on a dosing schedule suitable for the compound, e.g. daily for a period of five days or every third day for a period of two weeks or other schedule as determined from in vitro and in vivo data on potency, pharmacokinetics, and metabolism. The vehicle choice is determined based on the physical-chemical properties of the test compound. Exemplary vehicles include mixtures comprising DMSO, Cremaphor, and vegetable oils, e.g. 12.5% DMSO, 5% Cremaphor and 82.5% peanut oil; polysorbate:ethanol, e.g. 80:13; cremaphor:ethanol; and normal saline, e.g. phosphate-buffered saline. Body weight and tumor diameter are measured on a suitable schedule, e.g. every 3-4 days using calipers, and tumor volume determined by calculating the volume of an ellipsoid using the formula: length×width²×0.5. Antitumor activities can be expressed as percent inhibition of tumor growth and percent regression of the tumor.

For example, compounds having a structure represented by a formula:

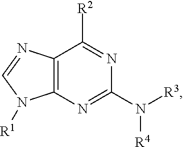

wherein R¹ is selected from H and (CH₂)ₘC═OR⁵, wherein m is an integer from 0-3; wherein R⁵ is selected from OR⁶ and NR⁷R⁸; wherein R⁶ is selected from hydrogen and C1-C8 alkyl; wherein each of R⁷ and R⁸ is independently selected from hydrogen and C1-C8 alkyl; wherein R² is selected from halogen, OR⁹, and NR¹⁰R¹¹; wherein R⁹ is an optionally substituted group selected from C1-C8 alkyl, C3-C6 cycloalkyl, C3-C6 heterocycloalkyl, (CH₂)ₙR¹², and Ar¹; wherein n is an integer from 0-3; wherein R¹² is an optionally substituted group selected from Ar¹, C3-C6 cycloalkyl and C3-C6 heterocycloalkyl; wherein Ar¹ is selected from an optionally substituted monocyclic aryl and monocyclic heteroaryl; wherein R¹⁰ is selected from hydrogen and an optionally substituted group selected from C1-C8 alkyl, C3-C6 cycloalkyl, C3-C6 heterocycloalkyl, (CH₂)ₚR¹³, and Ar²; wherein p is an integer from 0-3; wherein R¹³ is an optionally substituted group selected from Ar², C3-C6 cycloalkyl and C3-C6 heterocycloalkyl; wherein Ar² is selected from an optionally substituted monocyclic aryl and monocyclic heteroaryl; wherein R¹¹ is selected from hydrogen and C1-C8 alkyl; wherein R³ is selected from hydrogen and an optionally substituted group selected from C1-C8 alkyl, (CH₂)_{q}R¹⁴, and C═O(CH₂)_{q}R¹⁴; wherein q is an integer from 0-3; wherein R¹⁴ is selected from hydrogen and an optionally substituted group selected from C1-C8 alkyl, C3-C6 cycloalkyl, C3-C6 heterocycloalkyl, and Ar³; wherein Ar³ is selected from an optionally substituted monocyclic aryl and monocyclic heteroaryl; wherein R⁴ is selected from hydrogen and C═OOR¹⁵; wherein R¹⁵ is selected from hydrogen and optionally substituted C1-C8 alkyl; wherein R³ and R⁴ are optionally covalently bonded and, together with the intermediate carbon, comprise an optionally substituted 3- to 7-membered heterocycloalkyl; and wherein R¹⁰ and R¹¹ are optionally covalently bonded and, together with the intermediate carbon, comprise an optionally substituted 3- to 7-membered heterocycloalkyl; or a pharmaceutically acceptable salt, hydrate, solvate, or polymorph thereof, are expected to show such in vivo effects.

Moreover, compounds prepared using the disclosed synthetic methods are also expected to show such in vivo effects.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A compound having a structure represented by a formula: 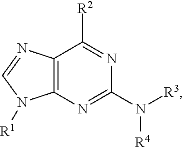
wherein R¹ is selected from H and (CH₂)ₘC═OR⁵, wherein m is an integer from 0-3;
wherein R⁵ is selected from OR⁶ and NR⁷R⁸;
wherein R⁶ is selected from hydrogen and C1-C8 alkyl;
wherein each of R⁷ and R⁸ is independently selected from hydrogen and C1-C8 alkyl;
wherein R² is selected from halogen, OR⁹, and NR¹⁰R¹¹;
wherein R⁹ is an optionally substituted group selected from C1-C8 alkyl, C3-C6 cycloalkyl, C3-C6 heterocycloalkyl, (CH₂)ₙR¹², and Ar¹;
wherein n is an integer from 0-3;
wherein R¹² is an optionally substituted group selected from Ar¹, C3-C6 cycloalkyl and C3-C6 heterocycloalkyl;
wherein Ar¹ is selected from an optionally substituted monocyclic aryl and monocyclic heteroaryl;
wherein R¹⁰ is selected from hydrogen and an optionally substituted group selected from C1-C8 alkyl, C3-C6 cycloalkyl, C3-C6 heterocycloalkyl, (CH₂)ₚR¹³, and Ar²;
wherein n is an integer from 0-3;
wherein R¹³ is an optionally substituted group selected from Ar², C3-C6 cycloalkyl and C3-C6 heterocycloalkyl;
wherein Ar² is selected from an optionally substituted monocyclic aryl and monocyclic heteroaryl;
wherein R¹¹ is selected from hydrogen and C1-C8 alkyl;
wherein R³ is selected from hydrogen and an optionally substituted group selected from C1-C8 alkyl, (CH₂)_{q}R¹⁴, and C═O(CH₂)_{q}R¹⁴;
wherein q is an integer from 0-3;
wherein R¹⁴ is selected from hydrogen and an optionally substituted group selected from C1-C8 alkyl, C3-C6 cycloalkyl, C3-C6 heterocycloalkyl, and Ar³;
wherein Ar³ is selected from an optionally substituted monocyclic aryl and monocyclic heteroaryl;
wherein R⁴ is selected from hydrogen and C═OOR¹⁵;
wherein R¹⁵ is selected from hydrogen and optionally substituted C1-C8 alkyl;
wherein R³ and R⁴ are optionally covalently bonded and, together with the intermediate carbon, comprise an optionally substituted 3- to 7-membered heterocycloalkyl; and
wherein R¹⁰ and R¹¹ are optionally covalently bonded and, together with the intermediate carbon, comprise an optionally substituted 3- to 7-membered heterocycloalkyl;
or a pharmaceutically acceptable salt, hydrate, solvate, or polymorph thereof.

2. The compound of claim 1, having a structure represented by a formula:

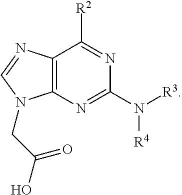

3. The compound of claim 1, having a structure represented by a formula: 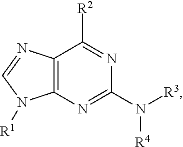
wherein R¹ is selected from hydrogen, 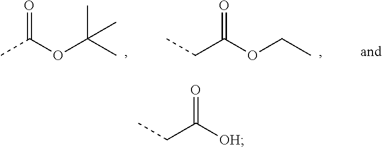
wherein R² is selected from halogen, 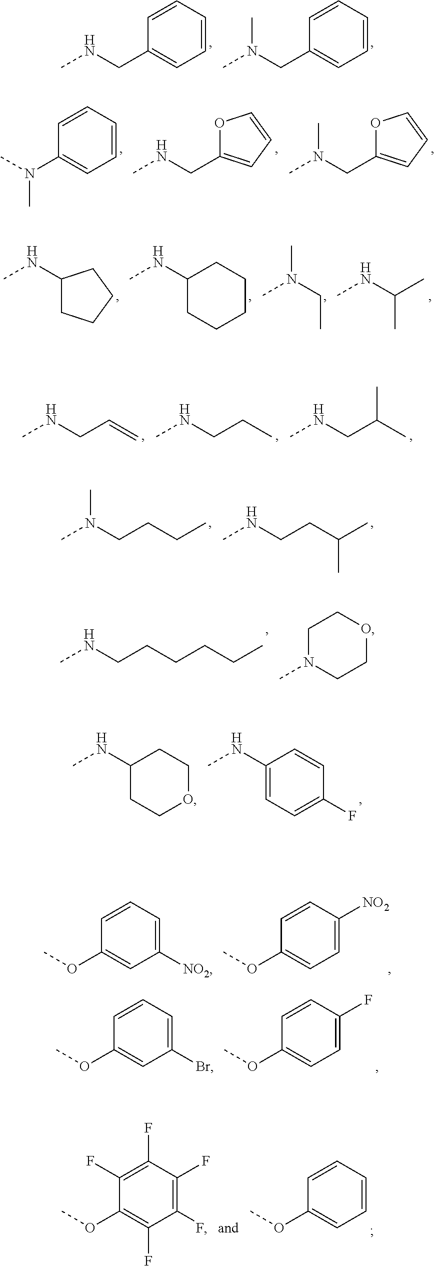
wherein R³ is selected from hydrogen 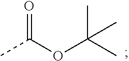
and,
wherein R⁴ is selected from hydrogen,

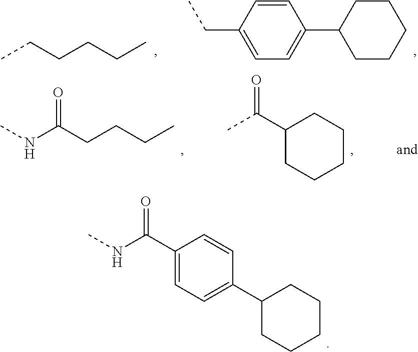

4. A method for the treatment of a disorder associated with STAT activity in a mammal comprising the step of administering to the mammal a therapeutically effective amount of a compound having a structure represented by a formula: 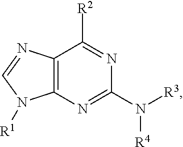
wherein R¹ is selected from H and (CH₂)ₘC═OR⁵, wherein m is an integer from 0-3;
wherein R⁵ is selected from OR⁶ and NR⁷R⁸;
wherein R⁶ is selected from hydrogen and C1-C8 alkyl;
wherein each of R⁷ and R⁸ is independently selected from hydrogen and C1-C8 alkyl;
wherein R² is selected from halogen, OR⁹, and NR¹⁰R¹¹;
wherein R⁹ is an optionally substituted group selected from C1-C8 alkyl, C3-C6 cycloalkyl, C3-C6 heterocycloalkyl, (CH₂)ₙR¹², and Ar¹;
wherein n is an integer from 0-3;
wherein R¹² is an optionally substituted group selected from Ar¹, C3-C6 cycloalkyl and C3-C6 heterocycloalkyl;
wherein Ar¹ is selected from an optionally substituted monocyclic aryl and monocyclic heteroaryl;
wherein R¹⁰ is selected from hydrogen and an optionally substituted group selected from C1-C8 alkyl, C3-C6 cycloalkyl, C3-C6 heterocycloalkyl, (CH₂)ₚR¹³, and Ar²;
wherein n is an integer from 0-3;
wherein R¹³ is an optionally substituted group selected from Ar², C3-C6 cycloalkyl and C3-C6 heterocycloalkyl;
wherein Ar² is selected from an optionally substituted monocyclic aryl and monocyclic heteroaryl;
wherein R¹¹ is selected from hydrogen and C1-C8 alkyl;
wherein R³ is selected from hydrogen and an optionally substituted group selected from C1-C8 alkyl, (CH₂)_{q}R¹⁴, and C═O(CH₂)_{q}R¹⁴;
wherein q is an integer from 0-3;
wherein R¹⁴ is selected from hydrogen and an optionally substituted group selected from C1-C8 alkyl, C3-C6 cycloalkyl, C3-C6 heterocycloalkyl, and Ar³;
wherein Ar³ is selected from an optionally substituted monocyclic aryl and monocyclic heteroaryl;
wherein R⁴ is selected from hydrogen and C═OOR¹⁵;
wherein R¹⁵ is selected from hydrogen and optionally substituted C1-C8 alkyl;
wherein R³ and R⁴ are optionally covalently bonded and, together with the intermediate carbon, comprise an optionally substituted 3- to 7-membered heterocycloalkyl; and
wherein R¹⁰ and R¹¹ are optionally covalently bonded and, together with the intermediate carbon, comprise an optionally substituted 3- to 7-membered heterocycloalkyl;
or a pharmaceutically acceptable salt, hydrate, solvate, or polymorph thereof.

5. The method of claim 4, wherein the compound administered is a compound of claim 2 or 3.

6. The method of claim 4, wherein the STAT is STAT3.

7. The method of claim 4, wherein the compound exhibits inhibition with an IC₅₀ of less than about 250 μM.

8. The method of claim 4, wherein the compound exhibits inhibition with an IC₅₀ of less than about 100 μM.

9. The method of claim 4, wherein the compound exhibits inhibition with an IC₅₀ of less than about 50 μM.

10. The method of claim 4, wherein the compound exhibits inhibition with an IC₅₀ of less than about 10 μM.

11. The method of claim 4, wherein the compound exhibits inhibition with an IC₅₀ of less than about 1 μM.

12. The method of claim 7, wherein the inhibition is inhibition of STAT activity in an electrophoretic gel shift assay.

13. The method of claim 7, wherein the inhibition is inhibition of cell growth.

14. The method of claim 13, wherein the IC₅₀ is determined using a cell selected from MDA-MB-231, Panc-1 and DU 145.

15. The method of claim 4, wherein the mammal has been diagnosed with a need for treatment of the disorder prior to the administering step.

16. The method of claim 4, further comprising the step of identifying a mammal in need of treatment of the disorder.

17. The method of claim 4, wherein the disorder is associated with constitutively active STAT3.

18. A method for inhibiting STAT activity in at least one cell, comprising the step of contacting the at least one cell with an effective amount of least one compound having a structure represented by a formula: 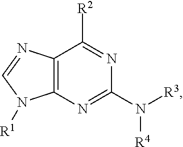
wherein R¹ is selected from H and (CH₂)ₘC═OR⁵, wherein m is an integer from 0-3;
wherein R⁵ is selected from OR⁶ and NR⁷R⁸;
wherein R⁶ is selected from hydrogen and C1-C8 alkyl;
wherein each of R⁷ and R⁸ is independently selected from hydrogen and C1-C8 alkyl;
wherein R² is selected from halogen, OR⁹, and NR¹⁰R¹¹;
wherein R⁹ is an optionally substituted group selected from C1-C8 alkyl, C3-C6 cycloalkyl, C3-C6 heterocycloalkyl, (CH₂)ₙR¹², and Ar¹;
wherein n is an integer from 0-3;
wherein R¹² is an optionally substituted group selected from Ar¹, C3-C6 cycloalkyl and C3-C6 heterocycloalkyl;
wherein Ar¹ is selected from an optionally substituted monocyclic aryl and monocyclic heteroaryl;
wherein R¹⁰ is selected from hydrogen and an optionally substituted group selected from C1-C8 alkyl, C3-C6 cycloalkyl, C3-C6 heterocycloalkyl, (CH₂)ₚR¹³, and Ar²;
wherein n is an integer from 0-3;
wherein R¹³ is an optionally substituted group selected from Ar², C3-C6 cycloalkyl and C3-C6 heterocycloalkyl;
wherein Ar² is selected from an optionally substituted monocyclic aryl and monocyclic heteroaryl;
wherein R¹¹ is selected from hydrogen and C1-C8 alkyl;
wherein R³ is selected from hydrogen and an optionally substituted group selected from C1-C8 alkyl, (CH₂)_{q}R¹⁴, and C═O(CH₂)_{q}R¹⁴;
wherein q is an integer from 0-3;
wherein R¹⁴ is selected from hydrogen and an optionally substituted group selected from C1-C8 alkyl, C3-C6 cycloalkyl, C3-C6 heterocycloalkyl, and Ar³;
wherein Ar³ is selected from an optionally substituted monocyclic aryl and monocyclic heteroaryl;
wherein R⁴ is selected from hydrogen and C═OOR¹⁵;
wherein R¹⁵ is selected from hydrogen and optionally substituted C1-C8 alkyl;
wherein R³ and R⁴ are optionally covalently bonded and, together with the intermediate carbon, comprise an optionally substituted 3- to 7-membered heterocycloalkyl; and
wherein R¹⁰ and R¹¹ are optionally covalently bonded and, together with the intermediate carbon, comprise an optionally substituted 3- to 7-membered heterocycloalkyl;
or a pharmaceutically acceptable salt, hydrate, solvate, or polymorph thereof.

19. The method of claim 18, wherein the compound administered is a compound of claim 2 or 3.

20. The method of claim 18, wherein the STAT is STAT3.
